(19) United States
(12) Patent Application Publication
(10) Pub. No.: US 2019/0275108 A1
(43) Pub. Date: Sep. 12, 2019

(54) COMPOSITIONS IN THE FORM OF AN INJECTABLE AQUEOUS SOLUTION COMPRISING AMYLIN, AN AMYLIN RECEPTOR AGONIST OR AN AMYLIN ANALOGUE AND A CO-POLYAMINO ACID

(71) Applicant: ADOCIA, Lyon (FR)

(72) Inventor: Alexandre GEISSLER, Lyon (FR)

(73) Assignee: ADOCIA, Lyon (FR)

(21) Appl. No.: 16/213,881

(22) Filed: Dec. 7, 2018

Related U.S. Application Data
(60) Provisional application No. 62/606,139, filed on Dec. 7, 2017.

(30) Foreign Application Priority Data
Jun. 29, 2018 (FR) 18/55959

(51) Int. Cl. A61K 38/22; A61K 47/34; A61K 9/00; A61K 9/08

(52) U.S. Cl. CPC A61K 38/22; A61K 9/08; A61K 9/0019; A61K 47/34

(57) ABSTRACT

A composition in the form of an injectable aqueous solution, the pH of which is comprised from 6.0 to 8.0, including at least:
a) amylin, an amylin receptor agonist or an amylin analogue;
b) a co-polyamino acid bearing carboxylate charges and hydrophobic radicals Hy, said co-polyamino acid being constituted of glutamic or aspartic units and said hydrophobic radicals Hy being according to formula I below:

*GpRᵣGpAₐGpC)ₚ Formula I

wherein the composition does not comprise a basal insulin the isoelectric point pI of which is comprised from 5.8 to 8.5. It also relates to a composition wherein it further includes prandial insulin.

The present invention relates to amylin, amylin receptor agonist or amylin analogue injection therapies for treating diabetes.

The invention relates to a composition in the form of an injectable aqueous solution, the pH of which is comprised from 6.0 to 8.0, comprising at least amylin, an amylin receptor agonist or an amylin analogue and a co-polyamino acid bearing carboxylate charges and hydrophobic radicals according to the invention and compositions further comprising insulin (excluding basal insulins the isoelectric point p1 of which is comprised from 5.8 to 8.5). The invention also relates to pharmaceutical formulations comprising the compositions according to the invention. Finally, the invention also relates to a use of co-polyamino acids bearing carboxylate charges and hydrophobic radicals according to the invention for stabilizing amylin, amylin receptor agonist or amylin analogue compositions as well as amylin, amylin receptor agonist or amylin analogue compositions further comprising insulin.

Type 1 diabetes is an autoimmune disease which leads to the destruction of beta cells in the pancreas. These cells are known to produce insulin, the primary function of which is to regulate glucose utilization in the peripheral tissues (Gerich 1993 Control of glycaemia). Consequently, patients with type 1 diabetes suffer from chronic hyperglycemia and are required to self-administer exogenous insulin in order to reduce this hyperglycemia. Insulin therapy has made it possible to obtain a drastic change in the life expectancy of these patients. However, the glycemia control ensured by exogenous insulin is not optimal, especially after a meal. This is due to the fact that these patients produce glucagon after taking a meal, which leads to the decrease of a part of the glucose stored in the liver, which is not the case in a healthy person. This glucagon-mediated glucose production worsens the problem of glycemia regulation in these patients.

It has been demonstrated that amylin, another hormone produced by the beta cells of the pancreas, and thus also deficient in type 1 diabetes patients, plays a key role in the regulation of post-prandial glycemia. Amylin, also known as “islet amyloid polypeptide” or IAPP, is a peptide of 37 amino acids, which is co-stored and co-secreted with insulin (Schmitz 2004 Amylin Agonists). This peptide is described to block the production of glucagon by alpha cells of the pancreas. Insulin and amylin thus have complementary and synergistic roles, insulin reduces blood glucose while amylin reduces the entry of endogenous glucose into the blood by inhibiting the production (secretion) of endogenous glucagon.

This problem of regulation of postprandial glucose is quite similar in patients with type 2 diabetes treated with insulin given that their disease has led to a very significant loss in their beta cell mass and consequently, their ability to produce insulin and amylin.

Human amylin has properties that are not compatible with pharmaceutical requirements in terms of solubility and stability (Goldsbury C S, Cooper G J, Goldie K N, Muller S A, Saafi E L, W T Gruijters, Misur M P, Engel A, AeBIU, Kistler J: Polymorphic Fibrillar Assembly of Human Amylin. J Struct Biol 119: 17-27, 1997). Amylin is known to form amyloid fibers which lead to the formation of water-insoluble plaques. Although amylin is the natural hormone, it was necessary to develop an analogue in order to solve these solubility problems.

The physicochemical properties of amylin thus make its use impossible: amylin is stable for only about fifteen minutes at acidic pH and for less than a minute at neutral pH.

The Amylin company has developed an amylin analogue, pramlintide, for remedying the lack of stability of human amylin. This product, which is marketed under the name Symlin®, was approved in 2005 by the FDA for the treatment of type 1 and type 2 diabetes. It should be administered subcutaneously three times a day, one hour before the meal to improve control of postprandial blood glucose. This peptide is formulated at acidic pH and is described to fibrillate when the pH of the solution is greater than 5.5. Analogue variants are described in U.S. Pat. No. 5,686,411.

This analogue is thus not satisfactory in terms of stability when a formulation at neutral pH is envisaged.

To date, there is no existing means for stabilizing human amylin in order to make a pharmaceutical product therewith. Yet, it would be advantageous for patients to have access to the human form of this physiological hormone. It would also be advantageous to be able to formulate an amylin analogue or an amylin receptor agonist at neutral pH.

In addition, there would be a benefit of being able to mix an amylin analogue, or an amylin receptor agonist, in aqueous solution amylin, with a prandial insulin since these two products are to be administered before a meal. This would also make it possible to mimic the physiology since these two hormones are co-secreted by beta cells in response to a meal to improve the control of postprandial blood glucose.

However, considering that prandial insulin solutions have a pH close to neutrality for chemical stability reasons, it is not possible to obtain an aqueous solution that meets the pharmaceutical requirements in terms of solubility and stability.

For this reason, patent application US2016/001002 from ROCHE describes a pump containing two separate reservoirs to enable the co-administration of these two hormones with a single medical device. However, this patent does not solve the problem of mixing these two hormones in solution thereby making it possible to administer them with the one-reservoir conventional pumps already on the market.

Patent application WO2013067022 from XERIS provides a solution to the problem of amylin stability and its compatibility with insulin by using an organic solvent instead of water. The absence of water seems to solve the stability problems but the use of an organic solvent raises chronic use safety issues in diabetic patients as well as compatibility issues with common medical devices, on the tubes, seals and plasticizers used.

Patent application WO2007104786 from NOVO NORDISK describes a method for stabilizing a solution of pramlintide, which is an analogue of amylin, and of insulin by the addition of phospholipid, derived from glycerophosphoglycerol, especially dimyristoyl glycerophosphoglycerol (DMPG). However, this solution requires the use of large quantities of DMPG which can pose a local tolerance problem. Furthermore, DMPG leads to compositions having quite poor physical stabilities at 0-4° C. as described in application WO2018122278.

To the applicant's knowledge, there is no satisfactory means for combining a prandial insulin and human amylin, an amylin receptor agonist or an amylin analogue in aqueous solution in order to be suitable for being administered with conventional devices.

The formulation at acidic pH and rapid fibrillation are obstacles to obtain apharmaceutical formulation at neutral pH based on amylin and pramlintide, but also hinder the combination of amyline or pramlintide with other active pharmaceutical ingredients, in particular peptides or proteins.

The applicant has observed that, surprisingly, co-polyamino acids according to the invention stabilize amylin, amylin receptor agonist or amylin analogue compositions at a pH ranging from 6 to 8. As a matter of fact, compositions comprising amylin, an amylin receptor agonist or an amylin analogue in combination with a co-polyamino acid according to the invention exhibit greater stability over time, which is of great interest for pharmaceutical development.

The applicant has found that co-polyamino acids according to the invention also make it possible to obtain a composition comprising prandial insulin and amylin, amylin receptor agonist or amylin analogue, said composition being clear and having an improved fibrillation stability.

A conventional method for measuring the stability of proteins or peptides is to measure the formation of fibrils using Thioflavin T, also known as ThT. This method makes it possible to measure the latency time before the formation of fibrils by measuring the increase in fluorescence, under temperature and stirring conditions which permit an acceleration of the phenomenon. The compositions according to the invention have a latency time before fibril formation significantly greater than that of amylin, an amylin receptor agonist or an amylin analogue at the pH of interest.

The compositions according to the invention have a satisfactory physical stability, and possibly a chemical stability, at the desired pH.

In one embodiment, the invention relates to a composition in the form of an injectable aqueous solution, the pH of which is comprised from 6.0 to 8.0, comprising at least:
a) amylin, an amylin receptor agonist or an amylin analogue;
b) a co-polyamino acid bearing carboxylate charges and hydrophobic radicals Hy, said co-polyamino acid being constituted of glutamic or aspartic units and said hydrophobic radicals Hy being according to formula I below:

*GpRᵣGpAₐGpC)ₚ Formula I

wherein
GpR is a radical according to formulas II, II′ or II″:

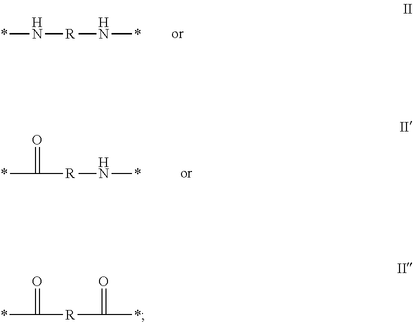

GpA is a radical according to formulas III or III′:

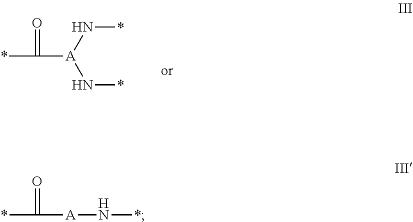

GpC is a radical according to formula IV:

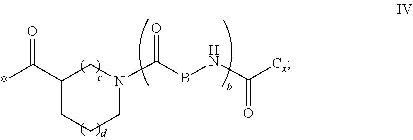

* indicate the attachment sites of the various groups;
a is an integer equal to 0 or 1;
b is an integer equal to 0 or 1;
p is an integer equal to 1 or 2 and
   if p is equal to 1 then a is equal to 0 or 1 and GpA is a radical according to formula III′ and,
   if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
   if r is equal to 0, then the hydrophobic radical according to formula I is bound to the co-polyamino acid via a covalent bond between a carbonyl of the hydrophobic radical and a nitrogen atom in the N-terminal position of the co-polyamino acid, thus forming an amide function from the reaction of an amine function at the N-terminal position of the precursor of the co-polyamino acid and an acid function borne by the precursor of the hydrophobic radical, and
   if r is equal to 1 or 2, then the hydrophobic radical according to formula I is bound to the co-polyamino acid:
      via a covalent bond between a nitrogen atom of the hydrophobic radial and a carbonyl of the co-polyamino acid, thus forming an amide function from the reaction of an amine function of the precursor of the hydrophobic radical and an acid function borne by the precursor of the co-polyamino acid or
      via a covalent bond between a carbonyl of the hydrophobic radical and a nitrogen atom in N-terminal position of the co-polyamino acid, thus forming an amide function from the reaction of an acid function of the precursor of the hydrophobic radical and an amine function in N-terminal position borne by the precursor of the co-polyamino acid;
R is a radical chosen from the group consisting of a linear or branched divalent alkyl radical comprising from 1 to 12 carbon atoms, a divalent linear or branched alkyl radical comprising from 1 to 12 carbon atoms bearing one or more —CONH2 functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms;
more precisely, R is a radical chosen from the group consisting of:
   a divalent alkyl radical, linear or branched, comprising from 2 to 12 carbon atoms if GpR is a radical according to formula II, from 1 to 11 carbon atoms if GpR is a radical according to formula II' or from 0 to 10 carbon atoms if GpR is a radical according to formula II″;
   a divalent, linear or branched alkyl radical, comprising from 2 to 11 carbon atoms if GpR is a radical according to formula II, from 1 to 11 carbon atoms if GpR is a radical according to formula II' or from 0 to 10 carbon atoms if GpR is a radical according to formula II″, said alkyl radical bearing one or more —CONH₂ functions, and
   an unsubstituted ether or polyether radical comprising from 4 to 14 carbon atoms and from 1 to 5 oxygen atoms;
A is a linear or branched alkyl radical, and optionally substituted by a radical resulting from a saturated, unsaturated or aromatic ring, comprising from 1 to 8 carbon atoms;
B is a linear or branched alkyl radical, optionally comprising an aromatic ring comprising from 1 to 9 carbon atoms or an unsubstituted ether or polyether radical comprising from 4 to 14 carbon atoms and from 1 to 5 oxygen atoms;
Cₓ is a linear or branched monovalent alkyl radical, optionally comprising a cyclic part, wherein x indicates the number of carbon atoms and:
   if p is equal to 1, x is comprised from 9 to 25 (9≤x≤25):
   if p is equal to 2, xis comprised from 9 to 15 (9≤x≤15),
the ratio i between the number of hydrophobic radicals and the number of glutamic or aspartic units being comprised between 0<i≤0.5;
when several hydrophobic radicals are borne by a co-polyamino acid they are therefore identical or different,
the degree of polymerization DP of glutamic or aspartic units is comprised from 5 to 250;
the free acid functions being in the form of an alkaline cation salt chosen from the group consisting of Na⁺ and K⁺;
characterized in that the composition does not comprise a basal insulin the isoelectric point p1 of which is comprised from 5.8 to 8.5.

In one embodiment, the invention relates to a composition in the form of an injectable aqueous solution, the pH of which is comprised from 6.0 to 8.0, comprising at least:
a) amylin, an amylin receptor agonist or an amylin analogue;
b) a co-polyamino acid bearing carboxylate charges and hydrophobic radicals Hy, said co-polyamino acid being constituted of glutamic or aspartic units and said hydrophobic radicals Hy being according to formula I below:

*GpRᵣGpAₐGpC)ₚ Formula I

wherein
GpR is a radical according to formulas II, II′ or II″:

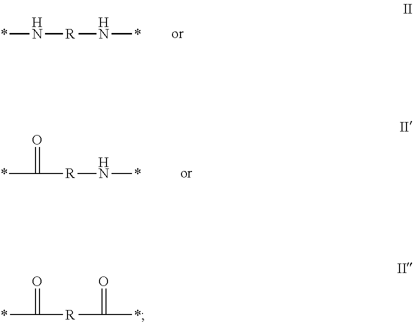

GpA is a radical according to formulas III or III′:

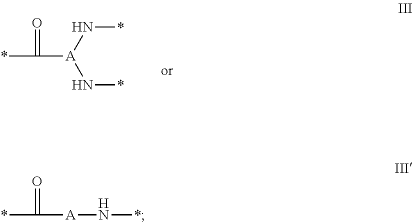

GpC is a radical according to formula IV:

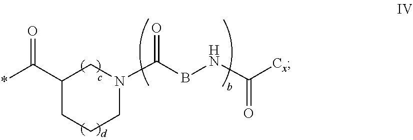

* indicate the attachment sites of the various groups;
a is an integer equal to 0 or 1;
b is an integer equal to 0 or 1;
p is an integer equal to 1 or 2 and
   if p is equal to 1 then a is equal to 0 or 1 and GpA is a radical according to formula III' and,
   if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
   if r is equal to 0, then the hydrophobic radical according to formula I is bound to the co-polyamino acid via a covalent bond between a carbonyl of the hydrophobic radical and a nitrogen atom in the N-terminal position of the co-polyamino acid, thus forming an amide function from the reaction of an amine function at the N-terminal position of the precursor of the co-polyamino acid and an acid function borne by the precursor of the hydrophobic radical, and
   if r is equal to 1 or 2, then the hydrophobic radical according to formula I is bound to the co-polyamino acid:
      via a covalent bond between a nitrogen atom of the hydrophobic radial and a carbonyl of the co-polyamino acid, thus forming an amide function from the reaction of an amine function of the precursor of the hydrophobic radical and an acid function borne by the precursor of the co-polyamino acid or
      via a covalent bond between a carbonyl of the hydrophobic radical and a nitrogen atom in the N-terminal position of the co-polyamino acid, thus forming an amide function from the reaction of an acid function of the precursor of the hydrophobic radical and an amine function in N-terminal position borne by the precursor of the co-polyamino acid;
R is a radical chosen from the group consisting of a linear or branched divalent alkyl radical comprising from 1 to 12 carbon atoms, a divalent linear or branched alkyl radical comprising from 1 to 12 carbon atoms bearing one or more —CONH2 functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms;
more precisely, R is a radical chosen from the group consisting of:
   a divalent alkyl radical, linear or branched, comprising from 2 to 12 carbon atoms if GpR is a radical according to formula II, or from 1 to 11 carbon atoms if GpR is a radical according to formula II' or from 0 to 10 carbon atoms if GpR is a radical according to formula II″;
   a divalent, linear or branched alkyl radical, comprising from 2 to 11 carbon atoms if GpR is a radical according to formula II, or from 1 to 11 carbon atoms if GpR is a radical according to formula II′ or from 0 to 10 carbon atoms if GpR is a radical according to formula II″, said alkyl radical bearing one or more functions —CONH₂ functions, and
   an unsubstituted ether or polyether radical comprising from 4 to 14 carbon atoms and from 1 to 5 oxygen atoms;
A is a radical chosen from the group consisting of an unsubstituted ether or polyether radical comprising from 4 to 14 carbon atoms and from 1 to 5 oxygen atoms or a linear or branched alkyl radical comprising from 1 to 8 carbon atoms and optionally substituted by a radical from a saturated, unsaturated or aromatic ring;
B is a linear or branched alkyl radical, optionally comprising an aromatic ring, comprising 1 to 9 carbon atoms;
Cₓ is a linear or branched monovalent alkyl radical, optionally comprising a cyclic part, wherein x indicates the number of carbon atoms and:
   if p is equal to 1, x is comprised from 9 to 25 (9≤x≤25):
   if p is equal to 2, xis comprised from 9 to 15 (9≤x ≤15),
the ratio i between the number of hydrophobic radicals and the number of glutamic or aspartic units being comprised from 0<i=0.5;
when several hydrophobic radicals are borne by a co-polyamino acid they are therefore identical or different,
the degree of polymerization DP of glutamic or aspartic units is comprised from 5 to 250;
the free acid functions being in the form of an alkaline cation salt chosen from the group consisting of Na+ and K+;
characterized in that the composition does not comprise a basal insulin the isoelectric point pI of which is comprised from 5.8 to 8.5.

According to the invention, compositions in the form of an injectable aqueous solution are clear solutions. “Clear solution” is understood to mean compositions which satisfy the criteria described in the American and European pharmacopoeias concerning injectable solutions. In the American pharmacopoeia, the solutions are defined in section <1151> referring to the injection (<1>) (referring to <788> according to USP 35 and specified in <788> according to USP 35 and in <787>, <788> and <790> USP 38 (from Aug. 1, 2014), according to USP 38). In the European pharmacopoeia, injectable solutions have to meet the criteria given in sections 2.9.19 and 2.9.20.

Said co-polyamino acid bearing carboxylate charges and hydrophobic radicals Hy is soluble in aqueous solution at a pH ranging from 6.0 to 8.0, at a temperature of 25° C. and at a concentration of less than 100 mg/mL.

Co-polyamino acids bearing carboxylate charges and hydrophobic radicals according to formula I are soluble in distilled water at a pH ranging from 6 to 8, at a temperature of 25° C. and at a concentration of less than 100 mg/mL.

In one embodiment, the composition according to the invention is characterized in that Hy comprises between 15 and 100 carbon atoms.

In one embodiment, Hy comprises more than 30 carbon atoms.

In one embodiment, the composition according to the invention is characterized in that Hy comprises between 30 and 70 carbon atoms.

In one embodiment, the composition according to the invention is characterized in that Hy comprises between 40 and 60 carbon atoms.

In one embodiment, the composition according to the invention is characterized in that Hy comprises between 20 and 30 carbon atoms

In one embodiment, Hy comprises more than 15 carbon atoms.

In one embodiment, Hy comprises more than 30 carbon atoms.

In one embodiment, the composition is characterized in that the pH is comprised from 6.6 to 7.8.

In one embodiment, the composition is characterized in that the pH is comprised from 7.0 to 7.8.

In one embodiment, the composition is characterized in that the pH is comprised from 6.8 to 7.4.

In one embodiment, when r=2, then the GpR group bound to PLG is chosen among the GpR according to formula II.

In one embodiment, when r=2 then the GpR group bound to PLG is chosen among the GpR according to formula II and the second GpR is chosen among the GpR according to formula II″.

In one embodiment, when r=2 then the GpR group bound to PLG is chosen among the GpR according to formula II″.

In one embodiment, when r=2 then the GpR group bound to PLG is chosen among the GpR according to formula II″ and the second GpR is chosen among the GpR according to formula II.

In one embodiment, GpR is a radical according to formula II:

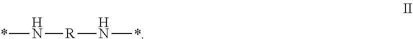

In one embodiment, at least one hydrophobic radical -Hy is chosen among the radicals according to formula I wherein r=2 according to formula Xc′, as defined below:

*-GPR₁-GpR-(GpA)ₐ-(GpC)ₚ Formula Xc′

wherein GpR₁ is a radical according to formula II.

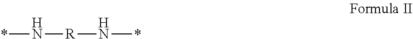

wherein GpR, GpA, GpC, R, a, and p have the definitions given above.

In one embodiment, at least one hydrophobic radical -Hy is chosen among the radicals according to formula I wherein r=2 according to formula Xc′, as defined below:

*-GpR₁-GpR-(GpA)ₐ-(GpC)ₚ Formula Xc′

wherein GpR₁ is a radical according to formula II″.

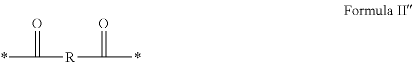

wherein GpR, GpA, GpC, R, a, and p have the definitions given above.

L

In one embodiment, the composition is characterized in that the said hydrophobic radicals are chosen among the hydrophobic radicals according to formula I wherein if p is equal to 1 and if x is less than or equal to 14 (x≤14) then r=0 or r=1.

In one embodiment, the composition is characterized in that said hydrophobic radicals are chosen among hydrophobic radicals according to formula I wherein, if p is equal to 1 and if x is comprised from 15 to 16 (15≤x≤16), then r=1.

In one embodiment, the composition is characterized in that the said hydrophobic radicals are chosen among the hydrophobic radicals according to formula I wherein if p is equal to 1 and if x is greater than 17 (17≤x) then r=1 and R is an ether or polyether radical.

In one embodiment, the composition is characterized in that the said hydrophobic radicals are chosen among the hydrophobic radicals according to formula I wherein, if p is equal to 1, then x is comprised from 17 to 25 (17≤x≤25).

In one embodiment, the composition is characterized in that the hydrophobic radical is a radical according to formula I wherein r=1, a=1, p=1, GpR is according to formula II, GpA is according to formula III' wherein A corresponds to formula Y9, GpC corresponds to formula IVd.

In one embodiment, the composition is characterized in that the hydrophobic radical is a radical according to formula I wherein r=1, a=1, p=1, GpR is according to formula II wherein R is a divalent linear alkyl, GpA corresponds to formula III′ wherein A is according to formula Y9, GpC corresponds to formula IVd.

In one embodiment, the composition is characterized in that the hydrophobic radical is a radical according to formula I wherein r=1, a=1, p=1, GpR corresponds to formula II wherein R is —CH2-CH2 GpA is according to formula III′ wherein A is according to formula Y9, GpC is according to formula IVd

In one embodiment, the composition is characterized in that the hydrophobic radical is a radical according to formula I wherein r=1, a=1, p=1, GpR is according to formula II wherein R is —CH2-CH2 GpA is according to formula III′ wherein A is according to formula Y9, GpC is according to formula IVd wherein x=13 and Cx is

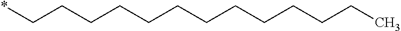 [SMILES: *CCCCCCCCCCCCC]

In one embodiment, the composition is characterized in that the hydrophobic radical according to formula I is a radical wherein R is a divalent linear alkyl radical comprising from 2 to 12 carbon atoms.

In one embodiment, the composition is characterized in that the hydrophobic radical according to formula I is a radical wherein R is a divalent alkyl radical comprising from 2 to 6 carbon atoms.

In one embodiment, the composition is characterized in that the hydrophobic radical according to formula I is a radical wherein R is a divalent linear alkyl radical comprising from 2 to 6 carbon atoms.

In one embodiment, the composition is characterized in that the hydrophobic radical according to formula I is a radical wherein R is a divalent alkyl radical comprising from 2 to 4 carbon atoms.

In one embodiment, the composition is characterized in that the hydrophobic radical according to formula I is a radical wherein R is a divalent linear alkyl radical comprising from 2 to 4 carbon atoms.

In one embodiment, the composition is characterized in that the hydrophobic radical according to formula I is a radical wherein R is a divalent alkyl radical comprising 2 carbon atoms.

In one embodiment, the composition is characterized in that the hydrophobic radical according to formula I is a radical wherein R is a divalent linear alkyl radical comprising from 1 to 11 carbon atoms.

In one embodiment, the composition is characterized in that the hydrophobic radical according to formula I is a radical wherein R is a divalent alkyl radical comprising from 1 to 6 carbon atoms.

In one embodiment, the composition is characterized in that the hydrophobic radical according to formula I is a radical wherein R is a divalent alkyl radical comprising from 2 to 5 carbon atoms and bearing one or more amide functions (—CONH2).

In one embodiment, the composition is characterized in that the hydrophobic radical according to formula I is a radical wherein R is a divalent linear alkyl radical comprising from 2 to 5 carbon atoms and bearing one or more amide functions (—CONH2).

In one embodiment, the composition is characterized in that the hydrophobic radical according to formula I is a radical wherein R is a radical chosen among the group consisting of the radicals represented by the formulas below:

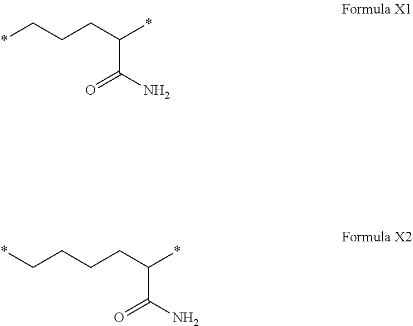 [SMILES: *CCCC(*)C(N)=O ; *CCCCC(*)C(N)=O]

In one embodiment, the composition is characterized in that the hydrophobic radical according to formula VI is a radical wherein R is a radical according to formula X1.

In one embodiment, the composition is characterized in that the hydrophobic radical according to formula I is a radical wherein R is a radical according to formula X2.

In one embodiment, the composition is characterized in that the hydrophobic radical according to formula I is a radical wherein R is bound to the co-polyamino acid via an amide function borne by the carbon in the delta or epsilon position (or in position 4 or 5) with respect to the amide function (—CONH2).

In one embodiment, the composition is characterized in that the hydrophobic radical according to formula I is a radical wherein R is an unsubstituted linear ether or polyether radical comprising from 4 to 14 carbon atoms and from 1 to 5 oxygen atoms.

In one embodiment, the composition is characterized in that the hydrophobic radical according to formula I is a radical wherein R is an ether radical.

In one embodiment, the composition is characterized in that the hydrophobic radical according to formula I is a radical wherein R is a divalent alkyl radical comprising from 4 to 6 carbon atoms.

In one embodiment, the composition is characterized in that the hydrophobic radical according to formula I is a radical wherein R is a divalent alkyl radical comprising 6 carbon atoms.

In one embodiment, the composition is characterized in that the hydrophobic radical according to formula I is a radical wherein R is an ether radical represented by the formula.

In one embodiment, the composition is characterized in that the hydrophobic radical according to formula I is a radical wherein R is a polyether radical.

In one embodiment, the composition is characterized in that the hydrophobic radical according to formula I is a radical wherein R is a linear polyether radical comprising from 6 to 10 carbon atoms and from 2 to 3 oxygen atoms.

In one embodiment, the composition is characterized in that the hydrophobic radical according to formula I is a radical wherein R is a radical chosen among the group consisting of the radicals represented by the formulas below:

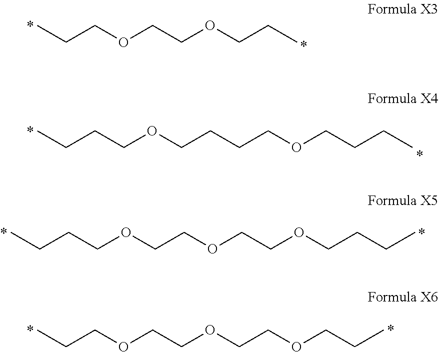

In one embodiment, the composition is characterized in that the hydrophobic radical according to formula I is a radical wherein R is a radical according to formula X3.

In one embodiment, the composition is characterized in that the hydrophobic radical according to formula I is a radical wherein R is a radical according to formula X4.

In one embodiment, the composition is characterized in that the hydrophobic radical according to formula I is a radical wherein R is a polyether radical chosen among the group consisting of the radicals represented by formulas X5 and X6 below:

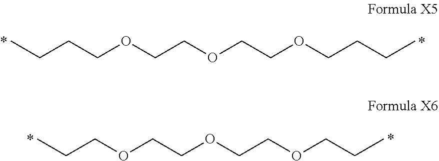

In one embodiment, the composition is characterized in that the hydrophobic radical according to formula I is a radical wherein R is a polyether radical according to formula X5.

In one embodiment, the composition is characterized in that the hydrophobic radical according to formula I is a radical wherein R is a polyether radical according to formula X6.

In one embodiment, the composition is characterized in that the hydrophobic radical is a radical according to formula I wherein a is equal to 0 (a=0) and r is equal to 0 (r=0).

In one embodiment, the composition is characterized in that the hydrophobic radical is a radical according to formula I wherein a is equal to 1 (a=1) and the radical GpA according to formula III′ is chosen among the group consisting of radicals represented by the formulas below:

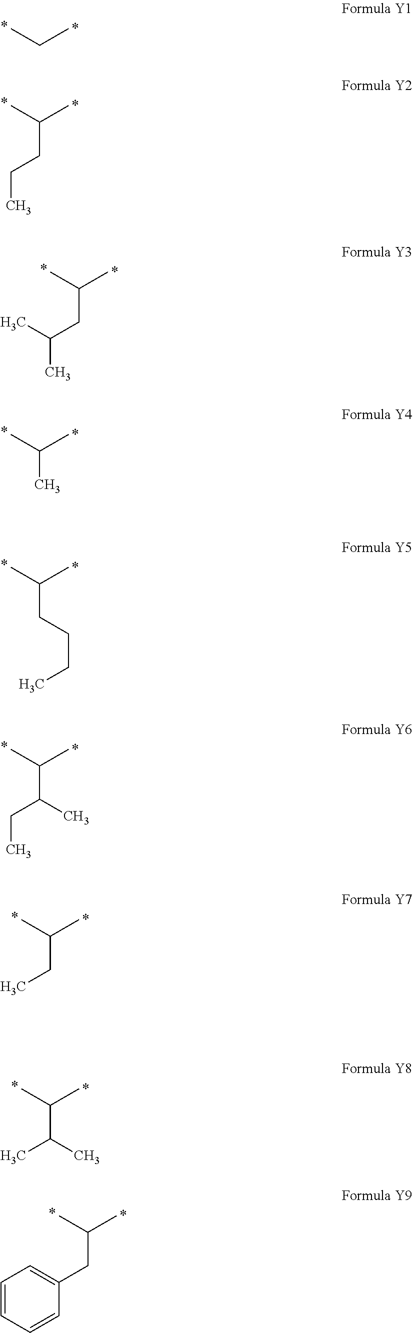

In one embodiment, the composition is characterized in that the hydrophobic radical is a radical according to formula I wherein a is equal to 1 (a=1) and the GpA radical according to formula III′ is a radical according to formula Y1.

In one embodiment, the composition is characterized in that the hydrophobic radical is a radical according to formula I wherein a is equal to 1 (a=1) and the GpA radical according to formula III′ is a radical according to formula Y2.

In one embodiment, the composition is characterized in that the hydrophobic radical is a radical according to formula I wherein a is equal to 1 (a=1) and the GpA radical according to formula III′ is a radical according to formula Y3.

In one embodiment, the composition is characterized in that the hydrophobic radical is a radical according to formula I wherein a is equal to 1 (a=1) and the GpA radical according to formula III′ is a radical according to formula Y4.

In one embodiment, the composition is characterized in that the hydrophobic radical is a radical according to formula I wherein a is equal to 1 (a =1) and the GpA radical according to formula III′ is a radical according to formula Y5.

In one embodiment, the composition is characterized in that the hydrophobic radical is a radical according to formula I wherein a is equal to 1 (a=1) and the GpA radical according to formula III′ is a radical according to formula Y6.

In one embodiment, the composition is characterized in that the hydrophobic radical is a radical according to formula I wherein a is equal to 1 (a=1) and the GpA radical according to formula III′ is a radical according to formula Y7.

In one embodiment, the composition is characterized in that the hydrophobic radical is a radical according to formula I wherein a is equal to 1 (a=1) and the GpA radical according to formula III′ is a radical according to formula Y8.

In one embodiment, the composition is characterized in that the hydrophobic radical is a radical according to formula I wherein a is equal to 1 (a=1) and the GpA radical according to formula III′ is a radical according to formula Y9.

In one embodiment, the composition is characterized in that the hydrophobic radical is a radical according to formula I wherein the GpC radical according to formula IV is chosen among the group consisting of the radicals according to formulas IVa, IVb or IVc hereinafter represented:

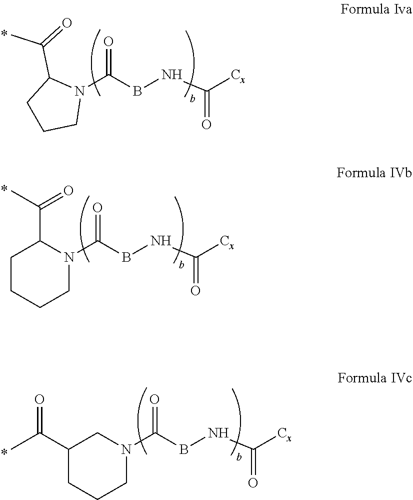

In one embodiment, the composition is characterized in that the hydrophobic radical is a radical according to formula I wherein the GpC radical is according to formula IVa.

In one embodiment, the composition is characterized in that the hydrophobic radical is a radical according to formula I wherein the GpC radical according to formula IV is chosen among the group consisting of the radicals according to formulas IVa, IVb or IVc wherein b is equal to 0, respectively corresponding to formulas IVd, IVe, and IVf hereinafter represented:

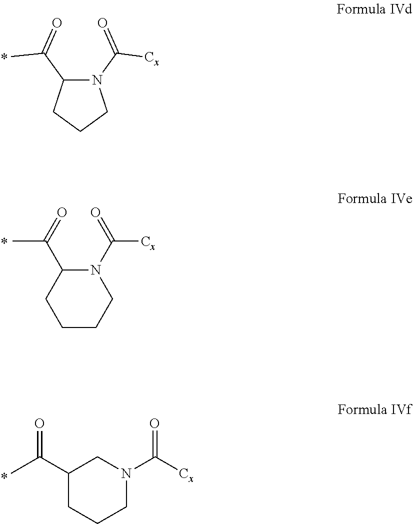

In one embodiment, the composition is characterized in that the hydrophobic radical is a radical according to formula I wherein the GpC radical corresponds to formula IV or IVa wherein b=0, and corresponds to formula IVd.

In one embodiment, the composition is characterized in that the hydrophobic radical is a radical according to formula I wherein the GpC radical according to formula IV wherein b=1 is chosen among the group consisting of radicals wherein B is a residue of amino acid chosen among the group consisting of the radicals represented by the formulas below:

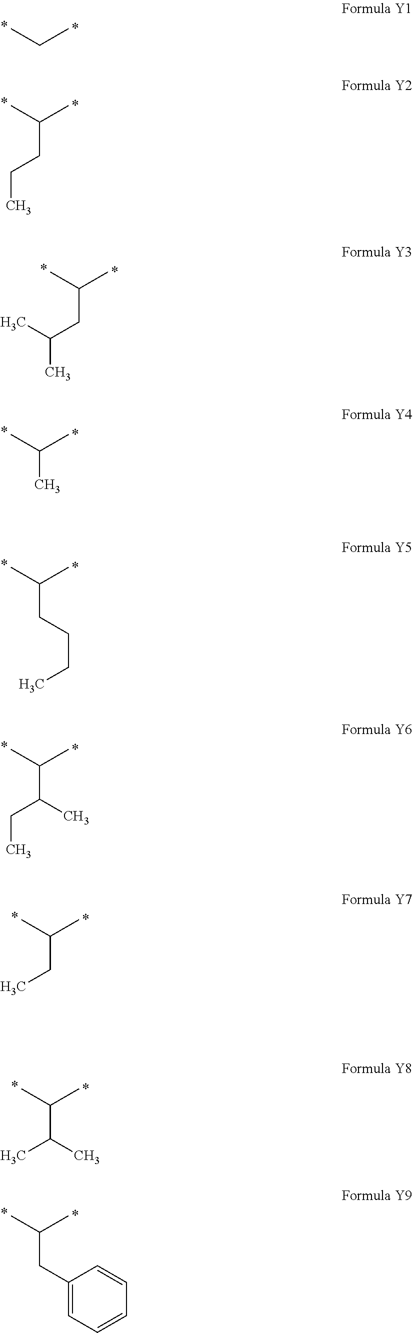

In one embodiment, the composition is characterized in that the hydrophobic radical is a radical according to formula I wherein the GpC radical according to formula IV or IVa wherein b=1 is chosen among the group consisting of radicals wherein B is an amino acid residue chosen among the group consisting of the radicals represented by the formulas below:

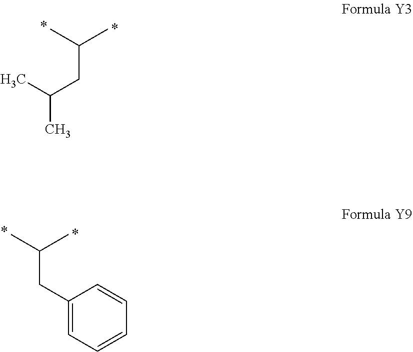

In one embodiment, the composition is characterized in that the hydrophobic radical is a radical according to formula I wherein the GpC radical according to formula IV is chosen among the group consisting of radicals wherein Cx is chosen among the group consisting of linear alkyl radicals.

In one embodiment, the composition is characterized in that the hydrophobic radical is a radical according to formula I wherein the GpC radical according to formula IV is chosen among the group consisting of radicals wherein Cx is chosen among the group consisting of branched alkyl radicals.

In one embodiment, the composition is characterized in that the hydrophobic radical is a radical according to formula I wherein the GpC radical according to formula IV is chosen among the group consisting of radicals wherein Cx is chosen among the group consisting of alkyl radicals comprising from 11 to 14 carbon atoms.

In one embodiment, the composition is characterized in that the hydrophobic radical is a radical according to formula I wherein the GpC radical according to formula IV is chosen among the group consisting of radicals wherein Cx is chosen among the group consisting of the radicals represented by the formulas below:

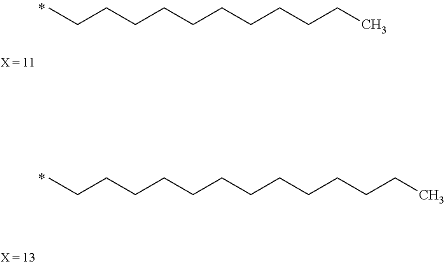

In one embodiment, the composition is characterized in that the hydrophobic radical is a radical according to formula I wherein the GpC radical according to formula IV is chosen among the group consisting of radicals wherein Cx is chosen among the group consisting of alkyl radicals comprising from 15 to 16 carbon atoms.

In one embodiment, the composition is characterized in that the hydrophobic radical is a radical according to formula I wherein the GpC radical according to formula IV is chosen among the group consisting of radicals wherein Cx is chosen among the group consisting of the radicals represented by the formulas below:

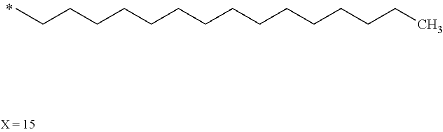 [SMILES: CCCCCCCCCCCCCCCC]

In one embodiment, the composition is characterized in that the hydrophobic radical is a radical according to formula I wherein the GpC radical according to formula IV is chosen among the group consisting of radicals wherein Cx is chosen among the group consisting of the radicals represented by the formulas below:

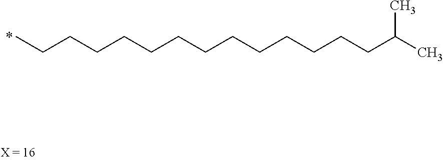 [SMILES: CCCCCCCCCCCCCCC(C)C]

In one embodiment, the composition is characterized in that the hydrophobic radical is a radical according to formula I wherein the GpC radical according to formula IV is chosen among the group consisting of radicals wherein Cx is chosen among the group consisting of alkyl radicals comprising from 17 to 25 carbon atoms.

In one embodiment, the composition is characterized in that the hydrophobic radical is a radical according to formula I wherein the GpC radical according to formula IV is chosen among the group consisting of radicals wherein Cx is chosen among the group consisting of alkyl radicals comprising from 17 to 18 carbon atoms.

In one embodiment, the composition is characterized in that the hydrophobic radical is a radical according to formula I wherein the GpC radical according to formula IV is chosen among the group consisting of radicals wherein Cx is chosen among the group consisting of alkyl radicals represented by the formulas below:

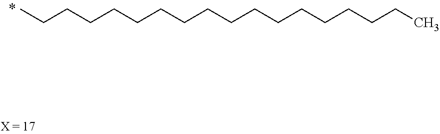 [SMILES: CCCCCCCCCCCCCCCCCC]

In one embodiment, the composition is characterized in that the hydrophobic radical is a radical according to formula I wherein the GpC radical according to formula IV is chosen among the group consisting of radicals wherein Cx is chosen among the group consisting of alkyl radicals comprising from 18 to 25 carbon atoms.

In one embodiment, the composition is characterized in that the hydrophobic radical is a radical according to formula I wherein the GpC radical according to formula IV is chosen among the group consisting of radicals wherein Cx is chosen among the group consisting of alkyl radicals represented by the formulas below:

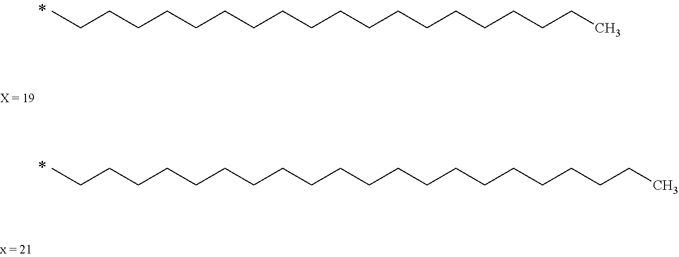

In one embodiment, the composition is characterized in that the hydrophobic radical is a radical according to formula I wherein GpR is a radical according to formula II, II′ or II″, wherein R is a divalent alkyl radical, comprising from 2 to 5 carbon atoms and bearing one or more amide functions (—CONH₂).

In one embodiment, the composition is characterized in that the hydrophobic radical is a radical according to formula I wherein GpR is a radical according to formula II, II′ or II″, wherein R is a divalent linear alkyl radical, comprising from 2 to 5 carbon atoms and bearing one or more amide functions (—CONH₂).

In one embodiment, the composition is characterized in that the hydrophobic radical is a radical according to formula I wherein GpR is a radical according to formula II, II′ or II″ wherein R is a radical chosen among the group consisting of the radicals represented by the formulas below:

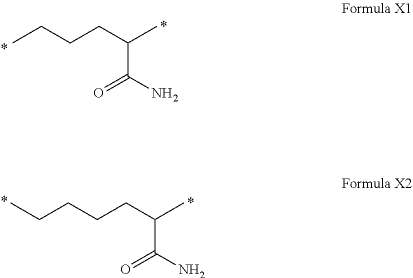

In one embodiment, the composition is characterized in that the R radical is bound to the co-polyamino acid via an amide function borne by the carbon in the delta or epsilon position (or in position 4 or 5) with respect to the amide function (—CONH₂).

In one embodiment, the composition is characterized in that the hydrophobic radical is a radical according to formula I wherein GpR is a radical according to formula II, II′ or II″, wherein R is a linear ether or non-substituted polyether radical comprising from 4 to 14 carbon atoms and 1 to 5 oxygen atoms.

In one embodiment, the composition is characterized in that the hydrophobic radical is a radical according to formula I wherein GpR is a radical according to formula II, II′ or II″, wherein R is an ether radical.

In one embodiment, the composition is characterized in that the hydrophobic radical is a radical according to formula I wherein GpR is a radical according to formula II, II′ or II″, wherein R is an ether radical comprising from 4 to 6 carbon atoms.

In one embodiment, the composition is characterized in that the hydrophobic radical is a radical according to formula I wherein GpR is a radical according to formula II, II′ or II″ wherein R is an ether radical represented by the formula

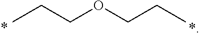

In one embodiment, the composition is characterized in that the hydrophobic radical is a radical according to formula I wherein GpR is a radical according to formula II, II′ or II″, wherein R is a polyether radical.

In one embodiment, the composition is characterized in that the hydrophobic radical is a radical according to formula I wherein GpR is a radical according to formula II, II′ or II″, wherein R is a linear polyether radical comprising from 6 to 10 carbon atoms and from 2 to 3 oxygen atoms.

In one embodiment, the composition is characterized in that the hydrophobic radical is a radical according to formula I wherein GpR is a radical according to formula II, II′ or II″ wherein R is a polyether radical chosen among the group consisting of the radicals represented by the formulas below:

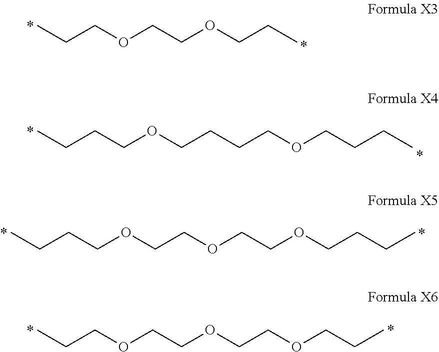

In one embodiment, the composition is characterized in that the said hydrophobic radicals are chosen among the hydrophobic radicals according to formula I wherein p=1, represented by formula V below:

*GpRᵣGpAₐGpC formula V

GpR, GpA, GpC, r and a have the definitions given above.

In one embodiment, the composition is characterized in that the hydrophobic radical is a radical according to formula V wherein: r is equal to 1 (r=1) and a is equal to 0 (a=0).

In one embodiment, the composition is characterized in that the hydrophobic radical is a radical according to formula V wherein: r is equal to 1 (r=1) and a is equal to 1 (a=1).

In one embodiment, the composition is characterized in that the hydrophobic radical is a radical according to formula V wherein r=1, a=1, GpR corresponds to formula II, GpA corresponds to formula III′ wherein A corresponds to formula Y9, GpC corresponds to formula IVd.

In one embodiment, the composition is characterized in that the hydrophobic radical is a radical according to formula V wherein r=1, a=1, GpR corresponds to formula II wherein R is a divalent linear alkyl, GpA corresponds to formula III′ wherein A corresponds to formula Y9, GpC corresponds to formula IVd.

In one embodiment, the composition is characterized in that the hydrophobic radical is a radical according to formula V wherein r=1, a=1, GpR corresponds to formula II wherein R is —CH2-CH2-, GpA corresponds to formula III′ wherein A corresponds to formula Y9, GpC corresponds to formula IVd

In one embodiment, the composition is characterized in that the hydrophobic radical is a radical according to formula V wherein r=1, a=1, GpR corresponds to formula II wherein R is —CH2-CH2-, GpA corresponds to formula III′ wherein A corresponds to formula Y9, GpC corresponds to formula IVd wherein x=13 and Cx is

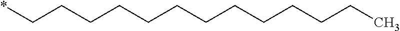 [SMILES: *CCCCCCCCCCCCC]

In one embodiment, the composition is characterized in that the hydrophobic radical according to formula V is a radical wherein R is a divalent linear alkyl radical comprising from 2 to 12 carbon atoms.

In one embodiment, the composition is characterized in that the hydrophobic radical according to formula V is a radical wherein R is a divalent alkyl radical comprising from 2 to 6 carbon atoms.

In one embodiment, the composition is characterized in that the hydrophobic radical according to formula V is a radical wherein R is a divalent linear alkyl radical comprising from 2 to 6 carbon atoms.

In one embodiment, the composition is characterized in that the hydrophobic radical according to formula V is a radical wherein R is a divalent alkyl radical comprising from 2 to 4 carbon atoms.

In one embodiment, the composition is characterized in that the hydrophobic radical according to formula V is a radical wherein R is a divalent linear alkyl radical comprising from 2 to 4 carbon atoms.

In one embodiment, the composition is characterized in that the hydrophobic radical according to formula V is a radical wherein R is a divalent alkyl radical comprising 2 carbon atoms.

In one embodiment, the composition is characterized in that the hydrophobic radical according to formula V is a radical wherein R is a divalent linear alkyl radical comprising from 1 to 11 carbon atoms.

In one embodiment, the composition is characterized in that the hydrophobic radical according to formula V is a radical wherein R is a divalent alkyl radical comprising from 1 to 6 carbon atoms.

In one embodiment, the composition is characterized in that the hydrophobic radical according to formula V is a radical wherein R is a divalent alkyl radical comprising from 2 to 5 carbon atoms and bearing one or more amide functions (—CONH2).

In one embodiment, the composition is characterized in that the hydrophobic radical according to formula V is a radical wherein R is a divalent linear alkyl radical comprising from 2 to 5 carbon atoms and bearing one or more amide functions (—CONH2).

In one embodiment, the composition is characterized in that the hydrophobic radical according to formula V is a radical wherein R is a radical chosen among the group consisting of the radicals represented by the formulas below:

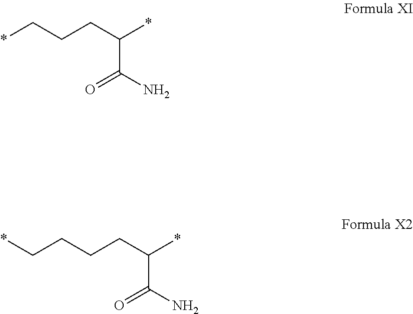 [SMILES: *CCCC(*)C(N)=O ; *CCCCC(*)C(N)=O]

In one embodiment, the composition is characterized in that the hydrophobic radical according to formula V is a radical wherein R is a radical according to formula X1.

In one embodiment, the composition is characterized in that the hydrophobic radical according to formula V is a radical wherein R is a radical according to formula X2.

In one embodiment, the composition is characterized in that the hydrophobic radical according to formula V is a radical wherein R is bound to the co-polyamino acid via an amide function borne by the carbon in the delta or epsilon position (or in position 4 or 5) with respect to the amide function (—CONH2).

In one embodiment, the composition is characterized in that the hydrophobic radical according to formula V is a radical wherein R is an unsubstituted linear ether or polyether radical comprising from 4 to 14 carbon atoms and from 1 to 5 oxygen atoms.

In one embodiment, the composition is characterized in that the hydrophobic radical according to formula V is a radical wherein R is an ether radical.

In one embodiment, the composition is characterized in that the hydrophobic radical according to formula V is a radical wherein R is a divalent alkyl radical comprising from 4 to 6 carbon atoms.

In one embodiment, the composition is characterized in that the hydrophobic radical according to formula V is a radical wherein R is a divalent alkyl radical comprising 6 carbon atoms.

In one embodiment, the composition is characterized in that the hydrophobic radical according to formula V is a radical wherein R is an ether radical represented by the formula.

In one embodiment, the composition is characterized in that the hydrophobic radical according to formula V is a radical wherein R is a polyether radical.

In one embodiment, the composition is characterized in that the hydrophobic radical according to formula V is a radical wherein R is a linear polyether radical comprising from 6 to 10 carbon atoms and from 2 to 3 oxygen atoms.

In one embodiment, the composition is characterized in that the hydrophobic radical according to formula V is a radical wherein R is a polyether radical chosen among the group consisting of the radicals represented by the formulas below:

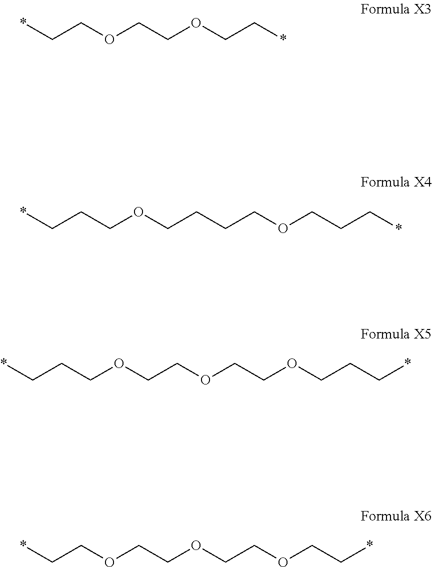 [SMILES: *CCOCCOCC* ; *CCCOCCCCOCCC* ; *CCCOCCOCCOCCC* ; *CCOCCOCCOCC*]

In one embodiment, the composition is characterized in that the hydrophobic radical according to formula V is a radical wherein R is a radical according to formula X3.

In one embodiment, the composition is characterized in that the hydrophobic radical according to formula V is a radical wherein R is a radical according to formula X4.

In one embodiment, the composition is characterized in that the hydrophobic radical according to formula V is a radical wherein R is a polyether radical chosen among the group consisting of the radicals represented by formulas X5 and X6 below:

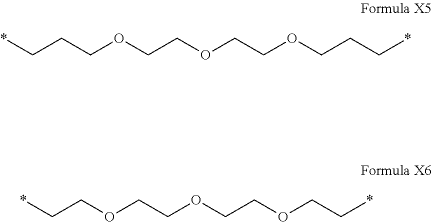 [SMILES: *CCCOCCOCCOCCC* ; *CCOCCOCCOCC*]

In one embodiment, the composition is characterized in that the hydrophobic radical according to formula V is a radical wherein R is a polyether radical according to formula X5.

In one embodiment, the composition is characterized in that the hydrophobic radical according to formula V is a radical wherein R is a polyether radical according to formula X6.

In one embodiment, the composition is characterized in that the hydrophobic radical is a radical according to formula V wherein GpR is a radical according to formula II.

In one embodiment, the composition is characterized in that the hydrophobic radical is a radical according to formula V wherein GpR is a radical according to formula II′.

In one embodiment, the composition is characterized in that the hydrophobic radical is a radical according to formula V wherein GpR is a radical according to formula II″.

In one embodiment, the composition is characterized in that the hydrophobic radical is a radical according to formula V wherein GpR is a radical according to formula II wherein R is a divalent alkyl radical comprising from 2 to 12 carbon atoms.

In one embodiment, the composition is characterized in that the hydrophobic radical is a radical according to formula V wherein GpR is a radical according to formula II wherein R is a divalent alkyl radical comprising from 2 to 6 carbon atoms.

In one embodiment, the composition is characterized in that the hydrophobic radical is a radical according to formula V wherein GpR is a radical according to formula II wherein R is a divalent linear alkyl radical comprising from 2 to 6 carbon atoms.

In one embodiment, the composition is characterized in that the hydrophobic radical is a radical according to formula V wherein GpR is a radical according to formula II wherein R is a divalent alkyl radical comprising from 2 to 4 carbon atoms.

In one embodiment, the composition is characterized in that the hydrophobic radical is a radical according to formula V wherein GpR is a radical according to formula II wherein R is a divalent linear alkyl radical comprising from 2 to 4 carbon atoms.

In one embodiment, the composition is characterized in that the hydrophobic radical is a radical according to formula V wherein GpR is a radical according to formula II wherein R is a divalent alkyl radical comprising 2 carbon atoms.

In one embodiment, the composition is characterized in that the hydrophobic radical is a radical according to formula V wherein GpR is a radical according to formula II′.

In one embodiment, the composition is characterized in that the hydrophobic radical is a radical according to formula V wherein GpR is a radical according to formula II′ wherein R is a divalent linear alkyl radical comprising from 1 to 11 carbon atoms.

In one embodiment, the composition is characterized in that the hydrophobic radical is a radical according to formula V wherein GpR is a radical according to formula II′ wherein R is a divalent alkyl radical comprising from 1 to 6 carbon atoms.

In one embodiment, the composition is characterized in that the hydrophobic radical according to formula V wherein GpR is a radical according to formula II or II′, wherein R is a radical according to formula X3.

In one embodiment, the composition is characterized in that the hydrophobic radical according to formula V wherein GpR is a radical according to formula II or II′, wherein R is a radical according to formula X4.

In one embodiment, the composition is characterized in that the hydrophobic radical according to formula V wherein GpR is a radical according to formula II or II′, wherein R is a radical according to formula X5.

In one embodiment, the composition is characterized in that the hydrophobic radical according to formula V wherein GpR is a radical according to formula II or II′, wherein R is a radical according to formula X6.

In one embodiment, the composition is characterized in that the hydrophobic radical is a radical according to formula I wherein GpR is a radical according to formula II, II′ or II″ wherein R is a polyether radical chosen among the group consisting of the radicals represented by the formulas below:

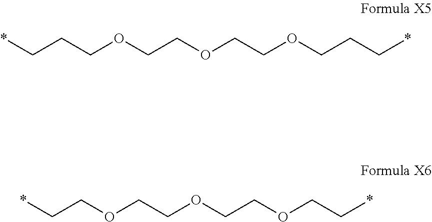 [SMILES: *CCCOCCOCCOCCC* ; *CCOCCOCCOCC*]

In one embodiment, the composition is characterized in that the hydrophobic radical is a radical according to formula V wherein GpR is a radical according to formula II, II′ or II″, wherein R is a divalent linear alkyl radical, comprising from 2 to 5 carbon atoms and bearing one or more amide functions (—CONH₂).

In one embodiment, the composition is characterized in that the hydrophobic radical is a radical according to formula V wherein GpR is a radical according to formula II′, II or II″, wherein R is a divalent linear alkyl radical, comprising from 2 to 5 carbon atoms and bearing one or more amide functions (—CONH₂).

In one embodiment, the composition is characterized in that the hydrophobic radical is a radical according to formula V wherein GpR is a radical according to formula II, II′ or II″ wherein R is a radical chosen among the group consisting of the radicals represented by the formulas below:

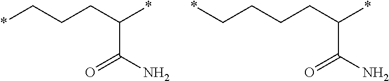

In one embodiment, the composition is characterized in that the R radical is bound to the co-polyamino acid via an amide function borne by the carbon in the delta or epsilon position (or in position 4 or 5) with respect to the amide function (—CONH₂).

In one embodiment, the composition is characterized in that the hydrophobic radical is a radical according to formula V wherein GpR is a radical according to formula II, II′ or II″, wherein R is a linear ether or non-substituted polyether radical comprising from 4 to 14 carbon atoms and 1 to 5 oxygen atoms.

In one embodiment, the composition is characterized in that the hydrophobic radical is a radical according to formula V wherein GpR is a radical according to formula II, II′ or II″, wherein R is an ether radical.

In one embodiment, the composition is characterized in that the hydrophobic radical is a radical according to formula V wherein GpR is a radical according to formula II, II′ or II″, wherein R is an ether radical comprising from 4 to 6 carbon atoms.

In one embodiment, the composition is characterized in that the hydrophobic radical is a radical according to formula V wherein GpR is a radical according to formula II, II′ or II″ wherein R is an ether radical represented by formula.

In one embodiment, the composition is characterized in that the hydrophobic radical is a radical according to formula V wherein GpR is a radical according to formula II, II′ or II″, wherein R is a polyether radical.

In one embodiment, the composition is characterized in that the hydrophobic radical is a radical according to formula V wherein GpR is a radical according to formula II, II′ or II″, wherein R is a linear polyether radical comprising from 6 to 10 carbon atoms and 2 to 3 oxygen atoms.

In one embodiment, the composition is characterized in that the hydrophobic radical is a radical according to formula V wherein GpR is a radical according to formula II, II′ or II″ wherein R is a polyether radical chosen among the group consisting of the radicals represented by the formulas below:

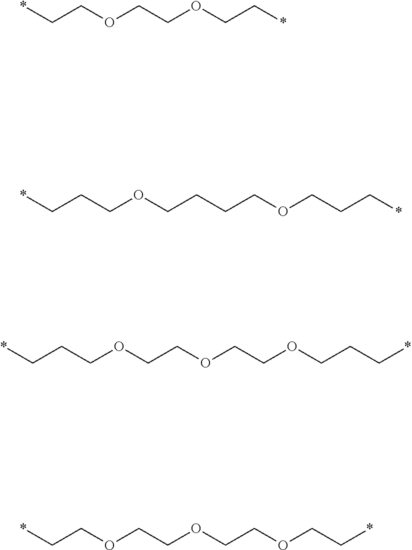 [SMILES: *CCOCCOCC* ; *CCCOCCCCOCCC* ; *CCCOCCOCCOCCC* ; *CCOCCOCCOCC*]

In one embodiment, the composition is characterized in that the hydrophobic radical is a radical according to formula V wherein GpR is a radical according to formula II, II′ or II″ wherein R is a polyether radical chosen among the group consisting of the radicals represented by the formulas below:

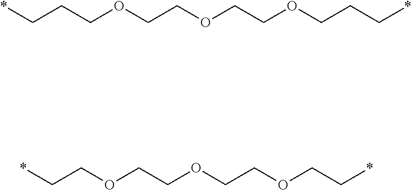 [SMILES: *CCCOCCOCCOCCC* ; *CCOCCOCCOCC*]

In one embodiment, the composition is characterized in that the hydrophobic radical according to formula V wherein GpR is a radical according to formula II wherein R is a polyether radical chosen among the group consisting of the radicals represented by the formulas below:

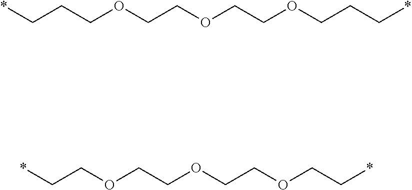 [SMILES: *CCCOCCOCCOCCC* ; *CCOCCOCCOCC*]

In one embodiment, the composition is characterized in that the hydrophobic radical is a radical according to formula V wherein a is equal to 0 (a=0) and r is equal to 0 (r=0).

In one embodiment, the composition is characterized in that the hydrophobic radical is a radical according to formula V wherein a is equal to 1 (a=1) and the radical GpA according to formula III′ is chosen among the group consisting of radicals represented by the formulas below:

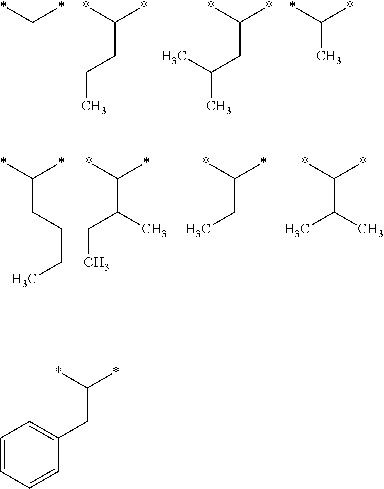

In one embodiment, the composition is characterized in that the hydrophobic radical is a radical according to formula V wherein the GpC radical according to formula IV is chosen among the group consisting of the radicals according to formulas IVa, IVb or IVc hereinafter represented:

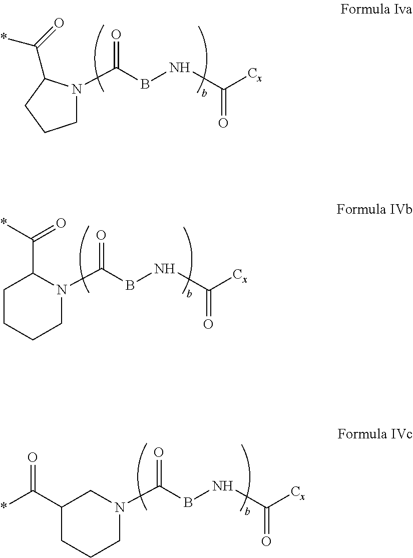

In one embodiment, the composition is characterized in that the hydrophobic radical is a radical according to formula V wherein the GpC radical is according to formula IVa.

In one embodiment, the composition is characterized in that the hydrophobic radical is a radical according to formula I wherein the GpC radical according to formula IV is chosen among the group consisting of the radicals according to formulas IVa, IVb or IVc wherein b is equal to 0, respectively corresponding to formulas IVd, IVe, and IVf hereinafter represented:

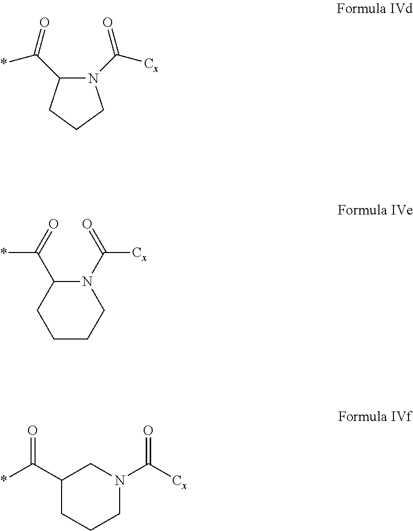

In one embodiment, the composition is characterized in that the hydrophobic radical is a radical according to formula V wherein the GpC radical corresponds to formula IV or IVa wherein b=0, and corresponds to formula IVd.

In one embodiment, the composition is characterized in that the hydrophobic radical is a radical according to formula V wherein the GpC radical according to formula IV wherein b=1 is chosen among the group consisting of radicals wherein B is a residue of amino acid chosen among the group consisting of the radicals represented by the formulas below:

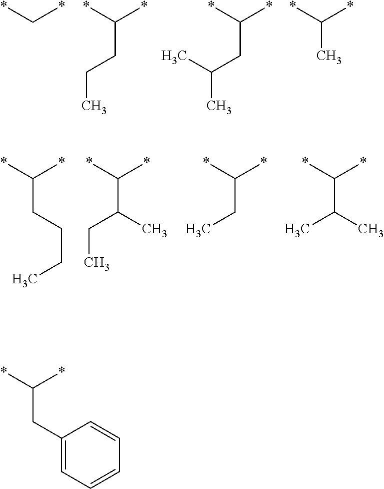

In one embodiment, the composition is characterized in that the hydrophobic radical is a radical according to formula V wherein the GpC radical according to formula IV or IVa wherein b=1 is chosen among the group consisting of radicals wherein B is an amino acid residue chosen among the group consisting of the radicals represented by the formulas below:

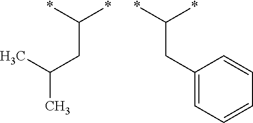

In one embodiment, the composition is characterized in that the hydrophobic radical is a radical according to formula V wherein the GpC radical according to formula IV is chosen among the group consisting of radicals wherein Cx is chosen among the group consisting of linear alkyl radicals.

In one embodiment, the composition is characterized in that the hydrophobic radical is a radical according to formula V wherein the GpC radical according to formula IV is chosen among the group consisting of radicals wherein Cx is chosen among the group consisting of branched alkyl radicals.

In one embodiment, the composition is characterized in that the hydrophobic radical is a radical according to formula V wherein the GpC radical according to formula IV is chosen among the group consisting of radicals wherein Cx is chosen among the group consisting of alkyl radicals comprising from 11 to 14 carbon atoms.

In one embodiment, the composition is characterized in that the hydrophobic radical is a radical according to formula V wherein the GpC radical according to formula IV is chosen among the group consisting of radicals wherein Cx is chosen among the group consisting of the radicals represented by the formulas below:

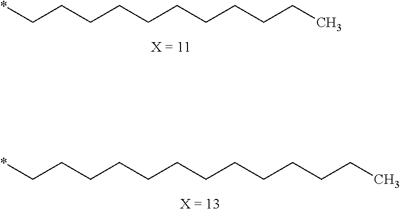 [SMILES: *CCCCCCCCCCC ; *CCCCCCCCCCCCC]

In one embodiment, the composition is characterized in that the hydrophobic radical is a radical according to formula V wherein the GpC radical according to formula IV is chosen among the group consisting of radicals wherein Cx is chosen among the group consisting of alkyl radicals comprising from 15 to 16 carbon atoms.

In one embodiment, the composition is characterized in that the hydrophobic radical is a radical according to formula V wherein the GpC radical according to formula IV is chosen among the group consisting of radicals wherein Cx is chosen among the group consisting of the radicals represented by the formulas below:

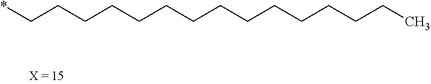 [SMILES: *CCCCCCCCCCCCCCC]

In one embodiment, the composition is characterized in that the hydrophobic radical is a radical according to formula V wherein the GpC radical according to formula IV is chosen among the group consisting of radicals wherein Cx is chosen among the group consisting of the radicals represented by the formulas below:

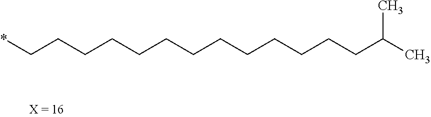 [SMILES: *CCCCCCCCCCCCCC(C)C]

In one embodiment, the composition is characterized in that the hydrophobic radical is a radical according to formula V wherein the GpC radical according to formula IV is chosen among the group consisting of radicals wherein Cx is chosen among the group consisting of alkyl radicals comprising from 17 to 25 carbon atoms.

In one embodiment, the composition is characterized in that the hydrophobic radical is a radical according to formula V wherein the GpC radical according to formula IV is chosen among the group consisting of radicals wherein Cx is chosen among the group consisting of alkyl radicals comprising from 17 to 18 carbon atoms.

In one embodiment, the composition is characterized in that the hydrophobic radical is a radical according to formula V wherein the GpC radical according to formula IV is chosen among the group consisting of radicals wherein Cx is chosen among the group consisting of the alkyl radicals represented by the formulas below:

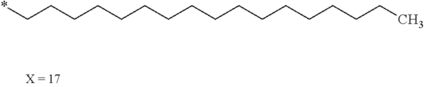 [SMILES: *CCCCCCCCCCCCCCCCC]

In one embodiment, the composition is characterized in that the hydrophobic radical is a radical according to formula V wherein the GpC radical according to formula IV is chosen among the group consisting of radicals wherein Cx is chosen among the group consisting of alkyl radicals comprising from 18 to 25 carbon atoms.

In one embodiment, the composition is characterized in that the hydrophobic radical is a radical according to formula V wherein the GpC radical according to formula IV is chosen among the group consisting of radicals wherein Cx is chosen among the group consisting of the alkyl radicals represented by the formulas below:

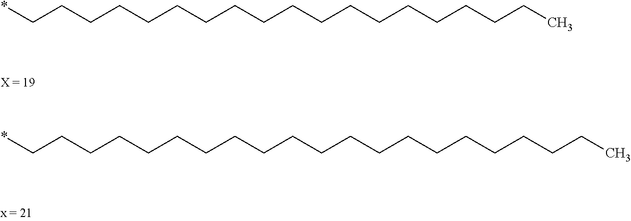 [SMILES: *CCCCCCCCCCCCCCCCCCC ; *CCCCCCCCCCCCCCCCCCCCC]

In one embodiment, the composition is characterized in that the said hydrophobic radicals are chosen among the hydrophobic radicals according to formula I wherein a=1 wherein p=2, represented by formula VI below:

*GpRᵣGpAGpC)₂ Formula VI

wherein

GpR, GpA, GpC, r and a have the definitions given above.

In one embodiment, the composition is characterized in that the hydrophobic radical is a radical according to formula VI wherein GpR is a radical according to formula II.

In one embodiment, the composition is characterized in that the hydrophobic radical is a radical according to formula VI wherein GpR is a radical according to formula II wherein R is a divalent linear alkyl radical comprising from 2 to 12 carbon atoms.

In one embodiment, the composition is characterized in that the hydrophobic radical is a radical according to formula VI wherein GpR is a radical according to formula II wherein R is a divalent alkyl radical comprising from 2 to 6 carbon atoms.

In one embodiment, the composition is characterized in that the hydrophobic radical is a radical according to formula VI wherein GpR is a radical according to formula II wherein R is a divalent linear alkyl radical comprising from 2 to 6 carbon atoms.

In one embodiment, the composition is characterized in that the hydrophobic radical is a radical according to formula VI wherein GpR is a radical according to formula II wherein R is an alkyl radical comprising from 2 to 4 carbon atoms.

In one embodiment, the composition is characterized in that the hydrophobic radical is a radical according to formula VI wherein GpR is a radical according to formula II wherein R is a divalent linear alkyl radical comprising from 2 to 4 carbon atoms.

In one embodiment, the composition is characterized in that the hydrophobic radical is a radical according to formula VI wherein GpR is a radical according to formula II wherein R is a divalent linear alkyl radical comprising 2 carbon atoms.

In one embodiment, the composition is characterized in that the hydrophobic radical is a radical according to formula VI wherein GpR is a radical according to formula

In one embodiment, the composition is characterized in that the hydrophobic radical is a radical according to formula VI wherein GpR is a radical according to formula II′ wherein R is a divalent linear alkyl radical comprising from 1 to 11 carbon atoms.

In one embodiment, the composition is characterized in that the hydrophobic radical is a radical according to formula VI wherein GpR is a radical according to formula II′ wherein R is a divalent alkyl radical comprising from 1 to 6 carbon atoms.

In one embodiment, the composition is characterized in that the hydrophobic radical is a radical according to formula VI wherein GpR is a radical according to formula II, II′ or II″, wherein R is a divalent linear alkyl radical, comprising from 2 to 5 carbon atoms and bearing one or more amide functions (—CONH2).

In one embodiment, the composition is characterized in that the hydrophobic radical is a radical according to formula VI wherein GpR is a radical according to formula II, II′ or II″, wherein R is a divalent linear alkyl radical, comprising from 2 to 5 carbon atoms and bearing one or more amide functions (—CONH2).

In one embodiment, the composition is characterized in that the hydrophobic radical is a radical according to formula VI wherein GpR is a radical according to formula II, II′ or II″ wherein R is a radical chosen among the group consisting of the radicals represented by the formulas below:

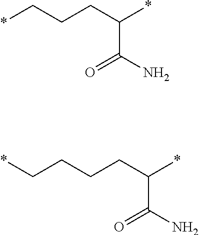 [SMILES: *CCCC(*)C(N)=O ; *CCCCC(*)C(N)=O]

In one embodiment, the composition is characterized in that the hydrophobic radical is a radical according to formula VI wherein the amine function of radical GpR involved in the formation of the amide function which binds said radical GpR to the co-polyamino acid is borne by a carbon in delta or epsilon position (or in position 4 or 5) with respect to the amide function (—CONH2).

In one embodiment, the composition is characterized in that the hydrophobic radical is a radical according to formula VI wherein GpR is a radical according to formula II, II′ or II″, wherein R is a linear ether or non-substituted polyether radical from 4 to 14 carbon atoms and from 1 to 5 oxygen atoms.

In one embodiment, the composition is characterized in that the hydrophobic radical is a radical according to formula VI wherein GpR is a radical according to formula II, II′ or II″, wherein R is an ether radical.

In one embodiment, the composition is characterized in that the ether radical R is a radical comprising from 4 to 6 carbon atoms.

In one embodiment, the composition is characterized in that the ether radical is

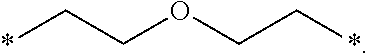

In one embodiment, the composition is characterized in that the hydrophobic radical is a radical according to formula VI wherein GpR is a radical according to formula II, II′ or II″, wherein R is a polyether radical.

In one embodiment, the composition is characterized in that the hydrophobic radical is a radical according to formula VI wherein GpR is a radical according to formula II, II′ or II″, wherein R is a linear polyether radical comprising from 6 to 10 carbon atoms and from 2 to 3 oxygen atoms.

In one embodiment, the composition is characterized in that the hydrophobic radical is a radical according to formula VI wherein GpR is a radical according to formula II, II′ or II″ wherein R is a linear polyether radical chosen among the group consisting of the radicals represented by the formulas below:

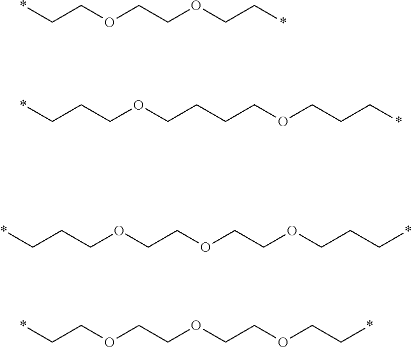 [SMILES: *CCOCCOCC* ; *CCCOCCCCOCCC* ; *CCCOCCOCCOCCC* ; *CCOCCOCCOCC*]

In one embodiment, the composition is characterized in that the hydrophobic radical is a radical according to formula VI wherein the GpA radical according to formula III is chosen among the group consisting of the radicals according to formulas Ma and IIIb represented below:

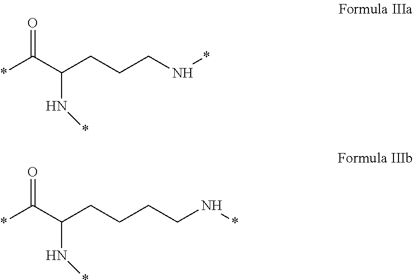 [SMILES: *NCCCC(N*)C(*)=O ; *NCCCCC(N*)C(*)=O]

In one embodiment, the composition is characterized in that the hydrophobic radical is a radical according to formula VI wherein the GpA radical according to formula III is a radical according to formula IIIb represented hereafter:

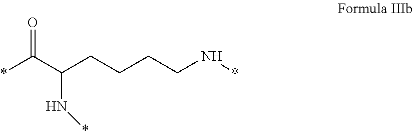 [SMILES: *NCCCCC(N*)C(*)=O]

In one embodiment, the composition is characterized in that the hydrophobic radical is a radical according to formula VI wherein the GpC radical according to formula IV is chosen among the group consisting of the radicals according to formulas IVa, IVb and IVc represented hereinafter:

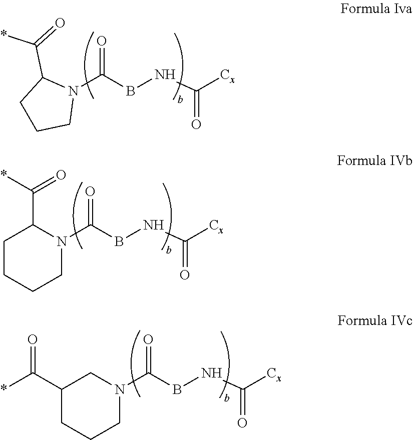

In one embodiment, the composition is characterized in that the hydrophobic radical is a radical according to formula VI wherein the GpC radical is according to formula IVa.

In one embodiment, the composition is characterized in that the hydrophobic radical is a radical according to formula I wherein the GpC radical according to formula IV is chosen among the group consisting of the radicals according to formulas IVa, IVb or IVc wherein b is equal to 0, respectively corresponding to formulas IVd, IVe, and IVf hereinafter represented:

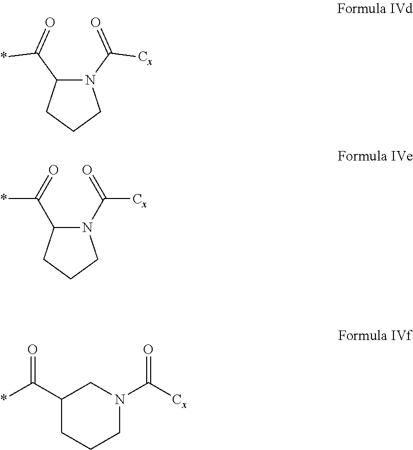

In one embodiment, the composition is characterized in that the hydrophobic radical is a radical according to formula VI wherein the GpC radical corresponds to formula IV or IVa wherein b=0, and corresponds to formula IVd.

In one embodiment, the composition is characterized in that the hydrophobic radical is a radical according to formula VI wherein the GpC radical according to formula IV is chosen among the group consisting of radicals wherein Cx is chosen among the group consisting of linear alkyl radicals comprising from 9 to 15 carbon atoms.

In one embodiment, the composition is characterized in that the hydrophobic radical is a radical according to formula VI wherein the GpC radical according to formula IV is chosen among the group consisting of radicals wherein Cx is chosen among the group consisting of branched alkyl radicals comprising from 9 to 15 carbon atoms.

In one embodiment, the composition is characterized in that the hydrophobic radical is a radical according to formula VI wherein the GpC radical according to formula IV is chosen among the group consisting of radicals wherein Cx is chosen among the group consisting of alkyl radicals comprising 9 or 10 carbon atoms.

In one embodiment, the composition is characterized in that the hydrophobic radical is a radical according to formula VI wherein the GpC radical according to formula IV is chosen among the group consisting of radicals wherein Cx is chosen among the group consisting of alkyl radicals comprising from 11 to 15 carbon atoms.

In one embodiment, the composition is characterized in that the hydrophobic radical is a radical according to formula VI wherein the GpC radical according to formula IV is chosen among the group consisting of radicals wherein Cx is chosen among the group consisting of alkyl radicals comprising from 11 to 13 carbon atoms.

In one embodiment, the composition is characterized in that the hydrophobic radical is a radical according to formula VI wherein the GpC radical according to formula IV is chosen among the group consisting of radicals wherein Cx is chosen among the group consisting of the radicals represented by the formulas below:

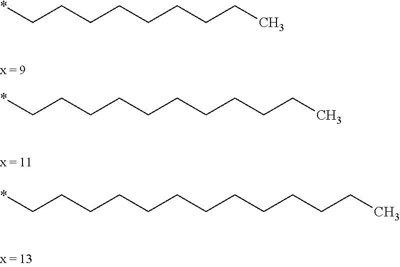

In one embodiment, the composition is characterized in that the hydrophobic radical is a radical according to formula VI wherein the GpC radical according to formula IV is chosen among the group consisting of radicals wherein Cx is chosen among the group consisting of alkyl radicals comprising 14 or 15 carbon atoms.

In one embodiment, the composition is characterized in that the hydrophobic radical is a radical according to formula VI wherein the GpC radical according to formula IV is chosen among the group consisting of radicals wherein Cx is chosen among the group consisting of the radicals represented by the formulas below:

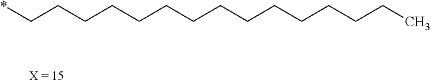 [SMILES: *CCCCCCCCCCCCCCC]

In one embodiment, the composition is characterized in that the co-polyamino acid bearing carboxylate charges and hydrophobic radicals is chosen among the co-polyamino acids according to formula VII below:

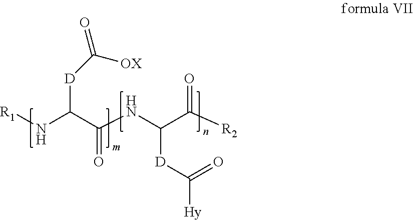

wherein,
D represents, independently, either a —CH₂— group (aspartic unit) or a —CH₂—CH₂-group (glutamic unit),
Hy is a hydrophobic radical chosen among hydrophobic radicals according to formula I, V or VI,
R₁ is a hydrophobic radical chosen among hydrophobic radicals according to formulas I, V or VI, or a radical chosen among the group consisting of a H, a C2 to C10 linear acyl group, a C3 to C10 branched acyl group, benzyl, a terminal “amino acid” unit and a pyroglutamate,
R₂ is a hydrophobic radical chosen among hydrophobic radicals according to formula I, V or VI, or a radical —NR′R″, R′ and R″ identical or different chosen among the group consisting of H, linear or branched alkyls or cyclical in C2 to C10, benzyl and said R′ and R″ alkyls may form together one or more saturated carbon rings, unsaturated and/or aromatic and/or may contain heteroatoms, chosen among the group consisting of O, N and S,
X represents an H or a cationic entity chosen among the group consisting of metal cations;
n+m represents the degree of polymerization DP of the co-polyamino acid, namely the average number of monomeric units per co-polyamino acid chain and 5≤n+m≤250;

The co-polyamino acid bearing carboxylate charges and at least one hydrophobic radical according to formula I can also be referred to as “co-polyamino acid” in the present description.

A “statistical co-polyamino acid” refers to a co-polyamino acid bearing carboxylate charges and at least one hydrophobic radical, a co-polyamino acid according to formula VIIa.

In one embodiment, the composition is characterized in that the co-polyamino acid bearing carboxylate charges and hydrophobic radicals is chosen among the co-polyamino acids according to formula VII wherein R₁═R′i and R₂═R′₂, according to formula VIIa below:

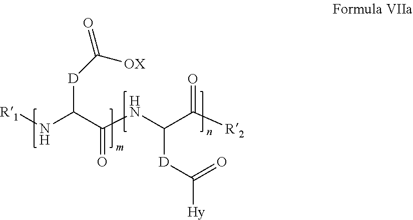

wherein,
m, n, X, D and Hy have the definitions provided above,
R′₁ is a radical chosen among the group consisting of H, linear C2-C10 acyl group, branched C3-C10 acyl group, benzyl, terminal “amino acid” unit and pyroglutamate,
R′2 is a radical —NR′R″, R′ and R″ identical or different chosen among the group consisting of H, linear or branched or cyclic C2 to C10 alkyls, benzyl and said R′ and R″ alkyls which may form together one or more saturated, unsaturated and/or aromatic carbon rings and/or which may contain heteroatoms chosen among the group consisting of O, N and S.

In one embodiment, the composition is characterized in that the co-polyamino acid bearing carboxylate charges and hydrophobic radicals is chosen among the co-polyamino acids according to formulas VIIa wherein Hyd is according to formula V, GpR is according to formula II, GpA is according to formula III′ wherein A is Y9 and GpC corresponds to formula IVd.

In one embodiment, the composition is characterized in that the co-polyamino acid bearing carboxylate charges and hydrophobic radicals is chosen among the co-polyamino acids according to formulas VIIa wherein Hyd is according to formula V, GpR is according to formula II wherein R is —CH2-CH2-, GpA is according to formula III′ wherein A is Y9 and GpC corresponds to formula IVd.

A “defined co-polyamino acid” refers to a co-polyamino acid bearing carboxylate charges and at least one hydrophobic radical, a co-polyamino acid according to formula VIIb.

In one embodiment, the composition is characterized in that the co-polyamino acid bearing carboxylate charges and hydrophobic radicals is chosen among the co-polyamino acids according to formula VII wherein n=0 according to formula VIIb below:

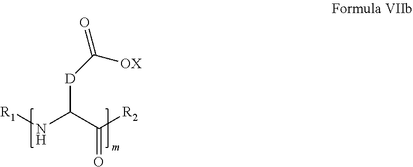

wherein m, X, D, R₁ and R₂ have the definitions given above and at least R₁ or R₂ is a hydrophobic radical according to formula I, V or VI.

In one embodiment, the composition is characterized in that the co-polyamino acid bearing carboxylate charges and hydrophobic radicals is chosen among the co-polyamino acids according to formula VII wherein n=0 according to formula VIIb and R₁ or R₂ is a hydrophobic radical according to formula I, V or VI.

In one embodiment, the composition is characterized in that the co-polyamino acid bearing carboxylate charges and hydrophobic radicals is chosen among the co-polyamino acids according to formula VII wherein Ri is a hydrophobic radical according to formula I, V or VI.

In one embodiment, the composition is characterized in that the co-polyamino acid bearing carboxylate charges and hydrophobic radicals is chosen among the co-polyamino acids according to formula VII wherein R2 is a hydrophobic radical according to formula I, V or VI.

In one embodiment, the composition is characterized in that R₁ is a radical chosen among the group consisting of a C₂ to C₁₀ linear acyl group, a C₃ to C₁₀ branched acyl group, a benzyl, a terminal “amino acid” unit and a pyroglutamate.

In one embodiment, the composition is characterized in that R₁ is a radical chosen among the group consisting of linear C₂ to C₁₀ acyl group or a C₃ to C₁₀ branched acyl group.

In one embodiment, the composition is characterized in that the co-polyamino acid bearing carboxylate charges and hydrophobic radicals is chosen among the co-polyamino acids according to formula VII, VIIa or VIIb wherein group D is a —CH₂— (aspartic unit) group.

In one embodiment, the composition is characterized in that the co-polyamino acid bearing carboxylate charges and hydrophobic radicals is chosen among the co-polyamino acids according to formula VII, VIIa or VIIb wherein the group D is a —CH₂—CH₂— (glutamic unit) group.

In one embodiment, the composition is characterized in that the ratio i between the number of hydrophobic radicals and the number of glutamic or aspartic units is comprised from 0.007 to 0.3.

In one embodiment, the composition is characterized in that the ratio i between the number of hydrophobic radicals and the number of glutamic or aspartic units is comprised from 0.01 to 0.3.

In one embodiment, the composition is characterized in that the ratio i between the number of hydrophobic radicals and the number of glutamic or aspartic units is comprised from 0.02 to 0.2.

In one embodiment, the composition is characterized in that the hydrophobic radical corresponds to formula VI and the ratio i between the number of hydrophobic radicals and the number of glutamic or aspartic units is comprised from 0.007 to 0.15.

In one embodiment, the composition is characterized in that the hydrophobic radical corresponds to formula VI and the ratio i between the number of hydrophobic radicals and the number of glutamic or aspartic units is comprised from 0.01 to 0.1.

In one embodiment, the composition is characterized in that the hydrophobic radical corresponds to formula VI and the ratio i between the number of hydrophobic radicals and the number of glutamic or aspartic units is comprised from 0.02 to 0.08. In one embodiment, the composition is characterized in that the hydrophobic radical corresponds to formula VI wherein radical Cx comprises from 9 to 10 carbon atoms and the ratio i between the number of hydrophobic radicals and the number of glutamic or aspartic units is comprised from 0.03 to 0.15.

In one embodiment, the composition is characterized in that the hydrophobic radical corresponds to formula VI wherein radical Cx comprises from 11 to 12 carbon atoms and the ratio i between the number of hydrophobic radicals and the number of glutamic or aspartic units is comprised from 0.015 to 0.1.

In one embodiment, the composition is characterized in that the hydrophobic radical corresponds to formula VI wherein radical Cx comprises from 11 to 12 carbon atoms and the ratio i between the number of hydrophobic radicals and the number of glutamic or aspartic units is comprised from 0.02 to 0.08.

In one embodiment, the composition is characterized in that the hydrophobic radical corresponds to formula VI wherein radical Cx comprises from 13 to 15 carbon atoms and the ratio i between the number of hydrophobic radicals and the number of glutamic or aspartic units is comprised from 0.01 to 0.1.

In one embodiment, the composition is characterized in that the hydrophobic radical corresponds to formula VI wherein radical Cx comprises from 13 to 15 carbon atoms and the ratio i between the number of hydrophobic radicals and the number of glutamic or aspartic units is comprised from 0.01 to 0.06.

In one embodiment, the composition is characterized in that the hydrophobic radical corresponds to formula V and the ratio i between the number of hydrophobic radicals and the number of glutamic or aspartic units is comprised from 0.007 to 0.3.

In one embodiment, the composition is characterized in that the hydrophobic radical corresponds to formula V and the ratio i between the number of hydrophobic radicals and the number of glutamic or aspartic units is comprised from 0.01 to 0.3.

In one embodiment, the composition is characterized in that the hydrophobic radical corresponds to formula V and the ratio i between the number of hydrophobic radicals and the number of glutamic or aspartic units is comprised from 0.015 to 0.2.

In one embodiment, the composition is characterized in that the hydrophobic radical corresponds to formula V wherein radical Cx comprises from 11 to 14 carbon atoms and the ratio i between the number of hydrophobic radicals and the number of glutamic or aspartic units is comprised from 0.1 to 0.2.

In one embodiment, the composition is characterized in that the hydrophobic radical corresponds to formula V wherein radical Cx comprises from 15 to 16 carbon atoms and the ratio i between the number of hydrophobic radicals and the number of glutamic or aspartic units is comprised from 0.04 to 0.15.

In one embodiment, the composition is characterized in that the hydrophobic radical corresponds to formula V wherein radical Cx comprises from 17 to 18 carbon atoms and the ratio i between the number of hydrophobic radicals and the number of glutamic or aspartic units is comprised from 0.02 to 0.06.

In one embodiment, the composition is characterized in that the hydrophobic radical corresponds to formula V wherein radical Cx comprises from 19 to 25 carbon atoms and the ratio i between the number of hydrophobic radicals and the number of glutamic or aspartic units is comprised from 0.01 to 0.06.

In one embodiment, the composition is characterized in that the hydrophobic radical corresponds to formula V wherein radical Cx comprises from 19 to 25 carbon atoms and the ratio i between the number of hydrophobic radicals and the number of glutamic or aspartic units is comprised from 0.01 to 0.05.

In one embodiment, the composition according to the invention is characterized in that n+m is comprised from 10 to 250.

In one embodiment, the composition according to the invention is characterized in that n+m is comprised from 10 to 200.

In one embodiment, the composition according to the invention is characterized in that n+m is comprised from 15 to 150.

In one embodiment, the composition according to the invention is characterized in that n+m is comprised from 15 to 100.

In one embodiment, the composition according to the invention is characterized in that n+m is comprised from 15 to 80.

In one embodiment, the composition according to the invention is characterized in that n+m is comprised from 15 to 65.

In one embodiment, the composition according to the invention is characterized in that n+m is comprised from 20 to 60.

In one embodiment, the composition according to the invention is characterized in that n+m is comprised from 20 to 50.

In one embodiment, the composition according to the invention is characterized in that n +m is comprised from 20 to 40.

In one embodiment, the concentration of co-polyamino acid bearing carboxylate charges and hydrophobic radicals is at most 40 mg/mL.

In one embodiment, the concentration of co-polyamino acid bearing carboxylate charges and hydrophobic radicals is at most 30 mg/mL.

In one embodiment, the concentration of co-polyamino acid bearing carboxylate charges and hydrophobic radicals is at most 20 mg/mL.

In one embodiment, the concentration of co-polyamino acid bearing carboxylate charges and hydrophobic radicals is at most 10 mg/mL.

In one embodiment, the concentration of co-polyamino acid bearing carboxylate charges and hydrophobic radicals is at most 5 mg/mL.

In one embodiment, the concentration of co-polyamino acid bearing carboxylate charges and hydrophobic radicals is at most 2.5 mg/mL.

In one embodiment, the concentration of co-polyamino acid bearing carboxylate charges and hydrophobic radicals is at most 1 mg/mL. In one embodiment, the concentration of co-polyamino acid bearing carboxylate charges and hydrophobic radicals is at most 0.5 mg/mL.

The invention also relates to the co-polyamino acid bearing carboxylate charges and hydrophobic radicals Hy, said co-polyamino acid being constituted of glutamic or aspartic units and said hydrophobic radicals Hy chosen among the radicals according to formula I as defined below:

*GpRᵣGpAₐGpC)ₚ Formula I

wherein
GpR is a radical according to formulas II, II′ or II″:

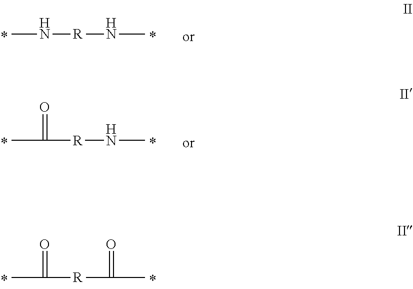

GpA is a radical according to formulas III or III′:

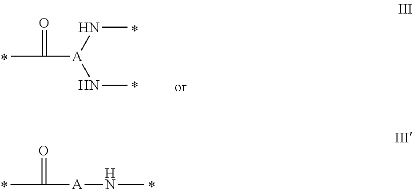

GpC is a radical according to formula IV:

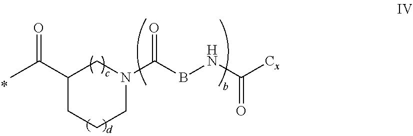

* indicate the attachment sites of the various groups;
a is an integer equal to 0 or 1;
b is an integer equal to 0 or 1;
p is an integer equal to 1 or 2 and
   if p is equal to 1 then a is equal to 0 or 1 and GpA is a radical according to formula III′ and,
   if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
   if r is equal to 0, then the hydrophobic radical according to formula I is bound to the co-polyamino acid via a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
   if r is equal to 1 or 2, then the hydrophobic radical according to formula I is bound to the co-polyamino acid:
      via a covalent bond between a nitrogen atom of the hydrophobic radial and a carbonyl of the co-polyamino acid, thus forming an amide function from the reaction of an amine function of the precursor of the hydrophobic radical and an acid function borne by the precursor of the co-polyamino acid or
      via a covalent bond between a carbonyl of the hydrophobic radical and a nitrogen atom in N-terminal position of the co-polyamino acid, thus forming an amide function from the reaction of an acid function of the precursor of the hydrophobic radical and an amine function in N-terminal position borne by the precursor of the co-polyamino acid;
R is a radical chosen among the group consisting of a linear or branched divalent alkyl radical comprising from 1 to 12 carbon atoms, a divalent linear or branched alkyl radical comprising from 1 to 12 carbon atoms bearing one or more —CONH₂ functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms, more precisely, R is a radical chosen among the group consisting of:
   a linear or branched divalent alkyl radical, comprising from 2 to 12 carbon atoms if GpR is a radical according to formula II or from 1 to 11 carbon atoms if GpR is a radical according to formula II′ or II″;
   a divalent alkyl radical, linear or branched, comprising from 2 to 11 carbon atoms if GpR is a radical according to formula II or from 1 to 11 carbon atoms if GpR is a radical according to formula II′ or II″, said radical alkyl bearing one or more —CONH₂ functions, and
   an unsubstituted ether or polyether radical comprising from 4 to 14 carbon atoms and from 1 to 5 oxygen atoms;
A is a linear or branched alkyl radical comprising from 1 to 8 carbon atoms and optionally substituted by a radical resulting from a saturated, unsaturated or aromatic ring;
B is a radical chosen among the group consisting of an unsubstituted ether or polyether radical comprising from 4 to 14 carbon atoms and from 1 to 5 oxygen atoms or a linear or branched alkyl radical, optionally comprising an aromatic ring, comprising from 1 to 9 carbon atoms;
B is a linear or branched alkyl radical, optionally comprising an aromatic ring, comprising 1 to 9 carbon atoms;
Cₓ is a linear or branched monovalent alkyl radical, optionally comprising a cyclic part, wherein x indicates the number of carbon atoms and:
   if p is equal to 1, x is comprised from 9 to 25 (9≤x≤25):
   if p is equal to 2, x is comprised from 9 to 15 (9≤x≤15),
the ratio i between the number of hydrophobic radicals and the number of glutamic or aspartic units being comprised from 0<i≤0.5;
when several hydrophobic radicals are carried by a co-polyamino acid they are therefore, identical or different;
the degree of polymerization DP of glutamic or aspartic units is comprised from 5 to 250;
   the free acid functions being in the form of an alkaline cation salt chosen among the group consisting of Na⁺ and K⁺.

In one embodiment, the copolyamino acid is a sodium poly-L-glutamate modified at one end according to the formula represented below as described in example AB24.

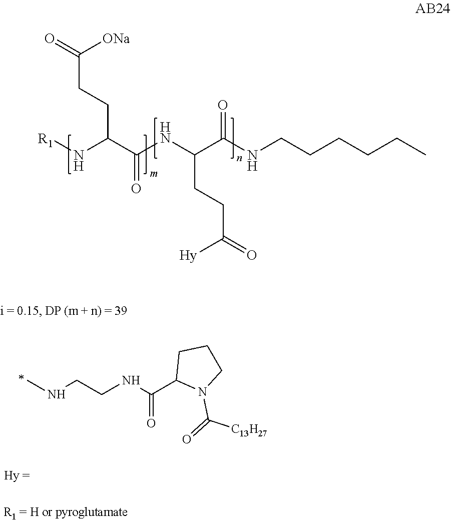

In one embodiment, copolyamino acid is a sodium poly-L-glutamate modified at one end according to the formula represented below as described in example AB32.

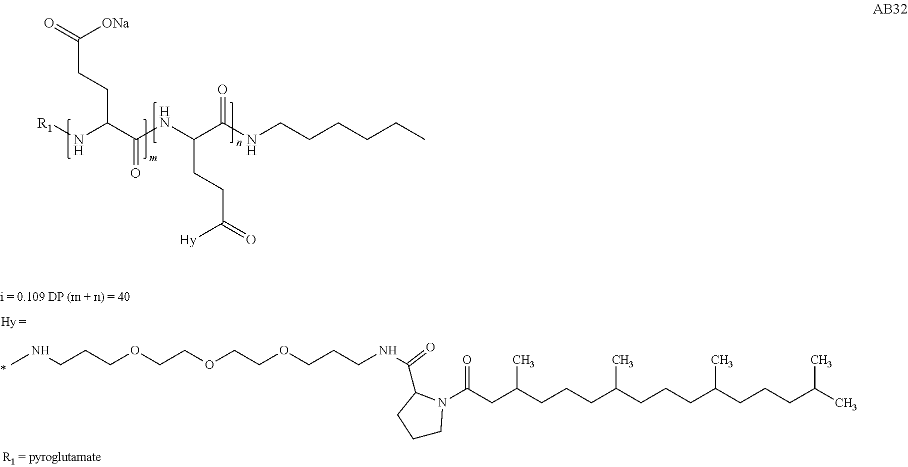

The invention also relates to the precursor Hy′ of the hydrophobic radical Hy according to formula I as defined below:

HGpRᵣGpAₐGpC)ₚ Formula I′

wherein
GpR is a radical according to formulas II, I′ or II″:

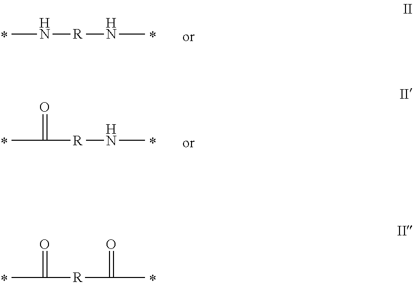

GpA is a radical according to formulas III or III′:

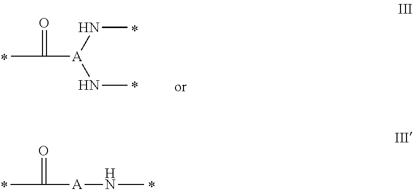

GpC is a radical according to formula IV:

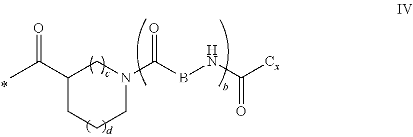

* indicate the attachment sites of the various groups;
a is an integer equal to 0 or 1;
b is an integer equal to 0 or 1;
p is an integer equal to 1 or 2 and
   if p is equal to 1 then a is equal to 0 or 1 and GpA is a radical according to formula III′ and,
   if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
   if r is equal to 0, then the hydrophobic radical according to formula I is bound to the co-polyamino acid via a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
   if r is equal to 1 or 2, then the hydrophobic radical according to formula I is bound to the co-polyamino acid:
      via a covalent bond between a nitrogen atom of the hydrophobic radial and a carbonyl of the co-polyamino acid, thus forming an amide function from the reaction of an amine function of the precursor of the hydrophobic radical and an acid function borne by the precursor of the co-polyamino acid or
      via a covalent bond between a carbonyl of the hydrophobic radical and a nitrogen atom in N-terminal position of the co-polyamino acid, thus forming an amide function from the reaction of an acid function of the precursor of the hydrophobic radical and an amine function in N-terminal position borne by the precursor of the co-polyamino acid;
R is a radical chosen among the group consisting of a linear or branched divalent alkyl radical comprising from 1 to 12 carbon atoms, a divalent linear or branched alkyl radical comprising from 1 to 12 carbon atoms bearing one or more —CONH2 functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms, more precisely, R is a radical chosen among the group consisting of:
   a linear or branched divalent alkyl radical, comprising from 2 to 12 carbon atoms if GpR is a radical according to formula II or from 1 to 11 carbon atoms if GpR is a radical according to formula II′ or II″;
   a divalent alkyl radical, linear or branched, comprising from 2 to 11 carbon atoms if GpR is a radical according to formula II or from 1 to 11 carbon atoms if GpR is a radical according to formula II′ or II″, said radical alkyl bearing one or more —CONH₂ functions, and
   an unsubstituted ether or polyether radical comprising from 4 to 14 carbon atoms and from 1 to 5 oxygen atoms;
A is a linear or branched alkyl radical comprising from 1 to 8 carbon atoms and optionally substituted by a radical resulting from a saturated, unsaturated or aromatic ring;
B is a radical chosen among the group consisting of an unsubstituted ether or polyether radical comprising from 4 to 14 carbon atoms and from 1 to 5 oxygen atoms or a linear or branched alkyl radical, optionally comprising an aromatic ring, comprising from 1 to 9 carbon atoms;
Cₓ is a linear or branched monovalent alkyl radical, optionally comprising a cyclic part, wherein x indicates the number of carbon atoms and:
   if p is equal to 1, x is comprised from 9 to 25 (9≤x≤25):
   if p is equal to 2, x is comprised from 9 to 15 (9≤x≤15),
the ratio i between the number of hydrophobic radicals and the number of glutamic or aspartic units being comprised from 0<i≤0.5;
when several hydrophobic radicals are carried by a co-polyamino acid they are therefore, identical or different;
the degree of polymerization DP of glutamic or aspartic units is comprised from 5 to 250;
   the free acid functions being in the form of an alkaline cation salt chosen among the group consisting of Na+ and K+.

In one embodiment, the invention also relates to the precursors of said hydrophobic radicals according to formula V′ and VI′:

HGpRᵣGpAₐGpC formule V′

HGpRᵣGpAGpC)₂ formule VI′

wherein GpR, GpA, GpC, r, a have the definitions given above.

In one embodiment, the composition according to the invention is characterized in that the co-polyamino acid is obtained from a polyamino acid obtained by polymerization.

In one embodiment, the composition according to the invention is characterized in that the co-polyamino acid is obtained from a polyamino acid obtained by ring opening polymerization of a glutamic acid N-carboxyanhydride derivative or an aspartic acid N-carboxyanhydride derivative.

In one embodiment, the composition according to the invention is characterized in that co-polyamino acid is obtained from a polyamino acid obtained by polymerization of a glutamic acid N-carboxyanhydride derivative or an aspartic acid N-carboxyanhydride derivative as described in Adv. Polym. Sci. 2006, 202, 1-18 (Deming, T J).

In one embodiment, the composition according to the invention is characterized in that co-polyamino acid is obtained from a polyamino acid obtained by polymerization of a glutamic acid N-carboxyanhydride derivative.

In one embodiment, the composition according to the invention is characterized in that co-polyamino acid is obtained from a polyamino acid obtained by polymerization of a glutamic acid N-carboxyanhydride derivative chosen among the group consisting of N-carboxyanhydride methyl glutamate (GluOMe-NCA), benzyl N-carboxyanhydride glutamate (GluOBzl-NCA) and t-butyl N-carboxyanhydride glutamate (GluOtBu-NCA).

In one embodiment, the glutamic acid N-carboxyanhydride derivative is methyl N-carboxyanhydride L-glutamate (L-GluOMe-NCA).

In one embodiment, the glutamic acid N-carboxyanhydride derivative is benzyl N-carboxyanhydride L-glutamate (L-GluOBzl-NCA).

In one embodiment, the composition according to the invention is characterized in that co-polyamino acid is obtained from a polyamino acid obtained by polymerization of a glutamic acid N-carboxyanhydride derivative or an aspartic acid N-carboxyanhydride derivative using an organometallic complex of a transition metal as initiator, as described in Nature 1997, 390, 386-389 (Deming, T J).

In one embodiment, the composition according to the invention is characterized in that co-polyamino acid is obtained from a polyamino acid obtained by polymerization of a glutamic acid N-carboxyanhydride derivative or an aspartic acid N-carboxyanhydride derivative using ammonia or a primary amine as initiator as described in FR 2,801,226 (Touraud, F. et al.) and references cited therein.

In one embodiment, the composition according to the invention is characterized in that co-polyamino acid is obtained from a polyamino acid obtained by polymerization of a glutamic acid N-carboxyanhydride derivative or an aspartic acid N-carboxyanhydride derivative using hexamethyldisilazane as initiator as described in J. Am. Chem. Soc. 2007, 129, 14114-14115 (Lu H., et al.) or a silylated amine as described in J. Am. Chem. Soc. 2008, 130, 12562-12563 (Lu H., et al.).

In one embodiment, the composition according to the invention is characterized in that the process for synthesizing the polyamino acid obtained by polymerization of a glutamic acid N-carboxyanhydride derivative or an aspartic acid N-carboxyanhydride derivative acid from which the co-polyamino acid is obtained, comprises a ester function hydrolysis step.

In one embodiment, this ester hydrolysis step may consist of hydrolysis in an acidic medium or hydrolysis in a basic medium or may be carried out by hydrogenation.

In one embodiment, this ester group hydrolysis step is a hydrolysis in an acidic medium.

In one embodiment, this ester group hydrolysis step is carried out by hydrogenation.

In one embodiment, the composition according to the invention is characterized in that co-polyamino acid is obtained from a polyamino acid obtained by depolymerization of a polyamino acid of higher molecular weight.

In one embodiment, the composition according to the invention is characterized in that co-polyamino acid is obtained from a polyamino acid obtained by enzymatic depolymerization of a polyamino acid of higher molecular weight.

In one embodiment, the composition according to the invention is characterized in that co-polyamino acid is obtained from a polyamino acid obtained by chemical depolymerization of a polyamino acid of higher molecular weight.

In one embodiment, the composition according to the invention is characterized in that co-polyamino acid is obtained from a polyamino acid obtained by enzymatic and chemical depolymerization of a polyamino acid of higher molecular weight.

In one embodiment, the composition according to the invention is characterized in that co-polyamino acid is obtained from a polyamino acid obtained by depolymerization of a polyamino acid of higher molecular weight chosen among the group consisting of sodium polyglutamate and sodium polyaspartate.

In one embodiment, the composition according to the invention is characterized in that co-polyamino acid is obtained from a polyamino acid obtained by depolymerization of a sodium polyglutamate of higher molecular weight.

In one embodiment, the composition according to the invention is characterized in that co-polyamino acid is obtained from a polyamino acid obtained by depolymerization of a sodium polyaspartate of higher molecular weight.

In one embodiment, the composition according to the invention is characterized in that co-polyamino acid is obtained by grafting a hydrophobic group onto an acid poly-L-glutamic acid or poly-L-aspartic acid using amide bond formation processes well known to the person skilled in the art.

In one embodiment, the composition according to the invention is characterized in that co-polyamino acid is obtained by grafting a hydrophobic group onto a poly-L-glutamic acid or poly-L-aspartic acid using amide bond formation processes used for peptide synthesis.

In one embodiment, the composition according to the invention is characterized in that co-polyamino acid is obtained by grafting a hydrophobic group on a poly-L-glutamic acid or poly-L-aspartic acid as described in patent FR 2,840,614 (Chan, YP et al.).

Amylin, or islet amyloid polypeptide (IAPP), is a 37-residue peptide hormone. It is co-secreted with insulin from pancreatic beta cells in the ratio of about 100:1. Amylin plays a role in glycemic regulation by stopping the secretion of endogenous glucagon and slowing down gastric emptying and promoting satiety, thereby reducing postprandial glucose excursions in blood glucose levels.

IAPP is processed from a coding sequence of 89 residues. Proislet amyloid polypeptide (proIAPP, proamyline, proislet protein) is produced in pancreatic beta cells (beta cells) in the form of a pro-peptide of SRO 67 amino acids, 7404 Dalton, and undergoes post-translational modifications including protease cleavage to produce amylin.

In the current application, amylin as mentioned refers to the compounds described in U.S. Pat. No. 5,124,314 and U.S. Pat. No. 5,234,906.

The term “analogue”, when used in reference to a peptide or a protein, is understood to mean a peptide or a protein, wherein one or more constituent amino acid residues of the primary sequence have been substituted by other residues of amino acids and/or wherein one or more constituent amino acid residues have been deleted and/or wherein one or more constituent amino acid residues have been added. The percentage of homology allowed for the current definition of an analogue is 50%. In the case of amylin, an analogue may for example be derived from the primary amino acid sequence of amylin by substituting one or more natural or unnatural amino acids or peptidomimetics.

“Derivative”, when it is used in reference to a peptide or a protein, is understood to mean a peptide or protein or an analogue chemically modified with a sub stituent that is not present in the peptide or protein or analogue reference, in other words, a peptide or protein that has been modified by covalent bonding to introduce non-amino acid sub stituents.

An amylin receptor agonist refers to a compound that mimics one or more characteristics of amylin activity.

Amylin derivatives are described in Yan et al., PNAS Vol. 103, No. 7, p 2046-2051, 2006.

In one embodiment, the substituent is chosen among the group consisting of fatty chains.

Amylin analogues are described in U.S. Pat. No. 5,686,411, U.S. Pat. No. 6,114,304 or U.S. Pat. No. 6,410,511.

In one embodiment, the composition is characterized in that amylin, amylin receptor agonist or the amylin analogue is amylin.

In one embodiment, the composition is characterized in that the amylin analogue is pramlintide (Symlin®) marketed by ASTRAZENECA AB.

In one embodiment, the composition is characterized in that it comprises amylin, amylin receptor agonist or amylin analogue in a concentration ranging from 0.1 to 5 mg/mL.

In one embodiment, the composition is characterized in that it comprises amylin, amylin receptor agonist or amylin analogue in a concentration ranging from 0.2 to 4 mg/mL.

In one embodiment, the composition is characterized in that it comprises amylin, amylin receptor agonist or amylin analogue in a concentration ranging from 0.3 to 3 mg/mL.

In one embodiment, the composition is characterized in that it comprises amylin, amylin receptor agonist or amylin analogue in a concentration ranging from 0.4 to 2 mg/mL.

In one embodiment, the composition is characterized in that it comprises amylin, amylin receptor agonist or amylin analogue in a concentration ranging from 0.5 to 1.5 mg/mL.

In one embodiment, the composition is characterized in that it comprises amylin, amylin receptor agonist or amylin analogue in a concentration ranging from 0.5 to 1 mg/mL.

In one embodiment, the composition is characterized in that it comprises amylin, amylin agonist or receptor or amylin analogue at a concentration of 0.6 mg/mL.

In one embodiment, the composition is characterized in that it comprises amylin, amylin agonist or receptor or amylin analogue at a concentration of 0.6 mg/mL·mg/mL

In one embodiment, the molar ratio of co-polyamino acid/amylin, agonist to amylin receptor or amylin analogue is greater than or equal to 1.

In one embodiment, co-polyamino acid/amylin molar ratios, amylin receptor agonist or amylin analogue are comprised from 1.5 to 75.

In one embodiment, co-polyamino acid/amylin molar ratios, amylin receptor agonist or amylin analogue are comprised from 1.8 to 50.

In one embodiment, co-polyamino acid/amylin molar ratios, amylin receptor agonist or amylin analogue are comprised from 2 to 35.

In one embodiment, co-polyamino acid/amylin molar ratios, amylin receptor agonist or amylin analogue are comprised from 2.5 to 30.

In one embodiment, co-polyamino acid/amylin molar ratios, amylin receptor agonist or amylin analogue are comprised from 3 to 30.

In one embodiment, co-polyamino acid/amylin molar ratios, amylin receptor agonist or amylin analogue are comprised from 3.5 to 30.

In one embodiment, co-polyamino acid/amylin molar ratios, amylin receptor agonist or amylin analogue are comprised from 4 to 30.

In one embodiment, co-polyamino acid/amylin molar ratios, amylin receptor agonist or amylin analogue are comprised from 5 to 30.

In one embodiment, co-polyamino acid/amylin molar ratios, amylin receptor agonist or amylin analogue are comprised from 7 to 30.

In one embodiment, co-polyamino acid/amylin molar ratios, amylin receptor agonist or amylin analogue are comprised from 9 to 30.

In one embodiment, co-polyamino acid/amylin molar ratios are comprised from 3 to 75.

In one embodiment, co-polyamino acid/amylin molar ratios are comprised from 7 to 50.

In one embodiment, co-polyamino acid/amylin molar ratios are comprised from 10 to 30.

In one embodiment, co-polyamino acid/amylin molar ratios are comprised from 15 to 30.

In one embodiment, co-polyamino acid/pramlintide molar ratios are comprised from 1.5 to 75.

In one embodiment, co-polyamino acid/pramlintide molar ratios are comprised from 2 to 50.

In one embodiment, co-polyamino acid/pramlintide molar ratios are comprised from 3 to 30.

In one embodiment, co-polyamino acid/pramlintide molar ratios are comprised from 4 to 30.

In one embodiment, co-polyamino acid/pramlintide molar ratios are comprised from 5 to 30.

In one embodiment, co-polyamino acid/pramlintide molar ratios are comprised from 8 to 30.

In one embodiment, co-polyamino acid/pramlintide molar ratios are comprised from 10 to 30.

In one embodiment, hydrophobic radical Hy/amylin, amylin receptor agonist or amylin analogue molar ratios are comprised from 1.5 to 150.

In one embodiment, hydrophobic radical Hy/amylin, amylin receptor agonist or amylin analogue molar ratios are comprised from 1.8 to 100.

In one embodiment, hydrophobic radical Hy/amylin, amylin receptor agonist or amylin analogue molar ratios are comprised from 2 to 70.

In one embodiment, hydrophobic radical Hy/amylin, amylin receptor agonist or amylin analogue molar ratios are comprised from 2.5 to 60.

In one embodiment, hydrophobic radical Hy/amylin, amylin receptor agonist or amylin analogue molar ratios are comprised from 3 to 60.

In one embodiment, hydrophobic radical Hy/amylin, amylin receptor agonist or amylin analogue molar ratios are comprised from 3.5 to 60.

In one embodiment, hydrophobic radical Hy/amylin, amylin receptor agonist or amylin analogue molar ratios are comprised from 4 to 60.

In one embodiment, hydrophobic radical Hy/amylin, amylin receptor agonist or amylin analogue molar ratios are comprised from 5 to 60.

In one embodiment, hydrophobic radical Hy/amylin, amylin receptor agonist or amylin analogue molar ratios are comprised from 7 to 60.

In one embodiment, hydrophobic radical Hy/amylin, amylin receptor agonist or amylin analogue molar ratios are comprised from 9 to 60.

In one embodiment, hydrophobic radical Hy/amylin molar ratios are comprised from 5 to 60.

In one embodiment, hydrophobic radical Hy/amylin molar ratios are comprised from 10 to 60.

In one embodiment, hydrophobic radical Hy/amylin molar ratios are comprised from 15 to 60.

In one embodiment, hydrophobic radical Hy/pramlintide ratios are comprised from 1.5 to 60.

In one embodiment, hydrophobic radical Hy/pramlintide ratios are comprised from 2 to 60.

In one embodiment, hydrophobic radical Hy/pramlintide ratios are comprised from 3 to 60.

In one embodiment, hydrophobic radical Hy/pramlintide ratios are comprised from 4 to 60.

In one embodiment, hydrophobic radical Hy/pramlintide ratios are comprised from 5 to 60.

In one embodiment, hydrophobic radical Hy/pramlintide ratios are comprised from 8 to 60.

In one embodiment, hydrophobic radical Hy/pramlintide ratios are comprised from 10 to 60.

In one embodiment, co-polyamino acid/amylin mass ratios, amylin receptor agonist or amylin analogue are comprised from 1.0 to 70.

In one embodiment, co-polyamino acid/amylin mass ratios, amylin receptor agonist or amylin analogue are comprised from 1.2 to 45.

In one embodiment, co-polyamino acid/amylin mass ratios, amylin receptor agonist or amylin analogue are comprised from 1.3 to 30.

In one embodiment, co-polyamino acid/amylin mass ratios, amylin receptor agonist or amylin analogue are comprised from 1.7 to 27.

In one embodiment, co-polyamino acid/amylin mass ratios, amylin receptor agonist or amylin analogue are comprised from 2.0 to 27.

In one embodiment, co-polyamino acid/amylin mass ratios, amylin receptor agonist or amylin analogue are comprised from 2.3 to 27.

In one embodiment, co-polyamino acid/amylin mass ratios, amylin receptor agonist or amylin analogue are comprised from 2.7 to 27.

In one embodiment, co-polyamino acid/amylin mass ratios, amylin receptor agonist or amylin analogue are comprised from 3.3 to 27.

In one embodiment, co-polyamino acid/amylin mass ratios, amylin receptor agonist or amylin analogue are comprised from 4.7 to 27.

In one embodiment, co-polyamino acid/amylin mass ratios, amylin receptor agonist or amylin analogue are comprised from 6.0 to 27.

In one embodiment, co-polyamino acid/amylin mass ratios are comprised from 2.0 to 67.

In one embodiment, co-polyamino acid/amylin mass ratios are comprised from 4.7 to 27.

In one embodiment, co-polyamino acid/amylin mass ratios are comprised from 6.7 to 27.

In one embodiment, co-polyamino acid/amylin mass ratios are comprised from 10 to 27.

In one embodiment, co-polyamino acid/pramlintide mass ratios are comprised from 1.0 to 67.

In one embodiment, co-polyamino acid/pramlintide mass ratios are comprised from 1.3 to 45.

In one embodiment, co-polyamino acid/pramlintide mass ratios are comprised from 2.7 to 27.

In one embodiment, co-polyamino acid/pramlintide mass ratios are comprised from 3.3 to 27.

In one embodiment, co-polyamino acid/pramlintide mass ratios are comprised from 5.3 to 27.

In one embodiment, co-polyamino acid/pramlintide mass ratios are comprised from 6.7 to 27.

In one embodiment, the composition is characterized in that it further comprises insulin.

In one embodiment, the composition is characterized in that the insulin is a prandial insulin. Prandial insulins are soluble at pH 7.

Prandial insulin refers to fast-acting or “regular” insulin.

Prandial insulins denote or so-called ‘fast-acting’ are insulins that must satisfy the needs caused by the ingestion of proteins and carbohydrates during a meal, they must react in less than 30 minutes.

In one embodiment, “regular” prandial insulin is human insulin.

In one embodiment, prandial insulin is a recombinant human insulin as described in the European Pharmacopoeia and the American Pharmacopoeia.

Human insulin is for example marketed under the brand names Humulin® (ELI LILLY) and Novolin® (NOVO NORDISK).

Fast-acting prandial insulins are insulins that are obtained by recombination and the primary sequence of which has been modified to reduce their duration of action.

In one embodiment, the said (fast-acting) prandial insulins are chosen among the group comprising insulin lispro (Humalog®), insulin glulisine (Apidra®) and insulin aspart (NovoLog).

In one embodiment, prandial insulin is insulin lispro.

In one embodiment, prandial insulin is insulin glulisine.

In one embodiment, prandial insulin is insulin aspart.

The units recommended by pharmacopoeias for insulins are presented in table 40 below with their equivalents in mg:

TABLE 40
Pharmacopoeia recommended units for insulins

| | EP Pharmacopoeia 8.0 | US Pharmacopoeia - |
|---|---|---|
| Insulin | (2014) | USP38 (2015) |
| aspart | 1 U = 0.0350 mg of | 1 USP = 0.0350 mg of |
| | insulin aspart | insulin aspart |
| Lispro | 1 U = 0.0347 mg of | 1 USP = 0.0347 mg of |
| | insulin lispro | insulin lispro |
| Human | 1 IU = 0.0347 mg of | 1 USP = 0.0347 mg of |
| | human insulin | human insulin |

In the case of insulin glulisine, 100U=3.49 mg of insulin glulisine (according to “Annex 1—Summary of product characteristics” for ADIPRA®).

However, in the rest of the text, U is systematically used indifferently for the quantities and the concentrations in all the insulins. The corresponding respective values in mg are those provided above for values expressed in U, UI or USP.

In one embodiment, it relates to a pharmaceutical formulation characterized in that the insulin concentration is comprised from 240 to 3000 μM (40 to 500 U/ml).

In one embodiment, it relates to a pharmaceutical formulation characterized in that the insulin concentration is comprised from 600 to 3000 μM (100 to 500 U/ml).

In one embodiment, it relates to a pharmaceutical formulation characterized in that the insulin concentration is comprised from 600 to 2400 μM (100 to 400 U/ml).

In one embodiment, it relates to a pharmaceutical formulation characterized in that the insulin concentration is comprised from 600 to 1800 μM (100 to 300 U/ml).

In one embodiment, it relates to a pharmaceutical formulation characterized in that the insulin concentration is comprised from 600 to 1200 μM (100 to 200 U/ml).

In one embodiment, it relates to a pharmaceutical formulation characterized in that the insulin concentration is 600 (100 U/mL).

In one embodiment, it relates to a pharmaceutical formulation characterized in that the insulin concentration is 1200 (200 U/mL).

In one embodiment, it relates to a pharmaceutical formulation characterized in that the insulin concentration is 1800 (300 U/mL).

In one embodiment, it relates to a pharmaceutical formulation characterized in that the insulin concentration is 2400 (400 U/mL).

In one embodiment, it relates to a pharmaceutical formulation characterized in that the insulin concentration is 3000 (500 U/mL).

In one embodiment, the molar ratio of co-polyamino acid/amylin, agonist to amylin receptor or amylin analogue is greater than or equal to 1.

In one embodiment comprising prandial insulin, the co-polyamino acid/amylin, amylin receptor agonist or amylin analogue molar ratios are comprised from 1.5 to 75.

In one embodiment comprising prandial insulin, the co-polyamino acid/amylin, amylin receptor agonist or amylin analogue molar ratios are comprised from 1.8 to 50.

In one embodiment comprising prandial insulin, the co-polyamino acid/amylin, amylin receptor agonist or amylin analogue molar ratios are comprised from 2 to 35.

In one embodiment comprising prandial insulin, the co-polyamino acid/amylin, amylin receptor agonist or amylin analogue molar ratios are comprised from 2.5 to 30.

In one embodiment comprising prandial insulin, the co-polyamino acid/amylin, amylin receptor agonist or amylin analogue molar ratios are comprised from 3 to 30.

In one embodiment comprising prandial insulin, the co-polyamino acid/amylin, amylin receptor agonist or amylin analogue molar ratios are comprised from 3.5 to 30.

In one embodiment comprising prandial insulin, the co-polyamino acid/amylin, amylin receptor agonist or amylin analogue molar ratios are comprised from 4 to 30.

In one embodiment comprising prandial insulin, the co-polyamino acid/amylin, amylin receptor agonist or amylin analogue molar ratios are comprised from 5 to 30.

In one embodiment comprising prandial insulin, the co-polyamino acid/amylin, amylin receptor agonist or amylin analogue molar ratios are comprised from 7 to 30.

In one embodiment comprising prandial insulin, the co-polyamino acid/amylin, amylin receptor agonist or amylin analogue molar ratios are comprised from 9 to 30.

In one embodiment comprising prandial insulin, the co-polyamino acid/amylin molar ratios are comprised from 5 to 75.

In one embodiment comprising prandial insulin, the co-polyamino acid/amylin molar ratios are comprised from 10 to 50.

In one embodiment comprising prandial insulin, the co-polyamino acid/amylin molar ratios are comprised from 15 to 30.

In one embodiment comprising prandial insulin, the co-polyamino acid/amylin molar ratios are comprised from 1.5 to 75.

In one embodiment comprising prandial insulin, the co-polyamino acid/amylin molar ratios are comprised from 2 to 50.

In one embodiment comprising prandial insulin, the co-polyamino acid/amylin molar ratios are comprised from 3 to 30.

In one embodiment comprising prandial insulin, the co-polyamino acid/amylin molar ratios are comprised from 4 to 30.

In one embodiment comprising prandial insulin, the co-polyamino acid/amylin molar ratios are comprised from 5 to 30.

In one embodiment comprising prandial insulin, the co-polyamino acid/amylin molar ratios are comprised from 8 to 30.

In one embodiment comprising prandial insulin, the co-polyamino acid/amylin molar ratios are comprised from 10 to 30.

In one embodiment comprising prandial insulin, the co-polyamino acid/amylin, amylin receptor agonist or amylin analogue molar ratios are comprised from 1.5 to 150.

In one embodiment comprising prandial insulin, the co-polyamino acid/amylin, amylin receptor agonist or amylin analogue molar ratios are comprised from 1.8 to 100.

In one embodiment comprising prandial insulin, the co-polyamino acid/amylin, amylin receptor agonist or amylin analogue molar ratios are comprised from 2 to 70.

In one embodiment comprising prandial insulin, the co-polyamino acid/amylin, amylin receptor agonist or amylin analogue molar ratios are comprised from 2.5 to 60.

In one embodiment comprising prandial insulin, the co-polyamino acid/amylin, amylin receptor agonist or amylin analogue molar ratios are comprised from 3 to 60.

In one embodiment comprising prandial insulin, the co-polyamino acid/amylin, amylin receptor agonist or amylin analogue molar ratios are comprised from 3.5 to 60.

In one embodiment comprising prandial insulin, the co-polyamino acid/amylin, amylin receptor agonist or amylin analogue molar ratios are comprised from 4 to 60.

In one embodiment comprising prandial insulin, the co-polyamino acid/amylin, amylin receptor agonist or amylin analogue molar ratios are comprised from 5 to 60.

In one embodiment comprising prandial insulin, the co-polyamino acid/amylin, amylin receptor agonist or amylin analogue molar ratios are comprised from 7 to 60.

In one embodiment comprising prandial insulin, the co-polyamino acid/amylin, amylin receptor agonist or amylin analogue molar ratios are comprised from 9 to 60.

In one embodiment comprising prandial insulin, the co-polyamino acid/amylin molar ratios are comprised from 5 to 60.

In one embodiment comprising prandial insulin, the co-polyamino acid/amylin molar ratios are comprised from 10 to 60.

In one embodiment comprising prandial insulin, the co-polyamino acid/amylin molar ratios are comprised from 15 to 60.

In one embodiment comprising prandial insulin, the co-polyamino acid/amylin molar ratios are comprised from 1.5 to 60.

In one embodiment comprising prandial insulin, the co-polyamino acid/amylin molar ratios are comprised from 2 to 60.

In one embodiment comprising prandial insulin, the co-polyamino acid/amylin molar ratios are comprised from 3 to 60.

In one embodiment comprising prandial insulin, the co-polyamino acid/amylin molar ratios are comprised from 4 to 60.

In one embodiment comprising prandial insulin, the co-polyamino acid/amylin molar ratios are comprised from 5 to 60.

In one embodiment comprising prandial insulin, the co-polyamino acid/amylin molar ratios are comprised from 8 to 60.

In one embodiment comprising prandial insulin, the co-polyamino acid/amylin molar ratios are comprised from 10 to 60.

In one embodiment comprising prandial insulin, the co-polyamino acid/amylin, amylin receptor agonist or amylin analogue molar ratios are comprised from 1.0 to 70.

In one embodiment comprising prandial insulin, the co-polyamino acid/amylin, amylin receptor agonist or amylin analogue molar ratios are comprised from 1.2 to 45.

In one embodiment comprising prandial insulin, the co-polyamino acid/amylin, amylin receptor agonist or amylin analogue molar ratios are comprised from 1.3 to 30.

In one embodiment comprising prandial insulin, the co-polyamino acid/amylin, amylin receptor agonist or amylin analogue molar ratios are comprised from 1.7 to 27.

In one embodiment comprising prandial insulin, the co-polyamino acid/amylin, amylin receptor agonist or amylin analogue molar ratios are comprised from 2.0 to 27.

In one embodiment comprising prandial insulin, the co-polyamino acid/amylin, amylin receptor agonist or amylin analogue molar ratios are comprised from 2.3 to 27.

In one embodiment comprising prandial insulin, the co-polyamino acid/amylin, amylin receptor agonist or amylin analogue molar ratios are comprised from 2.7 to 27.

In one embodiment comprising prandial insulin, the co-polyamino acid/amylin, amylin receptor agonist or amylin analogue molar ratios are comprised from 3.3 to 27.

In one embodiment comprising prandial insulin, the co-polyamino acid/amylin, amylin receptor agonist or amylin analogue molar ratios are comprised from 4.7 to 27.

In one embodiment comprising prandial insulin, the co-polyamino acid/amylin, amylin receptor agonist or amylin analogue molar ratios are comprised from 6.0 to 27.

In one embodiment comprising prandial insulin, the co-polyamino acid/amylin molar ratios are comprised from 3.3 to 67.

In one embodiment comprising prandial insulin, the co-polyamino acid/amylin molar ratios are comprised from 6.6 to 27.

In one embodiment comprising prandial insulin, the co-polyamino acid/amylin molar ratios are comprised from 10 to 27.

In one embodiment comprising prandial insulin, the co-polyamino acid/amylin molar ratios are comprised from 1.0 to 67.

In one embodiment comprising prandial insulin, the co-polyamino acid/amylin molar ratios are comprised from 1.2 to 45.

In one embodiment comprising prandial insulin, the co-polyamino acid/amylin molar ratios are comprised from 1.3 to 27.

In one embodiment comprising prandial insulin, the co-polyamino acid/amylin molar ratios are comprised from 1.7 to 27.

In one embodiment comprising prandial insulin, the co-polyamino acid/amylin molar ratios are comprised from 2.0 to 27.

In one embodiment comprising prandial insulin, the co-polyamino acid/amylin molar ratios are comprised from 2.3 to 27.

In one embodiment comprising prandial insulin, the co-polyamino acid/amylin molar ratios are comprised from 2.7 to 27.

In one embodiment comprising prandial insulin, the co-polyamino acid/amylin molar ratios are comprised from 3.3 to 27.

In one embodiment comprising prandial insulin, the co-polyamino acid/amylin molar ratios are comprised from 4.7 to 27.

In one embodiment comprising prandial insulin, the co-polyamino acid/amylin molar ratios are comprised from 6.0 to 27.

Moreover, it is particularly advantageous to combine amylin, an amylin receptor agonist or an amylin analogue, in combination or not, with prandial insulin, GLP-1, GLP-1 analogues, GLP-1 receptor agonists (these are commonly referred to as GLP-1 RA). In particular, this helps maximize the effect of insulin and is recommended in some types of diabetes treatment.

In one embodiment, GLP-1, GLP-1 analogues, or GLP-1 RA are considered “fast-acting”. “Fast-acting” refers to GLP-1, GLP-1 analogues, or GLP-1 RA, the apparent elimination half-life of which after subcutaneous injection in humans is less than 8 hours, particularly less than 5 hours, preferably less than 4 hours or even less than 3 hours, as, for example, exenatide and lixisenatide.

In one embodiment, GLP-1, GLP-1 analogues, or GLP-1 RA are chosen among the group consisting of exenatide or Byetta (ASTRA-ZENECA), lixisenatide or Lyxumia® (SANOFI), their analogues or derivatives and their pharmaceutically acceptable salts.

In one embodiment, GLP-1, GLP-1 analogue, or GLP-1 RA is exenatide or Byetta®, its analogues or derivatives and their pharmaceutically acceptable salts.

In one embodiment, GLP-1, GLP-1 analogue, or GLP-1 RA is lixisenatide or Lyxumia®, its analogues or derivatives and their pharmaceutically acceptable salts.

In one embodiment, the concentration of exenatide, its analogues or derivatives and their pharmaceutically acceptable salts is in a range of 0.01 to 1.0 mg per 100 U of insulin.

In one embodiment, the concentration of exenatide, its analogues or derivatives and their pharmaceutically acceptable salts is 0.01 to 0.5 mg per 100 U of insulin.

In one embodiment, the concentration of exenatide, its analogues or derivatives and their pharmaceutically acceptable salts is 0.02 to 0.4 mg per 100 U of insulin.

In one embodiment, the concentration of exenatide, its analogues or derivatives and their pharmaceutically acceptable salts is 0.03 to 0.3 mg per 100 U of insulin.

In one embodiment, the concentration of exenatide, its analogues or derivatives and their pharmaceutically acceptable salts is 0.04 to 0.2 mg per 100 U of insulin.

In one embodiment, the concentration of exenatide, its analogues or derivatives and their pharmaceutically acceptable salts is 0.04 to 0.15 mg per 100 U of insulin.

In one embodiment, the concentration of lixisenatide, its analogues or derivatives and their pharmaceutically acceptable salts is in a range of 0.01 to 1 mg per 100 U of insulin.

In one embodiment, the concentration of lixisenatide, its analogues or derivatives and their pharmaceutically acceptable salts is 0.01 to 0.5 mg per 100 U of insulin.

In one embodiment, the concentration of lixisenatide, its analogues or derivatives and their pharmaceutically acceptable salts is 0.02 to 0.4 mg per 100 U of insulin.

In one embodiment, the concentration of lixisenatide, its analogues or derivatives and their pharmaceutically acceptable salts is 0.03 to 0.3 mg per 100 U of insulin.

In one embodiment, the concentration of lixisenatide, its analogues or derivatives and their pharmaceutically acceptable salts is 0.04 to 0.2 mg per 100 U of insulin.

In one embodiment, the concentration of lixisenatide, its analogues or derivatives and their pharmaceutically acceptable salts is 0.04 to 0.15 mg per 100 U of insulin.

In one embodiment, the compositions according to the invention are produced by mixing solutions of amylin and commercial solutions of GLP-1, GLP-1 analogue or GLP-1 receptor agonist volume ratios in the range of 10/90 to 90/10 in the presence of a co-polyamino acid.

The invention also relates to compositions which further comprise ionic species, the said ionic species improve the stability of the compositions.

The invention also relates to the use of ionic species chosen among the group of anions, cations and/or zwitterions to improve the physicochemical stability of the compositions.

In one embodiment, the ionic species comprise less than 10 carbon atoms.

The said ionic species are chosen among the group of anions, cations and/or zwitterions. Zwitterion refers to a species bearing at least one positive charge and at least one negative charge on two non-adjacent atoms.

The said ionic species are used alone or as a mixture and preferably as a mixture.

In one embodiment, the anions are chosen among organic anions.

In one embodiment the organic anions comprise less than 10 carbon atoms.

In one embodiment, the organic anions are chosen among the group consisting of acetate, citrate and succinate

In one embodiment, the anions are chosen among anions of mineral origin.

In one embodiment, the anions of mineral origin are chosen among the group consisting of sulfates, phosphates and halides, in particular chlorides.

In one embodiment, the cations are chosen among organic cations.

In one embodiment, the organic cations comprise less than 10 carbon atoms.

In one embodiment, the organic cations are chosen among the group consisting of ammoniums, for example 2-Amino-2-(hydroxymethyl) propane-1,3-diol where the amine is in ammonium form.

In one embodiment, cations are chosen among cations of mineral origin.

In one embodiment, the cations of mineral origin are chosen among the group consisting of zinc, in particular Zn2+ and alkali metals, especially Na+ and K+,

In one embodiment, the zwitterions are chosen among zwitterions of organic origin.

In one embodiment, zwitterions of organic origin are chosen among amino acids.

In one embodiment the amino acids are chosen among aliphatic amino acids in the group consisting of glycine, alanine, valine, isoleucine and leucine.

In one embodiment, the amino acids are chosen among cyclic amino acids in the group consisting of proline.

In one embodiment the amino acids are chosen among hydroxylated or sulfur amino acids in the group consisting of cysteine, serine, threonine, and methionine.

In one embodiment, the amino acids are chosen among aromatic amino acids in the group consisting of phenylalanine, tyrosine and tryptophan.

In one embodiment, the amino acids are chosen among amino acids whose carboxyl function of the side chain is amidated in the group consisting of asparagine and glutamine.

In one embodiment, zwitterions of organic origin are chosen among the group consisting of amino acids having an unfilled side chain.

In one embodiment, zwitterions of organic origin are chosen among the group consisting of amino diacids or acidic amino acids.

In one embodiment, the amino diacids are chosen among the group consisting of glutamic acid and aspartic acid, possibly in the form of salts.

In one embodiment, zwitterions of organic origin are chosen among the group consisting of basic or so-called “cationic” amino acids.

In one embodiment, “cationic” amino acids are chosen among arginine, histidine and lysine, especially arginine and lysine.

In particular, zwitterions comprise as many negative charges as positive charges and therefore a nil overall charge at the isoelectric point and/or at a pH ranging from 6 to 8.

Said ionic species are introduced into the compositions in the form of salts. The introduction of these can be in solid form before dissolution in the compositions, or in the form of a solution, in particular of concentrated solution.

For example, mineral-based cations are provided in the form of salts chosen among sodium chloride, zinc chloride, sodium phosphate, sodium sulfate, etc.

For example, anions of organic origin are provided in the form of salts chosen among sodium or potassium citrate, sodium acetate.

For example, amino acids are added in the form of salts chosen among arginine hydrochloride, histidine hydrochloride or in non-salified form, for example histidine or arginine.

In one embodiment, the total molar concentration of ionic species in the composition is greater than or equal to 10 mM.

In one embodiment, the total molar concentration of ionic species in the composition is greater than or equal to 20 mM.

In one embodiment, the total molar concentration of ionic species in the composition is greater than or equal to 30 mM.

In one embodiment, the total molar concentration of ionic species in the composition is greater than or equal to 50 mM.

In one embodiment, the total molar concentration of ionic species in the composition is greater than or equal to 75 mM.

In one embodiment, the total molar concentration of ionic species in the composition is greater than or equal to 100 mM.

In one embodiment, the total molar concentration of ionic species in the composition is greater than or equal to 200 mM.

In one embodiment, the total molar concentration of ionic species in the composition is greater than or equal to 300 mM.

In one embodiment, the total molar concentration of ionic species in the composition is greater than or equal to 500 mM.

In one embodiment, the total molar concentration of ionic species in the composition is greater than or equal to 600 mM.

In one embodiment, the total molar concentration of ionic species in the composition is greater than or equal to 700 mM.

In one embodiment, the total molar concentration of ionic species in the composition is greater than or equal to 800 mM.

In one embodiment, the total molar concentration of ionic species in the composition is greater than or equal to 900 mM.

In one embodiment, the total molar concentration of ionic species in the composition is less than or equal to 1000 mM.

In one embodiment, the total molar concentration of ionic species in the composition is less than or equal to 1500 mM.

In one embodiment, the total molar concentration of ionic species in the composition is less than or equal to 1200 mM.

In one embodiment, the total molar concentration of ionic species in the composition is less than or equal to 1000 mM.

In one embodiment, the total molar concentration of ionic species in the composition is less than or equal to 900 mM.

In one embodiment, the total molar concentration of ionic species in the composition is less than or equal to 800 mM.

In one embodiment, the total molar concentration of ionic species in the composition is less than or equal to 700 mM.

In one embodiment, the total molar concentration of ionic species in the composition is less than or equal to 600 mM.

In one embodiment, the total molar concentration of ionic species in the composition is less than or equal to 500 mM.

In one embodiment, the total molar concentration of ionic species in the composition is less than or equal to 400 mM.

In one embodiment, the total molar concentration of ionic species in the composition is less than or equal to 300 mM.

In one embodiment, the total molar concentration of ionic species in the composition is less than or equal to 200 mM.

In one embodiment, the total molar concentration of ionic species in the composition is less than or equal to 100 mM.

In one embodiment, the total molar concentration of ionic species in the composition is comprised from 10 to 1000 mM.

In one embodiment, the total molar concentration of ionic species in the composition is comprised from 20 to 1000 mM.

In one embodiment, the total molar concentration of ionic species in the composition is comprised from 30 to 1000 mM.

In one embodiment, the total molar concentration of ionic species in the composition is comprised from 50 to 1000 mM.

In one embodiment, the total molar concentration of ionic species in the composition is comprised from 75 to 1000 mM.

In one embodiment, the total molar concentration of ionic species in the composition is comprised from 100 to 1000 mM.

In one embodiment, the total molar concentration of ionic species in the composition is comprised from 200 to 1000 mM.

In one embodiment, the total molar concentration of ionic species in the composition is comprised from 300 to 1000 mM.

In one embodiment, the total molar concentration of ionic species in the composition is comprised from 400 to 1000 mM.

In one embodiment, the total molar concentration of ionic species in the composition is comprised from 500 to 1000 mM.

In one embodiment, the total molar concentration of ionic species in the composition is comprised from 600 to 1000 mM.

In one embodiment, the total molar concentration of ionic species in the composition is comprised from 10 to 900 mM.

In one embodiment, the total molar concentration of ionic species in the composition is comprised from 20 to 900 mM.

In one embodiment, the total molar concentration of ionic species in the composition is comprised from 30 to 900 mM.

In one embodiment, the total molar concentration of ionic species in the composition is comprised from 50 to 900 mM.

In one embodiment, the total molar concentration of ionic species in the composition is comprised from 75 to 900 mM.

In one embodiment, the total molar concentration of ionic species in the composition is comprised from 100 to 900 mM.

In one embodiment, the total molar concentration of ionic species in the composition is comprised from 200 to 900 mM.

In one embodiment, the total molar concentration of ionic species in the composition is comprised from 300 to 900 mM.

In one embodiment, the total molar concentration of ionic species in the composition is comprised from 400 to 900 mM.

In one embodiment, the total molar concentration of ionic species in the composition is comprised from 500 to 900 mM.

In one embodiment, the total molar concentration of ionic species in the composition is comprised from 600 to 900 mM.

In one embodiment, the total molar concentration of ionic species in the composition is comprised from 10 to 900 mM.

In one embodiment, the total molar concentration of ionic species in the composition is comprised from 20 to 800 mM.

In one embodiment, the total molar concentration of ionic species in the composition is comprised from 30 to 800 mM.

In one embodiment, the total molar concentration of ionic species in the composition is comprised from 50 to 800 mM.

In one embodiment, the total molar concentration of ionic species in the composition is comprised from 75 to 800 mM.

In one embodiment, the total molar concentration of ionic species in the composition is comprised from 100 to 800 mM.

In one embodiment, the total molar concentration of ionic species in the composition is comprised from 200 to 800 mM.

In one embodiment, the total molar concentration of ionic species in the composition is comprised from 300 to 800 mM.

In one embodiment, the total molar concentration of ionic species in the composition is comprised from 400 to 800 mM.

In one embodiment, the total molar concentration of ionic species in the composition is comprised from 500 to 800 mM.

In one embodiment, the total molar concentration of ionic species in the composition is comprised from 600 to 800 mM.

In one embodiment, the total molar concentration of ionic species in the composition is comprised from 10 to 700 mM.

In one embodiment, the total molar concentration of ionic species in the composition is comprised from 20 to 700 mM.

In one embodiment, the total molar concentration of ionic species in the composition is comprised from 30 to 700 mM.

In one embodiment, the total molar concentration of ionic species in the composition is comprised from 50 to 700 mM.

In one embodiment, the total molar concentration of ionic species in the composition is comprised from 75 to 700 mM.

In one embodiment, the total molar concentration of ionic species in the composition is comprised from 100 to 700 mM.

In one embodiment, the total molar concentration of ionic species in the composition is comprised from 200 to 700 mM.

In one embodiment, the total molar concentration of ionic species in the composition is comprised from 300 to 700 mM.

In one embodiment, the total molar concentration of ionic species in the composition is comprised from 400 to 700 mM.

In one embodiment, the total molar concentration of ionic species in the composition is comprised from 500 to 700 mM.

In one embodiment, the total molar concentration of ionic species in the composition is comprised from 600 to 700 mM.

In one embodiment, the total molar concentration of ionic species in the composition is comprised from 10 to 600 mM.

In one embodiment, the total molar concentration of ionic species in the composition is comprised from 20 to 600 mM.

In one embodiment, the total molar concentration of ionic species in the composition is comprised from 30 to 600 mM.

In one embodiment, the total molar concentration of ionic species in the composition is comprised from 50 to 600 mM.

In one embodiment, the total molar concentration of ionic species in the composition is comprised from 75 to 600 mM.

In one embodiment, the total molar concentration of ionic species in the composition is comprised from 100 to 600 mM.

In one embodiment, the total molar concentration of ionic species in the composition is comprised from 200 to 600 mM.

In one embodiment, the total molar concentration of ionic species in the composition is comprised from 300 to 600 mM.

In one embodiment, the total molar concentration of ionic species in the composition is comprised from 400 to 600 mM.

In one embodiment, the total molar concentration of ionic species in the composition is comprised from 500 to 600 mM.

In one embodiment, the total molar concentration of ionic species in the composition is comprised from 10 to 500 mM.

In one embodiment, the total molar concentration of ionic species in the composition is comprised from 20 to 500 mM.

In one embodiment, the total molar concentration of ionic species in the composition is comprised from 30 to 500 mM.

In one embodiment, the total molar concentration of ionic species in the composition is comprised from 50 to 500 mM.

In one embodiment, the total molar concentration of ionic species in the composition is comprised from 75 to 500 mM.

In one embodiment, the total molar concentration of ionic species in the composition is comprised from 100 to 500 mM.

In one embodiment, the total molar concentration of ionic species in the composition is comprised from 200 to 500 mM.

In one embodiment, the total molar concentration of ionic species in the composition is comprised from 300 to 500 mM.

In one embodiment, the total molar concentration of ionic species in the composition is comprised from 400 to 500 mM.

In one embodiment, the total molar concentration of ionic species in the composition is comprised from 10 to 400 mM.

In one embodiment, the total molar concentration of ionic species in the composition is comprised from 20 to 400 mM.

In one embodiment, the total molar concentration of ionic species in the composition is comprised from 30 to 400 mM.

In one embodiment, the total molar concentration of ionic species in the composition is comprised from 50 to 400 mM.

In one embodiment, the total molar concentration of ionic species in the composition is comprised from 75 to 400 mM.

In one embodiment, the total molar concentration of ionic species in the composition is comprised from 100 to 400 mM.

In one embodiment, the total molar concentration of ionic species in the composition is comprised from 200 to 400 mM.

In one embodiment, the total molar concentration of ionic species in the composition is comprised from 300 to 400 mM.

In one embodiment, the total molar concentration of ionic species in the composition is comprised from 10 to 300 mM.

In one embodiment, the total molar concentration of ionic species in the composition is comprised from 20 to 300 mM.

In one embodiment, the total molar concentration of ionic species in the composition is comprised from 30 to 300 mM.

In one embodiment, the total molar concentration of ionic species in the composition is comprised from 50 to 300 mM.

In one embodiment, the total molar concentration of ionic species in the composition is comprised from 75 to 300 mM.

In one embodiment, the total molar concentration of ionic species in the composition is comprised from 100 to 300 mM.

In one embodiment, the total molar concentration of ionic species in the composition is comprised from 200 to 300 mM.

In one embodiment, the total molar concentration of ionic species in the composition is comprised from 10 to 200 mM.

In one embodiment, the total molar concentration of ionic species in the composition is comprised from 20 to 200 mM.

In one embodiment, the total molar concentration of ionic species in the composition is comprised from 30 to 200 mM.

In one embodiment, the total molar concentration of ionic species in the composition is comprised from 50 to 200 mM.

In one embodiment, the total molar concentration of ionic species in the composition is comprised from 75 to 200 mM.

In one embodiment, the total molar concentration of ionic species in the composition is comprised from 100 to 200 mM.

In one embodiment, the total molar concentration of ionic species in the composition is comprised from 10 to 100 mM.

In one embodiment, the total molar concentration of ionic species in the composition is comprised from 20 to 100 mM.

In one embodiment, the total molar concentration of ionic species in the composition is comprised from 30 to 100 mM.

In one embodiment, the total molar concentration of ionic species in the composition is comprised from 50 to 100 mM.

In one embodiment, the total molar concentration of ionic species in the composition is comprised from 75 to 100 mM.

In one embodiment, the total molar concentration of ionic species in the composition is comprised from 10 to 75 mM.

In one embodiment, the total molar concentration of ionic species in the composition is comprised from 20 to 75 mM.

In one embodiment, the total molar concentration of ionic species in the composition is comprised from 30 to 75 mM.

In one embodiment, the total molar concentration of ionic species in the composition is comprised from 50 to 75 mM.

In one embodiment, the total molar concentration of ionic species in the composition is comprised from 10 to 50 mM.

In one embodiment, the total molar concentration of ionic species in the composition is comprised from 20 to 50 mM.

In one embodiment, the total molar concentration of ionic species in the composition is comprised from 30 to 50 mM.

In one embodiment, said ionic species are present in a concentration ranging from 5 to 400 mM.

In one embodiment, said ionic species are present in a concentration ranging from 5 to 300 mM.

In one embodiment, said ionic species are present in a concentration ranging from 5 to 200 mM.

In one embodiment, said ionic species are present in a concentration ranging from 5 to 100 mM.

In one embodiment, said ionic species are present in a concentration ranging from 5 to 75 mM.

In one embodiment, said ionic species are present in a concentration ranging from 5 to 50 mM.

In one embodiment, said ionic species are present in a concentration ranging from 5 to 25 mM.

In one embodiment, said ionic species are present in a concentration ranging from 5 to 20 mM.

In one embodiment, said ionic species are present in a concentration ranging from 5 to 10 mM.

In one embodiment, said ionic species are present in a concentration ranging from 10 to 400 mM.

In one embodiment, said ionic species are present in a concentration ranging from 10 to 300 mM.

In one embodiment, said ionic species are present in a concentration ranging from 10 to 200 mM.

In one embodiment, said ionic species are present in a concentration ranging from 10 to 100 mM.

In one embodiment, said ionic species are present in a concentration ranging from 10 to 75 mM.

In one embodiment, said ionic species are present in a concentration ranging from 10 to 50 mM.

In one embodiment, said ionic species are present in a concentration ranging from 10 to 25 mM.

In one embodiment, said ionic species are present in a concentration ranging from 10 to 20 mM.

In one embodiment, said ionic species are present in a concentration ranging from 20 to 300 mM.

In one embodiment, said ionic species are present in a concentration ranging from 20 to 200 mM.

In one embodiment, said ionic species are present in a concentration ranging from 20 to 100 mM.

In one embodiment, said ionic species are present in a concentration ranging from 20 to 75 mM.

In one embodiment, said ionic species are present in a concentration ranging from 20 to 50 mM.

In one embodiment, said ionic species are present in a concentration ranging from 20 to 25 mM.

In one embodiment, said ionic species are present in a concentration ranging from 50 to 300 mM.

In one embodiment, said ionic species are present in a concentration ranging from 50 to 200 mM.

In one embodiment, said ionic species are present in a concentration ranging from 50 to 100 mM.

In one embodiment, said ionic species are present in a concentration ranging from 50 to 75 mM.

In the case of cations of mineral origin and in particular Zn2+, its molar concentration in the composition may be comprised from 0.25 to 20 mM, in particular from 0.25 to 10 mM or from 0.25 to 5 mM.

In one embodiment, the composition comprises zinc.

In one embodiment, the composition comprises from 0.2 to 2 mM of zinc.

In one embodiment, the composition comprises NaCl.

In one embodiment, the composition comprises from 10 to 250 mM of NaCl.

In one embodiment, the composition comprises from 15 to 200 mM of NaCl.

In one embodiment, the composition comprises from 20 to 150 mM of NaCl.

In one embodiment, the composition comprises from 25 to 100 mM of NaCl.

In one embodiment, the compositions according to the invention also comprise zinc salts at a concentration comprising from 0 to 500 μM per 100 U of insulin.

In one embodiment, the compositions according to the invention also comprise zinc salts at a concentration comprising from 0 to 400 μM per 100 U of insulin.

In one embodiment, the compositions according to the invention also comprise zinc salts at a concentration comprising from 0 to 300 μM per 100 U of insulin.

In one embodiment, the compositions according to the invention also comprise zinc salts at a concentration comprising from 0 to 200 μM per 100 U of insulin.

In one embodiment, the compositions according to the invention also comprise zinc salts at a concentration comprising from 0 to 100 μM per 100 U of insulin.

In one embodiment, the compositions according to the invention further comprise buffers.

In one embodiment, the compositions according to the invention comprise buffers at concentrations ranging from 0 to 100 mM.

In one embodiment, the compositions according to the invention comprise buffers at concentrations ranging from 15 to 50 mM.

In one embodiment, the compositions of the invention comprise a buffer chosen among the group consisting of a phosphate buffer, Tris (trishydroxymethylaminomethane) and sodium citrate.

In one embodiment, the buffer is sodium phosphate.

In one embodiment, the buffer is Tris (trishydroxymethylaminomethane).

In one embodiment, the buffer is sodium citrate.

In one embodiment, the compositions according to the invention further comprise preservatives.

In one embodiment, the preservatives are chosen among the group consisting of m-cresol and phenol, alone or in a mixture.

In one embodiment, the concentration of preservatives is comprised from 10 to 50 mM.

In one embodiment, the concentration of preservatives is comprised from 10 to 40 mM.

In one embodiment, the compositions according to the invention further comprise a surfactant.

In one embodiment, the surfactant is chosen among the group consisting of propylene glycol and polysorbate.

The compositions according to the invention may further comprise additives such as tonicity agents.

In one embodiment, the tonicity agents are chosen among the group consisting of glycerol, sodium chloride, mannitol and glycine.

The compositions according to the invention may furthermore comprise any excipients conforming to pharmacopoeia and compatible with the insulins used at usage concentrations.

The invention also relates to a pharmaceutical formulation according to the invention, characterized in that it is obtained by drying and/or lyophilization.

In the case of local and systemic releases, the proposed modes of administration are intravenous, subcutaneous, intradermal or intramuscular.

Transdermal, oral, nasal, vaginal, ocular, oral, and pulmonary routes of administration are also envisaged.

The invention also relates to an implantable or transportable pump comprising a composition according to the invention.

The invention also relates to the use of a composition according to the invention intended to be placed in an implantable or transportable pump.

The invention also relates to single-dose formulations with a pH ranging from 6.0 to 8.0 comprising amylin, an amylin receptor agonist or an amylin analogue and a co-polyamino acid according to the invention.

The invention also relates to single-dose formulations at pH ranging from 6.0 to 8.0 comprising amylin, an amylin receptor agonist or amylin analogue, a co-polyamino acid according to the invention and a GLP-1, a GLP-1 analogue or a GLP-1 RA, as defined above.

The invention also relates to single-dose formulations with a pH ranging from 6.6 to 7.8 comprising amylin, an amylin receptor agonist or an amylin analogue and a co-polyamino acid according to the invention.

The invention also relates to single-dose formulations at pH ranging from 6.6 to 7.8 comprising amylin, an amylin receptor agonist or an amylin analogue, a co-polyamino acid according to the invention and prandial insulin, as defined above.

The invention also relates to single-dose formulations with a pH ranging from 6.6 to 7.6 comprising amylin, an amylin receptor agonist or an amylin analogue and a co-polyamino acid according to the invention.

The invention also relates to single-dose formulations at pH ranging from 6.6 to 7.6 comprising amylin, an amylin receptor agonist or an amylin analogue, a co-polyamino acid according to the invention and prandial insulin, as defined above.

In one embodiment, the single-dose formulations further comprise a co-polyamino acid as defined above.

In one embodiment, the formulations are in the form of an injectable solution.

The preparation of a composition according to the invention has the advantage of being able to be achieved by simple mixing of an aqueous solution of amylin, amylin receptor agonist or amylin analogue, and a co-polyamino acid bearing carboxylate charges and at least one hydrophobic radical according to the invention, in aqueous solution or in freeze-dried form. If necessary, the pH of the preparation is adjusted to a pH ranging from 6 to 8.

The preparation of a composition according to the invention has the advantage of being able to be achieved by simple mixing of an aqueous solution of amylin, amylin receptor agonist or amylin analogue, prandial insulin, and a co-polyamino acid bearing carboxylate charges and at least one hydrophobic radical according to the invention, in aqueous solution or in freeze-dried form. If necessary, the pH of the preparation is adjusted to a pH ranging from 6 to 8.

In one embodiment, the mixture of prandial insulin and co-polyamino acid is concentrated by ultrafiltration.

If necessary, the composition of the mixture is adjusted with excipients such as glycerol, m-cresol, zinc chloride, and polysorbate (Tween) by adding concentrated solutions of these excipients to the mixture. If necessary, the pH of the preparation is adjusted to a pH ranging from 6 to 8.

In one embodiment, the compositions are characterized in that said compositions have greater ThT-measured stability than a reference composition comprising amylin, amylin receptor agonist or amylin analogue but not comprising a co-polyamino acid bearing carboxylate charges and hydrophobic radicals Hy.

In one embodiment, the compositions are characterized in that said compositions have greater ThT-measured stability than a reference composition comprising amylin, amylin receptor agonist or amylin analogue in combination with an insulin but not including a co-polyamino acid bearing carboxylate charges and hydrophobic radicals Hy.

In one embodiment, the compositions are characterized in that said compositions have greater ThT-measured stability than a reference composition comprising amylin, amylin receptor agonist or amylin analogue in combination with a GLP-1, a GLP-1 analogue or a GLP-1 receptor agonist, but not comprising a co-polyamino acid bearing carboxylate charges and hydrophobic radicals Hy.

In one embodiment, the compositions are characterized in that said compositions have greater ThT-measured stability than a reference composition comprising amylin, amylin receptor agonist or amylin analogue in combination with insulin and GLP-1, a GLP-1 analogue or a GLP-1 receptor agonist, but not comprising a co-polyamino acid bearing carboxylate charges and hydrophobic radicals Hy.

The invention also relates to a use of a co-polyamino acid bearing carboxylate charges and hydrophobic radicals Hy for stabilizing a composition comprising amylin, an amylin receptor agonist or an amylin analogue.

The invention also relates to a use of a co-polyamino acid bearing carboxylate charges and hydrophobic radicals Hy for stabilizing a composition comprising amylin, an amylin receptor agonist or an amylin analogue and prandial insulin and optionally a GLP-1, GLP-1 analogue or GLP-1 receptor agonist.

The invention also relates to a method for stabilizing a composition comprising amylin, an amylin receptor agonist or amylin analogue or a method for stabilizing a composition comprising amylin, an agonist at the receptor of the amylin, amylin or an amylin analogue and prandial insulin, and optionally a GLP-1, GLP-1 analogue or GLP-1 receptor agonist.

### DESCRIPTION OF FIGURES

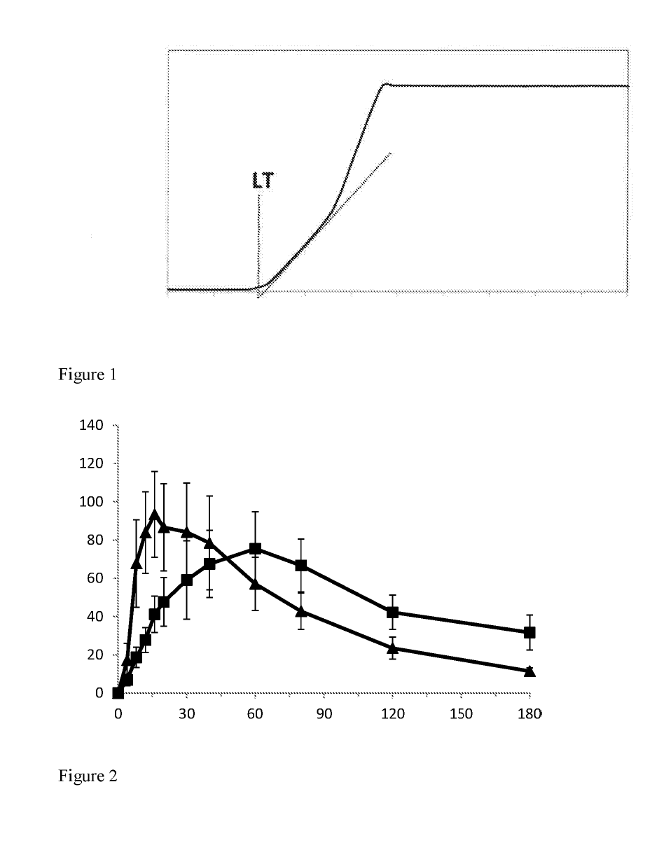

### FIG. 1:

This figure is a graphic representation of latency time determination (LT) by monitoring the fluorescence of Thioflavin T, on a curve showing the fluorescence value (in a.u. arbitrary units) on the y-axis and the time in minutes on the horizontal axis.

### FIG. 2:

Results of pramlintide pharmacokinetics obtained with the compositions described in examples CA1/CA2 and CA3 are shown in FIG. 2. The analysis of these profiles indicates that the composition of example CA3 comprising co-polyamino acid BB15, 100 IU/mL insulin and 0.6 mg/mL pramlintide (curve plotted with the squares corresponding to example CA3), makes it possible to obtain a pramlintide absorption which is slower than that of the composition of the double-injection example comprising only pramlintide and insulin (curve drawn with the triangles corresponding to the CA1/CA2) double injection example).

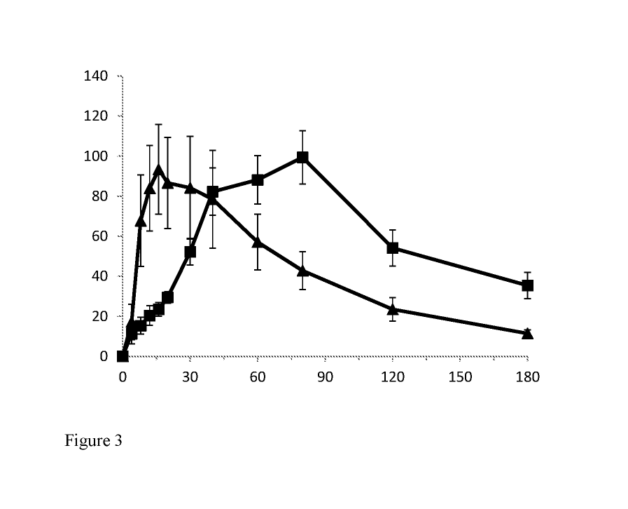

### FIG. 3:

Results of pramlintide pharmacokinetics obtained with the compositions described in examples CA1/CA2 and CA4 are shown in FIG. 3. The analysis of these profiles indicates that the composition of example CA4 comprising co-polyamino acid AB24, 100 IU/mL of insulin and 0.6 μg/ml of pramlintide (curve plotted with the squares corresponding to example CA4) makes it possible to obtain a pramlintide absorption which is slower than that of the composition of the double-injection example comprising only pramlintide and insulin (curve drawn with the triangles corresponding to the double injection example CA1/CA2).

The following examples illustrate the invention in a non-limiting manner.

### Part A

AA: Synthesis of Intermediate Hydrophobic Hy Compounds to Obtain the -Hy Radicals wherein p=1

The hydrophobic intermediate compounds are represented in the following table by the corresponding hydrophobic molecule before co-polyamino acid grafting.

TABLE 1A
list and structures of the hydrophobic molecules synthesized according to the
invention.

| N° | HYDROPHOBIC INTERMEDIATE COMPOUNDS |
|---|---|
| AA1 | 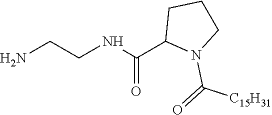 |
| AA2 | 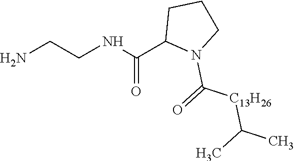 |
| AA3 | 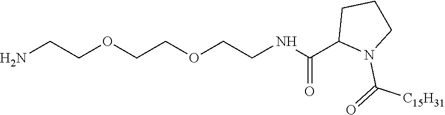 |
| AA4 | 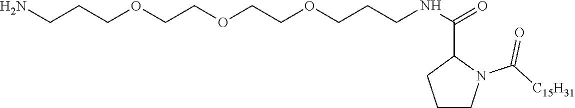 |
| AA5 | 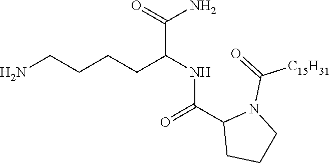 |
| AA6 | 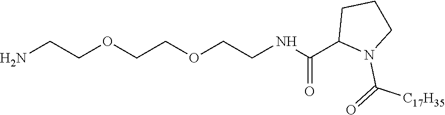 |
| AA7 | 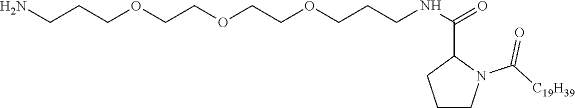 |
| AA8 | 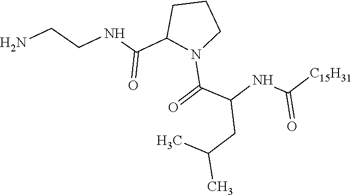 |
| AA9 | 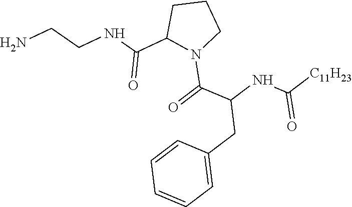 |
| AA10 | 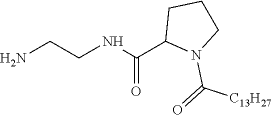 |
| AA11 | 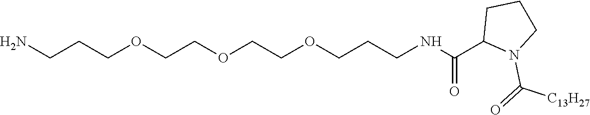 |
| AA12 | 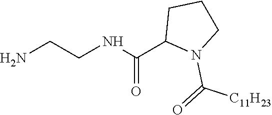 |
| AA13 | 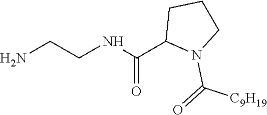 |
| AA14 | 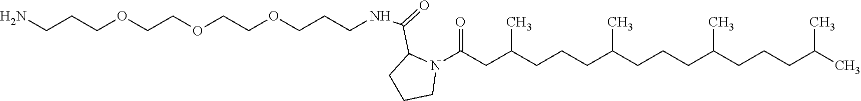 [SMILES: CC(C)CCCC(C)CCCC(C)CCCC(C)CC(=O)N1CCCC1C(=O)NCCCOCCOCCOCCCN] |
| AA15 | 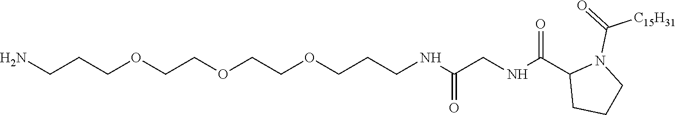 |
| AA16 | 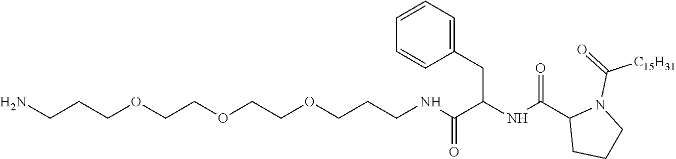 |
| AA17 | 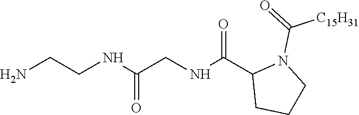 |
| AA18 | 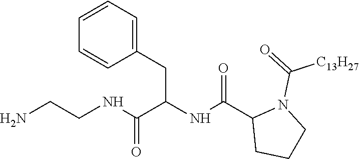 |

### EXAMPLE AA1

### Molecule AA1

### Molecule A1: Product Obtained by the Reaction Between Palmitoyl Chloride and L-proline.

To a solution of L-proline (10.6 g, 92.1 mmol) in aqueous sodium hydroxide 1

N (230 mL; 230 mmol) a solution of palmitoyl chloride (23.0 g., 83.7 mmol) in acetone (167 mL) is added dropwise for 90 minutes. After stirring for 14 hours at room temperature, the heterogeneous mixture is cooled to 0° C., then filtered through a sintered frit to give a white solid which is washed with water (2×100 mL), then diisopropyl ether (100 mL). The solid is dried under reduced pressure. The solid is then dissolved under reflux in 200 mL of water, then 8 mL of a 37% hydrochloric acid solution are added to obtain a pH=1. The opalescent reaction medium is then cooled to 0° C. The precipitate obtained is filtered through a sintered frit , then washed with water (5×50 mL) until filtrates of physiological pH ranging from 6.0 to 8.0 are obtained, then dried in an oven at 50° C. under vacuum overnight. The product is purified by recrystallization in diisopropyl ether. A white solid is obtained.

Yield: 22.7 g (77%).

NMR ¹H (CDCl₃, ppm): 0.88 (3H); 1.19-1.45 (24H); 1.58-1.74 (2H); 1.88-2.14 (3H); 2.15-2.54 (3H); 3.47 (1H); 3.58 (1H); 4.41 (0.1H); 4.61 (0.9H) 6.60-8.60 (1H).

### Molecule A2: Product Obtained by Reaction Between Molecule A1 and N-Boc-ethylenediamine.

N,N-diisopropylethylamine (DIPEA) (68.8 g, 532.3 mmol), 1-hydroxybenzotriazole (HOBt) (37.1 g, 274.6 mmol), then N-(3-dimethylaminopropyl)-N′-ethylcarbodiimide (EDC) (53.1 g, 277.0 mmol) are successively added at room temperature to a solution of molecule A1 (75.1 g, 212.4 mmol) in 1500 mL of chloroform. After stirring for 15 minutes at room temperature, a solution of N-Boc-ethylenediamine (BocEDA) (37.6 g, 234.7 mmol) in 35 ml of chloroform is added. After stirring for 18 hours at room temperature, a 0.1 N solution of HCl (2.1 L), then a saturated solution of NaCl (1 L) are added. The phases are separated then the organic phase is washed successively with a solution of 0.1 N HO/saturated NaCl (2.1 L/1 L), a saturated solution of NaCl (2 L), a saturated NaHCO₃ (2 L) solution, then a saturated NaCl (2 L) solution. The organic phase is dried over anhydrous sodium sulphate, filtered, then concentrated under reduced pressure. The obtained solid is purified by trituration in diisopropyl ether (3×400 mL), to yield a solid after drying under vacuum at 40° C.

Yield: 90.4 g (86%).

NMR ¹H (CDCl₃, ppm): 0.88 (3H); 1.20-1.37 (24H); 1.44 (9H); 1.54-1.70 (2H); 1.79-1.92 (1H); 1.92-2.04 (1H); 2.03-2.17 (1H); 2.17-2.44 (3H); 3.14-3.36 (4H); 3.43 (1H); 3.56 (1H); 4.29 (0.1H); 4.51 (0.9 H); 4.82 (0.1H); 5.02 (0.9H); 6.84 (0.1H); 7.22 (0.9H).

### Molecule AA1

A 4 N solution of hydrochloric acid in dioxane (100 ml, 400 mmol) is added dropwise and at 0° C. to a solution of molecule A2 (20.1 g, 40.5 mmol) in 330 ml of dichloromethane. After stirring for 3 h30 at room temperature, the solution is concentrated under reduced pressure. The residue is purified by flash chromatography (methanol, dichloromethane) to yield a white solid of molecule AA1 in the form of a hydrochloride salt.

Yield: 16.3 g (93%).

NMR ¹H (CDCl₃, ppm): 0.88 (3H); 1.07-1.40 (24H); 1.49-1.63 (2H); 1.77-2.18 (4H); 2.18-2.45 (2H); 3.14-3.32 (2H); 3.42-3.63 (2H); 3.63-3.84 (2H); 4.37 (0.1H); 4.48 (0.9H); 6.81-8.81 (4H).

LC/MS (ESI): 396.5; (calculated ([M+H]+): 396.4).

### EXAMPLE AA2

### Molecule AA2

Molecule A3: 15-methylhexadecan-1-ol.

Magnesium in chips (9.46 g, 389 mmol) is introduced into a three-neck flask under argon. The magnesium is covered with anhydrous THF (40 mL), and a few drops of 1-bromo-3-methylbutane are added at room temperature to initiate the reaction. After the observation of an exotherm and a slight turbidity of the medium, the rest of the 1-bromo-3-methylbutane (53.87 g, 357 mmol) is added dropwise over 90 minutes while the temperature of the medium remains stable between 50 and 60° C. The reaction medium is then heated at 70° C. for 2 hours.

In a three-necked under argon, a solution of CuCl (482 mg, 4.86 mmol) dissolved in NMP (62 ml) at 0° C. is added dropwise into a solution of 12-bromo-1-dodecanol (43 g, 162.1 mmol) in THF (60 mL). To this solution is then added dropwise, the hot organomagnesium solution, freshly prepared in order to maintain the temperature of the medium below 20° C. The mixture is then stirred at room temperature for 16 hours. The medium is cooled to 0° C. and the reaction is stopped by addition of a 1 N HCl aqueous solution to pH 1 and the medium is extracted with ethyl acetate. After washing the organic phase with saturated NaCl solution and drying over Na₂SO₄, the solution is filtered and concentrated under vacuum to produce an oil. After purification by DCVC on silica gel (cyclohexane, ethyl acetate), an oil which crystallizes at room temperature is obtained.

Yield: 32.8 g (74%)

NMR ¹H (CDCl₃, ppm): 0.87 (6H); 1.14 (2H); 1.20-1.35 (22H); 1.50-1.55 (3H); 3.64 (2H).

Molecule A4: 15-methylhexadecanoic Acid.

Small portions of potassium permanganate (38.2 g, 241.5 mmol)are added to a solution of molecule A3 (20.65 g, 80.5 mmol) and tetrabutylammonium bromide (14.02 g, 42.5 mmol) in a mixture of acetic acid/dichloroethane/water (124/400/320 ml) at room temperature. After stirring at reflux for 5 hours and returning to room temperature, the medium is acidified to pH 1 by progressive addition of 5N HCl. Na₂SO₃ (44.6 g, 354.3 mmol) is then gradually added until the medium is faded. The aqueous phase is extracted with dichloromethane, and the combined organic phases are dried over Na₂SO₄, filtered and concentrated under vacuum. After purification by chromatography on silica gel (cyclohexane, ethyl acetate, acetic acid), a white solid is obtained.

Yield: 19.1 g (quantitative)

NMR ¹H (CDCl₃, ppm): 0.87 (6H); 1.14 (2H); 1.22-1.38 (20H); 1.51 (1H); 1.63 (2H); 2.35 (2H).

### Molecule A5: Product Obtained by Reaction Between the Molecule A4 and L-Proline.

Dicyclohexyl carbodiimide (DCC) (8.01 g, 38.8 mmol) and N-hydroxysuccinimide (NHS) (4.47 g, 38.8 mmol) are successively added to a solution of molecule A4 (10 g, 37 mmol) in THF (360 ml) at 0° C. After stirring for 17 hours at room temperature, the medium is cooled to 0° C. for 20 minutes, filtered through a sintered frit. L-Proline (4 g, 37.7 mmol), triethylamine (34 mL) and water (30 mL) are added to the filtrate. After stirring for 20 h at room temperature, the medium is treated with a 1N HCl aqueous solution to pH 1. The aqueous phase is extracted with dichloromethane (2×125 mL). The combined organic phases are washed with an aqueous solution of 1 N HCl (2×100 mL), water (100 mL), then a saturated aqueous solution of NaCl (100 mL). After drying over Na₂SO₄, the organic phase is filtered, concentrated under vacuum, and the residue is purified by chromatography on silica gel (cyclohexane, ethyl acetate, acetic acid)

Yield: 9.2 g (72%)

NMR ¹H (CDCl₃, ppm): 0.86 (6H); 1.14 (2H); 1.22-1.38 (20H); 1.50 (1H); 1.67 (2H); 1.95-2.10 (3H); 2.34 (2H); 2.49 (1H); 3.47 (1H); 3.56 (1H); 4.61 (1H).

LC/MS (ESI): 368.3; (calculated ([M+H]+): 368.6).

### Molecule A6: Product Obtained by Reaction Between Molecule A5 and N-Boc-ethylenediamine.

Triethylamine (TEA) (5.23 mL) and 2-(1H-benzotriazol-1-yl)-1,1,3,3-tetramethyluronium tetrafluoroborate (TBTU) at room temperature are added to a solution of molecule A5 (9.22 g, 25.08 mmol) in a THF/DMF mixture (200/50 mL). After 10 minutes of stirring, BocEDA (4.42 g, 27.6 mmol) is added. After stirring at room temperature for 17 h, the mixture is diluted with water (300 mL) at 0° C. and stirred cold for 20 minutes. The precipitate formed is filtered through a sintered frit and the filtrate is extracted with ethyl acetate. The combined organic phases are washed with saturated NaHCO₃, solution, dried over Na₂SO₄, filtered, concentrated under vacuum and the residue purified by flash chromatography (ethyl acetate, methanol).

Yield: 6.9 g (54%)

NMR ¹H (CDCl₃, ppm): 0.86 (6H); 1.15 (2H); 1.22-1.38 (20H); 1.43 (9H); 1.50 (1H); 1.64 (4H); 1.85 (1H); 1.95 (1H); 2.10 (1H); 2.31 (2H); 3.20-3.35 (3H); 3.45 (1H); 3.56 (1H); 4.51 (1H); 5.05 (1H); 7.24 (1H).

LC/MS (ESI): 510.6; (calculated ([M+H]+): 510.8).

### Molecule AA2

A 4 N HCl solution in dioxane (13 mL) is added to a solution of molecule A6 (5.3 g, 10.40 mmol) in dichloromethane (50 mL) at 0° C. After stirring for 5 hours at 0° C., the medium is concentrated under vacuum, returned to water and freeze-dried to give a white solid of molecule AA2 in the form of hydrochloride salt.

Yield: 4.6 g (99%)

NMR ¹H (D₂O, ppm): 0.91 (6H); 1.22 (2H); 1.22-1.50 (20H); 1.63 (3H); 1.98 (1H); 2.10 (2H); 2.26 (1H); 2.39 (1H); 2.43 (1H); 3.22 (2H); 3.45-3.60 (3H); 3.78 (1H); 4.42 (1H).

LC/MS (ESI): 410.4; (calculated ([M+H]+): 410.7).

### EXAMPLE AA3

### Molecule AA3

Molecule A7: Product Obtained by the Reaction Between Molecule A1 and Boc-tri(ethylene glycol)diamine.

By a process similar to that used in the preparation of molecule A2 applied to molecule A1 (4.0 g, 11.3 mmol) and Boc-tri (ethylene glycol) diamine (3.1 g, 12.4 mmol), a colorless oil is obtained after purification by flash chromatography (methanol, toluene).

Yield: 5.5 g (84%).

NMR ¹H (CDCl₃, ppm): 0.88 (3H); 1.09-1.39 (24H); 1.44 (9H); 1.64 (2H); 1.79-2.01 (2H); 2.06-2.43 (4H); 3.23-3.68 (14H); 4.33 (0.2H); 4.56 (0.8H); 5.25 (1H); 6.49 (0.2H); 7.13-7.50 (0.8H).

### Molecule AA3

By a process similar to that used in the preparation of molecule AA1 applied to molecule A7 (5.5 g, 9.4 mmol), a white solid of molecule AA3 in the form of a hydrochloride salt is obtained after purification by flash chromatography (methanol, dichloromethane).

Yield: 4.3 g (92%).

NMR ¹H (DMSO-d6, ppm): 0.85 (3H); 1.08-1.40 (24H); 1.40-1.52 (2H); 1.71-2.02 (4H); 2.02-2.31 (2H); 2.90-2.98 (2H); 3.15-3.47 (5H); 3.50-3.66 (7H); 4.24 (0.6H); 4.32 (0.4H); 7.83 (0.6H); 7.95 (3H); 8.17 (0.4H).

LC/MS (ESI): 484.6; (calculated ([M+H]+): 484.4).

### EXAMPLE AA4

### Molecule AA4

Molecule A8: Product Obtained by Reaction Between Molecule A1 and Boc-1-amino-4,7,10-trioxa-13-tridecane amine.

By a process similar to that used in the preparation of molecule A2 applied to molecule A1 (4.5 g, 12.7 mmol) and to Boc-1-amino-4,7,10-trioxa-13-tridecane amine (4.5 g, 14.0 mmol), a yellow oil is obtained after purification by flash chromatography (methanol, dichloromethane).

Yield: 7.7 g (92%).

NMR ¹H (CDCl₃, ppm): 0.88 (3H); 1.22-1.37 (24H); 1.44 (9H); 1.59-1.67 (2H); 1.67-2.00 (6H); 2.06-2.45 (4H); 3.18-3.76 (18H); 4.28 (0.2H); 4.52 (0.8H); 4.69-5.04 (1H); 6.77 (0.2H); 7.20 (0.8H).

### Molecule AA4

By a process similar to that used in the preparation of molecule AA1 applied to molecule A8 (7.7 g, 11.8 mmol), a yellow oil is obtained after purification by flash chromatography (methanol, dichloromethane). A co-evaporation with diisopropyl ether facilitates the obtention of the AA4 molecule in the form of a hydrochloride salt as a white solid which is dried under vacuum at 50° C.

Yield: 5.4 g (76%).

NMR ¹H (CDCl₃, ppm): 0.88 (3H); 1.08-1.40 (24H); 1.49-1.65 (2H); 1.76-2.39 (10H); 3.07-3.28 (3H); 3.34-3.80 (15H); 4.34 (0.05H); 4.64 (0.95H); 7.35 (0.05H); 7.66-8.58 (3.95H).

LC/MS (ESI): 556.7; (calculated ([M+H]+): 556.5).

### EXAMPLE AA5

### Molecule AA5

Molecule A9: Product Obtained by Reaction between Molecule A1 and the Methyl Ester of N-Boc-L-lysine.

By a process similar to that used for the preparation of molecule A2 applied to molecule A1 (4 g, 11.3 mmol) and to the methyl ester of N-Boc-L-lysine (3.2 g, 12.4 mmol), a colorless oil is obtained after purification by flash chromatography (methanol, dichloromethane).

Yield: 4.9 g (73%).

NMR ¹H (CDCl₃, ppm): 0.88 (3H); 0.99-1.54 (37H); 1.54-1.75 (3H); 1.75-2.04 (3H); 2.04-2.41 (4H); 2.94-3.19 (2H); 3.19-3.81 (5H); 4.28-4.64 (2H); 4.94 (1H); 6.45 (0.1H); 7.36 (0.9H).

LC/MS (ESI): 596.7; (calculated ([M+H]+): 596.5).

Molecule A10: Product Obtained by Treatment of Molecule A9 with Ammonia.

320 mL of a 7 N ammonia solution in methanol are added to a suspension of molecule A9 (4.9 g, 8.2 mmol) in 10 mL of methanol. After stirring for 19 hours at room temperature in a closed atmosphere, an additional 100 ml of ammonia solution are added. After stirring for 24 hours at room temperature in a closed atmosphere, the reaction medium is concentrated under reduced pressure. The residue is purified by trituration in refluxing diisopropyl ether (100 mL), to produce a white solid which is dried under vacuum at 50° C.

Yield: 4.1 g (85%).

NMR ¹H (CDCl₃, ppm): 0.88 (3H); 1.06-1.57 (37H); 1.57-1.79 (3H); 1.88-2.41 (7H); 3.09 (2H); 3.49 (1H); 3.62 (1H); 4.34 (1H); 4.51 (1H); 4.69-4.81 (1H); 5.43 (0.95H); 5.57 (0.05H); 6.25 (0.05H); 6.52 (0.95H); 6.83 (0.05H); 7.11 (0.95H).

### Molecule AA5

By a process similar to that used in the preparation of molecule AA1 applied to molecule A10 (388 mg, 0.67 mmol), a white solid of molecule AA5 in the form of a hydrochloride salt is obtained after purification by trituration in diisopropyl ether.

Yield: 292 mg (85%).

NMR ¹H (DMSO-d6, ppm): 0.85 (3H); 1.06-2.34 (38H); 2.61-2.81 (2H); 3.29-3.68 (2H); 4.05-4.17 (1.7H); 4.42 (0.3H); 7.00 (1H); 7.16 (0.7H); 7.43 (0.3H); 7.73-8.04 (3.7H); 8.16 (0.3H).

LC/MS (ESI): 481.6; (calculated ([M+H]+): 481.4).

### EXAMPLE AA6

### Molecule AA6

Molecule A11: Product Obtained by the Reaction Between stearoyl chloride and L-proline.

By a process similar to that used in the preparation of molecule A1 applied to L-proline (5.0 g, 43.4 mmol) and to stearoyl chloride (12.0 g, 39.6 mmol), a white solid is obtained after purification by flash chromatography (methanol, dichloromethane).

Yield: 5.37 g (36%)

NMR ¹H (CDCl₃, ppm): 0.88 (3H); 1.26-1.37 (28H); 1.64-1.70 (2H); 1.88-2.10 (3H); 2.36 (2H); 2.54-2.58 (1H); 3.46 (1H); 3.56 (1H); 4.62 (1H).

LC/MS (ESI): 382.6; (calculated ([M+H]+): 382.3).

Molecule A12: Product Obtained by Reaction Between Molecule A11 and Boc-tri(ethylene glycol)diamine.

By a process similar to that used in the preparation of molecule A6 applied to molecule A11 (33.81 g, 88.6 mmol) and Boc-tri (ethylene glycol) diamine (26.4 g, 106.3 mmol) in THF using DIPEA instead of TEA, a white solid is obtained after purification by flash chromatography (ethyl acetate, methanol).

Yield: 43.3 g (80%)

NMR ¹H (CDCl₃, ppm): 0.87 (3H); 1.24 (30H); 1.43 (9H); 1.61 (2H); 1.82 (1H); 1.96 (1H); 2.25-2.45 (2H); 3.25-3.65 (14H); 4.30 (0.15H); 4.53 (0.85H); 5.25 (1H); 6.43 (0.15H); 7.25 (0.85H).

LC/MS (ESI): 612.6; (calculated ([M+H]+): 612.9).

### Molecule AA6

By a process similar to that used in the preparation of molecule AA2 applied to molecule A12 (43 g, 70.3 mmol), the residue obtained after concentration under vacuum is triturated in acetonitrile. The suspension is filtered, and the solid is washed with acetonitrile then with acetone. After drying under vacuum, a white solid of molecule AA6 in the form of a hydrochloride salt is obtained.

Yield: 31.2 g (81%)

NMR ¹H (DMSO-d₆, ppm): 0.85 (3H); 1.23 (28H); 1.45 (2H); 1.70-2.05 (4H); 2.13 (1H); 2.24 (1H); 2.95 (2H); 3.10-3.25 (2H); 3.30-3.65 (10H); 4.20-4.45 (1H); 7.85-8.25 (4H).

LC/MS (ESI): 512.4; (calculated ([M+H]+): 512.8).

### EXAMPLE AA7

### Molecule AA7

Molecule A13: Product Obtained by Reaction Between arachidonic acid and L-proline.

By a process similar to that used in the preparation of molecule A5 applied to arachidic acid (15.51 g, 49.63 mmol) and L-proline (6 g, 52.11 mmol) using DIPEA in place of TEA, a white solid is obtained after purification by chromatographic column on silica gel (cyclohexane, ethyl acetate, acetic acid).

Yield: 12.9 g (63%)

NMR ¹H (CDCl₃, ppm): 0.88 (3H); 1.28 (34H); 1.66 (2H); 1.95-2.15 (2H); 2.34 (2H); 2.45 (1H); 3.47 (1H); 3.56 (1H); 4.60 (1H).

LC/MS (ESI): 410.4; (calculated ([M+H]+): 410.6).

Molecule A14: Product Obtained by Reaction Between Molecule A13 and Boc-1-amino-4,7,10-trioxa-13-tridecane amine.

By a process similar to that used in the preparation of molecule A12 applied to molecule A13 (10.96 g, 26.75 mmol) and Boc-1-amino-4,7,10-trioxa-13-tridecane (10.29 g, 32.11 mmol), a solid is obtained after purification by chromatographic column on silica gel (cyclohexane, ethyl acetate, methanol).

Yield: 14.2 g (75%)

NMR ¹H (CDCl₃, ppm): 0.88 (3H); 1.24 (32H); 1.43 (9H); 1.61 (2H); 1.80 (1H); 1.96 (1H); 2.10-2.45 (4H); 3.20-3.75 (18H); 4.30 (0.20H); 4.55 (0.80H); 5.03 (1H); 6.75 (0.20H); 7.20 (0.80H).

LC/MS (ESI): 712.8; (calculated ([M+H]⁺): 713.1).

### Molecule AA7

After a process similar to that used for the preparation of molecule AA2 applied to molecule A14 (14.25 g, 20.01 mmol), the residue obtained after concentration under vacuum of the reaction medium is dissolved in methanol and evaporated under reduced pressure, the process being repeated 4 times to yield a white solid of molecule AA7 in the form of a hydrochloride salt.

Yield: 12.7g (98%)

NMR ¹H (DMSO-d₆, ppm): 0.85 (3H); 1.23 (32H); 1.45 (2H); 1.64 (2H); 1.70-2.05 (6H); 2.10-2.30 (2H); 2.82 (2H); 3.08 (2H); 3.30-3.60 (15H); 4.15-4.30 (1H); 7.73-8.13 (4H).

LC/MS (ESI): 612.7; (calculated ([M+H]+): 612.9).

### EXAMPLE AA8

### Molecule AA8

Molecule A15: Product Obtained by the Reaction Between L-leucine and palmitoyl chloride.

By a process similar to that used in the preparation of molecule A1 applied to L-leucine (15.0 g, 114.4 mmol) and to palmitoyl chloride (34.5 g, 125 mmol), a white solid is obtained by trituration in diisopropyl ether.

Yield: 13.0 g (31%)

NMR ¹H (CDCl₃, ppm): 0.88 (3H); 0.96 (6H); 1.16-1.35 (24H); 1.55-1.77 (5H); 2.23 (2H); 4.55-4.60 (1H); 5.88 (1H).

Molecule A16: Product Obtained by the Reaction Between Molecule A15 and the methyl ester of L-proline

By a process similar to that used for the preparation of molecule A2 applied to molecule A15 (6.00 g, 16.2 mmol) and to the methyl ester of L-proline (3.23 g, 19.5 mmol), a slightly yellow oil is obtained after purification by flash chromatography (methanol, dichloromethane).

Yield: 5.8 g (74%)

NMR ¹H (CDCl₃, ppm): 0.83-1.00 (9H); 1.18-1.32 (24H); 1.40-1.73 (5H); 1.84-2.33 (6H); 3.47-3.89 (2H); 3.70 (1.14H); 3.71 (1.21H); 3.74 (0.53H); 3.76 (0.12H); 4.40-4.56 (1H); 4.63-4.67 (0.04H); 4.84 (0.38); 4.90 (0.40); 5.06 (0.18); 5.99 (0.18H); 6.08-6.21 (0.82).

LC/MS (ESI): 481.6; (calculated ([M+H]⁺): 481.4).

Molecule A17: Product Obtained by the Saponification of the methyl ester of Molecule A16.

Sodium hydroxide 1 N (13.5 mL, 13.5 mmol) is added to a solution of molecule A16 (5.8 g, 12.06 mmol) in 30 mL of methanol. After stirring for 20 h at room temperature, the solution is diluted with water, then acidified with 20 mL of 1N hydrochloric acid at 0° C. The precipitate is filtered, then rinsed with water (50 ml) before being solubilized in 50 ml of dichloromethane. The organic phase is dried over Na₂SO₄, filtered, then concentrated under reduced pressure to yield a colorless oil.

Yield: 4.5 g (80%)

NMR ¹H (CDCl₃, ppm): 0.85-0.99 (9H); 1.14-1.41 (24H); 1.43-1.72 (5H); 1.87-2.47 (7H); 3.48-3.55 (0.6H); 3.56-3.62 (0.4H); 3.83-3.90 (0.4H); 3.90-3.96 (0.6H); 4.52-4.56 (0.6H); 4.56-4.59 (0.4H); 4.80-4.86 (0.4H); 4.86-4.91 (0.6H); 6.05 (0.4H); 6.11 (0.6H).

LC/MS (ESI): 467.6; (calculated ([M+H]+): 467.4).

### Molecule A18: Product Obtained by Reaction Between N-Boc-ethylenediamine and Molecule A17.

By a process similar to that used for the preparation of molecule A2 applied to molecule A17 (4.5 g, 9.64 mmol) and to BocEDA (1.70 g, 10.61 mmol), a colorless oil is obtained after purification by flash chromatography (methanol, dichloromethane).

Yield: 2.0 g (34%)

NMR ¹H (CDCl₃, ppm): 0.83-0.99 (9H); 1.19-1.32 (24H); 1.44 (9H); 1.48-2.37 (14H); 3.09-3.99 (4H); 4.28-5.01 (2H); 5.64-6.04 (1H); 6.87-7.06 (1H).

LC/MS (ESI): 609.7; (calculated ([M+H]+): 609.5).

### Molecule AA8

By a process similar to that used in the preparation of molecule AA1 applied to molecule A18 (2 g, 3.28 mmol), a white solid of molecule AA8 in the form of a hydrochloride salt is obtained after purification by flash chromatography (methanol, dichloromethane).

Yield: 1.5 g (90%)

NMR ¹H (CDCl₃, ppm): 0.83-1.00 (9H); 1.18-1.32 (24H); 1.37-1.77 (5H); 1.93-2.41 (6H); 3.07-3.97 (6H); 4.44-4.77 (2H); 7.66-8.21 (2H).

LC/MS (ESI): 509.6; (calculated ([M+H]+): 509.4).

### EXAMPLE AA9

### Molecule AA9

### Molecule A19: Product Obtained by the Reaction Between Lauric Acid and L-phenylalanine.

By a process similar to that used for the preparation of molecule A5 applied to lauric acid (8.10 g, 40.45 mmol) and L-phenylalanine (7 g, 42.38 mmol), a white solid is obtained.

Yield: 12.7g (98%)

NMR ¹H (DMSO-d₆, ppm): 0.86 (3H); 1.10-1.30 (16H); 1.36 (2H); 2.02 (2H); 2.82 (1H); 3.05 (1H); 4.42 (1H); 7.15-7.30 (5H); 8.05 (1H); 12.61 (1H).

LC/MS (ESI): 348.2; (calculated ([M+H]+): 348.5).

Molecule A20: Product Obtained by the Reaction Between Molecule A19 and L-proline methyl ester hydrochloride Salt.

By a process similar to that used in the preparation of molecule A6 applied to molecule A19 (9.98 g, 28.72 mmol) and to L-proline methyl ester hydrochloride salt (5.23 g, 31.59 mmol), a colorless oil is obtained after purification by chromatographic column on silica gel (cyclohexane, ethyl acetate).

Yield: 5.75 g (44%)

NMR ¹H (CDCl₃, ppm): 0.88 (3H); 1.10-1.30 (16H); 1.50-1.75 (3H); 1.80-2.02 (3H); 2.17 (2H); 2.65 (0.5H); 2.95 (1H); 3.05-3.20 (1.5H); 3.50-3.65 (1H); 3.75 (3H); 4.29 (0.5H); 4.46 (0.5H); 4.70 (0.1H); 4.95 (0.9H); 6.20-6.30 (1H); 7.15-7.30 (5H).

LC/MS (ESI): 459.2; (calculated ([M+H]+): 459.6).

Molecule A21: Product Obtained by saponification of Molecule A20.

Lithium hydroxide (LiOH) (600.49 mg, 25.07 mmol) is added to a solution of molecule A20 (5.75 g, 12.54 mmol) in a THF/methanol/water mixture (40/40/40 mL) at 0° C., then the mixture is stirred at room temperature for 20 hours. After evaporation of the organic solvents under vacuum, the aqueous solution is diluted in water, acidified with an 1N HCl aqueous solution to a pH of 1. The product is then extracted with ethyl acetate. The combined organic phases are washed with a saturated aqueous NaCl solution, dried over Na₂SO₄, filtered and concentrated under reduced pressure to yield a colorless oil.

Yield: 5.7 g (quantitative)

NMR ¹H (CDCl₃, ppm): 0.88 (3H); 1.10-1.30 (16H); 1.50-1.80 (3H); 1.67-2.02 (2H); 2.20 (2H); 2.25 (0.4H); 2.60 (0.6H); 2.85-3.10 (2.6H); 3.55-3.65 (1.4H);; 4.35 (0.6H); 4.55 (0.4H); 4.94 (1H); 6.28 (0.4H); 6.38 (0.6H); 7.20-7.30 (5H).

LC/MS (ESI): 445.2; (calculated ([M+H]+): 445.6).

### Molecule A22: Product Obtained by Reaction Between N-Boc-ethylenediamine and Molecule A21.

By a process similar to that used in the preparation of molecule A6 applied to molecule A21 (5.67 g, 12.75 mmol) and BocEDA (2.25 g, 14.03 mmol), a colorless oil is obtained after purification by chromatography column on silica gel (dichloromethane, methanol).

Yield: 5.7 g (76%)

NMR ¹H (CDCl₃, ppm): 0.88 (3H); 1.25 (16H); 1.43 (9H); 1.58 (2.6H); 1.75-1.95 (1.4H); 2.15-2.30 (3H); 2.64 (0.5H); 2.95-3.10 (2.5H); 3.20-3.40 (4H); 3.45 (0.5H); 3.55 (0.2H); 3.66 (1H); 4.44 (1H); 4.50 (0.2H); 4.60 (0.6H); 4.99 (0.7H); 5.54 (0.5H); 5.95 (0.2H); 6.17 (1H); 6.60 (0.5H); 7.07 (0.5H); 7.20-7.40 (5H).

LC/MS (ESI): 587.4; (calculated ([M+H]+): 587.8).

### Molecule AA9

Following a process similar to that used for the preparation of molecule AA2 applied to molecule A22 (5.66 g, 9.65 mmol), the residue obtained after concentration of the reaction medium under vacuum is dissolved in methanol and evaporated under reduced pressure; the process being repeated 4 times to produce a white foam of molecule AA9 in the form of hydrochloride salt.

Yield: 4.9 g (97%)

NMR ¹H (DMSO-d₆, 120° C., ppm): 0.89 (3H); 1.26 (16H); 1.43 (2H); 1.68 (0.6H); 1.75-2.00 (3H); 2.05-2.25 (2.4H); 2.82-3.05 (5H); 3.38 (2H); 3.50-3.70 (1.4H); 4.25 (0.6H); 4.63 (0.4H); 4.77 (0.6H); 7.25-7.50 (5H), 7.55-8.20 (4H).

LC/MS (ESI): 487.4; (calculated ([M+H]+): 487.7).

### EXAMPLE AA10

### Molecule AA10

### Molecule A23: Product Obtained by the Reaction Between Molecule B7 and N-Boc-ethylenediamine

HOBt (8.94 g, 58.37 mmol), then BocEDA (112.20 g, 700.00 mmol) in solution in DCM (150 ml) at 0° C. are successively added to a solution of molecule B7 (190.00 g, 583.73 mmol) in DCM (2.9 L). EDC (123.10 g, 642.00 mmol) is added, then the mixture is stirred for 17 hours between 0° C. and room temperature. The reaction mixture is then washed with a saturated aqueous NaHCO₃ (2×1.5 L) solution, an 1N HCl aqueous solution (2×1.5 L), then a saturated aqueous NaCl solution (1.5 L), dried over Na₂SO₄, filtered and concentrated under reduced pressure. A white solid is obtained after recrystallization in acetonitrile.

Yield: 256.50 g (93%)

NMR ¹H (CDCl₃, ppm): 0.88 (3H); 1.16-1.38 (20H); 1.44 (9H); 1.56-1.71 (2H); 1.78-2.45 (6H); 3.11-3.72 (6H); 4.30 (0.1H); 4.51 (0.9H); 4.87 (0.1H); 5.04 (0.9H); 6.87 (0.1H); 7.23 (0.9H).

LC/MS (ESI): 468.0; (calculated ([M+H]⁺): 468.4).

### Molecule AA10

Following a process similar to that used in the preparation of molecule AA1 applied to molecule A23 (256.50 g, 548.43 mmol), a white solid of molecule AA10 in the form of a hydrochloride salt is obtained by trituration in pentane (1.6 L) and drying under reduced pressure at 40° C.

Yield: 220.00 g (99%)

¹H NMR (MeOD-d4, ppm): 0.90 (3H); 1.21-1.43 (20H); 1.54-1.66 (2H); 1.85-2.28 (4H); 2.39 (2H); 3.00-3.17 (2H); 3.30-3.40 (1H); 3.43-3.71 (3H); 4.29 (0.94H); 4.48 (0.06H).

LC/MS (ESI): 368.2; (calculated ([M+H]+): 368.3).

### EXAMPLE AA11

### Molecule AA11

Molecule A24: Product Obtained by Reaction Between Molecule B7 and Boc-1-amino-4,7,10-trioxa-13-tridecane amine.

By a process similar to that used in the preparation of molecule A23 applied to molecule B7 (24.00 g, 73.73 mmol) and Boc-1-amino-4,7,10-trioxa-13-tridecane amine (28.35 g, 88.48 mmol), an orange oil of molecule A24 is obtained.

Yield: 44.50 g (96%)

1H NMR (CDCl₃, ppm): 0.87 (3H); 1.08-1.56 (20H); 1.43 (9H); 1.58-1.67 (2H); 1.70-2.00 (6H); 2.04-2.41 (4H); 3.16-3.77 (18H); 4.26-4.29 (0.2H); 4.50-4.54 (0.8H); 4.68-5.10 (1H); 6.74 (0.2H); 7.19 (0.8H).

LC/MS (ESI): 628.4; (calculated ([M+H]+): 628.5).

### Molecule AA11

Following a process similar to that used in the preparation of AMolecule A1 applied to molecule A24 (43.40 g, 69.12 mmol), a white solid of molecule AA11 in the form of hydrochloride salt is obtained after trituration 3 times in diethyl ether, solubilization of the residue in water and lyophilization.

Yield: 38.70 g (98%)

1H NMR (DMSO, ppm): 0.85 (3H); 1.07-1.38 (20H); 1.41-1.52 (2H); 1.55-1.66 (2H); 1.70-2.02 (6H); 2.08-2.30 (2H); 2.78-2.87 (2H); 3.00-3.16 (2H); 3.29-3.66 (14H); 4.16-4.22 (0.65 H); 4.25-4.30 (0.35H); 7.74 (0.65H); 7.86 (3H); 8.10 (0.35H).

LC/MS (ESI): 528.4; (calculated ([M+H]+): 528.4).

### EXAMPLE AA12

### Molecule AA12

### Molecule A25: Product Obtained by the Reaction Between Molecule B4 and N-Boc-ethylenediamine.

By a process similar to that used in the preparation of molecule A23 applied to molecule B4 (12.00 g, 40.35 mmol) and to Boc-ethylenediamine (7.76 g, 48.42 mmol), a colorless oil is obtained and used without further purification.

Yield: 17.40 g (94%)

1H NMR (CDCl3, ppm): 0.86 (3H); 1.11-1.68 (18H); 1.41 (9H); 1.80-2.38 (6H); 3.06-3.35 (4H); 3.37-3.49 (1H); 3.51-3.73 (1H); 4.26-4.31 (0.1H); 4.45-4.52 (0.9H); 4.91-5.19 (1H); 6.97 (0.1H); 7.23 (0.9H).LC/MS (ESI): 440.4 (calculated ([M+H]⁺): 440.3).

### Molecule AA12

Following a process similar to that used in the preparation of molecule AA1 applied to molecule A25 (8.85 g, 20.13 mmol), a white solid of molecule AA12 is obtained after basic washing, concentration under reduced pressure, then recrystallization in acetonitrile.

Yield: 6.53 g (96%)

1H NMR (DMSO, ppm): 0.85 (3H); 1.07-1.56 (20H); 1.68-2.03 (4H); 2.09-2.29 (2H); 2.50-2.58 (2H); 2.96-3.11 (2H); 3.21-3.59 (2H); 4.17-4.21 (0.65H); 4.25-4.29 (0.35H); 7.68 (0.65H); 8.00 (0.35H)

LC/MS (ESI): 340.3; (calculated ([M+H]+): 340.3).

### EXAMPLE AA13

### Molecule AA13

### Molecule A26: Product Obtained by Coupling Between Molecule B1 and the N-Boc-ethylenediamine.

By a process similar to that used in the preparation of molecule A23 applied to molecule B1 (30.00 g, 111.36 mmol) and BocEDA (21.41 g, 133.64 mmol), a white solid is obtained after recrystallization in acetonitrile.

Yield: 34.90 g (76%)

1H NMR (CDCl3, ppm): 0.88 (3H); 1.10-1.70 (14H); 1.43 (9H); 1.80-1.91 (1H); 1.92-2.01 (1H); 2.04-2.42 (4H); 3.13-3.70 (6H); 4.27-4.31 (0.15H); 4.47-4.53 (0.85H); 4.83 (0.15H); 5.02 (0.85H); 6.85 (0.15H); 7.21 (0.85H).

LC/MS (ESI): 412.2; (calculated ([M+H]+): 412.3).

### Molecule AA13

Following a process similar to that used in the preparation of molecule AA1 applied to molecule A26 (34.90 g, 84.79 mmol), a white solid of molecule AA13 in the form of hydrochloride salt is obtained after solubilization in a DCM/acetonitrile mixture and concentration under reduced pressure.

Yield: 29.50 g (99%)

1H NMR (DMSO, ppm): 0.85 (3H); 1.07-1.61 (14H); 1.70-2.06 (4H); 2.10-2.35 (2H); 2.76-2.87 (2H); 3.24-3.47 (3.25H); 3.56-3.64 (0.75H); 4.13-4.19 (0.75H); 4.31-4.36 (0.25H); 8.05-8.36 (3.75H); 8.50 (0.25H).

LC/MS (ESI): 312.2; (calculated ([M+H]+): 312.3).

### EXAMPLE AA14

### Molecule AA14

Molecule A27: Product Obtained by Hydrogenation of phytol.

Platinum oxide (PtO₂, 1.15 g, 6.61 mmol) is added to a solution of phytol (30.00 g, 101.20 mmol) in THF (450 mL) under argon and the medium is placed under 1 bar of dihydrogen, then stirred for 4 hours at room temperature. After filtration through celite rinsed with THF, a black oil of molecule A27 is obtained after concentration under reduced pressure.

Yield: 29.00 g (96%)

NMR ¹H (CDCl₃, ppm): 0.84 (6H); 0.86 (6H); 0.89 (3H); 1.00-1.46 (22H); 1.46-1.68 (3H); 3.61-3.73 (2H).

### Molecule A28: Product Obtained by Oxidation of Molecule A27

Tetrabutylammonium bromide (16.90 g, 52.45 mmol), acetic acid (150 mL, 2.62 mol), then KMnO4₄ (46.05 g, 291.40 mmol) are successively added in small fractions, while maintaining the temperature between 16 and 19° C., to a solution of molecule A27 (29.0 g, 97.13 mmol) in a mixture of dichloroethane/water (485 mL/388 mL). The reaction mixture is then stirred for 4 hours 30 at reflux, cooled to 10° C., then acidified to pH 1 with a 6 N HCl solution (20 mL). Na₂SO₃ (53.90 g) is then added gradually while maintaining the temperature at 10° C., and the mixture is stirred until completely discolored. Water (200 mL) is added, the phases are separated, and the aqueous phase is extracted with DCM (2×400 ml). The combined organic phases are washed with 10% HCl aqueous solution (20 mmL), water (2×200 ml), saturated aqueous NaCl solution (200 ml), dried over Na₂SO₄, filtered and concentrated under reduced pressure. A yellow oil of molecule A28 is obtained after purification by flash chromatography (eluent: cyclohexane, AcOEt).

Yield: 28.70 g (94%)

NMR ¹H (CDCl₃, ppm): 0.84 (6H); 0.86 (6H); 0.97 (3H); 1.00-1.41 (20H); 1.52 (1H); 1.96 (1H); 2.14 (1H); 2.35 (1H); 11.31 (1H).

LC/MS (ESI): 311.1 (calculated ([M−H]⁻): 311.3).

Molecule A29: Product Obtained by Coupling Between Molecule A28 and methyl L-prolinate.

By a process similar to that used in the preparation of molecule A2 applied to molecule A28 (18.00 g, 57.59 mmol) and methyl L-prolinate hydrochloride (14.31 g, 86.39 mmol) a yellow oil of molecule A29 is obtained after washing the organic phase with a NaHCO₃ (2×150 mL) saturated aqueous solution, a 10% aqueous solution of HCl (2×150 mL), a saturated aqueous solution of NaCl (2×150 mL), then drying on Na₂SO₄, filtration and concentration under reduced pressure.

Yield: 23.20 g (95%)

NMR ¹H (DMSO-d₆, ppm): 0.78-0.89 (15H); 0.97-1.43 (20H); 1.43-1.56 (1H); 1.70-1.96 (4H); 1.96-2.32 (3H); 3.33-3.56 (2H); 3.59 (0.6H); 3.67 (2.4H); 4.27 (0.8H); 4.57 (0.2H). LC/MS (ESI): 424.4 (calculated ([M+H]+): 424.4).

### Molecule A30: Product Obtained by the Saponification of Molecule A29.

By a process similar to that used in the preparation of molecule A21 applied to molecule A29 (21.05 g, 49.68 mmol), a yellow oil of molecule A30 is obtained.

Yield: 20.40 g (99%)

NMR ¹H (DMSO-d₆, ppm): 0.77-0.91 (15H); 0.97-1.43 (20H); 1.43-1.56 (1H); 1.67-1.96 (4H); 1.96-2.29 (3H); 3.26-3.56 (2H); 4.20 (0.8H); 4.41 (0.2H).

LC/MS (ESI): 410.3 (calculated ([M+H]⁺): 410.4).

Molecule A31: Product Obtained by Reaction Between Molecule A30 and Boc-1-amino-4,7,10-trioxa-13-tridecane amine.

By a process similar to that used in the preparation of molecule A23 applied to molecule A30 (8.95 g, 21.85 mmol) and Boc-1-amino-4,7,10-trioxa-13-tridecane amine (8.40 g, 26.21 mmol), a colorless oil of molecule A31 is obtained after purification by flash chromatography (eluent: DCM, AcOEt, methanol).

Yield: 10.08 g (65%)

NMR ¹H (DMSO-d₆, ppm): 0.78-0.89 (15H); 0.97-1.43 (29H); 1.43-1.55 (1H); 1.55-1.66 (4H); 1.71-2.30 (7H); 2.95 (2H); 3.00-3.19 (2H); 3.34-3.58 (14H); 4.17-4.29 (1H); 6.30-6.79 (1H); 7.67 (0.65H); 8.00 (0.35H).

LC/MS (ESI): 712.6 (calculated ([M+H]+): 712.6).

### Molecule AA14

Following a process similar to that used in the preparation of Amolecule A1 applied to molecule A31 (10.08 g, 14.16 mmol), the residue obtained after concentration under reduced pressure is solubilized in DCM (200 ml). The organic phase is washed with a 2N aqueous solution of NaOH (2×100 ml), dried over Na₂SO₄, filtered and concentrated under reduced pressure. A colorless oil of molecule AA14 in neutral amine form is obtained.

Yield: 8.23 g (95%)

NMR ¹H (DMSO-d₆, ppm): 0.78-0.89 (15H); 0.97-1.43 (20H); 1.43-1.69 (6H); 1.69-2.30 (8H); 2.56 (2H); 2.99-3.19 (2H); 3.31-3.58 (14H); 4.15-4.29 (1H); 7.70 (0.65H); 8.04 (0.35H).

LC/MS (ESI): 612.5 (calculated ([M+H]+): 612.5).

### EXAMPLE AA15

### Molecule AA15

Molecule AA15 is obtained by the conventional method of solid phase peptide synthesis (SPPS) on 2-chlorotrityl resin.

DIPEA (8.64 mL, 49.60 mmol) is added to a solution of 4,7,10-trioxa-1,13-tridecanediamine (TOTA, 10.87 mL, 49.60 mmol) in DCM (50 mL). This solution is then poured onto 2-chlorotrityl resin (4.00 g, 1.24 mmol/g) previously washed with DCM in a reactor adapted to SPPS. After stirring for 2 hours at room temperature, methanol (0.8 ml/g, 3.2 ml) is added and the medium is stirred for 15 minutes. The resin is filtered, washed successively with DCM (3×50 ml), DMF (2×50 ml), DCM (2×50 ml), isopropanol (1×50 ml) and DCM (3×50 ml). Protected amino acids N-Fmoc-L-glycine and N-Fmoc-L-proline, then palmitic acid (3 equivalents) are coupled successively using 1-[bis (dimethylamino) methylene]-1H-1,2,3-triazolo [4,5-b] pyridinium 3-oxide hexafluorophosphate (HATU, 3 equivalents) as coupling agent in the presence of DIPEA (6 equivalents) in a 1:1 DCM/DMF mixture. A solution of 20% piperidine in DMF is used for the cleavage steps of the Fmoc protecting group. The resin is washed with DCM, DMF and isopropanol after each coupling and deprotection step. Cleavage of the resin product is carried out using a 1:1 TFA/DCM mixture. The solvents are evaporated under reduced pressure, the residue is solubilized in DCM (50 mL) and the organic phase is washed with a 1N aqueous solution of NaOH (1×50 mL), then a saturated solution of NaCl (2×50 mL). After drying over Na₂SO₄, the organic phase is filtered, concentrated under reduced pressure and the residue is purified by chromatography on silica gel (dichloromethane, methanol, NH₄OH).

Yield: 1.65 g (54% overall over 7 steps).

NMR ¹H (CDCl₃, ppm): 0.88 (3H); 1.18-2.39 (38H); 2.79 (2H); 3.23-3.44 (2H); 3.47-3.69 (14H); 3.76 (0.92H); 3.82 (0.08H); 3.98 (0.08H); 4.03 (0.92H); 4.34 (0.08H); 4.39 (0.92H); 7.00-7.40 (2H).

LC/MS (ESI): 613.7; (calculated ([M+H]+): 613.5).

### EXAMPLE AA16

### Molecule AA16

By a SPPS process similar to that used in the preparation of molecule AA15 and using the N-Fmoc-L-phenylalanine (3 equivalents) instead of N-Fmoc-L-glycine, molecule AA16 is obtained in the form of a yellow oil.

Yield: 14.07 g (69%)

NMR ¹H (CDCl₃, ppm): 0.88 (3H); 1.19-1.34 (24H); 1.41-1.61 (2H); 1.68-2.28 (12H); 2.84 (2H); 3.14 (2H); 3.23-3.67 (16H); 4.19-4.25 (0.1H); 4.38-4.45 (0.9H); 4.59-4.69 (1H); 6.86 (1H); 7.03 (1H); 7.12-7.30 (5H).

LC/MS (ESI): 703.5; (calculated ([M+H]+): 703.5).

### EXAMPLE AA17

### Molecule AA17

Molecule AA17 is obtained in the form of a white solid by a SPPS process similar to that used in the preparation of molecule AA15 and using EDA (30.48 g, 0.456 mol) instead of TOTA.

Yield: 9.19 g (89%)

¹H NMR (MeOD-d4, ppm): 0.90 (3H); 1.22-1.43 (24H); 1.55-1.67 (2H); 1.91-2.04 (2H); 2.04-2.15 (1H); 2.17-2.29 (1H); 2.39 (2H); 2.69-2.82 (2H); 3.25-3.36 (2H); 3.58-3.70 (2H); 3.70-3.97 (2H); 4.25-4.34 (0.9H); 4.44-4.50 (0.1H).

LC/MS (ESI): 453.3; (calculated ([M+H]+): 453.4).

### EXAMPLE AA18

### Molecule AA18

By a SPPS process similar to that used in the preparation of molecule AA15 and successively using ethylenediamine (20 equivalents), N-Fmoc-L-phenylalanine (1.5 equivalents) and molecule B7 (1.5 equivalents), molecule AA18 is obtained in the form of a white solid.

Yield: 12.76 g (85%)

¹H NMR (MeOD-d4, ppm): 0.90 (3H); 1.14-1.65 (22H); 1.73-2.41 (6H); 2.56-2.70 (2H); 2.91-3.26 (4H); 3.41-3.63 (2H); 4.30 (0.8H); 4.39 (0.2H); 4.53 (0.8H); 4.61 (0.2H); 7.19-7.31 (5H).

LC/MS (ESI): 515.4; (calculated ([M+H]+): 515.4).

AB: Synthesis of Co-Polyamino Acids Modified by Hydrophobic Molecules wherein p=1

### Statistical Co-Polyamino Acids According to Formula VII or VIIa

TABLE 1B
List of co-polyamino acids according to formula VII or VIIa synthesized
according to the invention

| No | CO-POLYAMINOACIDES BEARING CARBOXYLATE CHARGES AND HYDROPHOBIC RADICALS |
|---|---|
| AB1 | 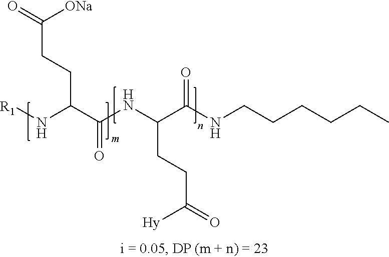 |
| | 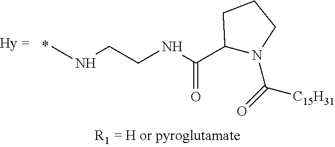 |
| AB2 | 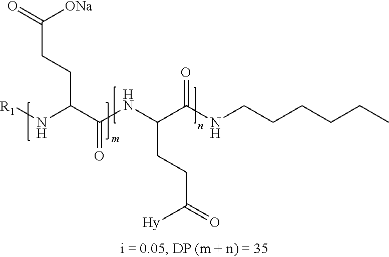 |
| | 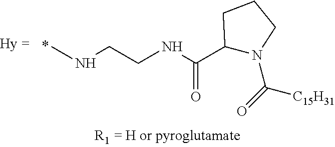 |
| AB3 | 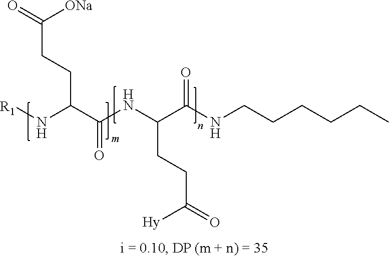 |
| | 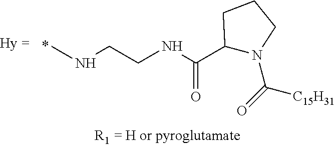 |
| AB4 | 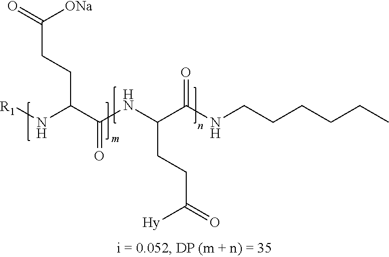 |
| | 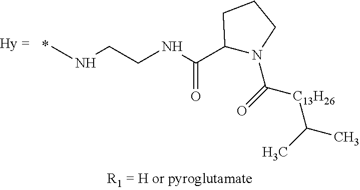 |
| AB5 | 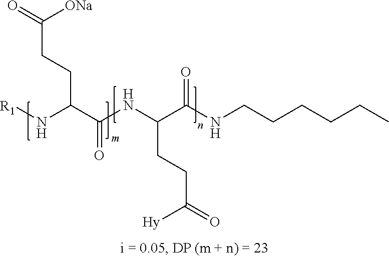 |
| | 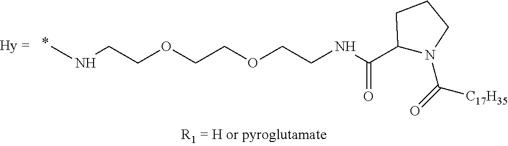 |
| AB6 | 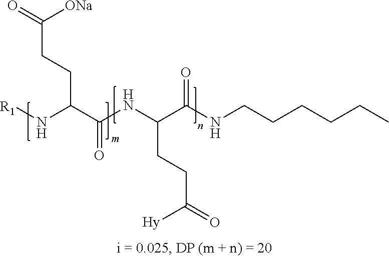 |
| | 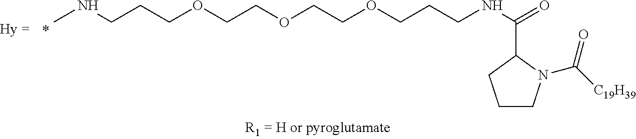 |
| AB7 | 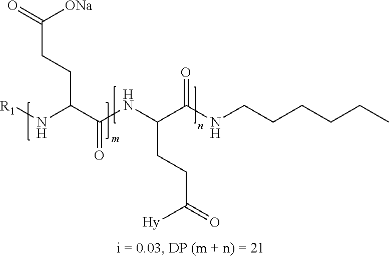 |
| | 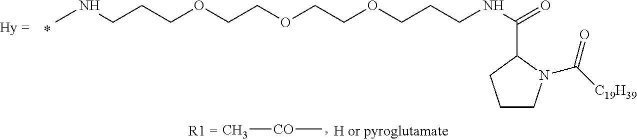 |
| AB8 | 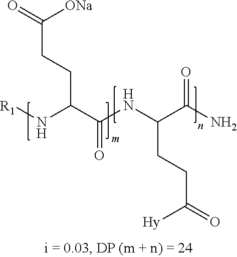 |
| | 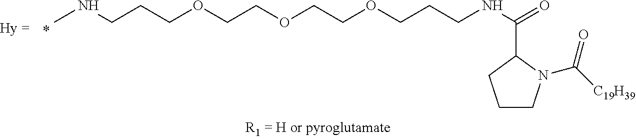 |
| AB9 | 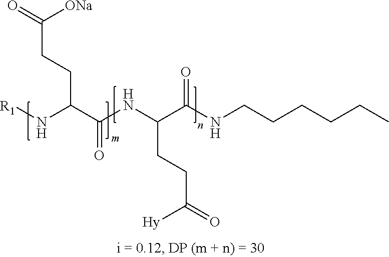 |
| | 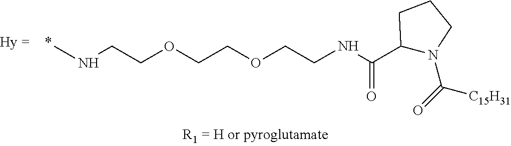 |
| AB10 | 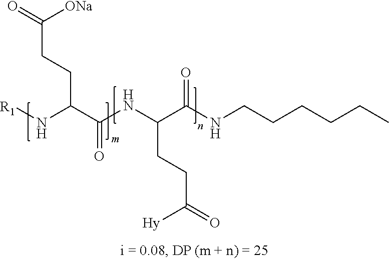 |
| | 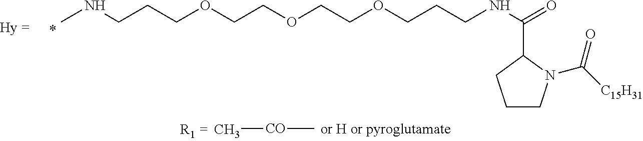 |
| AB11 | 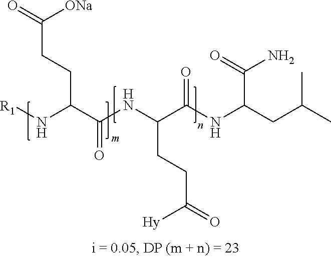 |
| | 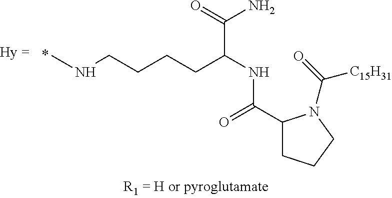 |
| AB12 | 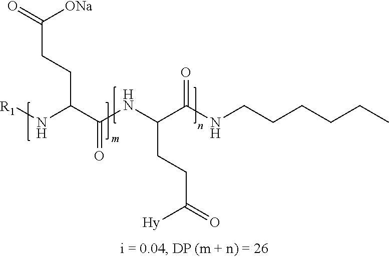 |
| | 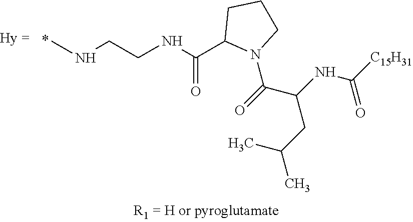 |
| AB13 | 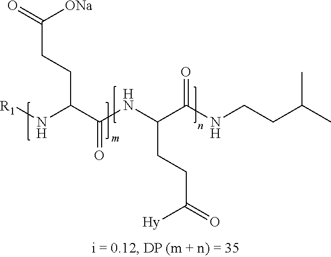 |
| | 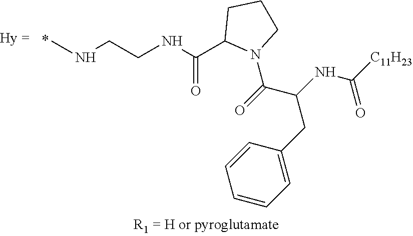 |
| AB21 | 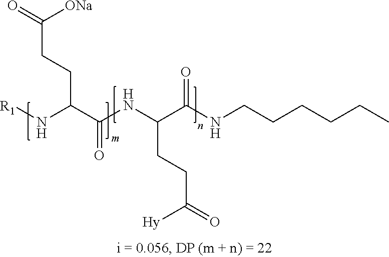 |
| | 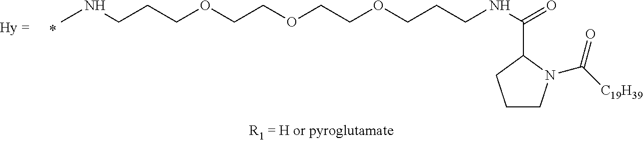 |
| AB22 | 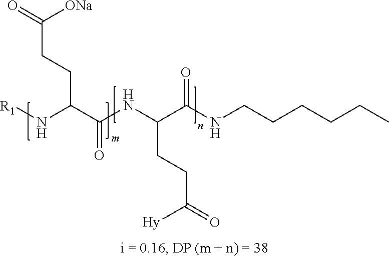 |
| | 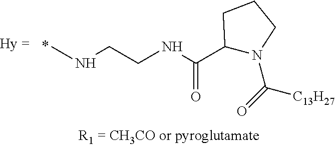 |
| AB23 | 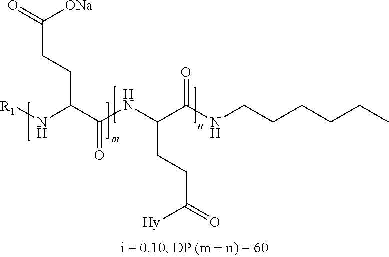 |
| | 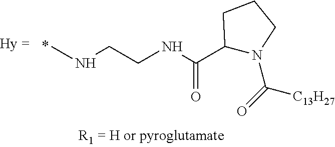 |
| AB24 | 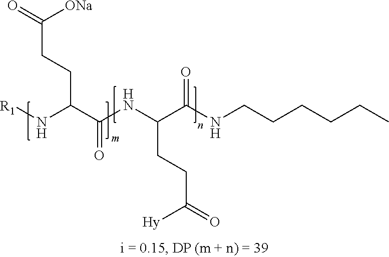 |
| | 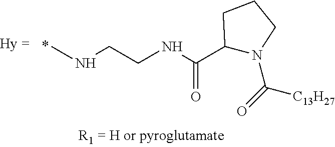 |
| AB25 | 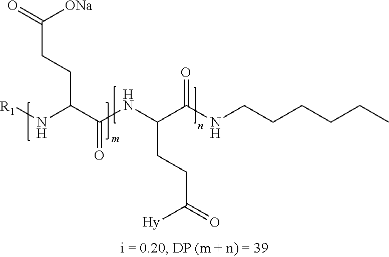 |
| | 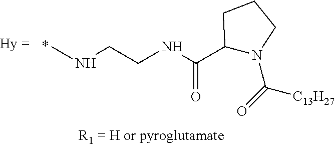 |
| AB26 | 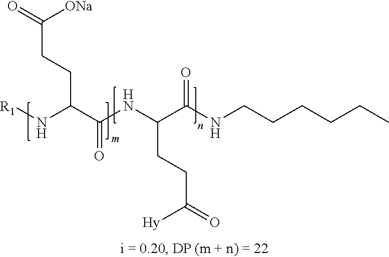 |
| | 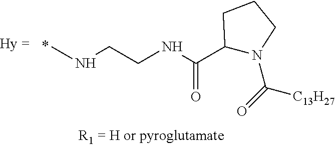 |
| AB27 | 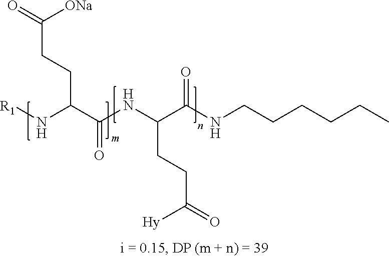 |
| | 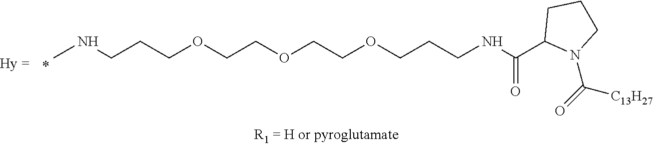 |
| AB28 | 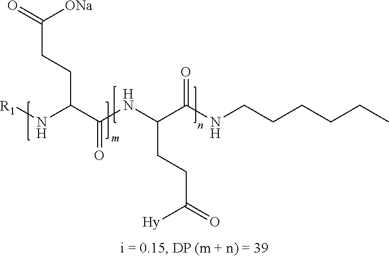 |
| | 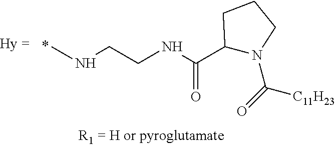 |
| AB29 | 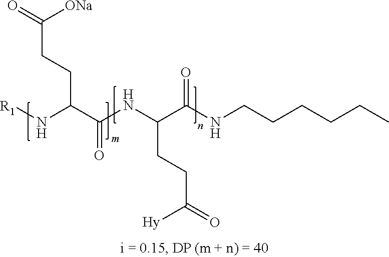 |
| | 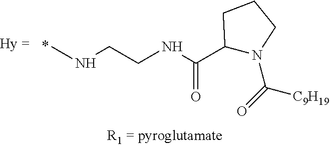 |
| AB30 | 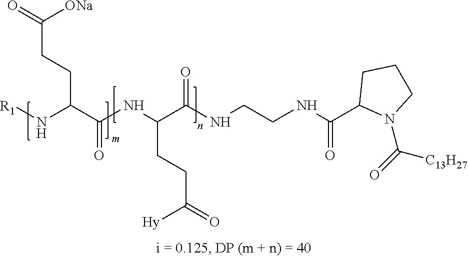 |
| | 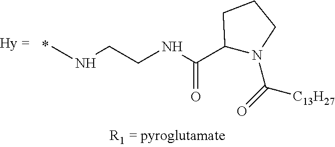 |
| AB31 | 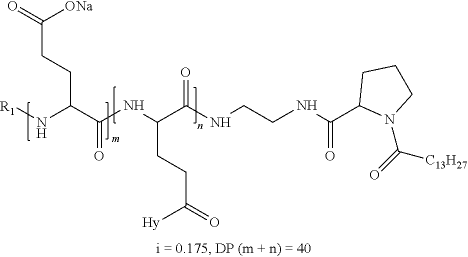 |
| | 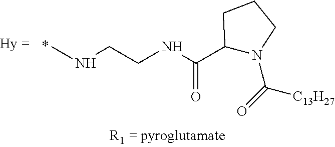 |
| AB32 | 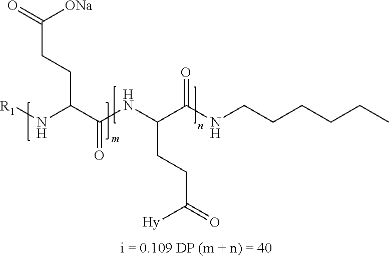 |
| | 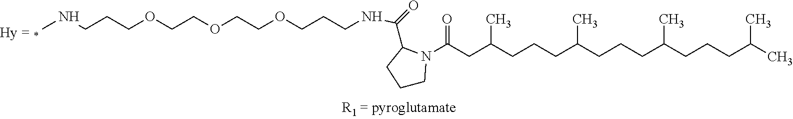 [SMILES: CNCCCOCCOCCOCCCNC(=O)C1CCCN1C(=O)CC(C)CCCC(C)CCCC(C)CCCC(C)C] |
| AB38 | 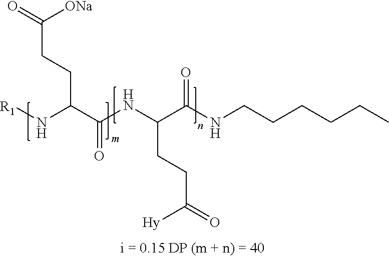 |
| | 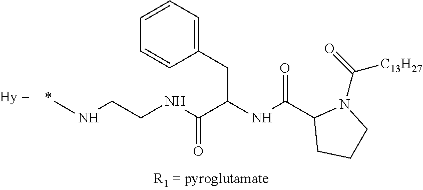 |

### Co-Polyamino Acids According to Formula VII or VIIb:

TABLE 1C
List of co-polyamino acids according to formula VII or VIIb synthesized
according to the invention.

| No | CO-POLYAMINOACIDES BEARING CARBOXYLATE CHARGES AND HYDROPHOBIC RADICALS |
|---|---|
| AB14 | 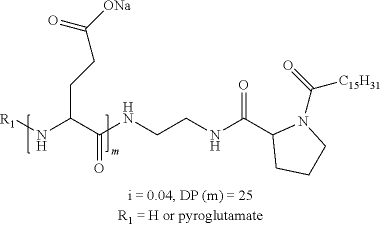 |
| AB15 | 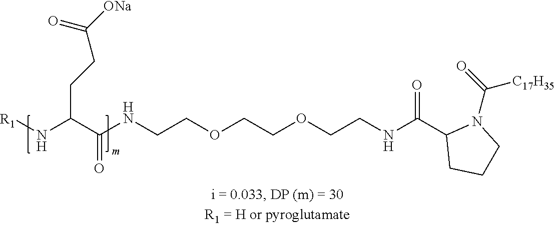 |
| AB16 | 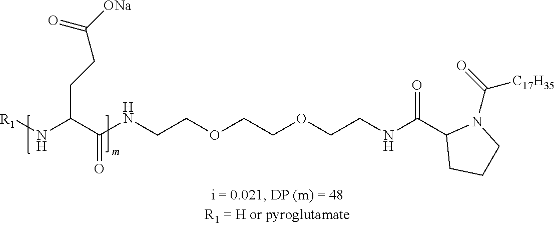 |
| AB17 | 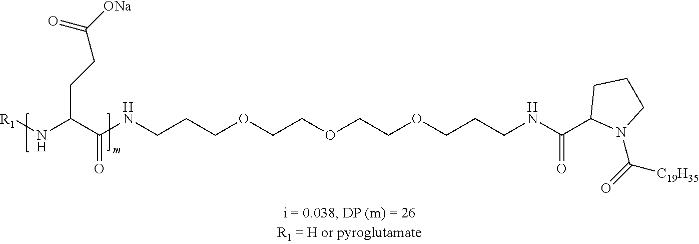 |
| AB18 | 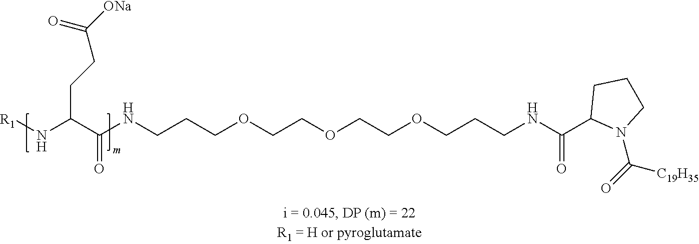 |
| AB19 | 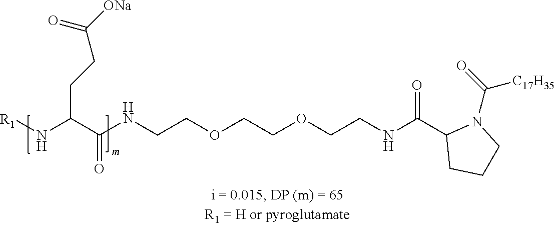 |
| AB20 | 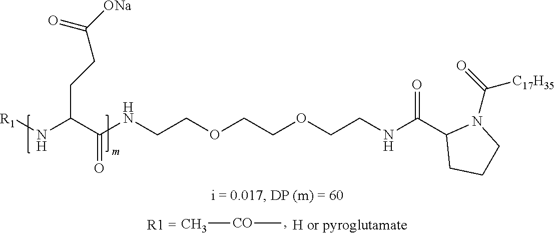 |
| AB33 | 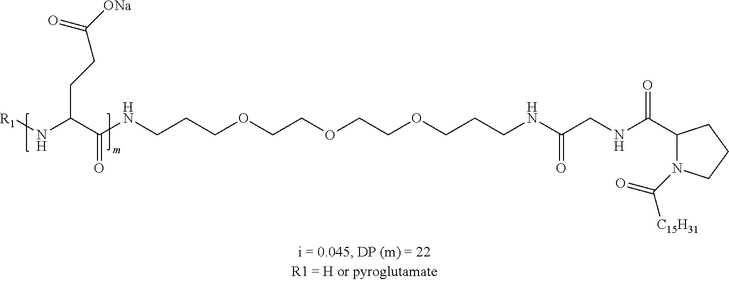 |
| AB34 | 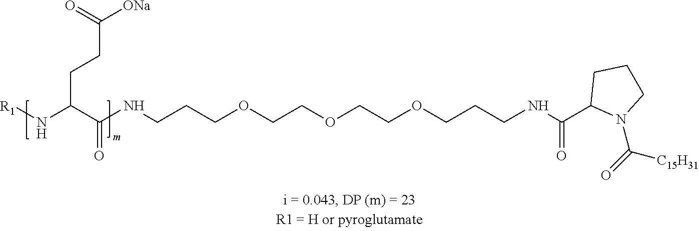 |
| AB35 | 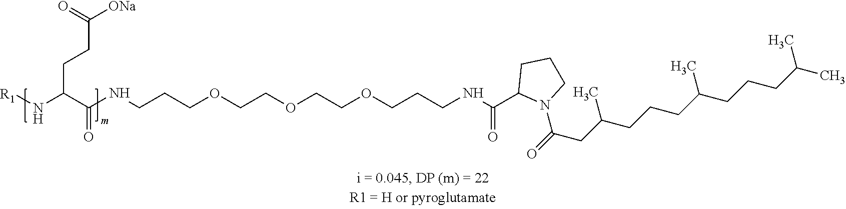 |
| AB36 | 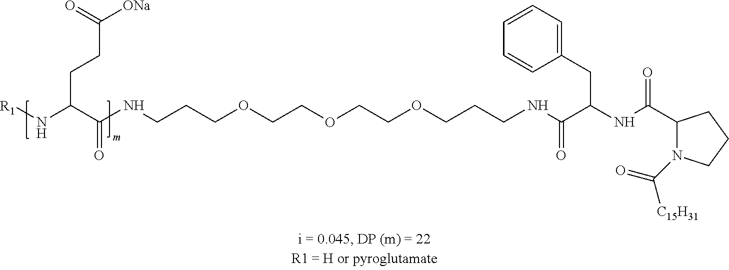 |
| AB37 | 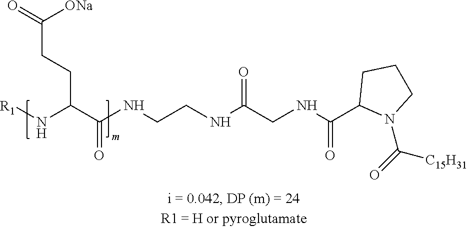 |

Part AB: Synthesis of Co-Polyamino Acids Modified by Hydrophobic Molecules wherein p=1

### Co-Polyamino Acids According to Formula VII or VIIa

### EXAMPLE AB1

### Co-Polyamino Acid AB1—Sodium poly-L-glutamate Modified by Molecule AA1 and Having a Number-Average Molecular Weight (Mn) of 2900 g/mol

Co-Polyamino Acid AB1-1: poly-L-glutamic Acid of Number-Average Molecular Weight (Mn) 3861 g/mol from the Polymerization of γ-benzyl-L-glutamate N-carboxyanhydride Initiated by Hexylamine

In a previously oven-dried flask, γ-benzyl-L-glutamate N-carboxyanhydride (89.9 g, 341 mmol) is placed under vacuum for 30 minutes, then anhydrous DMF (200 ml) is added. The mixture is then stirred under argon until complete dissolution, cooled to 4° C., then hexylamine (2.05 mL 15.5 mmol) is quickly introduced. The mixture is stirred at 4° C.? and room temperature for 2 days. The reaction medium is then heated at 65° C.? for 2 hours, cooled to room temperature, then poured dropwise into diisopropyl ether (3 L) with stirring. The white precipitate is collected by filtration, washed with diisopropyl ether (2×200 mL), then dried under vacuum at 30° C. to give a poly (gamma-benzyl-L-glutamic acid) (PBLG).

A hydrobromic acid solution (HBr) at 33% in acetic acid (240 mL, 1.37 mol) is added dropwise to a solution of PBLG (74.8 g) in trifluoroacetic acid (TFA, 340 ml) at 4° C. The mixture is stirred at room temperature for 2 hours, then poured dropwise onto a 1:1 (v/v) mixture of diisopropyl ether and water with stirring (4 L). After stirring for 2 hours, the heterogeneous mixture is allowed to stand overnight. The white precipitate is collected by filtration, washed with a 1:1 (v/v) mixture of diisopropyl ether and water (340 ml), then with water (340 ml).

The obtained solid is solubilized in water (1.5 mL) by adjusting the pH to 7 by adding 10 N aqueous sodium hydroxide solution, then 1N aqueous sodium hydroxide solution. After solubilization, the theoretical concentration is adjusted to 20 g/L theoretical by addition of water to obtain a final volume of 2.1 mL.

The solution is filtered through a 0.45 μm filter, then purified by ultrafiltration against a solution of NaCl 0.9%, then water until the conductimetry of the permeate is less than 50 μS/cm. The co-polyamino acid solution is then concentrated until a final volume of 1.8 L is obtained.

The aqueous solution is then acidified by adding a 37% hydrochloric acid solution until a pH of 2 is reached. After stirring for 4 hours, the precipitate obtained is filtered, washed with water (2×340 mL), then dried under vacuum at 30° C. to give a poly-L-glutamic acid of number-average molecular weight (Mn) 3861 g/mol with respect to a standard of polyoxyethylene (PEG).

### Co-Polyamino Acid AB1

Co-polyamino acid AB1-1 (10.0 g) is solubilized in DMF (700 mL) at 30° C.-40° C., then cooled to 0° C. molecule AA1 in the form of hydrochloride salt (1.64 g, 3.8 mmol) is suspended in DMF (23 mL) and triethylamine (0.39 g, 3.8 mmol) is then added and the mixture is slightly heated with stirring until complete dissolution. N-methylmorpholine (NMM, 7.6 g, 75 mmol) in DMF (14 mL) and ethyl chloroformate (ECF, 8.2 g, 75 mmol) are added to a co-polyamino acid solution at 0° C. After 10 minutes at 0° C., the solution containing the molecule AA1 is added and the medium maintained at 30° C. for 2 hours. The reaction mixture is poured dropwise over 5.5 L of water containing 15% NaCl weight and HCl (pH 2), and left to stand overnight. The precipitate is collected by filtration and dried under vacuum for about 30 minutes. The white solid obtained is taken up in water (500 ml) and the pH is adjusted to 7 by slow addition of a 1N aqueous solution of NaOH. After filtration on a 0.45 μm filter, the clear solution obtained is purified by ultrafiltration against 0.9% NaCl solution, then with water, until the conductimetry of the permeate is less than 50 μS/cm. After removal, the solution is filtered through a 0.2 μm filter and stored at 2-8° C.

Dry Extract: 24.9 mg/g

An average degree of polymerization (DP) of 23 is estimated by ¹H NMR in D₂O comparing the integration of the signals from the grafted hydrophobe to that of the signals from the main chain.

Based on ¹H NMR: i=0.05

The calculated average molecular weight of co-polyamino acid AB1 is calculated on the basis of the molar masses of radicals R1 and R2, aspartate and/or glutamate residues (including an amide linkage), hydrophobic radical, DS and DP.

The calculated average molecular weight of co-polyamino acid AB1 is 3945 g/mol. Aqueous HPLC-SEC (PEG calibrant): Mn=2900 g/mol.

### EXAMPLE AB2

### Co-Polyamino Acid AB2—Sodium poly-L-glutamate Modified by Molecule AA1 and Having a Number-Average Molecular Weight (Mn) of 3700 g/mol

A sodium poly-L-glutamate modified with molecule AA1 is obtained by a process similar to that used for the preparation of the co-polyamino acid AB1 applied to the hydrochloride salt of molecule AA1 (1.64 g, 3.8 mmol) and a poly-L-glutamic acid of Mn relative 5200 g/mol (10.0 g) obtained by a process similar to that used for the preparation of the co-polyamino acid AB1-1.

Dry extract: 14.1 mg/g

DP (estimated based on ¹H NMR): 35

Based on ¹H NMR: i=0.05

The calculated average molecular weight of co-polyamino acid AB2 is 5972 g/mol.

Aqueous HPLC-SEC (PEG calibrant): Mn=3700 g/mol.

### EXAMPLE AB3

### Co-Polyamino Acid AB3—Sodium poly-L-glutamate Modified by Molecule AA1 and Having a Number-Average Molecular Weight (Mn) of 4900 g/mol

A sodium poly-L-glutamate modified with molecule AA1 is obtained by a process similar to that used in the preparation of the co-polyamino acid AB1 applied to the hydrochloride salt of molecule AA1 (3.30 g, 7.6 mmol) and to a poly-L-glutamic acid of relative number-average molecular weight (Mn) 5200 g/mol (10.0 g) obtained by a process similar to that used in the preparation of co-polyamino acid AB1-1.

Dry extract: 23.4 mg/g

DP (estimated based on ¹H NMR): 35

The calculated average molecular weight of co-polyamino acid AB3 is 6594 g/mol.

Based on ¹H NMR: i=0.10

Aqueous HPLC-SEC (PEG calibrant): Mn=4900 g/mol.

### EXAMPLE AB4

### Co-Polyamino Acid AB4—Sodium poly-L-glutamate Modified by Molecule AA2 and Having a Number-Average Molecular Weight (Mn) of 1800 g/mol

By a process similar to that used in the preparation of co-polyamino acid AB1 applied to the hydrochloride salt of molecule AA2 (1.09 g, 2.4 mmol) and a poly-L-glutamic acid of average mass Mn=5600 g/mol (6.3 g) obtained by a process similar to that used in the preparation of co-polyamino acid AB1-1 but with a benzyl ester deprotection step using trimethylsilane iodide according to the protocol described in publication J. Am. Chem. Soc. 2000, 122, 26-34 (Subramanian G., et al.), a sodium poly-L-glutamate modified with molecule AA2 is obtained.

Dry extract: 21.5 mg/g

DP (estimated based on ¹H NMR): 35

Based on ¹H NMR: i=0.052

The calculated average molecular weight of co-polyamino acid AB4 is 6022 g/mol.

Aqueous HPLC-SEC (PEG calibrant): Mn=1800 g/mol.

### EXAMPLE AB5

### Co-Polyamino Acid AB5—Sodium poly-L-glutamate Modified by Molecule AA6 and Having a Number-Average Molecular Weight (Mn) of 2600 g/mol

A sodium poly-L-glutamate modified with molecule AA6 is obtained by a process similar to that used in the preparation of co-polyamino acid AB1 applied to the hydrochloride salt of molecule AA6 (2.06 g, 3.8 mmol) and to a poly-L-glutamic acid (9.8 g) obtained by a process similar to that used in the preparation of polyamino acid AB1-1.

Dry extract: 20.9 mg/g

DP (estimated based on ¹H NMR): 23

Based on ¹H NMR: i=0.05

The calculated average molecular weight of co-polyamino acid ABS is 4079 g/mol.

Aqueous HPLC-SEC (PEG calibrant): Mn=2600 g/mol.

### EXAMPLE AB6

### Co-Polyamino Acid AB6—Sodium poly-L-glutamate Modified by Molecule AA7 and Having a Number-Average Molecular Weight (Mn) of 4000 g/mol

A poly-L-glutamic acid of average mass Mn=3500 g/mol and a polymerization degree of 22 (10.0 g) obtained by a process similar to that used in the preparation of co-polyamino acid AB1-1 is solubilized in DMF (420 ml) at 30-40° C. and maintained at this temperature. In parallel, the hydrochloride salt of the AA7 molecule (1.47 g, 2.3 mmol) is suspended in DMF (12 ml) and triethylamine (0.23 g, 2.3 mmol) is added then the mixture is gently heated with stirring until completely dissolved. NMM (7.6 g, 75 mmol), AA7 solution, then 2-hydroxypyridine N-oxide (HOPO, 0.84 g, 7.5 mmol) are successively added to the co-polyamino acid solution in DMF. The reaction medium is then cooled to 0° C., then EDC (1.44 g, 7.5 mmol) is added and the medium is raised to room temperature for 2 hours. The reaction medium is filtered through a 0.2 mm woven filter and poured drop by drop onto 3.5 L of water containing NaCl 15% by weight and HCl (pH 2) with stirring. At the end of the addition, the pH is readjusted to 2 with 37% HCl solution, and the suspension is allowed to stand overnight. The precipitate is collected by filtration, then rinsed with 100 mL of water. The white solid obtained is solubilized in 500 ml of water by slowly adding a 1N aqueous NaOH solution to pH 7 with stirring, then the solution is filtered through a 0.45 μm filter. The clear solution obtained is purified by ultrafiltration against 0.9% NaCl solution, then with water, until the conductimetry of the permeate is less than 50 μS/cm. The solution is filtered through a 0.2 μm filter and stored at 2-8° C.

Dry extract: 21.6 mg/g

DP (estimated based on ¹H NMR): 20

Based on ¹H NMR: i=0.025

The calculated average molecular weight of co-polyamino acid AB6 is 3369 g/mol.

Aqueous HPLC-SEC (PEG calibrant): Mn=4000 g/mol.

### EXAMPLE AB7

### Co-Polyamino Acid AB7—Sodium poly-L-glutamate Capped at One of Its Ends by an Acetyl Group and Modified by Molecule AA7 and Having a Number-Average Molecular Weight (Mn) of 3300 g/mol

Co-polyamino acid AB7-1: poly-L-glutamic Acid of Relative Number-Average Molecular Weight (Mn) of 3600 g/mol and of DP 21 Resulting from the Polymerization of γ-benzyl-L-glutamate N-carboxyanhydride Initiated by the Hexylamine and Capped at One of its Ends by an Acetyl Group

In a previously oven-dried flask, γ-benzyl-L-glutamate N-carboxyanhydride (Glu (OBn)-NCA, 100.0 g, 380 mmol) is placed under vacuum for 30 minutes then anhydrous DMF (225 ml) is added. The mixture is then stirred under argon until complete dissolution, cooled to 4° C., then hexylamine (1.78 g, 17 mmol) is quickly introduced. The mixture is stirred between 4° C. and room temperature for 2 days, then precipitated in diisopropyl ether (3.4 L). The precipitate is collected by filtration, washed twice with diisopropyl ether (225 ml), then dried to give a white solid which is dissolved in 450 ml of THF. DIPEA (31 mL, 176 mmol), then acetic anhydride (17 mL, 176 mmol) are successively added to this solution. After stirring overnight at room temperature, the solution is slowly poured into diisopropyl ether (3 L) under stirring. After stirring for 1 hour, the precipitate is filtered off, washed twice with diisopropyl ether (250 ml), then dried under vacuum at 30° C. to give a poly (gamma-benzyl-L-glutamic acid) capped at one of its ends by an acetyl group.

A solution of hydrobromic acid (HBr) at 33% in acetic acid (235 ml) is added dropwise to a solution of the above co-polyamino acid (72 g) in trifluoroacetic acid (TFA, 335 ml) at 4° C. The mixture is stirred at room temperature for 3h30, then poured dropwise onto a 1:1 (v/v) mixture of diisopropyl ether and water with stirring (4 L). After stirring for 2 hours, the heterogeneous mixture is allowed to stand overnight. The white precipitate is collected by filtration, washed with a 1:1 (v/v) mixture of diisopropyl ether and water (340 ml), then with water (340 ml).

The obtained solid is then solubilized in water (1.5 L) by adjusting the pH to 7 by adding a 10N aqueous solution of sodium hydroxide, then a 1N aqueous sodium hydroxide solution. After solubilization, the solution is diluted by adding water to obtain a final volume of 2.1 L. The solution is filtered through a 0.45 μm filter, then purified by ultrafiltration against a solution of NaCl 0.9%, then water until the conductimetry of the permeate is less than 50 μS/cm. The co-polyamino acid solution is then concentrated until a final volume of 1.8 L is obtained.

The aqueous solution is then acidified by adding a 37% hydrochloric acid solution until a pH of 2 is reached. After stirring for 4 hours, the precipitate obtained is filtered, washed with water (330 mL), then dried under vacuum at 30° C. to give a poly-L-glutamic acid of number-average molecular weight (Mn) 3600 g/mol relative to a standard of polyoxyethylene (PEG) and of average degree of polymerization 21.

### Co-polyamino Acid AB7:

A sodium poly-L-glutamate acid modified with the molecule AA7 is obtained by a process similar to that used in the preparation of the co-polyamino acid AB6 applied to the hydrochloride salt of molecule AA7 (1.43 g, 2.2 mmol) and the co-polyamino acid AB7-1 (10.0 g).

Dry extract: 24.3 mg/g

DP (estimated based on ¹H NMR): 21

Based on ¹H NMR: i=0.03

The calculated average molecular weight of co-polyamino acid AB7 is 3677 g/mol.

Aqueous HPLC-SEC (PEG calibrant): Mn=3300 g/mol.

### EXAMPLE AB8

### Co-Polyamino Acid AB8—Sodium poly-L-glutamate Modified by Molecule AA7 and Having a Number-Average Molecular Weight (Mn) of 3600 g/mol

Co-Polyamino Acid AB8-1: poly-L-glutamic Acid of Number-Average Molecular Weight (Mn) 3800 g/mol and Degree of Polymerization 24 Resulting from the Polymerization of γ-methyl-L-glutamate N-carboxyanhydride Initiated by Ammonia

A poly-L-glutamic acid is obtained by a process similar to that described in patent application FR-A-2 801 226 applied to γ-methyl-L-glutamic acid N-carboxyanhydride (25.0 g, 133.6 mmol) and 0.5 N ammonia solution in dioxane (12.1 ml, 6.05 mmol).

### Co-Polyamino Acid AB8:

A sodium poly-L-glutamate modified with the molecule AA7 is obtained by a process similar to that used in the preparation of the co-polyamino acid AB6 applied to the hydrochloride salt of molecule AA7 (2.1 g, 3.24 mmol) and co-polyamino acid AB8-1 (14.3 g).

Dry extract: 25.2 mg/g

DP (estimated based on ¹H NMR): 24

Based on ¹H NMR: i=0.03

The calculated average molecular weight of co-polyamino acid AB8 is 4099 g/mol.

Aqueous HPLC-SEC (PEG calibrant): Mn=3600 g/mol.

### EXAMPLE AB9

### Co-polyamino acid AB9—Sodium poly-L-glutamate Modified by Molecule AA3 and Having a Number-Average Molecular Weight (Mn) of 3200 g/mol

A sodium poly-L-glutamate modified by molecule AA3 is obtained by a process similar to that used in the preparation of co-polyamino acid AB1 applied to the hydrochloride salt of molecule AA3 and to a poly-L-glutamic acid obtained by a process similar to that used in the preparation of co-polyamino acid AB1-1.

Dry extract: 14.7 mg/g

DP (estimated based on ¹H NMR): 30

Based on ¹H NMR: i=0.12

The calculated average molecular weight of co-polyamino acid AB9 is 6192 g/mol.

Aqueous HPLC-SEC (PEG calibrant): Mn=3200 g/mol.

### EXAMPLE AB10

### Co-Polyamino Acid AB10—Sodium poly-L-glutamate Modified by Molecule AA4 and Having a Number-Average Molecular Weight (Mn) of 2600 g/mol

A sodium poly-L-glutamate modified by molecule AA4 is obtained by a process similar to that used in the preparation of co-polyamino acid AB7 applied to the hydrochloride salt of molecule AA4 and to a poly-L-glutamic acid obtained by a process similar to that used in the preparation of co-polyamino acid AB1-1.

Dry extract: 18.3 mg/g

DP (estimated based on ¹H NMR): 25

Based on ¹H NMR: i=0.08

The calculated average molecular weight of co-polyamino acid AB10 is 4870 g/mol.

Aqueous HPLC-SEC (PEG calibrant): Mn=2600 g/mol.

### EXAMPLE AB11

### Co-Polyamino Acid AB11—Sodium poly-L-glutamate Modified by Molecule AA5 and Having a Number-Average Molecular Weight (Mn) of 2700 g/mol

A sodium poly-L-glutamate modified by molecule AA5 is obtained by a process similar to that used in the preparation of co-polyamino acid AB6 applied to the hydrochloride salt of molecule AA5 and to a poly-L-glutamic acid obtained by a process similar to that used in the preparation of co-polyamino acid AB1-1.

Dry extract: 20.2 mg/g

DP (estimated based on ¹H NMR): 23

Based on ¹H NMR: i=0.05

The calculated average molecular weight of co-polyamino acid AB11 is 4072 g/mol.

Aqueous HPLC-SEC (PEG calibrant): Mn=2700 g/mol.

### EXAMPLE AB12

### Co-Polyamino Acid AB12—Sodium poly-L-glutamate Modified by Molecule AA8 and Having a Number-Average Molecular Weight (Mn) of 3000 g/mol

A sodium poly-L-glutamate modified by molecule AA8 is obtained by a process similar to that used in the preparation of co-polyamino acid AB1 applied to the hydrochloride salt of molecule AA8 and to a poly-L-glutamic acid obtained by a process similar to that used in the preparation of co-polyamino acid AB1-1.

Dry extract: 19.5 mg/g

DP (estimated based on ¹H NMR): 26

Based on ¹H NMR: i=0.04

The calculated average molecular weight of co-polyamino acid AB12 is 4477 g/mol.

Aqueous HPLC-SEC (PEG calibrant): Mn=3000 g/mol.

### EXAMPLE AB13

### Co-Polyamino acid AB13—Sodium poly-L-glutamate Modified by Molecule AA9 and Having a Number-Average Molecular Weight (Mn) of 3300 g/mol

By a process similar to that used in the preparation of co-polyamino acid AB6 applied to the hydrochloride salt of molecule AA9 and a poly-L-glutamic acid obtained by a process similar to that used in the preparation of co-polyamino acid AB1-1 Using isoamylamine as the initiator in place of hexylamine, a sodium poly-L-glutamate modified with molecule AA9 is obtained.

Dry extract: 22.3 mg/g

DP (estimated based on ¹H NMR): 35

Based on ¹H NMR: i=0.12

The calculated average molecular weight of co-polyamino acid AB13 is 7226 g/mol.

Aqueous HPLC-SEC (PEG calibrant): Mn=3300 g/mol.

### EXAMPLE AB21

### Co-Polyamino Acid AB21—Sodium poly-L-glutamate Modified by Molecule AA7 and Having a Number-Average Molecular Weight (Mn) of 3400 g/mol

A sodium poly-L-glutamate modified with molecule AA7 is obtained by a process similar to that used in the preparation of co-polyamino acid AB6 applied to the hydrochloride salt of molecule AA7 (2.44 g, 2.4 mmol) and to a poly-L-glutamic acid (10 g) obtained by a process similar to that used in the preparation of polyamino acid AB1-1.

Dry extract: 22.7 mg/g

DP (estimated based on ¹H NMR): 22

Based on ¹H NMR: i=0.056

The calculated average molecular weight of co-polyamino acid AB21 is 4090 g/mol.

Aqueous HPLC-SEC (PEG calibrant): Mn=3400 g/mol.

### EXAMPLE AB22

### Co-Polyamino Acid AB22—Sodium poly-L-glutamate Capped at One of its Ends by an Acetyl Group and Modified by Molecule AA10 and Having a Number-Average Molecular Weight (Mn) of 4000 g/mol

The hydrochloride salt of molecule AA10 (4.56 g, 11.29 mmol) is dissolved in chloroform (60 ml) and triethylamine (1.14 g, 11.29 mmol) is added. NMM (7.6 g, 75.26 mmol), then HOPO (2.51 g, 22.58 mmol) are successively added to a co-polyamino acid (10.0 g, 75.3 mmol) solution obtained according to a process similar to that used in the preparation of co-polyamino acid B7-1 in DMF (420 ml). The reaction medium is then cooled to 0° C., then EDC (4.33 g, 22.58 mmol) is added, the medium is stirred for 1 h at 0° C., then the solution of the molecule AA10 is added. The reaction mixture is stirred for 2 hours at between 0° C. and room temperature. The reaction medium is filtered through a 0.2 mm woven filter and poured drop by drop onto 3.95 L of water containing NaCl 15% by weight and HCl (pH 2) with stirring. At the end of the addition, the pH is readjusted to 2 with 37% HCl solution, and the suspension is allowed to stand overnight. The precipitate is collected by filtration, then solubilized in 780 mL of water by slow addition of a 1N aqueous NaOH solution to pH 7 with stirring. After filtration through a 0.45 μm filter, the solution is diluted by adding water, then acetone is added to obtain a solution containing 30% by weight of acetone. This solution is filtered through an activated charcoal filter, then the acetone is distilled (40° C., 100 mbar). After filtration through a 0.45 μm filter, the product is purified by ultrafiltration against a 0.9% NaCl aqueous solution, a carbonate buffer solution (150 mM), a 0.9% NaCl aqueous solution, a phosphate buffer (150 mM) solution, a 0.9% NaCl aqueous solution, then water until the conductimetry of the permeate is less than 50 μS/cm. The solution is then concentrated, filtered through a 0.2 μm filter and stored at 2-8° C.

Dry extract: 19.7 mg/g

DP (estimated based on ¹H NMR): 38

Based on ¹H NMR: i=0.16

The calculated average molecular weight of co-polyamino acid AB22 is 7877 g/mol.

Organic HPLC-SEC (PEG Calibrator): Mn=4000 g/mol.

### EXAMPLE AB23

### Co-Polyamino Acid AB23—Sodium poly-L-glutamate Modified by Molecule AA10 and Having a Number-Average Molecular Weight (Mn) of 7600 g/mol

Co-Polyamino acid AB23-1: poly-L-glutamic Acid from the Polymerization of γ-benzyl-L-glutamate N-carboxyanhydride Initiated by Hexylamine and Capped at One of Its Ends by a Pyroglutamate Group

A poly-L-glutamic acid (20.0 g) obtained by a process similar to that used in the preparation of the co-polyamino acid AB1-1 is solubilized in DMF at 80° C., then maintained at this temperature. After 24 hours, the reaction medium is poured into a solution of NaCl at 15% and at pH 2. After 4 hours, the white solid is collected by filtration, rinsed with water, then dried under vacuum at 30° C.

### Co-Polyaminoamide AB23

A sodium poly-L-glutamate acid modified with the molecule AA10 is obtained by a process similar to that used in the preparation of the co-polyamino acid AB22 applied to the hydrochloride salt of molecule AA10 (2.742 g, 6.79 mmol) and the co-polyamino acid AB23-1 (9.0 g).

Dry extract: 21.9 mg/g

DP (estimated based on ¹H NMR): 60

Based on ¹H NMR: i=0.1

The calculated average molecular weight of co-polyamino acid AB23 is 11034 g/mol.

Organic HPLC-SEC (PEG Calibrator): Mn=7600 g/mol.

### EXAMPLE AB24

### Co-Polyamino Acid AB24—Sodium poly-L-glutamate Modified by Molecule AA10 and Having a Number-Average Molecular Weight (Mn) of 4300 g/mol

A sodium poly-L-glutamate modified by molecule AA10 is obtained by a process similar to that used in the preparation of co-polyamino acid AB23 applied to the hydrochloride salt of molecule AA10 and to a poly-L-glutamic acid obtained by a process similar to that used in the preparation of co-polyamino acid AB23-1.

Dry extract: 22.9 mg/g

DP (estimated based on ¹H NMR): 39

Based on ¹H NMR: i=0.15

The calculated average molecular weight of co-polyamino acid AB24 is 7870 g/mol.

Organic HPLC-SEC (PEG Calibrator): Mn=4300 g/mol.

### EXAMPLE AB25

### Co-Polyamino Acid AB25—Sodium poly-L-glutamate Modified by Molecule AA10 and Having a Number-Average Molecular Weight (Mn) of 4200 g/mol

A sodium poly-L-glutamate modified by molecule AA10 is obtained by a process similar to that used in the preparation of co-polyamino acid AB23 applied to the hydrochloride salt of molecule AA10 and to a poly-L-glutamic acid obtained by a process similar to that used in the preparation of co-polyamino acid AB23-1.

Dry extract: 25.9 mg/g

DP (estimated based on ¹H NMR): 39

Based on ¹H NMR: i=0.2

The calculated average molecular weight of co-polyamino acid AB25 is 8509 g/mol.

Organic HPLC-SEC (PEG Calibrator): Mn=4200 g/mol.

### EXAMPLE AB26

### Co-Polyamino Acid AB26—Sodium poly-L-glutamate Modified by Molecule AA10 and Having a Number-Average Molecular Weight (Mn) of 2700 g/mol

A sodium poly-L-glutamate modified by molecule AA10 is obtained by a process similar to that used in the preparation of co-polyamino acid AB23 applied to the hydrochloride salt of molecule AA10 and to a poly-L-glutamic acid obtained by a process similar to that used in the preparation of co-polyamino acid AB23-1.

Dry extract: 23.9 mg/g

DP (estimated based on ¹H NMR): 22

Based on ¹H NMR: i=0.21

The calculated average molecular weight of co-polyamino acid AB26 is 4899 g/mol.

Organic HPLC-SEC (PEG Calibrator): Mn=2700 g/mol.

### EXAMPLE AB27

### Co-Polyamino Acid AB27—Sodium poly-L-glutamate Modified by Molecule AA11 and Having a Number-Average Molecular Weight (Mn) of 4500 g/mol

A sodium poly-L-glutamate modified by molecule AA11 is obtained by a process similar to that used in the preparation of co-polyamino acid AB23 applied to the hydrochloride salt of molecule AA11 and to a poly-L-glutamic acid obtained by a process similar to that used in the preparation of co-polyamino acid AB23-1.

Dry extract: 26.8 mg/g

DP (estimated based on ¹H NMR): 39

Based on ¹H NMR: i=0.15

The calculated average molecular weight of co-polyamino acid AB27 is 8808 g/mol.

Organic HPLC-SEC (PEG Calibrator): Mn=4500 g/mol.

### EXAMPLE AB28

### Co-Polyamino Acid AB28—Sodium poly-L-glutamate Modified by Molecule AA12 and Having a Number-Average Molecular Weight (Mn) of 4000 g/mol

A sodium poly-L-glutamate modified by molecule AA12 is obtained by a process similar to that used in the preparation of co-polyamino acid AB23 applied to the hydrochloride salt of molecule AA12 and to a poly-L-glutamic acid obtained by a process similar to that used in the preparation of co-polyamino acid AB23-1.

Dry extract: 22.9 mg/g

DP (estimated based on ¹H NMR): 39

Based on ¹H NMR: i=0.15

The calculated average molecular weight of co-polyamino acid AB28 is 7706 g/mol. Organic HPLC-SEC (PEG Calibrator): Mn=4000 g/mol.

### EXAMPLE AB29

### Co-Polyamino Acid AB29—Sodium poly-L-glutamate Modified by Molecule AA13 and Having a Number-Average Molecular Weight (Mn) of 4000 g/mol

Co-Polyamino Acid AB29-1: poly-L-glutamic Acid from the Polymerization of γ-benzyl-L-glutamate N-carboxyanhydride Initiated by Hexylamine

In a double-jacket reactor, γ-benzyl-L-glutamate N-carboxyanhydride (500 g, 1.90 mol) is solubilized in anhydrous DMF (1100 mL). The mixture is then stirred until complete dissolution, cooled to 0° C., then hexylamine (6.27 ml, 47.5 mmol) is introduced rapidly. The mixture is stirred at 0° C. for 5 hours, between 0° C. and 20° C. for 7 hours, then at 20° C. for 7 hours. The reaction medium is then heated at 65° C. for 2 hours, cooled to 55° C., and methanol (3300 mL) is introduced after 1 h 30. The reaction mixture is then cooled to 0° C. and left under stirring for 18 hours. The white precipitate is collected by filtration, washed with diisopropyl ether (2×800 ml), then dried under vacuum at 30° C. to give a poly (gamma-benzyl-L-glutamic) acid (PBLG).

Pd/Al₂O₃ (36 g) is added to a PBLG (180 g) solution in N,N-dimethylacetamide (DMAc, 450 ml) under an argon atmosphere. The mixture is placed in a hydrogen atmosphere (10 bar) and stirred at 60° C. for 24 hours. After cooling to room temperature and filtration of the catalyst on P4 sintered filter and PTFE Omnipore hydrophilic membrane 0.2 μm, a water solution at pH 2 (2700 ml) is poured dropwise on the DMAc solution, on a 45 minute period with stirring. After stirring for 18 hours, the white precipitate is collected by filtration, washed with water, then dried under reduced pressure at 30° C.

### Co-Polyamino Acid AB29

A sodium poly-L-glutamate modified by molecule AA13 is obtained by a process similar to that used in the preparation of co-polyamino acid AB23 applied to the hydrochloride salt of molecule AA13 and co-polyamino acid AB29-1.

Dry extract: 16.1 mg/g

DP (estimated based on ¹H NMR): 40

Based on ¹H NMR: i=0.15

The calculated average molecular weight of co-polyamino acid AB29 is 7734 g/mol.

Organic HPLC-SEC (PEG Calibrator): Mn=4000 g/mol.

### EXAMPLE AB30

### Co-Polyamino Acid AB30—Sodium poly-L-glutamate Modified by Molecule AA10 and Having a Number-Average Molecular Weight (Mn) of 4300 g/mol

By a process similar to that used in the preparation of co-polyamino acid AB29 applied to the hydrochloride salt of molecule AA10 and a poly-L-glutamic acid obtained by a process similar to that used in the preparation of co-polyamino acid AB29-1 using molecule AA10 as the initiator in place of hexylamine, a sodium poly-L-glutamate modified with molecule AA10 is obtained.

Dry extract: 29.2 mg/g

DP (estimated based on ¹H NMR): 40

Based on ¹H NMR: i=0.125

The calculated average molecular weight of co-polyamino acid AB30 is 7682 g/mol.

Organic HPLC-SEC (PEG Calibrator): Mn=4300 g/mol.

### EXAMPLE AB31

### Co-Polyamino Acid AB31—Sodium poly-L-glutamate Modified by Molecule AA10 and Having a Number-Average Molecular Weight (Mn) of 6300 g/mol

By a process similar to that used in the preparation of co-polyamino acid AB29 applied to the hydrochloride salt of molecule AA10 and a poly-L-glutamic acid obtained by a process similar to that used in the preparation of co-polyamino acid AB29-1 using molecule AA10 as the initiator in place of hexylamine, a sodium poly-L-glutamate modified with molecule AA10 is obtained.

Dry extract: 23.1 mg/g

DP (estimated based on ¹H NMR): 40

Based on ¹H NMR: i=0.175

The calculated average molecular weight of co-polyamino acid AB31 is 8337 g/mol.

Organic HPLC-SEC (PEG Calibrator): Mn=6300 g/mol.

### EXAMPLE AB32

### Co-Polyamino Acid AB32—Sodium poly-L-glutamate Modified by Molecule AA14 and Having a Number-Average Molecular Weight (Mn) of 4700 g/mol

A sodium poly-L-glutamate modified by molecule AA14 is obtained by a process similar to that used in the preparation of co-polyamino acid AB29 applied to molecule AA14 and poly-L-glutamic acid AB29-1.

Dry extract: 13.5 mg/g

DP (estimated based on ¹H NMR): 40

Based on ¹H NMR: i=0.109

The calculated average molecular weight of co-polyamino acid AB32 is 8599 g/mol.

Organic HPLC-SEC (PEG Calibrator): Mn=4700 g/mol.

### EXAMPLE AB38

### Co-Polyamino Acid AB38—Sodium poly-L-glutamate Modified by Molecule AA18 and Having a Number-Average Molecular Weight (Mn) of 4700 g/mol

A sodium poly-L-glutamate modified by molecule AA18 is obtained by a process similar to that used in the preparation of co-polyamino acid AB29 applied to molecule AA18 and poly-L-glutamic acid AB29-1.

Dry extract: 25 mg/g

DP (estimated based on ¹H NMR): 40

Based on ¹H NMR: i=0.15

The calculated average molecular weight of co-polyamino acid AB38 is 8954 g/mol.

Organic HPLC-SEC (PEG Calibrator): Mn=4700 g/mol.

### Co-Polyamino Acids Defined According to Formula VII or VIIb

### EXAMPLE AB14

### Co-Polyamino Acid AB14—Sodium poly-L-glutamate Modified at One of Its Ends by Molecule AA1 and Having a Number-Average Molecular Weight (Mn) of 3400 g/mol

Hydrochloride salt of molecule AA1 (2.03 g, 4.70 mmol), chloroform (5 ml), molecular sieve 4 Å (1.3 g), as well as Amberlite IRN 150 ion exchange resin (1.3 g) are successively added to a suitable container. After stirring for 1 hour on rollers, the medium is filtered and the resin is rinsed with chloroform. The mixture is evaporated, then co-evaporated with toluene. The residue is solubilized in anhydrous DMF (30 mL) for direct use in the polymerization reaction.

In a previously oven-dried flask, γ-benzyl-L-glutamate N-carboxyanhydride (25.59 g, 97.2 mmol) is placed under vacuum for 30 minutes then anhydrous DMF (140 mL) is added. The mixture is stirred under argon until complete solubilization, cooled to 4° C., then the solution of molecule AA1 prepared as described above is quickly introduced. The mixture is stirred at 4° C. and room temperature for 2 days, then heated at 65° C. for 2 hours. The reaction mixture is then cooled to room temperature, then poured dropwise into diisopropyl ether (1.7 L) with stirring. The white precipitate is collected by filtration, washed twice with diisopropyl ether (140 mL), then dried under vacuum at 30° C. to obtain a white solid. The solid is diluted in TFA (160 ml), and a 33% hydrobromic acid (HBr) solution in acetic acid (62 ml, 354 mmol) is then added dropwise and at 0° C. The solution is stirred for 2 hours at room temperature and is then poured dropwise on a mixture of 1:1 (v/v) diisopropyl ether/water and with stirring (1.9 L). After stirring for 2 hours, the heterogeneous mixture is allowed to stand overnight. The white precipitate is collected by filtration, washed successively with a mixture 1: 1 (v/v) diisopropyl ether and water mixture (280 ml), then with water (140 ml). The obtained solid is solubilized in water (530 mL) by adjusting the pH to 7 by adding 10 N aqueous sodium hydroxide solution, then 1N aqueous sodium hydroxide solution. After solubilization, the theoretical concentration is adjusted to 20 g/L theoretical by addition of water to obtain a final volume of 800 mL. The mixture is filtered on a 0.45 μm filter, then purified by ultrafiltration against a 0.9% NaCl solution, then water until the conductimetry of the permeate is less than 50 μS/cm. The co-polyamino acid solution is then concentrated to about 30 g/L theoretical and the pH is adjusted to 7.0. The aqueous solution is filtered through a 0.2 μm filter and stored at 4° C.

Dry extract: 24.1 mg/g

DP (estimated by ¹H NMR)=25 where i=0.04

The calculated average molecular weight of co-polyamino acid AB14 is 3378 g/mol.

Aqueous HPLC-SEC (PEG calibrant): Mn=3400 g/mol.

### EXAMPLE AB15

### Co-Polyamino Acid AB15—Sodium poly-L-glutamate Modified at One of Its Ends by the Molecule AA6 and Having a Number-Average Molecular Weight (Mn) 4100 g/mol

By a process similar to that used in the preparation of AB14 co-polyamino acid applied to the hydrochloride salt of molecule AA6 (2.16 g, 3.94 mmol) and 25.58 g (97.2 mmol) of γ-benzyl-L-glutamate N-carboxyanhydride, a poly-L-glutamate sodium modified at one of its ends by molecule AA6 is obtained.

Dry extract: 45.5 mg/g

DP (estimated by ¹H NMR)=30 where i=0.033

The calculated average molecular weight of co-polyamino acid AB15 is 5005 g/mol.

Aqueous HPLC-SEC (PEG calibrant): Mn=4100 g/mol.

### EXAMPLE AB16

### Co-Polyamino Acid AB16—Sodium Poly-L-Glutamate Modified at One End by the Molecule AA6 and Having a Number-Average Molecular Weight (Mn) of 6500 g/mol

By a process similar to that used in the preparation of AB14 co-polyamino acid applied to the hydrochloride salt of molecule AA6 (2.39 g, 4.36 mmol) and 50.0 g (189.9 mmol) of γ-benzyl-L-glutamate N-carboxyanhydride, a poly-L-glutamate sodium modified at one of its ends by molecule AA6 is obtained.

Dry extract: 28.5 mg/g

DP (estimated by ¹H NMR)=48 where i=0.021

The calculated average molecular weight of co-polyamino acid AB16 is 7725 g/mol.

Aqueous HPLC-SEC (PEG calibrant): Mn=6500 g/mol.

### EXAMPLE AB17

### Co-Polyamino Acid AB17—Sodium poly-L-glutamate Modified at One End by the Molecule AA7 and Having a Number-Average Molecular Weight (Mn) of 3500 g/mol

By a process similar to that used in the preparation of AB14 co-polyamino acid applied to the hydrochloride salt of molecule AA7 (2.80 g, 4.32 mmol) and 25.0 g (94.9 mmol) of γ-benzyl-L-glutamate N-carboxyanhydride, a poly-L-glutamate sodium modified at one of its ends by molecule AA7 is obtained.

Dry extract: 25.2 mg/g

DP (estimated by ¹H NMR)=26 where i=0.038

The calculated average molecular weight of co-polyamino acid AB17 is 4500 g/mol.

Aqueous HPLC-SEC (PEG calibrant): Mn=3500 g/mol.

### EXAMPLE AB18

### Co-Polyamino Acid AB18—Sodium poly-L-glutamate Modified at One End by the Molecule AA7 and Having a Number-Average Molecular Weight (Mn) of 3700 g/mol

A sodium poly-L-glutamate modified on one end by molecule AA7 is obtained by polymerization of glutamic acid γ-methyl N-carboxyanhydride (25.0 g, 133.6 mmol) using the hydrochloride salt of molecule AA7 (2.80 g, 4.32 mmol) as initiator and by deprotecting methyl esters by using a 37% hydrochloric acid solution according to the process described in patent application FR-A-2 801 226.

Dry extract: 44.3 mg/g

DP (estimated by ¹H NMR)=22 where i=0.045

The calculated average molecular weight of co-polyamino acid AB18 is 3896 g/mol.

Aqueous HPLC-SEC (PEG calibrant): Mn=3700 g/mol.

### EXAMPLE AB19

### Co-Polyamino Acid AB19—Sodium poly-L-glutamate Modified at One End by the Molecule AA6 and Having a Number-Average Molecular Weight (Mn) of 10500 g/mol

A sodium poly-L-glutamate modified at one of its ends by molecule AA6 is obtained by a process similar to that used in the preparation of co-polyamino acid AB14 applied to the hydrochloride salt of molecule AA6 (1.64 g, 2.99 mmol) and to au γ-benzyl-L-glutamate N-carboxyanhydride (49.3 g, 187 mmol).

Dry extract: 23.4 mg/g

DP (estimated by ¹H NMR)=65 where i=0.015

The calculated average molecular weight of co-polyamino acid AB19 is 10293 g/mol.

Aqueous HPLC-SEC (PEG calibrant): Mn=10500 g/mol.

### EXAMPLE AB20

### Co-Polyamino Acid AB20—Sodium poly-L-glutamate Capped at One End by an Acetyl Group and Modified at One End by Molecule AA6 and Having a Number-Average Molecular Weight (Mn) of 10,400 g/mol

Hydrochloride salt of molecule AA6 (0.545 g, 1.00 mmol), chloroform (10 ml), molecular sieve 4 Å (3 g), as well as Amberlite IRN 150 ion exchange resin (3 g) are successively added to a suitable container. After stirring for 1 hour on rollers, the medium is filtered and the resin is rinsed with chloroform. The mixture is evaporated, then co-evaporated with toluene. The residue is solubilized in anhydrous DMF (10 mL) for direct use in the polymerization reaction.

In a previously oven-dried flask, γ-benzyl-L-glutamate N-carboxyanhydride (17.0 g, 64.6 mmol) is placed under vacuum for 30 minutes, then anhydrous DMF (30 mL) is added. The mixture is stirred under argon until complete solubilization, cooled to 4° C., then the solution of molecule AA6 prepared as described above is quickly introduced. The mixture is stirred between 4° C. and room temperature for 2 days, then precipitated in diisopropyl ether (0.6 L). The precipitate is collected by filtration, washed twice with diisopropyl ether (40 ml), then dried to give a white solid which is dissolved in 80 ml of THF. DIPEA (1.7 mL, 9.8 mmol) then acetic anhydride (0.9 mL, 9.5 mmol) are successively added to this solution. After stirring overnight at room temperature, the solution is slowly poured into diisopropyl ether (480 ml) over a period of 30 minutes with stirring. After 1 hour of stirring, the precipitate is filtered, washed twice with diisopropyl ether (80 ml), then dried under vacuum at 30° C. to obtain a poly (gamma-benzyl-L-glutamic acid) capped at one of its ends by an acetyl group and modified at the other of its ends by molecule AA6 in the form of a white solid.

The solid is diluted in TFA (65 ml), and a solution of 33% hydrobromic acid (HBr) in acetic acid (45 ml, 257.0 mmol) is then added dropwise and at 4° C. The solution is stirred for 2 hours at room temperature and is then poured dropwise on a 1:1 (v/v) diisopropyl ether/water mixture and with stirring (780 ml). After stirring for 2 hours, the heterogeneous mixture is allowed to stand overnight. The white precipitate is collected by filtration, washed successively with a 1:1 (v/v) diisopropyl ether and water mixture (70 ml), then with water (70 ml). The obtained solid is solubilized in water (300 ml) by adjusting the pH to 7 by adding 10 N aqueous sodium hydroxide solution, then 1N aqueous sodium hydroxide solution. After solubilization, the theoretical concentration is adjusted to 20 g/L theoretical by addition of water to obtain a final volume of 440 ml. The mixture is filtered on a 0.45 μm filter, then purified by ultrafiltration against a 0.9% NaCl solution, then water until the conductimetry of the permeate is less than 50 μS/cm. The co-polyamino acid solution is then concentrated to about 30 g/L theoretical and the pH is adjusted to 7.0. The aqueous solution is filtered through a 0.2 μm filter and stored at 4° C.

Dry extract: 21.5 mg/g

DP (estimated by ¹H NMR)=60 where i=0.017

The calculated average molecular weight of co-polyamino acid AB20 is 9619 g/mol.

Aqueous HPLC-SEC (PEG calibrant): Mn=10400 g/mol.

### EXAMPLE AB33

### Co-Polyamino Acid AB33—Sodium poly-L-glutamate Modified at One End by the Molecule AA15 and Having a Number-Average Molecular Weight (Mn) of 1800 g/mol

By a process similar to that used in the preparation of co-polyamino acid AB14 applied to molecule AA15 (0.82 g, 1.34 mmol) and 7.75 g (29.4 mmol) of γ-benzyl-L-glutamate N-carboxyanhydride, a solution of sodium poly-L-glutamate modified at one of its ends by molecule AA15 is obtained.

Dry extract: 16.8 mg/g

DP (estimated by ¹H NMR)=22 where i=0.045

The calculated average molecular weight of co-polyamino acid AB33 is 3897 g/mol.

Aqueous HPLC-SEC (PEG calibrant): Mn=1800 g/mol.

### EXAMPLE AB34

### Co-Polyamino Acid AB34—Sodium poly-L-glutamate Modified at One End by the Molecule AA4 and Having a Number-Average Molecular Weight (Mn) of 2600 g/mol

In a previously oven-dried flask, γ-benzyl-L-glutamate N-carboxyanhydride (70.9 g, 269.3 mmol) is solubilized in anhydrous DMF (125 ml). The mixture is cooled to 4° C., then a solution of molecule AA4 in the form of neutral amine (6.80 g, 12.23 mmol) in DMF (35 mL) is introduced rapidly. The mixture is stirred between 4° C. and room temperature for 18 h, then heated at 65° C. for 2 hours. The reaction mixture is then cooled to room temperature, then poured dropwise into diisopropyl ether (2.4 L) with stirring. The white precipitate is collected by filtration, washed with diisopropyl ether (2×125 mL), then dried under reduced pressure at 30° C. to give a white solid. The solid is solubilized in N,N-dimethylacetamide (DMAc, 150 ml), then Pd/Al₂O₃ (6 g) is added under an argon atmosphere. The mixture is placed in a hydrogen atmosphere (10 bar) and stirred at 60° C. for 24 hours. After cooling to room temperature and filtration of the catalyst on a P4 sintered filter then on a PTFE Omnipore hydrophilic membrane 0.2 μm, a solution of water at pH 2 (900 ml) is poured dropwise on the DMAc solution, on a 45 minute period and with stirring. After 18 hours of, the white precipitate is collected by filtration, washed with water, then dried under reduced pressure at 30° C. The obtained solid is solubilized in water (1.25 L) by adjusting the pH to 7 by addition of a 1N aqueous sodium hydroxide solution. The pH is then adjusted to pH 12 and the solution is kept under stirring for 1 h. After neutralization at pH 7, the solution is filtered through a 0.2 μm filter, diluted with ethanol to obtain a solution containing 30% mass of ethanol, then filtered through an activated carbon filter (3M R53 SLP). The solution obtained is filtered through a 0.45 μm filter and purified by ultrafiltration against a 0.9% NaCl solution, then water until the conductimetry of the permeate is less than 50 μS/cm. The co-polyamino acid solution is then concentrated to approximately 30 g/L theoretical and the pH is adjusted to 7. The aqueous solution is filtered through a 0.2 μm filter and stored at 4° C.

Dry extract: 38.1 mg/g

DP (estimated by ¹H NMR)=23 where i=0.043

The calculated average molecular weight of co-polyamino acid AB34 is 3991 g/mol.

Organic HPLC-SEC (PEG Calibrator): Mn=2600 g/mol.

### EXAMPLE AB35

### Co-Polyamino Acid AB35—Sodium poly-L-glutamate Modified at One End by the Molecule AA14 and having a Number-Average Molecular Weight (Mn) of 2600 g/mol

A solution of poly-L-glutamate modified at one of its ends by molecule AA14 is obtained by a process similar to that used in the preparation of AB34 co-polyamino acid applied to AA14 molecule (0.4 g, 0.65 mmol) in chloroform solution (6.5 ml) and to 3.79 g (14.4 mmol) of γ-benzyl-L-glutamate N-carboxyanhydride solution dissolved in DMF (6.5 ml), and by omitting the activated carbon filtration step. Dry extract: 21.0 mg/g DP (estimated by ¹H NMR)=22 where i=0.045

The calculated average molecular weight of co-polyamino acid AB35 is 3896 g/mol.

Organic HPLC-SEC (PEG Calibrator): Mn=2600 g/mol.

### EXAMPLE AB36

### Co-Polyamino Acid AB36—Sodium poly-L-glutamate Modified at One End by the Molecule AA16 and Having a Number-Average Molecular Weight (Mn) of 2800 g/mol

By a process similar to that used in the preparation of co-polyamino acid AB34 applied to molecule AA16 (3.28 g, 4.67 mmol) and 27.02 g (102.6 mmol) of γ-benzyl-L-glutamate N-carboxyanhydride, a solution of sodium poly-L-glutamate modified at one of its ends by molecule AA16 is obtained.

Dry extract: 23.9 mg/g

DP (estimated by ¹H NMR)=22 where i=0.045

The calculated average molecular weight of co-polyamino acid AB36 is 3987 g/mol.

Organic HPLC-SEC (PEG Calibrator): Mn=2800 g/mol.

### EXAMPLE AB37

### Co-Polyamino Acid AB37—Sodium poly-L-glutamate Modified at One of Its Ends by the Molecule AA17 and Having a Number-Average Molecular Weight (Mn) of 2800 g/mol

By a process similar to that used in the preparation of co-polyamino acid AB34 applied to molecule AA17 (4.50 g, 9.73 mmol) and 56.33 g (214.0 mmol) of γ-benzyl-L-glutamate N-carboxyanhydride, a solution of sodium poly-L-glutamate modified at one of its ends by molecule AA17 is obtained.

Dry extract: 26.8 mg/g

DP (estimated by ¹H NMR)=24 where i=0.042

The calculated average molecular weight of co-polyamino acid AB37 is 4049 g/mol.

Organic HPLC-SEC (PEG Calibrator): Mn=2800 g/mol.

BB: Synthesis of Intermediate Hydrophobic Hy Compounds to Obtain the -Hy Radicals Wherein p=2

Hydrophobic intermediate compounds are represented in the following table by the corresponding hydrophobic intermediate compound before grafting on co-polyamino acid.

TABLE 1D
List of hydrophobic intermediates synthesized according to the invention.

| N° | HYDROPHOBIC INTERMEDIATE COMPOUNDS |
|---|---|
| BA1 | 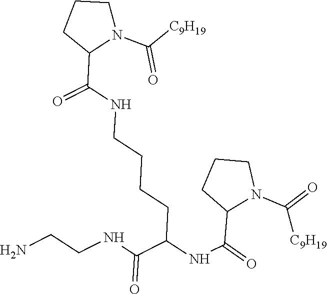 |
| BA2 | 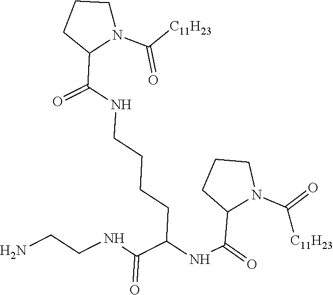 |
| BA3 | 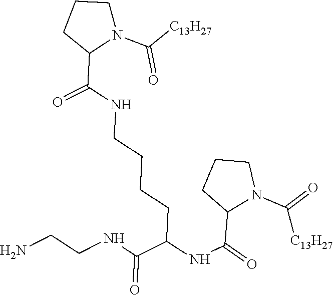 |
| BA4 | 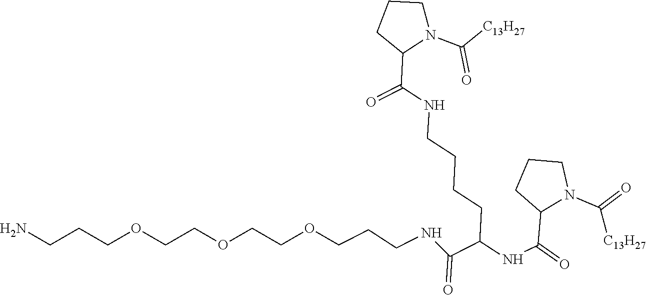 |
| BA5 | 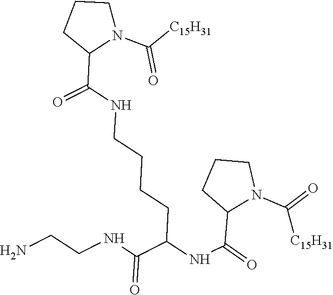 |
| BA6 | 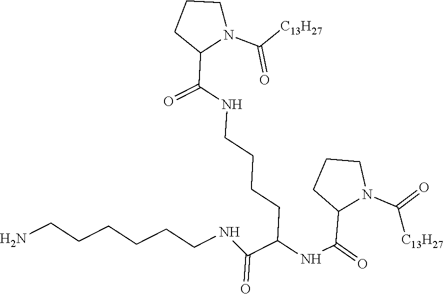 |
| BA7 | 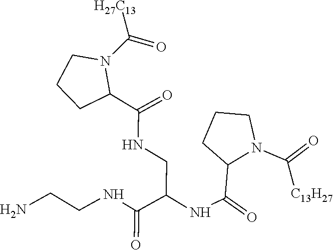 |

BA: Synthesis of Hydrophobic Intermediate Compounds Hy Making it Possible to Obtain -Hy Radicals Wherein p=2

### EXAMPLE BA1

### Molecule BA1

Molecule B1: Product Obtained by the Reaction between Decanoic Acid and L-proline.

Dicyclohexyl carbodiimide (DCC) (16.29 g, 78.96 mmol) and N-hydroxysuccinimide (NHS) (9.09 g, 78.96 mmol) are successively added to a solution of decanoic acid (14.28 g, 82.91 mmol) in THF (520 ml) at 0° C. After stirring for 60 hours at room temperature, the medium is cooled to 0° C. for 20 minutes, filtered through a sintered frit. L-Proline (10 g, 86.86 mmol), diisopropylethylamine (DIPEA) (68.8 mL) and water (60 mL) are added to the filtrate. After stirring for 24 hours at room temperature, the medium is diluted with water (300 ml). The aqueous phase is washed with ethyl acetate (2×250 ml), acidified to pH˜1 with a 1N HCl aqueous solution, then extracted with dichloromethane (3×150 ml). The combined organic phases are dried over Na₂SO₄, filtered, concentrated under vacuum, and the residue is purified by chromatography on silica gel (cyclohexane, ethyl acetate).

Yield: 14.6 g (69%)

NMR ¹H (CDCl₃, ppm): 0.87 (3H); 1.26 (12H); 1.65 (2H); 2.02 (3H); 2.34 (2H); 2.41 (1H); 3.48 (1H); 3.56 (1H); 4.58 (1H).

LC/MS (ESI): 270.2; (calculated ([M+H]⁺): 270.4).

### Molecule B2: Product Obtained by the Reaction Between Molecule B1 and L-lysine.

By a process similar to that used in the preparation of molecule B1 applied to molecule B1 (14.57 g, 54.07 mmol) and to L-lysine (4.15 g, 28.39 mmol), a yellow oil is obtained.

Yield: 16.4 g (93%)

NMR ¹H (CDCl₃, ppm): 0.88 (6H); 1.26 (24H); 1.35-1.65 (8H); 1.85-2.35 (12H); 2.53 (0.2H); 2.90 (0.8H); 3.45-3.75 (5H); 4.50-4.70 (3H); 7.82 (1H).

LC/MS (ESI): 649.6; (calculated ([M+H]+): 649.9).

### Molecule B3: Product Obtained by Reaction Between Molecule B2 and N-Boc-ethylenediamine.

DIPEA (8.80 mL) and 2-(1H-benzotriazol-1-yl)-1,1,3,3-tetramethyluronium tetrafluoroborate (TBTU, 8.52 g, 26.54 mmol) at room temperature are added to a solution of molecule B2 (16.4 g, 25.27 mmol) in THF (170 ml). After stirring for 30 minutes, BocEDA (4.45 g, 27.8 mmol) is added. After stirring at room temperature for 2 hours, the solvent is evaporated under reduced pressure and the residue is diluted with ethyl acetate (400 mL). The organic phase is washed with water (250 mL), a saturated aqueous solution of NaHCO₃ (250 mL), an aqueous solution of 1 N HCl (250 mL), a saturated aqueous solution of NaC1 (250 mL) and dried over Na₂SO₄. After filtration and concentration under vacuum, the residue obtained is purified by chromatography on silica gel (ethyl acetate, methanol) to produce a colorless oil.

Yield: 12.8 g (64%)

NMR ¹H (CDCl₃, ppm): 0.87 (6H); 1.25-1.60 (42H); 1.80-2.05 (4H); 2.15-2.45 (9H); 3.10-3.75 (10H); 4.30 (1H); 4.50 (2H); 5.50 (0.6H); 5.89 (0.2H); 6.15 (0.2H); 7.03 (1H); 7.47 (1H).

LC/MS (ESI): 791.8; (calculated ([M+H]+): 792.1).

### Molecule BA1

A solution of 4 N HCl in dioxane (20.2 ml) is added to a solution of molecule B3 (12.78 g, 16.15 mmol) in dichloromethane (110 ml) at 5° C. After 20 hours of stirring at 5° C., the medium is concentrated under vacuum. The residue obtained is dissolved in methanol and evaporated under vacuum, this process being repeated 4 times to give a white solid of molecule BA1 in the form of hydrochloride salt.

Yield: 11.4 g (97%)

NMR ¹H (DMSO-d₆, ppm): 0.85 (6H); 1.25-1.50 (33H); 1.57 (1H); 1.70-2.40 (12H); 2.82 (2H); 3.00 (2H); 3.25-3.70 (6H); 4.05-4.50 (3H); 7.75-8.45 (6H).

LC/MS (ESI): 691.6; (calculated ([M+H]+): 692.0).

### EXAMPLE BA2

### Molecule BA2

### Molecule B4: Product Obtained by the Reaction Between Lauric Acid and L-proline.

By a process similar to that used in the preparation of molecule B1, applied to lauric acid (31.83 g, 157.9 mmol) and to L-proline (20 g, 173.7 mmol), a yellow oil is obtained.

Yield: 34.3 g (73%)

NMR ¹H (CDCl₃, ppm): 0.87 (3H); 1.26 (16H); 1.70 (2H); 1.90-2.10 (3H); 2.35 (2H); 2.49 (1H); 3.48 (1H); 3.56 (1H); 4.60 (1H).

LC/MS (ESI): 298.2; (calculated ([M+H]+): 298.4).

### Molecule B5: Product Obtained by the Reaction Between Molecule B4 and L-lysine.

A white solid is obtained by a process similar to that used in the preparation of molecule B1 applied to molecule B4 (33.72 g, 113.36 mmol) and to L-lysine (8.70 g, 59.51 mmol).

Yield: 26.2 g (66%)

NMR ¹H (CDCl₃, ppm): 0.88 (6H); 1.26 (32H); 1.35-1.65 (8H); 1.85-2.35 (15H); 2.87 (1H); 3.40-3.75 (5H); 4.50-4.75 (3H); 7.87 (1H).

LC/MS (ESI): 705.6; (calculated ([M+H]+): 706.0).

### Molecule B6: Product Obtained by Reaction Between N-Boc-ethylenediamine and Molecule B5.

A colorless oil colorless is obtained by a process similar to that used in the preparation of molecule B3 applied to molecule B5 (25.74 g, 36.51 mmol) and BocEDA (6.43 g, 40.16 mmol).

Yield: 30.9 g (quantitative)

NMR ¹H (CDCl₃, ppm): 0.88 (6H); 1.35-1.65 (50H); 1.85-2.35 (13H); 3.05-3.75 (10H); 4.25-4.65 (3H); 5.50 (0.4H); 5.88 (0.2H); 6.16 (0.2H); 7.08 (1H); 7.26 (1H); 7.49 (0.2H).

LC/MS (ESI): 847.8; (calculated ([M+H]+): 848.2).

### Molecule BA2

Following a process similar to that used in the preparation of molecule BA1 applied to molecule B6 (30.9 g, 36.47 mmol), the residue obtained after concentration under vacuum is dissolved in methanol and evaporated under vacuum, this process being repeated 4 times to yield a white solid of molecule BA2 in the form of a hydrochloride salt after drying under reduced pressure.

Yield: 27.65 g (97%)

NMR ¹H (DMSO-d₆, ppm): 0.85 (6H); 1.10-2.40 (54H); 2.75-3.15 (4H); 3.25-3.60 (6H); 4.05-4.50 (3H); 7.50-8.50 (6H).

LC/MS (ESI): 747.6; (calculated ([M+H]+): 748.1).

### EXAMPLE BA3

### Molecule BA3

### Molecule B7: Product Obtained by the Reaction Between Myristic Acid and L-proline.

A yellow oil is obtained by a process similar to that used in the preparation of molecule B1, applied to lauric acid (18.93 g, 82.91 mmol) and to L-proline (10 g, 86.86 mmol).

Yield: 20 g (78%)

NMR ¹H (CDCl₃, ppm): 0.88 (3H); 1.28 (20H); 1.70 (2H); 1.90-2.10 (3H); 2.36 (2H); 2.51 (1H); 3.47 (1H); 3.56 (1H); 4.61 (1H).

LC/MS (ESI): 326.2; (calculated ([M+H]+): 326.6).

### Molecule B8: Product Obtained by the Reaction Between Molecule B7 and L-lysine

A white solid is obtained by a process similar to that used in the preparation of molecule B1 applied to molecule B7 (20.02 g, 61.5 mmol) and to L-lysine (4.72 g, 32.29 mmol).

Yield: 12.3 g (53%)

NMR ¹H (DMSO-d₆, ppm): 0.85 (6H); 1.26 (40H); 1.35-1.50 (6H); 1.50-2.10 (10H); 2.10-2.25 (4H); 3.01 (2H); 3.31-3.55 (4H); 4.10-4.40 (3H); 7.68 (0.6H); 7.97 (1H); 8.27 (0.4H); 12.50 (1H).

LC/MS (ESI): 761.8; (calculated ([M+H]+): 762.1).

### Molecule B9: Product Obtained by Reaction Between N-Boc-ethylenediamine and Molecule B8.

By a process similar to that used in the preparation of molecule B3 applied to molecule B8 (12 g, 15.77 mmol) and BocEDA (3.03 g, 18.92 mmol), a colorless oil is obtained after purification by chromatography column on silica gel (ethyl acetate, methanol).

Yield: 12.5 g (88%)

NMR ¹H (DMSO-d₆, ppm): 0.85 (6H); 1.20-1.55 (55H); 1.50-2.25 (14H); 2.95-3.10 (6H); 3.31-3.55 (4H); 4.10-4.40 (3H); 6.74 (1H); 7.60-8.25 (3H).

LC/MS (ESI): 904.1; (calculated ([M+H]+): 904.3).

### Molecule BA3

Following a process similar to that used in the preparation of molecule BA1 applied to molecule B9 (12.5 g, 13.84 mmol), the residue obtained after concentration under vacuum is dissolved in methanol and evaporated under vacuum, this process being repeated 4 times to yield a white solid of molecule BA3 in the form of a hydrochloride salt after drying under reduced pressure.

Yield: 9.2 g (79%)

NMR ¹H (DMSO-d₆, ppm): 0.85 (6H); 1.10-1.65 (48H); 1.70-2.35 (12H); 2.85 (2H); 3.01 (2H); 3.25-3.65 (6H); 4.10-4.50 (3H); 7.70-8.40 (6H).

LC/MS (ESI): 803.9; (calculated ([M+H]+): 804.2).

### EXAMPLE BA4

### Molecule BA4

Molecule B10: Product Obtained by Reaction Between Molecule B8 and Boc-1-amino-4,7,10-trioxa-13-tridecane amine.

By a process similar to that used in the preparation of molecule B3 applied to molecule B8 (29.80 g, 39.15 mmol) and Boc-1-amino-4,7,10-trioxa-13-tridecane amine (15.05 g, 46.96 mmol), a thick colorless oil is obtained.

Yield: 25.3 g (61%)

NMR ¹H (DMSO-d₆, ppm): 0.85 (6H); 1.25-2.35 (75H); 2.85-3.20 (6H); 3.25-3.65 (16H); 4.10-4.45 (3H); 6.38 (0.1H); 6.72 (0.9H); 7.50-8.25 (3H).

LC/MS (ESI): 1064.2; (calculated ([M+H]+): 1064.5).

### Molecule BA4

Following a process similar to that used in the preparation of molecule BA1 applied to molecule B10 (25.3 g, 23.8 mmol), the residue obtained after concentration under vacuum is dissolved in methanol and evaporated under vacuum, this process being repeated 4 times to yield a white solid of molecule BA4 in the form of a hydrochloride salt after drying under reduced pressure.

Yield: 20.02 g (84%)

NMR ¹H (DMSO-d₆, ppm): 0.85 (6H); 1.15-2.35 (66H); 2.80-3.20 (6H); 3.30-3.65 (16H); 4.10-4.45 (3H); 7.55-8.60 (6H).

LC/MS (ESI): 964.9; (calculated ([M+H]+): 964.6).

### EXAMPLE BA5

### Molecule BA5

### Molecule B11: Product Obtained by Reaction Between Molecule A1 and L-Lysine

By a process similar to that used in the preparation of molecule B1 applied to molecule A1 (19.10 g, 54.02 mmol) and L-lysine (4.15 g, 28.36 mmol), an oily residue is obtained after concentration of the reaction medium under reduced pressure. This residue is diluted in water (150 mL), washed with ethyl acetate (2×75 mL), then the aqueous phase is acidified to pH 1 by slow addition of 6N HCl. The product is extracted 3 times with dichloromethane, the organic phase is dried over Na₂SO₄ then filtered and concentrated under reduced pressure to give 11.2 g of yellow oily residue. Simultaneously, the organic phase of the above ethyl acetate is washed with an aqueous solution of 2N HCl (2×75 ml), a saturated aqueous solution of NaCl (75 ml), dried over Na₂SO₄, filtered and concentrated to give 10.2 g of yellow oily residue. A white solid is obtained after recrystallization of each of these residues in acetone.

Yield: 11.83 g (54%)

¹H RMN (CDCl₃, ppm): 0.87 (6H); 1.06-2.44 (70H); 2.78-2.96 (1H); 3.35-3.75 (5H); 4.28-4.43 (0.1H); 4.43-4.52 (0.2H); 4.52-4.61 (1.8H); 4.61-4.75 (0.9H); 7.74-8.02 (2H).

LC/MS (ESI): 818.0; (calculated ([M+H]+): 818.7).

### Molecule B12: Product Obtained by Coupling Between Molecule B11 and N-Boc-ethylenediamine

By a process similar to that used in the preparation of molecule B3 applied to molecule B11 (18.00 g, 22.02 mmol) solution in THF and Boc-ethylenediamine (4.23 g, 26.43 mmol), a white solid is obtained after double recrystallization in acetonitrile.

Yield: 17.5 g (83%)

1H NMR (DMSO-d6, ppm): 0.85 (6H); 1.15-2.29 (79H); 2.92-3.12 (6H); 3.30-3.59 (4H); 4.06-4.13 (0.65H); 4.16-4.29 (2H); 4.38-4.42 (0.35H); 6.71-6.76 (1H); 7.60-7.69 (1.3H); 7.76-7.81 (0.65H); 7.93-7.97 (0.35H); 8.00-8.04 (0.35H); 8.10-8.17 (0.35H).

LC/MS (ESI): 960.4 (calculated ([M+H]⁻): 960.8).

### Molecule BA5

By a process similar to that used in the preparation of molecule BA1 applied to molecule B12 (24.4 g, 25.43 mmol), the residue obtained after concentration under vacuum is solubilized in dichloromethane (150 ml), the organic phase is washed twice with aqueous 2N sodium hydroxide solution (90 ml). Acetonitrile (120 mL) is added and dichloromethane is removed by concentration under reduced pressure. The medium is then left standing for 72 hours and a white solid is obtained after filtration and rinsing with acetonitrile, then drying under reduced pressure. This process is repeated 4 times.

Yield: 14.28 g (65%)

1H NMR (DMSO-d6, ppm): 0.85 (6H); 1.06-2.32 (70H); 2.53-2.63 (2H); 2.89-3.61 (10H); 4.04-4.43 (3H); 7.55-7.62 (0.65H); 7.65-7.72 (0.65H); 7.80 (0.65H); 7.91 (0.35H); 8.03 (0.35H); 8.14-8.23 (0.35H).

LC/MS (ESI): 860.0 (calculated ([M+H]+): 860.8).

### EXAMPLE BA6

### Molecule BA6

Molecule B13: Product Obtained by the Reaction Between N-(tert-butoxycarbonyl)-1,6-diaminohexane and Molecule B8.

By a process similar to that used in the preparation of molecule B3 applied to molecule B8 (10 g, 13.14 mmol) and to N-(tent-butoxycarbonyl)-1.6-diaminohexane (3.41 g, 15.77 mmol) in dichloromethane, a white solid is obtained after recrystallization in acetonitrile.

Yield: 10.7 g (85%)

NMR ¹H (CDCl₃, ppm): 0.88 (6H); 1.17-2.40 (79H); 3.00-3.71 (10H); 4.26-4.58 (3H); 4.67 (1H); 6.74 (1H); 7.34-7.49 (2H).

LC/MS (ESI): 959.9; (calculated ([M+H]+): 959.8).

### Molecule BA6

Following a process similar to that used in the preparation of molecule BA1 applied to molecule B13 (10.5 g, 10.94 mmol), an aqueous solution of 2N NaOH is added dropwise to the reaction medium cooled to 0° C. The aqueous phase is extracted with dichloromethane and the organic phase is washed thrice with 5% aqueous NaCl solution. After drying over Na₂SO₄, the organic phase is filtered, concentrated under vacuum, and the residue is recrystallized in acetonitrile.

Yield: 5.4 g (58%)

NMR ¹H (CDCl₃, ppm): 0.88 (6H); 1.19-2.40 (72H); 2.67 (2H); 3.03-3.70 (8H); 4.26-4.57 (3H); 6.71 (1H); 7.39-7.49 (2H).

LC/MS (ESI): 859.8; (calculated ([M+H]+): 859.7).

### EXAMPLE BA7

### Molecule BA7

Molecule B14: Product Obtained by Coupling Between Molecule B7 and 2,3-diaminopropionic Acid

By a process similar to that used in the preparation of molecule B1 applied to molecule B7 (80.00 g, 245.78 mmol) and to the dihydrochloride of 2,3-diaminopropionic acid (22.84 g, 129.04 mmol), a white solid is obtained after recrystallization in acetonitrile.

Yield: 69 g (78%)

1H NMR (DMSO-d6, ppm): 0.86 (6H); 1.08-1.38 (40H); 1.40-1.55 (4H); 1.68-2.30 (12H); 3.16-3.66 (6H); 4.20-4.39 (3H); 7.67-8.31 (2H); 12.70 (1H).

LC/MS (ESI): 719.4; 741.5; (calculated ([M+H]⁺): 719.6; ([M+Na]⁺): 741.6).

### Molecule B15: Product Obtained by Coupling Between Molecule B14 and N-Boc-ethylenediamine

By a process similar to that used in the preparation of molecule B3 applied to molecule B14 (32.00 g, 44.50 mmol) and N-Boc-ethylenediamine (8.56 g, 53.40 mmol), a colorless oil is obtained after purification by chromatography column on silica gel (ethyl acetate, methanol).

Yield: 24.5 g (64%)

1H NMR (DMSO-d6, ppm): 0.85 (6H); 1.16-2.42 (65H); 2.89-3.14 (4H); 3.17-3.66 (6H); 4.11-4.43 (3H); 6.77 (1H); 7.38-8.23 (3H).

LC/MS (ESI): 861.7 (calculated ([M+H]+): 861.7).

### Molecule BA7

After a process similar to that used in the preparation of molecule BA1 applied to molecule B15 (24.50 g, 28.45 mmol), the reaction medium is concentrated under reduced pressure, the residue is solubilized in dichloromethane, the phase organic is washed with an aqueous solution of NaOH 2 N), dried over Na₂SO₄, filtered and concentrated under reduced pressure. A white solid is obtained after recrystallization in acetonitrile.

Yield: 19.7 g (91%)

1H NMR (DMSO-d6, ppm): 0.85 (6H); 1.10-2.40 (58H); 2.51-2.62 (2H); 2.90-3.16 (2H); 3.16-3.67 (6H); 4.04-4.47 (3H); 7.33-8.27 (3H).

LC/MS (ESI): 761.5 (calculated ([M+H]⁻): 761.6).

BB: Synthesis of Co-Polyamino Acids Modified by Hydrophobic Molecules Wherein p=2

### Co-Polyamino Acids According to Formula VII or VIIa

TABLE 1E
List of co-polyamino acids according to formula VII or VIIa synthesized
according to the invention wherein p = 2

| | CO-POLYAMINO ACIDS BEARING CARBOXYLATE CHARGES AND |
|---|---|
| N° | HYDROPHOBIC RADICALS |
| BB1 | 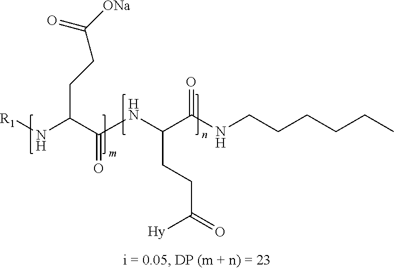 |
| | 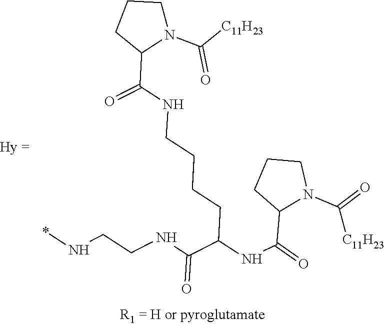 |
| BB2 | 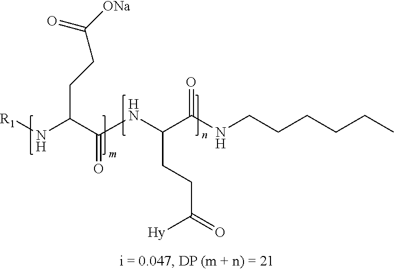 |
| | 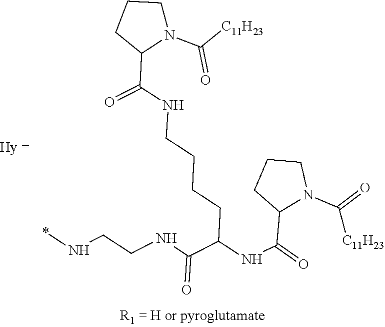 |
| BB3 | 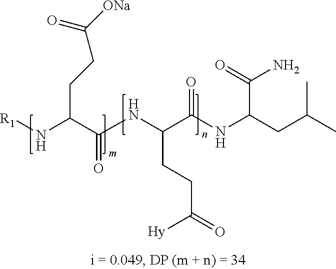 |
| | 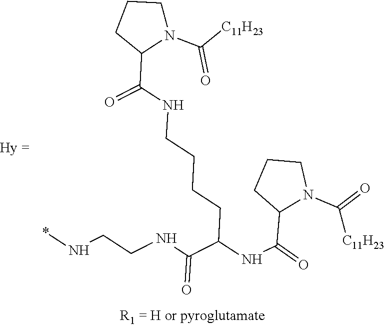 |
| BB4 | 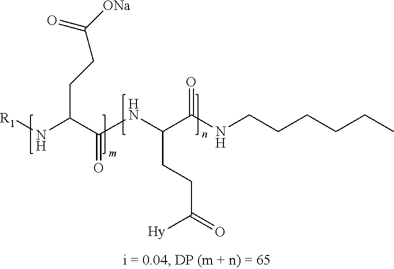 |
| | 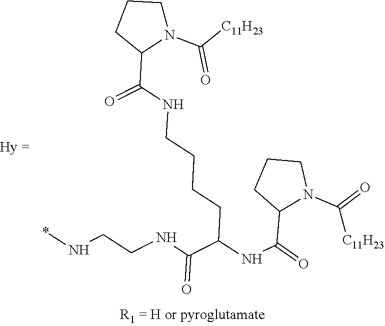 |
| BB5 | 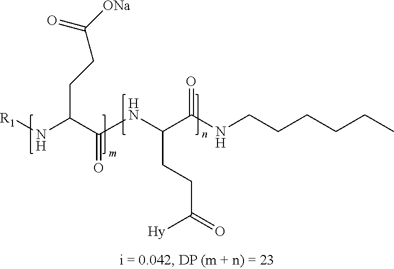 |
| | 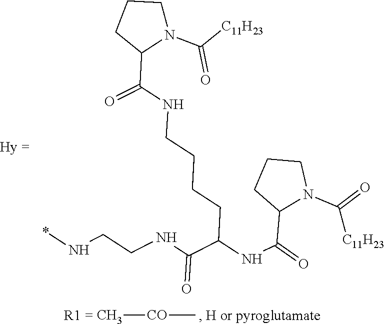 |
| BB6 | 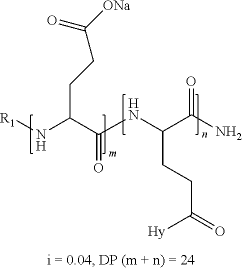 |
| | 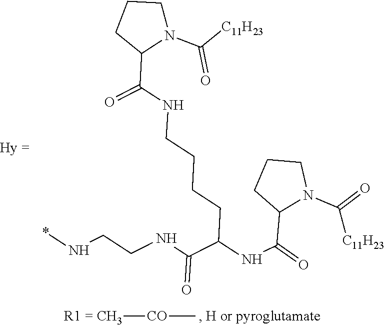 |
| BB7 | 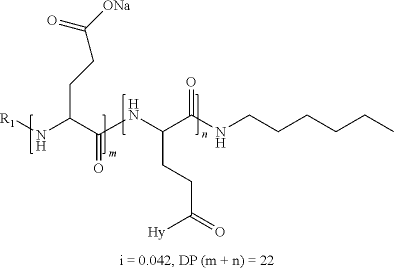 |
| | 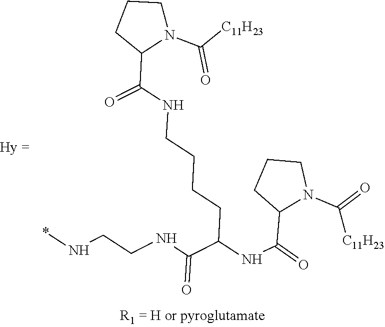 |
| BB8 | 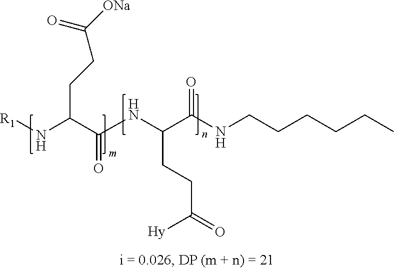 |
| | 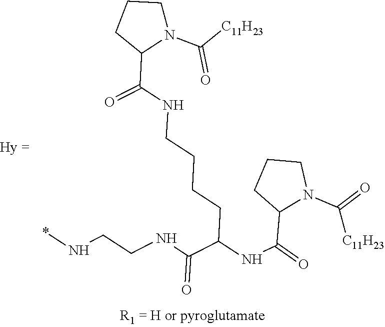 |
| BB9 | 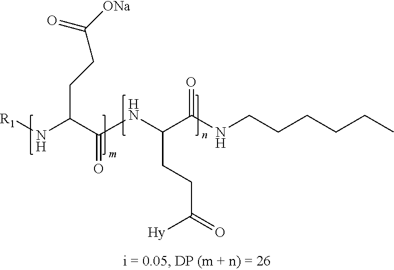 |
| | 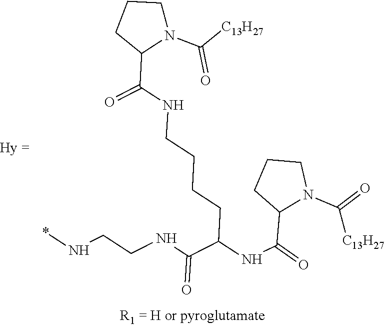 |
| BB10 | 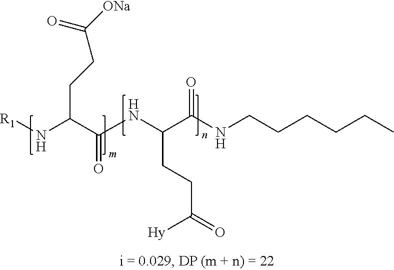 |
| | 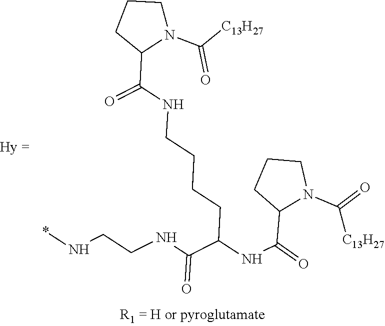 |
| BB11 | 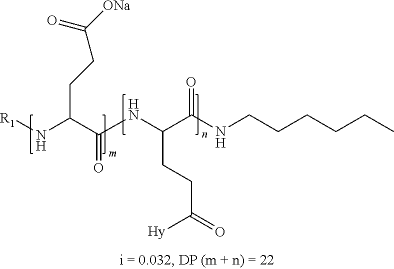 |
| | 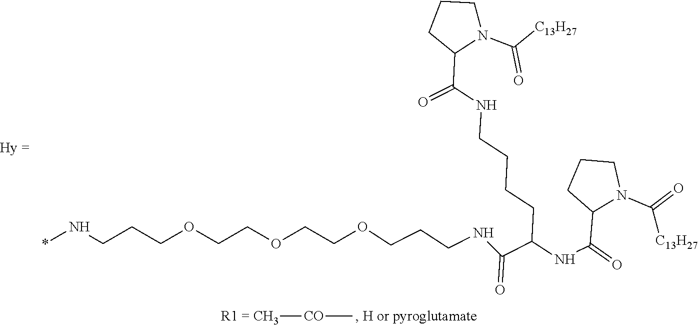 |
| BB12 | 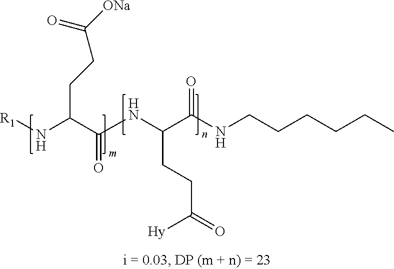 |
| | 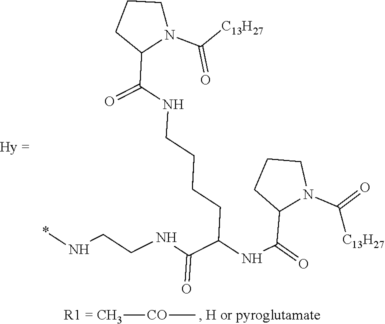 |
| BB13 | 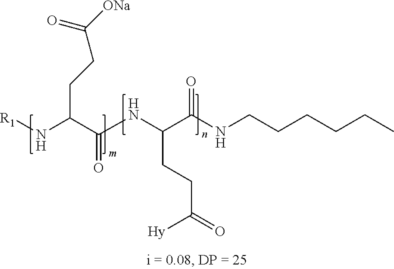 |
| | 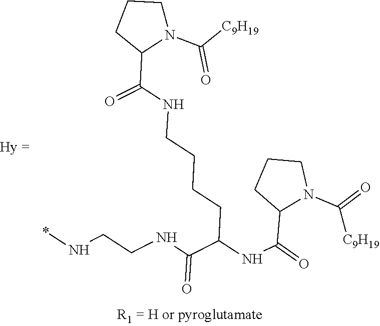 |

### Co-Polyamino Acids According to Formula VII or VIIb

TABLE 1F
List of co-polyamino acids according to formula VII or VIIb synthesized
according to the invention.

| | CO-POLYAMINOACIDES BEARING CARBOXYLATE LOADS AND |
|---|---|
| N° | HYDROPHOBIC RADICALS |
| BB14 | 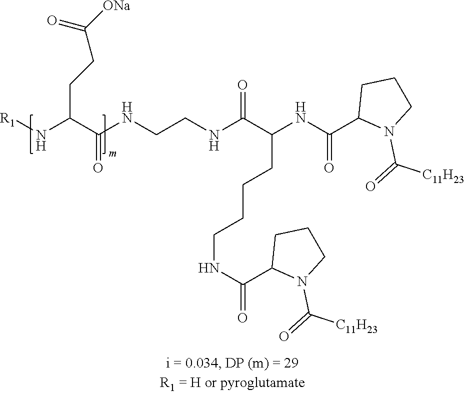 |
| BB15 | 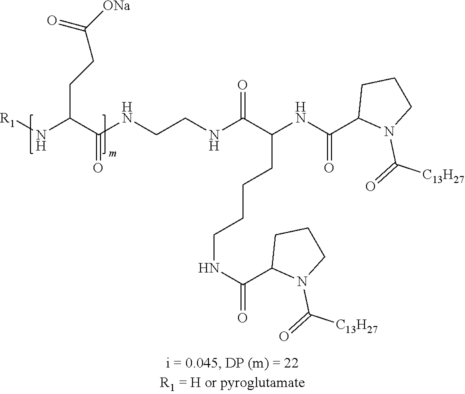 |
| BB15′ | i = 0.042, DP (m) = 24 |
| | R₁ = H or pyroglutamate |
| BB16 | 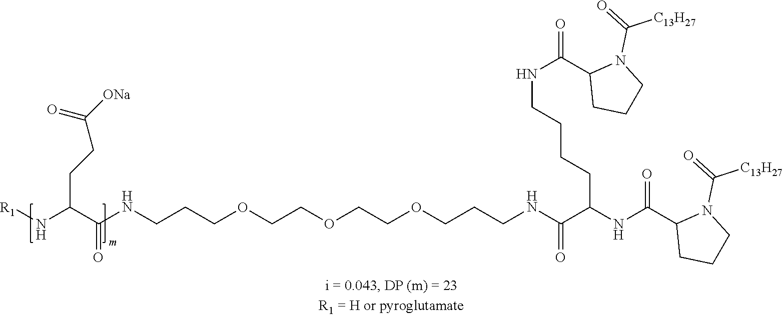 |
| BB17 | 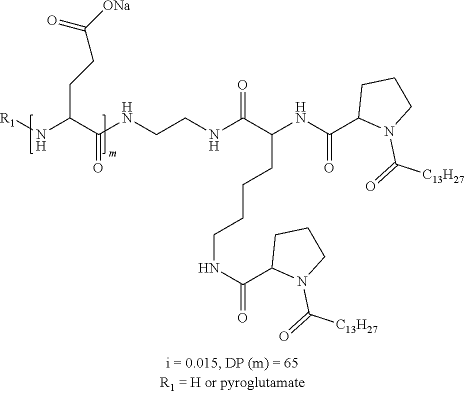 |
| BB18 | 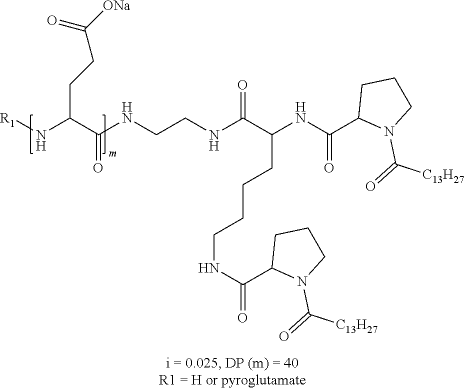 |
| BB19 | 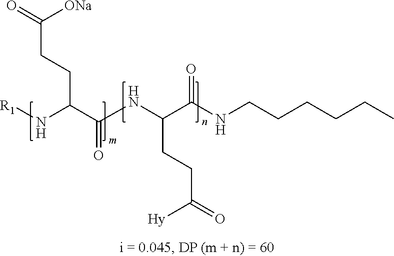 |
| | 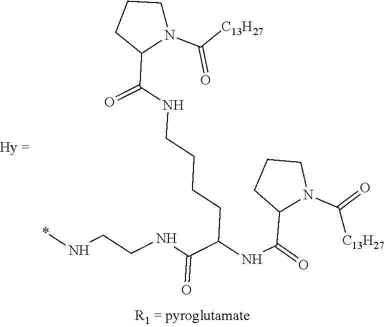 |
| BB20 | 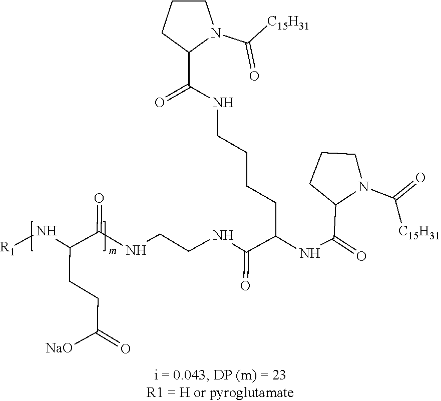 |
| BB21 | 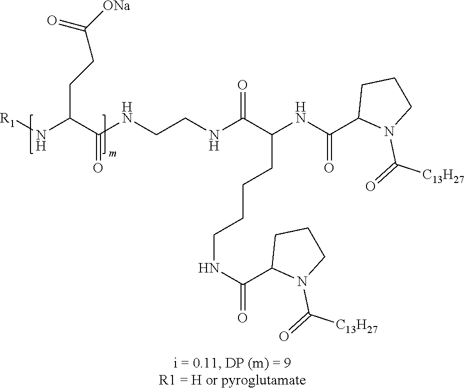 |
| BB22 | 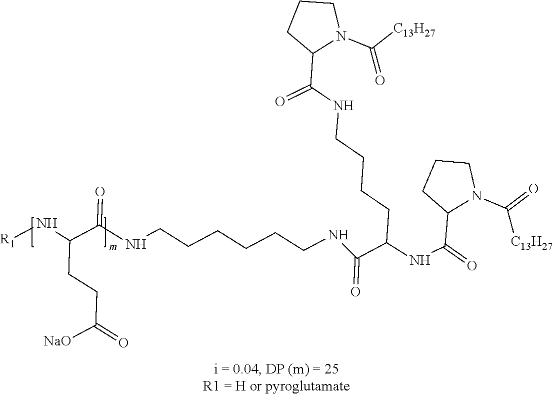 |
| BB23 | 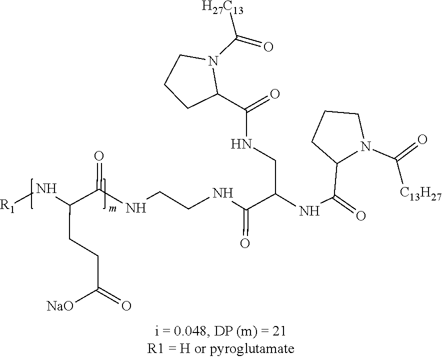 |
| BB24 | 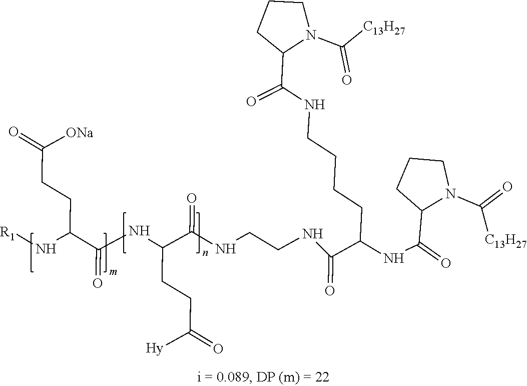 |
| | 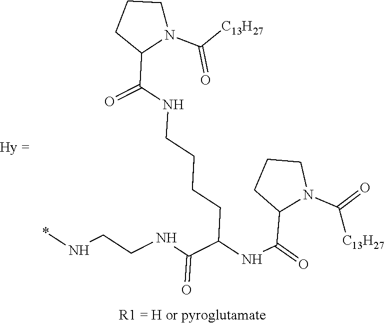 |
| BB25 | 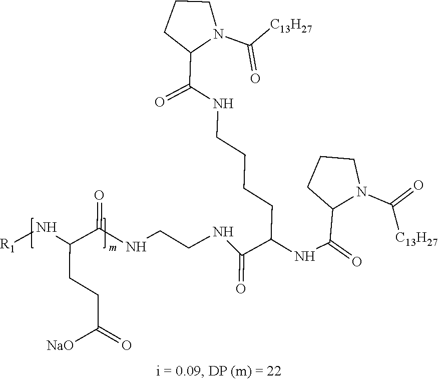 |
| | 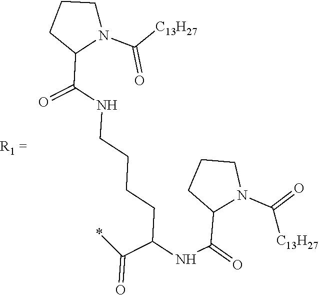 |

BB: Synthesis of Co-Polyamino Acids Modified by Hydrophobic Molecules Wherein p=2.

### EXAMPLE BB1

### Co-Polyamino Acid BB1—Sodium poly-L-glutamate Modified by Molecule BA2 and Having a Number-Average Molecular Weight (Mn) of 2400 g/mol

Co-Polyamino Acid BB1-1: poly-L-glutamic Acid Having an Average Relative Numerical Molecular Weight (Mn) 3860 g/mol from the Polymerization of γ-benzyl-L-glutamate N-carboxyanhydride Initiated by hexylamine

In a previously oven-dried flask, γ-benzyl-L-glutamate N-carboxyanhydride (90.0 g, 342 mmol) is placed under vacuum for 30 minutes, then anhydrous DMF (465 ml) is added. The mixture is then stirred under argon until complete dissolution, cooled to 4° C., then hexylamine (1.8 ml 14 mmol) is quickly introduced. The mixture is stirred at 4° C. and room temperature for 2 days. The reaction medium is then heated at 65° C. for 4 hours, cooled to room temperature, then poured dropwise into diisopropyl ether (6 L) with stirring. The white precipitate is collected by filtration, washed with diisopropyl ether (500 ml×250 ml), then dried under vacuum at 30° C. to produce a poly (γ-benzyl-L-glutamic acid) (PBLG).

A hydrobromic acid solution (HBr) at 33% in acetic acid (135 mL, 0.77 mol) is added dropwise to a solution of PBLG (42.1 g) in trifluoroacetic acid (TFA, 325 mL) at 4° C. The mixture is stirred at room temperature for 2 hours, then poured dropwise onto a 1:1 (v/v) mixture of diisopropyl ether and water with stirring (1.6 L). After stirring for 1 hour 30 minutes, the heterogeneous mixture is allowed to stand overnight. The white precipitate is collected by filtration, washed with a 1:1 (v/v) mixture of diisopropyl ether and water (200 mL).

The obtained solid is solubilized in water (1 mL) by adjusting the pH to 7 by adding 10 N aqueous sodium hydroxide solution, then 1N aqueous sodium hydroxide solution. After solubilization, the theoretical concentration is adjusted to 25 g/L theoretical by addition of water to obtain a final volume of 1.5 mL.

The solution is filtered through a 0.45 μm filter, then purified by ultrafiltration against a solution of NaCl 0.9%, then water until the conductimetry of the permeate is less than 50 μS/cm.

The aqueous solution is then acidified by adding 37% hydrochloric acid solution until a pH of 2 is reached. After stirring for 4 hours, the precipitate obtained is filtered, then dried under vacuum at 30° C. to give a poly-L-glutamic acid with a number-average molecular weight (Mn) of 3860 g/mol relative to a polyoxyethylene standard (PEG).

### Co-Polyamino Acid BB1

Co-polyamino acid BB1-1 (10.0 g) is solubilized in DMF (700 ml) at 30-40° C. and then cooled to 0° C. The hydrochloride salt of molecule BA2 (2.95 g, 3.8 mmol) is suspended in DMF (45 ml) and triethylamine (0.39 g, 3.8 mmol) is then added to this suspension and the mixture is slightly heated while stirring until complete dissolution. N-methylmorpholine (NMM, 7.6 g, 75 mmol) in DMF (14 mL) and ethyl chloroformate (ECF, 8.1 g, 75 mmol) are added to a solution of co-polyamino acid at 0° C. After 10 minutes at 0° C., the Molecule BA2 solution is added and the medium maintained at 30° C. for 1 h. The reaction mixture is poured dropwise over 6 L of water containing 15% NaCl weight and HCl (pH 2), and left to stand overnight. The precipitate is collected by filtration, washed with sodium chloride solution at pH 2 (1 L) and dried under vacuum for about 1 hour. The white solid obtained is returned to water (600 ml) and the pH is adjusted to 7 by slowly adding a 1N aqueous solution of NaOH. The volume is adjusted to 700 ml by addition of water. After filtering on a 0.45 μm filter, the clear solution obtained is purified by ultrafiltration against a solution of NaCl 0.9%, then water, until the conductimetry of the permeate is less than 50 μS/cm. After removal, the solution is filtered through a 0.2 μm filter and stored at 2-8° C.

Dry extract: 19.7 mg/g

DP (estimated based on ¹H NMR): 23

Based on ¹H NMR: i=0.05

The calculated average molecular weight of co-polyamino acid BB1 is 4350 g/mol.

Aqueous HPLC-SEC (PEG calibrant): Mn=2400 g/mol.

### EXAMPLE BB2

### Co-Polyamino Acid BB2—Sodium poly-L-glutamate Modified by Molecule BA2 and Having a Number-Average Molecular Weight (Mn) of 4900 g/mol

A poly-L-glutamic acid of number-average molecular weight (Mn) 4100 g/mol (5.0 g) obtained by a process similar to that used for the preparation of the co-polyamino acid BB1-1 is solubilized in DMF (205 ml) at 30-40° C. and maintained at this temperature. In parallel, the hydrochloride salt of the Molecule BA2 (1.44 g, 1.84 mmol) is suspended in DMF (10 ml) and triethylamine (0.19 g, 1.84 mmol) is added then the mixture is gently heated with stirring until completely dissolved. NMM (3.7 g, 36.7 mmol), the solution of molecule BA2 then the N--oxide of 2-hydroxypyridine (HOPO, 0.31 g, 2.76 mmol) are added successively to the solution of co-polyamino acid in DMF. The reaction medium is then cooled to 0° C., then EDC (0.53 g, 2.76 mmol) is added and the medium is raised to room temperature for 3 hours. The reaction mixture is poured dropwise over 1.55 L of water containing NaCl 15% by weight and HCl (pH 2) with stirring. At the end of the addition, the pH is readjusted to 2 with N I HCl solution, and the suspension is allowed to stand overnight. The precipitate is collected by filtration, then rinsed with 100 mL of water. The white solid obtained is solubilized in 200 ml of water by slowly adding a 1N aqueous NaOH solution to pH 7 with stirring, then the solution is filtered through a 0.45 μm filter. The clear solution obtained is purified by ultrafiltration against 0.9% NaCl solution, then with water, until the conductimetry of the permeate is less than 50 μS/cm. The obtained solution is filtered through a 0.2 μm filter and stored at 2-8° C.

Dry extract: 16.3 mg/g

DP (estimated based on ¹H NMR): 21

Based on ¹H NMR: i=0.047

The calculated average molecular weight of co-polyamino acid BB2 is 3932 g/mol.

Aqueous HPLC-SEC (PEG calibrant): Mn=4900 g/mol.

### EXAMPLE BB3

### Co-Polyamino Acid BB3—Sodium poly-L-glutamate Modified by Molecule BA2 and Having a Number-Average Molecular Weight (Mn) of 6400 g/mol

Co-Polyamino Acid BB3-1: poly-L-glutamic Acid with a Number-Average Molecular Weight (Mn) of 17500 g/mol from the Polymerization of γ-benzyl-L-glutamate N-carboxyanhydride Initiated by L-leucinamide

A poly-L-glutamic acid of number-average mass (Mn) 17500 g/mol relative to a standard polymethyl methacrylate (PMMA) is obtained by polymerization of γ-methyl N-carboxyanhydride of glutamic acid using L-leucinamide as an initiator and by deprotecting the methyl esters using a 37% hydrochloric acid solution according to the process described in patent application FR-A-2 801 226.

By a process similar to that used for the preparation of co-polyamino acid BB2 applied to the hydrochloride salt of molecule BA2 (3.23 g, 4.1 mmol) and to co-polyamino acid BB3-1 (11 g), a sodium poly-L-glutamate modified by molecule BA2 is obtained.

Dry extract: 27.5 mg/g

DP (estimated based on ¹H NMR): 34

Based on NMR ¹H: i=0.049

The calculated average molecular weight of co-polyamino acid BB3 is 6405 g/mol.

Aqueous HPLC-SEC (PEG calibrant): Mn=6400 g/mol.

### EXAMPLE BB4

### Co-Polyamino Acid BB4—Sodium poly-L-glutamate Modified by Molecule BA2 and Having a Number-Average Molecular Weight (Mn) of 10500 g/mol

By a process similar to that used for the preparation of co-polyamino acid BB2 applied to the hydrochloride salt of molecule BA2 (5 g, 6.35 mmol) and to a poly-L-glutamic acid with a number-average molecular weight Mn=10800 g/mol (21.7 g) obtained by a process similar to that used for the preparation of co-polyamino acid BB1-1, a sodium poly-L-glutamate modified by molecule BA2 is obtained.

Dry extract: 28.2 mg/g

DP (estimated based on ¹H NMR): 65

Based on ¹H NMR: i=0.04

The calculated average molecular weight of co-polyamino acid BB4 is 11721 g/mol.

Aqueous HPLC-SEC (PEG calibrant): Mn=10500 g/mol.

### EXAMPLE BB5

### Co-Polyamino Acid BB5—Sodium poly-L-glutamate Capped at One of Its Ends by an Acetyl Group and Modified by Molecule BA2 and Having a Number-Average Molecular Weight (Mn) of 3600 g/mol

Co-Polyamino Acid BB5-1: poly-L-glutamic Acid of Mn 3700 g/mol from the Polymerization of γ-benzyl-L-glutamate N-carboxyanhydride Initiated by hexylamine and Capped at One End by an Acetyl Group

In a previously oven-dried flask, γ-benzyl-L-glutamate N-carboxyanhydride (100.0 g, 380 mmol) is placed under vacuum for 30 minutes, then anhydrous DMF (250 ml) is added. The mixture is then stirred under argon until complete dissolution, cooled to 4° C., then hexylamine (2.3 ml 17 mmol) is quickly introduced. The mixture is stirred between 4° C. and room temperature for 2 days, then precipitated in diisopropyl ether (3.4 L). The precipitate is collected by filtration, washed twice with diisopropyl ether (225 ml), then dried to give a white solid which is dissolved in 450 ml of THF. N,N-diisopropylethylamine (DIPEA, 31 mL, 176 mmol), then acetic anhydride (17 mL,176 mmol) are successively added to this solution. After stirring overnight at room temperature, the solution is slowly poured into diisopropyl ether (3 mL) over a period of 30 minutes with stirring. After stirring for 1 hour, the precipitate is filtered off, washed twice with diisopropyl ether (200 ml), then dried under vacuum at 30° C. to give a poly (gamma-benzyl-L-glutamic acid) capped at one of its ends by an acetyl group.

A solution of hydrobromic acid (HBr) 33% in acetic acid (235 ml, 1.34 mol) is added dropwise to a solution of co-polyaminoacidcapped (72 g) in trifluoroacetic acid (TFA, 335 ml) at 4° C. The mixture is stirred at room temperature for 3 h 30, then poured dropwise onto a 1:1 (v/v) mixture of diisopropyl ether and water with stirring (4 L). After stirring for 2 hours, the heterogeneous mixture is allowed to stand overnight. The white precipitate is collected by filtration, washed with a 1:1 (v/v) mixture of diisopropyl ether and water (340 ml), then with water (340 ml). The obtained solid is then solubilized in water (1.5 L) by adjusting the pH to 7 by adding a 10N aqueous solution of sodium hydroxide, then a 1N aqueous sodium hydroxide solution. After solubilization, the solution is diluted to 20 g/L by adding water to obtain a final volume of 2.1 L. The solution is filtered through a 0.45 μm filter, then purified by ultrafiltration against a solution of NaCl 0.9%, then water until the conductimetry of the permeate is less than 50 μS/cm. The co-polyamino acid solution is then concentrated until a final volume of 1.8 L. The aqueous solution is then acidified by adding 37% hydrochloric acid solution until a pH of 2 is reached. After stirring for 4 hours, the precipitate obtained is filtered, washed with water (330 ml), then dried under vacuum at 30° C. to give a poly-L-glutamic acid of number-average molecular weight (Mn) 3700 g/mol relative to a standard of polyoxyethylene (PEG).

### Co-Polyamino Acid BB5

By a process similar to that used in the preparation of co-polyamino acid BB2 applied to the hydrochloride salt of molecule BA2 (6.92 g, 8.8 mmol) and co-polyamino acid BB5-1 (30.0 g), a sodium poly-L-glutamate capped at one end by an acetyl group and modified by molecule BA2 is obtained.

Dry extract: 29.4 mg/g

DP (estimated based on ¹H NMR): 23

Based on ¹H NMR: i=0.042

The calculated average molecular weight of co-polyamino acid BB5 is 4302 g/mol.

Aqueous HPLC-SEC (PEG calibrant): Mn=3600 g/mol.

### EXAMPLE BB6

### Co-Polyamino Acid BB6—Sodium poly-L-glutamate Capped at One of Its Ends by an Acetyl Group and Modified by Molecule BA2 and Having a Number-Average Molecular Weight (Mn) of 4100 g/mol

By a process similar to that used for the preparation of co-polyamino acid BB2 applied to the hydrochloride salt of molecule BA2 (5.8 g, 7.4 mmol) and to a poly-L-glutamic acid having a number-average molecular weight Mn=3800 g/mol (25 g) obtained by a process similar to that used for the preparation of co-polyamino acid BB5-1 using ammonia instead of hexylamine, a sodium poly-L-glutamate capped at one of its ends by an acetyl group and modified by molecule BA2 is obtained.

Dry extract: 27.6 mg/g

DP (estimated based on ¹H NMR): 24

Based on ¹H NMR: i=0.04

The calculated average molecular weight of co-polyamino acid BB6 is 4387 g/mol.

Aqueous HPLC-SEC (PEG calibrant): Mn=4100 g/mol.

### EXAMPLE BB7

### Co-Polyamino Acid BB7—Sodium poly-L-glutamate Modified by Molecule BA2 and Having a Number-Average Molecular Weight (Mn) of 4200 g/mol

By a process similar to that used for the preparation of co-polyamino acid BB2 applied to the hydrochloride acid of molecule BA2 (7.07 g, 9.0 mmol) and to a poly-L-glutamic acid having a number-average molecular weight Mn=3600 g/mol (30.0 g) obtained by a process similar to that used for the preparation of co-polyamino acid BB1-1, a sodium poly-L-glutamate modified by molecule BA2 is obtained.

Dry extract: 28.3 mg/g

DP (estimated based on ¹H NMR): 22

Based on ¹H NMR: i=0.042

The calculated average molecular weight of co-polyamino acid BB7 is 4039 g/mol.

Aqueous HPLC-SEC (PEG calibrant): Mn=4200 g/mol.

### EXAMPLE BB8

### Co-Polyamino Acid BB8—Sodium poly-L-glutamate Modified by Molecule BA2 and Having a Number-Average Molecular Weight (Mn) of 5200 g/mol

By a process similar to that used for the preparation of co-polyamino acid BB2 applied to the hydrochloride salt of molecule BA2 (0.85 g, 1.1 mmol) and to a poly-L-glutamic acid having a number-average molecular weight Mn=4100 g/mol (5.0 g) obtained by a process similar to that used for the preparation of co-polyamino acid BB1-1, a sodium poly-L-glutamate modified by molecule BA2 is obtained.

Dry extract: 28.6 mg/g

DP (estimated based on ¹H NMR): 21

Based on ¹h NMR: i=0.026

The calculated average molecular weight of co-polyamino acid BB8 is 3620 g/mol.

Aqueous HPLC-SEC (PEG calibrant): Mn=5200 g/mol.

### EXAMPLE BB9

### Co-Polyamino Acid BB9—Sodium poly-L-glutamate Modified by Molecule BA3 and Having a Number-Average Molecular Weight (Mn) of 4700 g/mol

By a process similar to that used for the preparation of co-polyamino acid BB2 applied to the hydrochloride salt of molecule BA3 (3.05 g, 3.6 mmol) and to a poly-L-glutamic acid having a number-average molecular weight Mn=4100 g/mol (10.0 g) obtained by a process similar to that used for the preparation of co-polyamino acid BB1-1, a sodium poly-L-glutamate modified by molecule BA3 is obtained.

Dry extract: 28.6 mg/g

DP (estimated based on ¹H NMR): 26

Based on ¹H NMR: i=0.05

The calculated average molecular weight of co-polyamino acid BB9 is 4982 g/mol.

Aqueous HPLC-SEC (PEG calibrant): Mn=4700 g/mol.

### EXAMPLE BB10

### Co-Polyamino Acid BB10—Sodium poly-L-glutamate Modified by Molecule BA3 and Having a Number-Average Molecular Weight (Mn) of 4200 g/mol

By a process similar to that used for the preparation of co-polyamino acid BB2 applied to the hydrochloride salt of molecule BA3 (1.90 g, 2.3 mmol) and a poly-L-glutamic acid having a number-average molecular weight Mn=3500 g/mol (10.0 g) by a process similar to that used for the preparation of co-polyamino acid BB1-1, a sodium poly-L-glutamate modified by molecule BA3 is obtained.

Dry extract: 25.9 mg/g

DP (estimated based on ¹H NMR): 22

Based on ¹H NMR: i=0.029

The calculated average molecular weight of co-polyamino acid BB10 is 3872 g/mol.

Aqueous HPLC-SEC (PEG calibrant): Mn=4200 g/mol.

### EXAMPLE BB11

### Co-Polyamino Acid BB11—Sodium poly-L-glutamate Capped at One of its Ends by an Acetyl Group and Modified by Molecule BA4 and Having a Number-Average Molecular Weight (Mn) of 3900 g/mol

By a process similar to that used for the preparation of co-polyamino acid BB2 applied to the hydrochloride salt of molecule BA4 (2.21 g, 2.2 mmol) and to a poly-L-glutamic acid having a number-average molecular weight Mn=3700 g/mol (10 g) obtained by a process similar to that used for the preparation of co-polyamino acid BB5-1, a sodium poly-L-glutamate capped at one of its ends by an acetyl group and modified by molecule BA4 is obtained.

Dry extract: 28.1 mg/g

DP (estimated based on ¹h NMR): 22

Based on ¹h NMR: i=0.032

The calculated average molecular weight of co-polyamino acid BB11 is 4118 g/mol.

Aqueous HPLC-SEC (PEG calibrant): Mn=3900 g/mol.

### EXAMPLE BB12

### Co-Polyamino Acid BB12—Sodium poly-L-glutamate Capped at One of Its Ends by an Acetyl Group and Modified by Molecule BA3 and Having a Number-Average Molecular Weight (Mn) of 3900 g/mol

By a process similar to that used for the preparation of co-polyamino acid BB2 applied to the hydrochloride salt of molecule BA3 (1.9 g, 2.3 mmol) and a poly-L-glutamic acid having a number-average molecular weight Mn=3600 g/mol (10 g) obtained by a process similar to that used for the preparation of co-polyamino acid BB5-1, a sodium poly-L-glutamate capped at one of its ends by an acetyl group and modified by molecule BA3 is obtained.

Dry extract: 26.7 mg/g

DP (estimated based on ¹H NMR): 23

Based on ¹H NMR: i=0.03

The calculated average molecular weight of co-polyamino acid BB12 is 4145 g/mol.

Aqueous HPLC-SEC (PEG calibrant): Mn=3900 g/mol.

### EXAMPLE BB13

### Co-Polyamino Acid BB13—Sodium poly-L-glutamate Modified by Molecule BA1 and Having a Number-Average Molecular Weight (Mn) of 2800 g/mol

By a process similar to that used for the preparation of co-polyamino acid BB1 applied to the hydrochloride salt of molecule BA1 (3.65 g, 5 mmol) and to a poly-L-glutamic acid with a number-average molecular weight Mn=3600 g/mol (10 g) obtained by a process similar to that used for the preparation of co-polyamino acid BB1-1, a sodium poly-L-glutamate modified by molecule BA1 is obtained.

Dry extract: 25.6 mg/g

DP (estimated based on ¹H NMR): 25

Based on ¹H NMR: i=0.08

The calculated average molecular weight of co-polyamino acid BB13 is 5253 g/mol.

Aqueous HPLC-SEC (PEG calibrant): Mn=2800 g/mol.

### EXAMPLE BB14

### Co-Polyamino Acid BB14—Sodium poly-L-glutamate Modified at One of Its Ends by Molecule BA2 and Having a Number-Average Molecular Weight (Mn) of 4020 g/mol

Hydrochloride salt of molecule BA2 (2.12 g, 2.70 mmol), chloroform (40 ml), molecular sieve 4 A (1.5 g), as well as Amberlite IRN 150 ion exchange resin (1.5 g) are successively added to a suitable container. After stirring for 1 hour on rollers, the medium is filtered and the resin is rinsed with chloroform. The mixture is evaporated, then co-evaporated with toluene. The residue is solubilized in anhydrous DMF (20 mL) for direct use in the polymerization reaction.

In a previously oven-dried flask, γ-benzyl-L-glutamate N-carboxyanhydride (18 g, 68.42 mmol) is placed under vacuum for 30 minutes, then anhydrous DMF (100 mL) is added. The mixture is stirred under argon until complete solubilization, cooled to 4° C., then the Molecule BA2 solution prepared as described above is quickly introduced. The mixture is stirred at 4° C. and room temperature for 2 days, then heated at 65° C. for 2 hours. The reaction mixture is then cooled to room temperature, then poured dropwise into diisopropyl ether (1.2 L) with stirring. The white precipitate is collected by filtration, washed twice with diisopropyl ether (100 mL), then dried under vacuum at 30° C. to obtain a white solid. The solid is diluted in TFA (105 ml), and a solution of 33% hydrobromic acid (HBr) in acetic acid (38 ml, 220 mmol) is then added dropwise and at 0° C. The solution is stirred for 2 hours at room temperature and is then poured dropwise on a mixture of 1:1 (v/v) diisopropyl ether/water and with stirring (600 ml). After stirring for 2 hours, the heterogeneous mixture is allowed to stand overnight. The white precipitate is collected by filtration, washed successively with a mixture of 1:1 (v/v) diisopropyl ether and water (200 ml), then with water (100 ml). The obtained solid is solubilized in water (450 mL) by adjusting the pH to 7 by adding 10 N aqueous sodium hydroxide solution, then 1 N aqueous sodium hydroxide solution. The mixture is filtered through a 0.45 μm filter, then is purified by ultrafiltration against 0.9% NaCl solution, then water until the conductimetry of the permeate is less than 50 μS/cm. The co-polyamino acid solution is then concentrated to about theoretical 30 g/L and the pH is adjusted to 7.0. The aqueous solution is filtered through a 0.2 μm filter and stored at 4° C.

Dry extract: 22.3 mg/g

DP (estimated by ¹H NMR)=29 where i=0.034

The calculated average molecular weight of co-polyamino acid BB14 is 5089 g/mol.

Aqueous HPLC-SEC (PEG calibrant): Mn=4020 g/mol.

### EXAMPLE BB15

### Co-Polyamino Acid BB15—Sodium poly-L-glutamate Modified at One of Its Ends by Molecule BA3 and Having a Number-Average Molecular Weight (Mn) of 3389 g/mol

By a process similar to that used for the preparation of co-polyamino acid BB14 applied to the hydrochloride salt of molecule BA3 (3.62 g, 4.32 mmol) and to 25.0 g (94.97 mmol) of γ-benzyl-L-glutamate N-carboxyanhydride, a sodium poly-L-glutamate modified at one of its ends by molecule BA3 is obtained.

Dry extract: 30.4 mg/g

DP (estimated by ¹H NMR)=24 where i=0.042

The calculated average molecular weight of co-polyamino acid BB15 is 4390 g/mol.

Aqueous HPLC-SEC (PEG calibrant): Mn=3389 g/mol.

### EXAMPLE BB16

### Co-Polyamino Acid BB16—Sodium poly-L-glutamate Modified at One of Its Ends by Molecule BA4 and Having a Number-Average Molecular Weight (Mn) of 3300 g/mol

By a process similar to that used for the preparation of co-polyamino acid BB14 applied to the hydrochloride salt of molecule BA4 (5.70 g, 5.70 mmol) and to 29.99 g (113.9 mmol) of γ-benzyl-L-glutamate N-carboxyanhydride, a sodium poly-L-glutamate modified at one of its ends by molecule BA4 is obtained.

Dry extract: 32.3 mg/g

DP (estimated by ¹H NMR)=23 where i=0.043

The calculated average molecular weight of co-polyamino acid BB16 is 4399 g/mol.

Aqueous HPLC-SEC (PEG calibrant): Mn=3300 g/mol.

### EXAMPLE BB17

### Co-Polyamino Acid BB17—Sodium poly-L-glutamate Modified at One of Its Ends by Molecule BA3 and Having a Number-Average Molecular Weight of 10700 g/mol

By a process similar to that used for the preparation of co-polyamino acid BB14 applied to the hydrochloride salt of molecule BA3 (2.51 g, 3 mmol) and to 52.7 g (200 mmol) of γ-benzyl-L-glutamate N-carboxyanhydride, a sodium poly-L-glutamate modified at one of its ends by molecule BA3 is obtained.

Dry extract: 24.5 mg/g

DP (estimated by ¹H NMR)=65 where i=0.015

The calculated average molecular weight of co-polyamino acid BB17 is 10585 g/mol.

Aqueous HPLC-SEC (PEG calibrant): Mn=10700 g/mol.

### EXAMPLE BB18

### Co-Polyamino Acid BB18—Sodium poly-L-glutamate Modified at One of Its Ends by Molecule BA3 and Having a Number-Average Molecular Weight of 6600 g/mol

By a process similar to that used for the preparation of co-polyamino acid BB14 applied to the hydrochloride salt of molecule BA3 (2.51 g, 3 mmol) and to 31.6 g (120 mmol) of γ-benzyl-L-glutamate N-carboxyanhydride, a sodium poly-L-glutamate modified at one of its ends by molecule BA3 is obtained.

Dry extract: 27.3 mg/g

DP (estimated by ¹H NMR)=40 where i=0.025

The calculated average molecular weight of co-polyamino acid BB18 is 6889 g/mol.

Aqueous HPLC-SEC (PEG calibrant): Mn=6600 g/mol.

### EXAMPLE BB19

### Co-Polyamino Acid BB19—Sodium poly-L-glutamate Modified by Molecule BA3 and Having a Number-Average Molecular Weight (Mn) of 7700 g/mol

A sodium poly-L-glutamate modified by molecule BA3 is obtained by a process similar to that used in the preparation of co-polyamino acid AB23 applied to the hydrochloride salt of molecule BA3 and co-polyamino acid AB23-1.

Dry extract: 25.3 mg/g

DP (estimated based on ¹H NMR): 60

Based on ¹H NMR: i=0.045

The calculated average molecular weight of co-polyamino acid BB19 is 11188 g/mol.

Organic HPLC-SEC (PEG Calibrator): Mn=7700 g/mol.

### EXAMPLE BB20

### Co-Polyamino Acid BB20—Sodium poly-L-glutamate Modified at One of Its Ends by Molecule BA5 and Having a Number-Average Molecular Weight (Mn) of 2800 g/mol

By a process similar to that used for the preparation of co-polyamino acid BB14 applied to molecule BA5 in the form of a free amine (1.70 g, 1.98 mmol) and to γ-benzyl-L-glutamate N-carboxyanhydride (11.46 g, 43.5 mmol), a sodium poly-L-glutamate modified at one of its ends by molecule BA5 is obtained.

Dry extract: 20.7 mg/g

DP (estimated by ¹H NMR)=23 where i=0.043

The calculated average molecular weight of co-polyamino acid BB20 is 4295 g/mol.

Aqueous HPLC-SEC (PEG calibrant): Mn=2800 g/mol.

### EXAMPLE BB21

### Co-Polyamino Acid BB21—Sodium poly-L-glutamate Modified at One of Its Ends by Molecule BA3 and Having a Number-Average Molecular Weight (Mn) of 1100 g/mol

By a process similar to that used for the preparation of co-polyamino acid BB14 applied to molecule BA3 in the form of a free amine (3.814 g, 4.75 mmol) and to γ-benzyl-L-glutamate N-carboxyanhydride (10.0 g, 38.0 mmol), a sodium poly-L-glutamate modified at one of its ends by molecule BA3 is obtained.

Dry extract: 16.1 mg/g

DP (estimated by ¹H NMR)=9 where i=0.11

The calculated average molecular weight of co-polyamino acid BB21 is 2123 g/mol.

Aqueous HPLC-SEC (PEG calibrant): Mn=1100 g/mol.

### EXAMPLE BB22

### Co-Polyamino Acid BB22—Sodium poly-L-glutamate Modified at One of Its Ends by Molecule BA6 and Having a Number-Average Molecular Weight (Mn) of 3300 g/mol

By a process similar to that used for the preparation of co-polyamino acid BB14 applied to molecule BA6 in the form of a free amine (4.45 g, 5.18 mmol) and to 30.0 g (113.96 mmol) of γ-benzyl-L-glutamate N-carboxyanhydride, a sodium poly-L-glutamate modified at one of its ends by molecule BA6 is obtained.

Dry extract: 29.0 mg/g

DP (estimated by ¹H NMR)=25 where i=0.04

The calculated average molecular weight of co-polyamino acid BB22 is 4597 g/mol.

Aqueous HPLC-SEC (PEG calibrant): Mn=3300 g/mol.

### EXAMPLE BB23

### Co-Polyamino Acid BB23—Sodium poly-L-glutamate de Modified at One of Its Ends by Molecule BA7 and Having a Number-Average Molecular Weight (Mn) of 2900 g/mol

By a process similar to that used for the preparation of co-polyamino acid BB14 applied to molecule BA7 in the form of a free amine (3.05 g, 4.01 mmol) and to γ-benzyl-L-glutamate N-carboxyanhydride (22.78 g, 86.5 mmol), a sodium poly-L-glutamate modified at one of its ends by molecule BA7 is obtained.

Dry extract: 16.9 mg/g

DP (estimated by ¹H NMR)=21 where i=0.048

The calculated average molecular weight of co-polyamino acid BB23 is 3894 g/mol.

Aqueous HPLC-SEC (PEG calibrant): Mn=2900 g/mol.

### EXAMPLE BB24

### Co-Polyamino Acid BB24—Sodium poly-L-glutamate Modified at One of Its Ends by Molecule BA3 and Modified by Molecule BA3 and Having a Number-Average Molecular Weight (Mn) of 2300 g/mol

Co-polyamino acid BB24-1: poly-L-glutamic acid modified at one of its ends by molecule BA3 and capped at the other end by pidolic acid.

In a previously oven-dried flask, γ-benzyl-L-glutamate N-carboxyanhydride (122.58 g, 466 mmol) is placed under vacuum for 30 minutes, then anhydrous DMF (220 mL) is added. The mixture is stirred under argon until complete solubilization, cooled to 10° C., then a solution of molecule BA3 in the form of free amine (17.08 g, 21.3 mmol) in chloroform (40 ml) is quickly introduced. The mixture is stirred at 0° C. and room temperature for 2 days, then heated at 65° C. for 4 hours. The reaction mixture is then cooled to 25° C., then pidolic acid (13.66 g, 105.8 mmol) is added, HOBt (2.35 g, 15.3 mmol) and EDC (20.28 g, 105.8 mmol) are added. After stirring for 24 hours at 25° C., the solution is concentrated under vacuum to eliminate chloroform and 50% of DMF. The reaction mixture is then heated to 55° C. and 1150 mL of methanol are introduced over 1 h. The reaction mixture is then cooled to 0° C. After 18 hours, the white precipitate is collected by filtration, washed three times with 270 mL of diisopropyl ether, then dried under vacuum at 30° C. to obtain a white solid. The solid is diluted in TFA (390 ml), and a solution of 33% hydrobromic acid (HBr) in acetic acid (271 ml, 1547 mmol) is then added dropwise and at 0° C. The solution is stirred for 2 hours at room temperature and is then poured dropwise on a mixture of 1:1 (v/v) diisopropyl ether/water and with stirring (970 ml). After stirring for 2 hours, the heterogeneous mixture is allowed to stand overnight. The white precipitate is collected by filtration, washed successively with diisopropyl ether (380 mL), then twice with water (380 ml). The obtained solid is solubilized in water (3.6 L) by adjusting the pH to 7 by adding a 10N aqueous solution of sodium hydroxide, then a 1N aqueous sodium hydroxide solution. The mixture is filtered through a 0.45 μm filter, then purified by ultrafiltration against 0.9% NaCl solution, 0.1N NaOH solution, 0.9% NaCl solution, phosphate buffer solution (150 mM), a solution of NaCl 0.9% then water until the conductimetry of the permeate is below 50 μS/cm. The co-polyamino acid solution is then concentrated to about 30 g/L theoretical, filtered through 0.2 microns and acidified to pH 2 with stirring by addition of a solution of HCl 37%. The precipitate is then collected by filtration, washed twice with water, then dried under vacuum at 30° C. to obtain a white solid.

### Co-Polyamino Acid BB24

By a process similar to that used for the preparation of co-polyamino acid BB2 applied to molecule BA3 in the form of a free amine (1.206 g, 1.50 mmol) and to co-polyamino acid BB24-1 (5.5 g, 33.4 mmol), a sodium poly-L-glutamate modified at one of its ends by molecule BA3 and modified by molecule BA3 is obtained.

Dry extract: 19.0 mg/g

DP (estimated based on ¹H NMR): 22

Based on ¹H NMR: i=0.089

The calculated average molecular weight of co-polyamino acid BB24 is 4826 g/mol.

Aqueous HPLC-SEC (PEG calibrant): Mn=2300 g/mol.

### EXAMPLE BB25

### Sodium Co-Polyamino Acid BB25—poly-L-glutamate Modified at One of Its Ends by Molecule BA3 and on the Other End by Molecule B8 and Having a Number-Average Molecular Weight (Mn) of 2000 g/mol

DCC (0.257 g, 1.24 mmol) and NHS (0.143 g, 1.24 mmol) are added to a solution of molecule B8 (0.946 g, 1.24 mmol) in DMF (8 ml). After stirring for 16 hours at room temperature, the solution is filtered to be used directly in the next reaction.

In a previously oven-dried flask, γ-benzyl-L-glutamate N-carboxyanhydride e (6.0 g, 22.8 mmol) is placed under vacuum for 30 minutes, then anhydrous DMF (14 mL) is added. The mixture is then stirred under argon until complete dissolution, cooled to 0° C., then a solution of molecule BA3 in the form of a free amine (0.832 g, 1.04 mmol) in chloroform (2.0 ml) is quickly introduced. After 18 hours of stirring at 0° C., the previously prepared solution of molecule B8 is added. The solution is stirred at between 0° C. and room temperature for 22 hours, then poured dropwise into diisopropylether (0.34 L) with stirring. The white precipitate is collected by filtration, washed with diisopropyl ether (7×15 mL), then dried under reduced pressure at 30° C. to give a white solid. The solid is diluted in TFA (23 ml), then the solution is cooled to 4° C. A solution of HBr at 33% in acetic acid (15 ml, 85.7 mmol) is then added dropwise. The mixture is stirred at room temperature for 2 hours, then poured dropwise onto a 1:1 (v/v) mixture of diisopropyl ether and water with stirring (0.28 L). After stirring for 2 hours, the heterogeneous mixture is allowed to stand overnight. The white precipitate is collected by filtration, washed twice with a 1:1 (v/v) mixture of diisopropyl ether and water (24 ml), then twice with water (24 ml). The obtained solid is then solubilized in water (0.16 L) by adjusting the pH to 12 by adding a 10N aqueous sodium hydroxide solution, then a 1N aqueous sodium hydroxide solution. After 30 minutes the pH is adjusted to 7 by slowly adding an aqueous solution of 1N HCl. The solution is filtered through a 0.45 μm filter, then purified by ultrafiltration against a solution of NaCl 0.9%, then water until the Permeate conductimetry is less than 50 μS/cm. The obtained solution is filtered through a 0.2 μm filter and stored at 2-8° C.

Dry extract: 18.9 mg/g

DP (estimated based on ¹H NMR): 22

Based on ¹H NMR: i1=0.09

The calculated average molecular weight of co-polyamino acid BB25 is 4871 g/mol.

Aqueous HPLC-SEC (PEG calibrant): Mn=2000 g/mol.

### C. Compositions

### EXAMPLE CV1

### Preparation of a Solution of Human Amylin at 0.6 mg/mL Containing m-Cresol (29 mM), glycerol (174 mM) at pH 7.4

A concentrated solution of human amylin at 3 mg/mL is prepared by dissolution of human amylin in the form of a powder purchased from AmbioPharm. This solution is added to a concentrated solution of excipients (m-cresol, glycerol) in order to obtain the intended final composition. The final pH is adjusted to 7.4 by addition of NaOH/HCL.

### EXAMPLE CV2

### Preparation of a Solution of Human Amylin at 0.6 mg/mL Containing Co-Polyamino Acid BB15, m-cresol (29 mM) and Glycerol (174 mM) at pH 7.4

A concentrated co-polyamino acid solution BB15 and excipients is prepared by adding concentrated solutions of excipients (m-cresol, glycerol) to a concentrated co-polyamino acid solution BB15.

The solution of human amylin concentrated to 3 mg/mL as described in example CV1 is added to this concentrated co-polyamino acid solution BB 15 and excipients in order to obtain the final compositions CV5 to CV11 (Table 1g). The final pH is adjusted to 7.4 by addition of NaOH/HCl.

TABLE 1g
Compositions and visual appearance of human amylin solutions at
pH 7.4 at different concentrations of co-polyamino acid BB15.

| | BB15/human | Co-polyamino acid | | |
|---|---|---|---|---|
| | amylin ratio | BB15 concentration | | Visual appearance |
| Solution | mol/mol | mg/mL | mM | of the solution |
| CV1 | — | — | — | Clear |
| CV5 | 5 | 3 | 0.73 | Clear |
| CV6 | 6 | 3.6 | 0.88 | Clear |
| CV7 | 7 | 4.2 | 1.03 | Clear |
| CV8 | 8 | 4.8 | 1.17 | Clear |
| CV9 | 9 | 5.4 | 1.32 | Clear |
| CV10 | 10 | 6 | 1.47 | Clear |
| CV11 | 17 | 10.5 | 2.57 | Clear |

### EXAMPLE CY1

### Preparation of a Pramlintide Solution at 0.9 mg/mL Containing m-cresol (29 mM) and Glycerin (174 mM) at pH 7.4

A concentrated solution of pramlintide at 5 mg/mL is prepared by dissolving pramlintide in powder form purchased from Hybio. This solution is added to a concentrated solution of excipients (m-cresol, glycerol) in order to obtain the intended final composition. The final pH is adjusted to 7.4 by addition of NaOH/HCl.

### EXAMPLE CW1

### Preparation of a Pramlintide Solution at 0.4 mg/mL Containing Phenol (30 mM), Glycerol (174 mM) and Glycylglycine (8 mM) at pH 7.4

By a process similar to that used in example CY1, a solution of pramlintide at 0.4 mg/mL containing phenol (30 mM), glycerol (174 mM) and glycylglycine (8 mM) at pH 7.4 is obtained.

### EXAMPLE CY0

### Preparation of a Pramlintide Solution at 0.9 mg/mL Containing BB15 Co-Polyamino Acid, m-cresol (29 mM) and Glycerin (174 mM) at pH 7.4

A concentrated co-polyamino acid solution BB15 and excipients is prepared by adding concentrated solutions of excipients (m-cresol, glycerol) to a concentrated co-polyamino acid solution BB15.

A concentrated pramlintide solution at 5 mg/mL is added to this concentrated co-polyamino acid solution BB 15 and excipients in order to obtain the final compositions CY2 to CY7 (table 3). The final pH is adjusted to 7.4 by addition of NaOH/HCl.

TABLE 3
Compositions and visual appearance of pramlintide solutions at
pH 7.4 at different concentrations of co-polyamino acid BB15.

| | BB15/pramlintide | Co-polyamino acid | | |
|---|---|---|---|---|
| | ratio | BB15 concentration | | Visual appearance |
| Solution | mol/mol | mg/mL | mM | of the solution |
| CY1 | — | — | — | Clear |
| CY2 | 2 | 1.8 | 0.44 | Clear |
| CY3 | 3 | 2.7 | 0.66 | Clear |
| CY4 | 4 | 3.6 | 0.88 | Clear |
| CY5 | 5 | 4.5 | 1.10 | Clear |
| CY6 | 6 | 5.4 | 1.32 | Clear |
| CY7 | 10 | 9 | 2.20 | Clear |

### EXAMPLE CW0

### Preparation of a Pramlintide Solution at 0.4 mg/mL Containing BB15 Co-Polyamino Acid, Phenol (30 mM), Glycerin (174 mM) and Glycylglycine (8 mM) at pH 7.4

By a protocol similar to that used in example CY0, from a solution of pramlintide at 0.4 mg/mL CW1, solutions of pramlintide at 0.4 mg/mL containing co-polyamino acid BB15, phenol (30 mM), glycerol (174 mM) and glycylglycine (8 mM) at pH 7.4 CW2 and CW3 are obtained.

TABLE 4
Compositions and visual appearance of pramlintide
solutionsat 0.4 mg/mL at pH 7.4 at different
concentrations of co-polyamino acid BB15.

| | Co-polyamino acid | | BB15/pramlintide | |
|---|---|---|---|---|
| | BB15 concentration | | ratio | Visual appearance |
| Solution | mg/mL | mM | mol/mol | of the solution |
| CW2 | 2.4 | 0.59 | 6 | Clear |
| CW3 | 4 | 0.98 | 10 | Clear |

### EXAMPLE CP0

### Preparation of a Pramlintide Solution at 0.9 mg/mL Containing Different Co-Polyamino Acids of the Invention, m-cresol (29 mM) and Glycerol (174 mM) at pH 7.4

By a protocol similar to that described in example CY0, solutions of pramlintide at 0.9 mg/mL containing various co-polyamino acids of the invention, m-cresol (29 mM) and glycerol (174 mM) at pH 7.4 CP2 to CP12 are obtained.

TABLE 8
Compositions and visual appearance of pramlintide solutions at 0.9
mg/mL at pH 7.4 in the presence of different co-polyamino acids.

| | | | | Ratio co- | |
|---|---|---|---|---|---|
| | | | | polyamino | Visual |
| | Co- | Co-polyamino acid | | acid/ | appearance |
| | polyamino | concentration | | pramlintide | of the |
| Solution | acid | mg/mL | mM | mol/mol | solution |
| CP2 | BB15 | 5 | 1.22 | 5.4 | Clear |
| | | 10 | 2.45 | 10.8 | Clear |
| CP3 | BB14 | 5 | 0.98 | 4.3 | Clear |
| | | 10 | 1.96 | 8.6 | Clear |
| CP4 | AB17 | 5 | 1.11 | 4.9 | Clear |
| | | 10 | 1.22 | 9.8 | Clear |
| CP5 | AB15 | 5 | 0.99 | 4.4 | Clear |
| | | 10 | 1.99 | 8.8 | Clear |
| CP6 | AB14 | 5 | 1.48 | 6.5 | Clear |
| | | 10 | 2.96 | 13 | Clear |
| CP10 | BB18 | 5 | 0.72 | 3.2 | Clear |
| | | 10 | 1.44 | 6.3 | Clear |
| CP11 | BB9 | 5 | 1 | 4.4 | Clear |
| | | 10 | 2.01 | 8.8 | Clear |
| CP12 | BB2 | 5 | 1.27 | 5.6 | Clear |
| | | 10 | 2.54 | 11.2 | Clear |

### EXAMPLE CH1

### Preparation of a Pramlintide Solution at 0.6 mg/mL Containing m-cresol (29 mM) and Glycerin (174 mM) at pH 6.6

A concentrated solution of pramlintide at 5 mg/mL is prepared by dissolving pramlintide in powder form purchased from Ambiopharm. This solution is added to a concentrated solution of excipients (m-cresol, glycerol) in order to obtain the intended final composition. The final pH is adjusted to 6.6 by addition of NaOH/HCl.

### EXAMPLE CH0

### Preparation of a Pramlintide Solution at 0.6 mg/mL Containing Co-Polyamino Acid BB15, m-cresol (29 mM) and Glycerin (174 mM) at pH 6.6

A concentrated co-polyamino acid solution BB15 and excipients is prepared by adding concentrated solutions of excipients (m-cresol, glycerol) to a concentrated co-polyamino acid solution BB15.

A concentrated pramlintide solution at 5 mg/mL at pH 4 is added to this concentrated co-polyamino acid solution BB15 and excipients in order to obtain the final compositions CH2 to CH8 (table 9). The final pH is adjusted to 6.6 by addition of NaOH/HCl.

TABLE 9
Compositions and visual appearance of pramlintide solutions at
pH 6.6 at different concentrations of co-polyamino acid BB15.

| | BB15/pramlintide | Co-polyamino acid | | |
|---|---|---|---|---|
| | ratio | BB15 concentration | | Visual appearance |
| Solution | mol/mol | mg/mL | mM | of the solution |
| CH1 | — | — | — | Clear |
| CH2 | 2 | 1.3 | 0.29 | Clear |
| CH3 | 3 | 2 | 0.45 | Clear |
| CH4 | 4 | 2.7 | 0.61 | Clear |
| CH5 | 6 | 4 | 0.90 | Clear |
| CH6 | 8 | 5.3 | 1.19 | Clear |
| CH7 | 10 | 6.7 | 1.50 | Clear |
| CH8 | 15 | 10 | 2.24 | Clear |

### EXAMPLE CI0

### Preparation of a Pramlintide Solution at 0.6 mg/mL Containing Different Co-Polyamino Acids of the Invention, m-cresol (29 mM) and Glycerol (174 mM) at pH 6.6

By a protocol similar to that described in example CH0, solutions of pramlintide at 0.6 mg/mL containing various co-polyamino acid of the invention, m-cresol (29 mM) and glycerol (174 mM) at pH 6.6, CI1 to CI14 and CI15 are obtained.

TABLE 10
Compositions and visual appearance of pramlintide solutions
at pH 6.6 in the presence of different co-polyamino acids.

| | | | | Ratio co- | |
|---|---|---|---|---|---|
| | | | | polyamino | Visual |
| | Co- | Co-polyamino acid | | acid/ | appearance |
| | polyamino | concentration | | pramlintide | of the |
| Solution | acid | mg/mL | mM | mol/mol | solution |
| CI1 | BB20 | 1.3 | 0.3 | 2 | Clear |
| | | 2.6 | 0.6 | 4 | Clear |
| CI2 | BB21 | 1.3 | 0.6 | 4 | Clear |
| CI3 | AB22 | 2.4 | 0.3 | 2 | Clear |
| CI4 | BB24 | 2.9 | 0.6 | 4 | Clear |
| CI5 | BB25 | 1.5 | 0.3 | 2 | Clear |
| | | 3 | 0.6 | 4 | Clear |
| CI6 | AB23 | 3.4 | 0.23 | 2 | Clear |
| CI7 | AB28 | 2.3 | 0.3 | 2 | Clear |
| | | 4.7 | 0.6 | 4 | Clear |
| CI8 | AB24 | 1.2 | 0.15 | 1 | Clear |
| | | 2.4 | 0.3 | 2 | Clear |
| CI9 | AB25 | 1.3 | 0.15 | 1 | Clear |
| | | 2.6 | 0.3 | 2 | Clear |
| CI10 | AB26 | 0.7 | 0.15 | 1 | Clear |
| | | 1.5 | 0.3 | 2 | Clear |
| CI11 | AB27 | 1.3 | 0.15 | 1 | Clear |
| | | 2.7 | 0.3 | 2 | Clear |
| CI12 | AB31 | 1.3 | 0.15 | 1 | Clear |
| | | 2.5 | 0.3 | 2 | Clear |
| CI13 | AB29 | 8.9 | 1.15 | 7.6 | Clear |
| CI14 | AB32 | 1.3 | 0.15 | 1 | Clear |
| | | 2.5 | 0.3 | 2 | Clear |
| CI15 | AB17 | 5.3 | 1.2 | 8 | Clear |

### EXAMPLE CT0

### Preparation of a Pramlintide Solution at 0.6 mg/mL Containing Co-Polyamino Acid AB14, m-cresol (29 mM) and Glycerine (174 mM) NaCl and Zinc Chloride at pH 6.6

A concentrated solution of AB14 co-polyamino acid and excipients is prepared by adding concentrated solutions of excipients (m-cresol, glycerol, NaCl, zinc chloride) to a concentrated solution of co-polyamino acid AB14.

A concentrated pramlintide solution at 5 mg/mL at pH 4 is added to this concentrated co-polyamino acid solution AB14 and excipients in order to obtain the final compositions CT1 to CT5 (table 11). The final pH is adjusted to 6.6 by addition of NaOH/HCl.

TABLE 11
Compositions and visual appearance of pramlintide solutions
at pH 6.6 in the presence of co-polyamino acid AB14 and different
contents of sodium chloride and zinc chloride.

| | | Co-polyamino acid | | | | |
|---|---|---|---|---|---|---|
| | Co-polyamino | concentration | | [NaCl] | [ZnCl₂] | Visual appearance |
| Solution | acid | mg/mL | mM | (mM) | (mM) | of the solution |
| CT1 | AB14 | 6.3 | 1.87 | — | 0.75 | Clear |
| CT2 | AB14 | 6.3 | 1.87 | 50 | — | Clear |
| CT3 | AB14 | 6.3 | 1.87 | 100 | — | Clear |
| CT4 | AB14 | 6.3 | 1.87 | 50 | 0.75 | Clear |
| CT5 | AB14 | 6.3 | 1.87 | 100 | 0.75 | Clear |

### EXAMPLE CS0

### Preparation of a Pramlintide Solution at 0.6 mg/mL Containing Co-Polyamino Acids of the Invention, m-cresol (29 mM), glycerin (174 mM) NaCl and zinc chloride at pH 6.6

By a protocol similar to that described in example CT0, solutions of pramlintide at 0.6 mg/mL containing various co-polyamino acids of the invention, m-cresol (29 mM) and glycerol (174 mM), sodium chloride and zinc chloride at pH 6.6 CS1 to CS11 and CS12 to CS27 are obtained.

TABLE 12
Compositions and visual appearance of pramlintide solutions
at pH 6.6 in the presence of different co-polyamino acids and
different contents of sodium chloride and zinc chloride.

| | | Co-polyamino acid | | | | |
|---|---|---|---|---|---|---|
| | Co-polyamino | concentration | | [NaCl] | [ZnCl₂] | Visual appearance |
| Solution | acid | mg/mL | mM | (mM) | (mM) | of the solution |
| CS1 | AB15 | 7.8 | 1.6 | — | — | Clear |
| CS2 | AB15 | 11.7 | 2.3 | — | — | Clear |
| CS3 | AB15 | 3.9 | 0.8 | 50 | — | Clear |
| CS4 | AB15 | 6.3 | 1.3 | 50 | — | Clear |
| CS5 | AB15 | 7.8 | 1.6 | 50 | — | Clear |
| CS6 | AB15 | 3.9 | 0.8 | 100 | — | Clear |
| CS7 | AB16 | 12.4 | 1.5 | — | — | Clear |
| CS8 | AB16 | 16.7 | 2.1 | — | — | Clear |
| CS9 | AB16 | 7.4 | 0.9 | 50 | — | Clear |
| CS10 | AB16 | 12.4 | 1.5 | 50 | — | Clear |
| CS11 | AB16 | 7.4 | 0.9 | 50 | 1 | Clear |

TABLE 12a
Compositions and visual appearance of pramlintide solutions at pH 6.6 in the presence of
different co-polyamino acids and different contents of sodium chloride and zinc chloride.

| | | Co-polyamino acid | | Ratio co-polyamino | | | |
|---|---|---|---|---|---|---|---|
| | Co-polyamino | concentration | | acid/pramlintide | [NaCl] | [ZnCl₂] | Visual appearance |
| Solution | acid | mg/mL | mM | mol/mol | (mM) | (mM) | of the solution |
| CS12 | AB16 | 12.4 | 1.6 | 10.5 | 25 | — | Clear |
| CS13 | AB16 | 12.4 | 1.6 | 10.5 | 25 | 1 | Clear |
| CS14 | AB16 | 10 | 1.3 | 8.4 | 50 | — | Clear |
| CS15 | AB16 | 10 | 1.3 | 8.4 | 100 | — | Clear |
| CS16 | AB34 | 6.1 | 1.5 | 10 | — | — | Clear |
| CS17 | AB34 | 6.1 | 1.5 | 10 | 100 | — | Clear |
| CS18 | AB34 | 6.1 | 1.5 | 10 | 100 | 1 | Clear |
| CS119 | AB33 | 5.7 | 1.5 | 10 | — | — | Clear |
| CS20 | AB33 | 5.7 | 1.5 | 10 | 50 | — | |
| CS21 | AB33 | 5.7 | 1.5 | 10 | 50 | 1 | Clear |
| CS22 | AB33 | 5.7 | 1.5 | 10 | 100 | — | Clear |
| CS23 | AB33 | 5.7 | 1.5 | 10 | 100 | 1 | Clear |
| CS24 | AB36 | 9.5 | 2.4 | 15.7 | — | — | Clear |
| CS25 | AB36 | 5.1 | 1.3 | 8 | 50 | — | Clear |
| CS26 | AB35 | 5.9 | 1.5 | 10 | — | — | Clear |
| CS27 | AB35 | 3.6 | 0.91 | 6 | 25 | — | Clear |

### EXAMPLE CX1

### Preparation of a Solution of Human Amylin at 0.6 mg/mL and Human Insulin at 100 IU/mL Containing m-cresol (29 mM), Glycerine (174 mM) and Zinc Chloride (229 μM) at pH 7.4.

The concentrated solution of human amylin at 3 mg/mL CV1 is added to a concentrated solution of excipients (m-cresol, glycerol). A solution of human insulin at 500 IU/ml is prepared by dissolving human insulin in powder form purchased from Amphastar. This solution is added to the concentrated solution of human amylin and excipients in order to obtain the intended final composition. The final pH is adjusted to 7.4 by addition of NaOH/HCl.

### EXAMPLE CX2

### Preparation of a Solution of Human Amylin at 0.6 mg/mL and Human Insulin at 100 IU/mL Containing Co-Polyamino Acid BB15, m-cresol (29 mM), Glycerin (174 mM) and Zinc Chloride (229 μM) at pH 7.4

A concentrated solution of BB15 co-polyamino acid and excipients is prepared by adding concentrated solutions of excipients (m-cresol, glycerol, zinc chloride) to a concentrated solution of co-polyamino acid BB15.

A concentrated solution of human amylin at 3 mg/mL, then a solution of human insulin at 500 IU/mL are added to the concentrated co-polyamino acid solution BB15 and excipients in such a manner as to obtain the intended final composition (table 13). The final pH is adjusted to 7.4 by addition of NaOH/HCl.

Solutions CX1, CX6, CX10 and CX11 are prepared according to the above protocol.

TABLE 13
Compositions and visual appearance of solutions of
human amylin and human insulin at pH 7.4 at different
concentrations of co-polyamino acid BB15.

| | BB15/human | Co-polyamino acid | | |
|---|---|---|---|---|
| | amylin ratio | BB15 concentration | | Visual appearance |
| Solution | mol/mol | mg/mL | mM | of the solution |
| CX1 | — | — | — | Turbid |
| CX6 | 6 | 3.6 | 0.88 | Clear |
| CX10 | 10 | 6 | 1.47 | Clear |
| CX11 | 17 | 10.5 | 2.57 | Clear |

In the presence of co-polyamino acid BB15, a clear solution of human amylin (0.6 mg/mL) and human insulin (100 IU/mL) is obtained at pH 7.4.

### EXAMPLE CN1

### Preparation of a Solution of Pramlintide at 0.4 mg/mL and Human Insulin at 100 IU/mL Containing Phenol (30 mM), Glycerine (174 mM), Glycylglycine (8 mM) and Zinc (229 μM) at pH 7.4.

A concentrated solution of pramlintide at 5 mg/mL is added to a concentrated solution of excipients (m-cresol, glycerol, glycylglycine, zinc chloride). A solution of human insulin at 500 IU/mL is added to this concentrated solution of pramlintide and excipients in order to obtain the intended final composition. The final pH is adjusted to 7.4 by addition of NaOH/HCl.

### EXAMPLE CR1

### Preparation of a Solution of Human Pramlintide at 0.9 mg/mL and Human Insulin at 100 IU/mL Containing m-cresol (29 mM), Gycerin (174 mM) and Zinc Chloride (229 μM) at pH 7.4

By a process similar to that used in example CN1, a solution of pramlintide at 0.9 mg/mL and human insulin at 100 IU/mL containing m-cresol (29 mM), glycerol (174 mM) and zinc chloride (229 μM) at pH 7.4 is obtained.

### EXAMPLE CN0

### Preparation of a Solution of Pramlintide at 0.4 mg/mL and Human Insulin at 100 IU/mL Containing Co-Polyamino Acid BB15, phenol (30 mM), glycerine (174 mM), Glycylglycine (8 mM) and Zinc Chloride (229 μM) at pH 7.4

A concentrated solution of co-polyamino acid BB15 and excipients is prepared by adding concentrated solutions of excipients (m-cresol, glycerol, glycylglycine, zinc chloride) to a concentrated solution of co-polyamino acid BB15.

A concentrated solution of human amylin at 5 mg/mL then a solution of human insulin at 500 IU/mL are added to this concentrated co-polyamino acid solution BB15 and excipients in order to obtain the intended final composition (Table 14). The final pH is adjusted to 7.4 by addition of NaOH/HCl.

CN2 and CN3 solutions are prepared according to the above protocol.

TABLE 14
Compositions and visual appearance of solutions of pramlintide
at 0.4 mg/mL and human insulin at 100 IU/mL at pH 7.4 at
different concentrations of co-polyamino acid BB15.

| | Co-polyamino acid | | Ratio | |
|---|---|---|---|---|
| | BB15 concentration | | BB15/pramlintide | Visual appearance |
| Solution | mg/mL | mM | mol/mol | of the mixture |
| CN1 | — | | — | Turbid |
| CN2 | 2.4 | 0.59 | 6 | Clear |
| CN3 | 4 | 0.98 | 10 | Clear |

In the presence of co-polyamino acid BB15, a clear solution of pramlintide (0.4 mg/mL) and human insulin (100 IU/ml) is obtained at pH 7.4.

### EXAMPLE CR0

### Preparation of a Solution of Pramlintide at 0.9 mg/mL and Human Insulin at 100 IU/mL Containing BB15 Co-Polyamino Acid, m-cresol (29 mM), Glycerin (174 mM) and Zinc Chloride (229 μM) at pH 7.4

By a process similar to that used in example CN0, a solution of pramlintide at 0.9 mg/mL and human insulin at 100 IU/mL containing co-polyamino acid BB15, m-cresol (29 mM), glycerol (174 mM) and zinc chloride (229 μM) at pH 7.4 is obtained.

Solutions CR2 to CR4 and CU2 to CU8 are prepared according to the above protocol.

TABLE 15
Compositions and appearance of solutions of pramlintide
at 0.9 mg/mL and human insulin at 100 IU/mL at pH 7.4 at
different concentrations of co-polyamino acid BB15.

| | Co-polyamino acid | | Ratio | |
|---|---|---|---|---|
| | BB15 concentration | | BB15/pramlintide | Visual appearance |
| Solution | mg/mL | mM | mol/mol | of the solution |
| CR1 | — | | — | Turbid |
| CR2 | 2.7 | 0.66 | 3 | Clear |
| CR3 | 3.6 | 0.88 | 4 | Clear |
| CR4 | 4.5 | 1.10 | 5 | Clear |
| CU2 | 0.9 | 0.22 | 1 | Clear |
| CU3 | 1.8 | 0.44 | 2 | Clear |
| CU7 | 5.4 | 1.32 | 6 | Clear |
| CU8 | 9 | 2.20 | 10 | Clear |

In the presence of co-polyamino acid BB15, a clear solution of pramlintide (0.9 mg/mL) and of human insulin (100 IU/mL) at pH 7.4 is obtained.

### EXAMPLE CG0

### Preparation of a Solution of Pramlintide at 0.9 mg/mL and Human Insulin at 100 IU/mL Containing Various Co-Polyamino Acids of the Invention, m-cresol (29 mM), Glycerine (174 mM) and Zinc Chloride (229 μM) at pH 7.4

By a process similar to example CN0, a solution of pramlintide at 0.9 mg/mL and human insulin at 100 IU/ml containing a co-polyamino acid of the invention, m-cresol (29 mM), glycerol (174 mM) and zinc chloride (229 μM) at pH 7.4 is obtained.

Solutions CG2 to CG12 are prepared according to the above-described protocol.

TABLE 16
Compositions and visual appearance of solutions of pramlintide
0.9 mg/mL and human insulin 100 IU/mL pH 7.4 at different
concentrations of co-polyamino acids.

| | | | | Ratio Co- | |
|---|---|---|---|---|---|
| | | | | polyamino | Visual |
| | Co- | Co-polyamino acid | | acid/ | appearance |
| | polyamino | concentration | | pramlintide | of the |
| Solution | acid | mg/mL | mM | mol/mol | solution |
| CG2 | BB15 | 5 | 1.22 | 5.4 | Clear |
| | | 10 | 2.45 | 10.8 | Clear |
| CG3 | BB14 | 5 | 0.98 | 4.3 | — |
| | | 10 | 1.96 | 8.6 | Clear |
| CG4 | AB17 | 5 | 1.11 | 4.9 | Clear |
| | | 10 | 1.22 | 9.8 | Clear |
| CG5 | AB15 | 5 | 0.99 | 4.4 | — |
| | | 10 | 1.99 | 8.8 | Clear |
| CG6 | AB14 | 5 | 1.48 | 6.5 | — |
| | | 10 | 2.96 | 13 | Clear |
| CG10 | BB18 | 5 | 0.72 | 3.2 | Clear |
| | | 10 | 1.44 | 6.3 | Clear |
| CG11 | BB9 | 5 | 1 | 4.4 | Clear |
| | | 10 | 2.01 | 8.8 | Clear |
| CG12 | BB2 | 5 | 1.27 | 5.6 | Clear |
| | | 10 | 2.54 | 11.2 | Clear |

### EXAMPLE CD1

### Preparation of a Solution of Human Pramlintide at 0.9 mg/mL and Insulin Lispro at 100 IU/mL Containing m-cresol (29 mM), Glycerin (174 mM) and Zinc Chloride (300 μM) at pH 7.4.

A concentrated solution of pramlintide at 5 mg/mL is added to a concentrated solution of excipients (m-cresol, glycerol, zinc chloride). A solution of insulin lispro at 500 IU/mL is added to this concentrated solution of pramlintide and excipients in order to obtain the intended final composition. The final pH is adjusted to 7.4 by addition of NaOH/HCl.

### EXAMPLE CD0

### Preparation of a Solution of Pramlintide at 0.9 mg/mL and Insulin Lispro at 100 IU/mL Containing BB15 Co-Polyamino Acid, m-cresol (29 mM), Glycerin (174 mM) and Zinc Chloride (300 μM) at pH 7.4

A concentrated solution of co-polyamino acid BB15 and excipients is prepared by adding concentrated solutions of excipients (m-cresol, glycerol, zinc chloride) to a concentrated solution of BB15 co-polyamino acid.

A concentrated solution of pramlintide at 5 mg/mL, then a solution of insulin lispro at 500 IU/mL are added to the concentrated co-polyamino acid solution BB15 and excipients in order to obtain the intended final composition. The final pH is adjusted to pH 7.4 by adding NaOH/HCl.

Solutions CD3 to CD9 are prepared according to the above-described protocol.

TABLE 17
Compositions and visual appearance of solutions of pramlintide
at 0.9 mg/mL and insulin lispro at 100 IU/mL at pH 7.4 at
different concentrations of co-polyamino acid BB15.

| | BB15/pramlintide | Co-polyamino acid | | |
|---|---|---|---|---|
| | ratio | BB15 concentration | | Visual appearance |
| Solution | mol/mol | mg/mL | mM | of the solution |
| CD1 | — | — | — | Turbid |
| CD3 | 2 | 1.8 | 0.44 | Clear |
| CD4 | 3 | 2.7 | 0.66 | Clear |
| CD5 | 4 | 3.6 | 0.88 | Clear |
| CD6 | 5 | 4.5 | 1.10 | Clear |
| CD7 | 6 | 5.4 | 1.32 | Clear |
| CD8 | 10 | 9 | 2.20 | Clear |
| CD9 | 15 | 13.5 | 3.30 | Clear |

In the presence of co-polyamino acid BB15, a clear solution of pramlintide (0.9 mg/mL) and of insulin lispro (100 IU/mL) at pH 7.4 is obtained.

### EXAMPLE CK1

### Preparation of a Solution of Human Pramlintide at 0.6 mg/mL and Insulin Lispro at 100 IU/mL Containing m-cresol (29 mM), Glycerol (174 mM) and Zinc Chloride (229 μM) at pH 6.6

By a process similar to that used in example CR1, a solution of pramlintide at 0.6 mg/mL and human insulin at 100 IU/mL containing m-cresol (29 mM), glycerol (174 mM) and zinc chloride (229 μM) at pH 6.6 is obtained.

### EXAMPLE CK0

### Preparation of a Solution of Pramlintide at 0.6 mg/mL and Human Insulin at 100 IU/mL Containing Co-Polyamino Acid BB15, m-cresol (29 mM), Glycerin (174 mM) and Zinc Chloride (229 μM) at pH 6.6

By a process similar to that used in example CR0, a solution of pramlintide at 0.6 mg/mL and human insulin at 100 IU/mL containing co-polyamino acid BB15, m-cresol (29 mM), glycerol (174 mM) and zinc chloride (229 μM) at pH 6.6 is obtained.

Solutions CK2 to CK8 are prepared according to the above protocol.

TABLE 18
Compositions and visual appearance of solutions of pramlintide
at 0.6 mg/mL and human insulin at 100 IU/mL at pH 6.6 at
different concentrations of co-polyamino acid BB15.

| | BB15/pramlintide | Co-polyamino acid | | |
|---|---|---|---|---|
| | ratio | BB15 concentration | | Visual appearance |
| Solution | mol/mol | mg/mL | mM | of the solution |
| CK1 | — | — | — | Turbid |
| CK2 | 2 | 1.3 | 0.29 | Clear |
| CK3 | 3 | 2 | 0.45 | Clear |
| CK4 | 4 | 2.7 | 0.61 | Clear |
| CK5 | 6 | 4 | 0.90 | Clear |
| CK6 | 8 | 5.3 | 1.19 | Clear |
| CK7 | 10 | 6.7 | 1.50 | Clear |
| CK8 | 15 | 10 | 2.24 | Clear |

In the presence of co-polyamino acid BB15, a clear solution of pramlintide (0.6 mg/mL) and of human insulin (100 IU/mL) at pH 6.6 is obtained.

### EXAMPLE CF1

### Preparation of Compositions Containing Variable Concentrations of Pramlintide, of Human Insulin at 100 IU/mL, m-cresol (29 mM), Glycerol (174 mM) and Zinc Chloride (229 μM) at pH 6.6.

By a process similar to that used in example CR1, solutions containing different concentrations of pramlintide, of human insulin at 100 IU/mL, m-cresol (29 mM), glycerol (174 mM) and zinc chloride (229 μM) at pH 6.6 are obtained.

TABLE 18a
Compositions and visual appearance of pramlintide solutions at different
concentrations and human insulin at 100 IU/mL at pH 6.6.

| | Concentration of pramlintide | Visual appearance |
|---|---|---|
| Solution | (mg/mL) | of the solution |
| CF1A | 0.9 | turbid |
| CF1B | 0.8 | turbid |
| CF1C | 0.6 | turbid |
| CF1D | 0.3 | turbid |
| CF1E | 0.2 | turbid |

### EXAMPLE CF0

### Preparation of Compositions Containing Variable Concentrations of Pramlintide and Human Insulin at 100 IU/mL in the Presence of Co-Polyamino Acid AB24, m-cresol (29 mM), Glycerol (174 mM) and Zinc Chloride (229 μM) at pH 6.6

By a process similar to that used in example CR0, solutions containing different concentrations of pramlintide, of human insulin at 100 IU/mL, co-polyamino acid AB24, m-cresol (29 mM), glycerol (174 mM) and zinc chloride (229 μM) at pH 6.6 are obtained.

TABLE 18b
Compositions and visual appearance of pramlintide solutions
at different concentrations and human insulin at 100 IU/mL
in the presence of co-polyamino acid AB24 at pH 6.6.

| | | | | Ratio co- | |
|---|---|---|---|---|---|
| | Concen- | | | polyamino | Visual |
| | tration of | Co-polyamino acid | | acid/ | appearance |
| | pramlintide | concentration AB24 | | pramlintide | of the |
| Solution | (mg/mL) | mg/mL | mM | mol/mol | solution |
| CF2 | 0.9 | 5.4 | 0.67 | 3 | clear |
| CF3 | 0.8 | 4.8 | 0.6 | 3 | clear |
| CF4 | 0.6 | 3.6 | 0.45 | 3 | clear |
| CF5 | 0.3 | 1.8 | 0.22 | 3 | clear |
| CF6 | 0.2 | 1 | 0.125 | 2.5 | clear |

### EXAMPLE CM0

### Preparation of a Solution of Pramlintide at 0.6 mg/mL and Human Insulin at 100 IU/mL Containing Various Co-Polyamino Acids of the Invention, m-cresol (29 mM), Glycerine (174 mM) and Zinc Chloride (229 μM) at pH 6.6

By a process similar to example CG0, solutions of pramlintide at 0.6 mg/mL and human insulin at 100 IU/ml containing various co-polyamino acids of the invention, m-cresol (29 mM), glycerol (174 mM) and zinc chloride (229 μM) at pH 6.6 are obtained.

Solutions CM1 to CM18 are prepared according to the above-described protocol.

TABLE 19
Compositions and visual appearance of solutions of pramlintide
at 0.6 mg/mL and human insulin at 100 IU/mL at pH 6.6
in the presence of different co-polyamino acids.

| | | | | Ratio co- | |
|---|---|---|---|---|---|
| | | | | polyamino | Visual |
| | Co- | Co-polyamino acid | | acid/ | appearance |
| | polyamino | concentration | | pramlintide | of the |
| Solution | acid | mg/mL | mM | mol/mol | solution |
| CM1 | BB20 | 2.6 | 0.61 | 4 | Clear |
| | | 5.3 | 1.22 | 8 | Clear |
| CM2 | BB21 | 1.3 | 0.6 | 4 | Clear |
| CM3 | AB22 | 2.4 | 0.3 | 2 | Clear |
| CM4 | BB24 | 2.9 | 0.6 | 4 | Clear |
| CM5 | BB23 | 3 | 0.76 | 5 | Clear |
| CM6 | BB25 | 1.5 | 0.3 | 2 | Clear |
| CM7 | BB22 | 2.7 | 0.6 | 4 | Clear |
| CM8 | AB23 | 7.7 | 0.69 | 4.6 | Clear |
| CM9 | BB19 | 4.7 | 0.4 | 2.8 | Clear |
| CM10 | AB28 | 2.3 | 0.3 | 2 | Clear |
| CM11 | AB24 | 1.2 | 0.15 | 1 | Clear |
| | | 2.4 | 0.3 | 2 | Clear |
| | | 3.6 | 0.45 | 3 | |
| CM12 | AB25 | 2.6 | 0.3 | 2 | Clear |
| CM13 | AB26 | 1.5 | 0.3 | 2 | Clear |
| | | 2.3 | 0.5 | 3 | Clear |
| CM14 | AB27 | 1.3 | 0.15 | 1 | Clear |
| | | 2.7 | 0.3 | 2 | Clear |
| CM15 | AB30 | 1.2 | 0.15 | 1 | Clear |
| | | 2.3 | 0.3 | 2 | Clear |
| CM16 | AB31 | 1.3 | 0.15 | 1 | Clear |
| | | 2.5 | 0.3 | 2 | Clear |
| CM17 | AB29 | 5.9 | 0.8 | 5 | Clear |
| | | 8.9 | 1.15 | 7.6 | Clear |
| CM18 | AB32 | 2.5 | 0.3 | 2 | Clear |

### EXAMPLE CQ1

### Preparation of a Solution of Pramlintide at 0.6 mg/mL and Human Insulin at 100 IU/mL at pH 6.6 Containing Co-Polyamino Acid AB14, m-cresol (29 mM), Glycerine (174 mM), Sodium Chloride (100 mM) and Zinc Chloride (1 mM)

A concentrated solution of co-polyamino acid AB14 and excipients is prepared by adding concentrated solutions of excipients (m-cresol, glycerol, sodium chloride, zinc chloride) to a concentrated solution of co-polyamino acid AB14.

A solution of pramlintide concentrated to 5 mg/mL at pH 4, then a human insulin solution at 500 IU/mL are added to this concentrated co-polyamino acid AB14 solution and excipients in order to obtain the intended final composition. The final pH is adjusted to 6.6 by addition of NaOH/HCl.

The CQ1 solution is prepared according to the above protocol.

### EXAMPLE CQ0

### Preparation of a Solution of Pramlintide at 0.6 mg/mL and Human Insulin at 100 IU/ml Containing Various Co-Polyamino Acids of the Invention, m-cresol (29 mM), Glycerine (174 mM), and Different Contents of Sodium Chloride and Zinc Chloride

By a process similar to example CQ0, solutions of pramlintide at 0.6 mg/mL and human insulin at 100 IU/ml containing various co-polyamino acids of the invention, m-cresol (29 mM), glycerol (174 mM), sodium chloride and zinc chloride at pH 6.6 are obtained.

Solutions CQ2 to CQ12 are prepared according to the above protocol.

TABLE 20
Compositions and visual appearance of solutions of pramlintide 0.6 mg/mL
and human insulin 100 IU/mL pH 6.6 in the presence of different co-polyamino
acids and different contents of sodium chloride and zinc chloride.

| | | Co-polyamino acid | | | | |
|---|---|---|---|---|---|---|
| | Co-polyamino | concentration | | [NaCl] | [ZnCl₂] | Visual appearance |
| Solution | acid | mg/mL | mM | (mM) | (mM) | of the solution |
| CQ1 | AB14 | 6.3 | 1.87 | 100 | 1 | Clear |
| CQ2 | AB15 | 7.8 | 1.6 | — | 0.23 | Clear |
| CQ3 | AB15 | 11.7 | 2.3 | — | 0.23 | Clear |
| CQ4 | AB15 | 3.9 | 0.8 | 50 | 0.23 | Clear |
| CQ5 | AB15 | 6.3 | 1.3 | 50 | 0.23 | Clear |
| CQ6 | AB15 | 7.8 | 1.6 | 50 | 0.23 | Clear |
| CQ7 | AB15 | 3.9 | 0.8 | 100 | 0.23 | Clear |
| CQ8 | AB15 | 6.3 | 1.3 | 100 | 0.23 | Clear |
| CQ9 | AB15 | 7.8 | 1.6 | 100 | 0.23 | Clear |
| CQ10 | AB16 | 7.4 | 0.9 | 50 | 0.23 | Clear |
| CQ11 | AB16 | 12.4 | 1.5 | 50 | 0.23 | Clear |
| CQ12 | AB16 | 7.4 | 0.9 | 50 | 1 | Clear |

### EXAMPLE CZ0

### Preparation of a Solution of Pramlintide 0.6 mg/mL Containing DMPG, m-cresol (29 mM), Glycerin (174 mM) at pH 6.6

A concentrated solution of DMPG and excipients is prepared by adding concentrated solutions of excipients (m-cresol, glycerol) to a concentrated solution of DMPG.

A solution of pramlintide concentrated at 10 mg/mL is added to this concentrated solution of DMPG and excipients in order to obtain the intended final composition. The final pH is adjusted to 6.6 by addition of NaOH/HCl.

### EXAMPLE CZ1

### Preparation of a Solution of Pramlintide 0.6 mg/mL Containing DMPG (4.5 mM), Phenol (30 mM), Glycerin (174 mM) and Glycylglycine (8 mM) at pH 7.4

TABLE 21
Compositions and visual appearance of pramlintide
solutions at 0.6 mg/mL in the presence of DMPG.

| | | | | | Visual |
|---|---|---|---|---|---|
| | | Concentration | | | appearance |
| | DMPG | pramlintide | | | of the |
| Solution | (MM) | (mg/mL) | pH | Excipients | solution |
| CZ0 | 4.5 | 0.6 | 6.6 | m-cresol 29 mM | Clear |
| | | | | glycerol 174 mM | |
| CZ1 | 4.5 | 0.6 | 7.4 | phenol 30 mM | Clear |
| | | | | glycylglycine | |
| | | | | 8 mM | |
| | | | | glycerol 174 mM | |

### EXAMPLE CA1

### Preparation of a Solution of Pramlintide at 1 mg/mL Containing m-cresol (20 mM), Mannitol (43 mg/mL), and Sodium Acetate Buffer, pH 4

A concentrated solution of pramlintide at 10 mg/mL is added to a concentrated solution of excipients (m-cresol, mannitol, sodium acetate) in order to obtain the intended final composition. The final pH is adjusted to 4 by addition of NaOH/HCl. The clear solution is filtered (0.22 μM) and introduced into 3 mL injector pen glass cartridges.

### EXAMPLE CA2

### Putting a Commercial Solution of Human Insulin at 100 IU/mL (Humulin®) Containing m-cresol (23 mM), Glycerol (174 mM) and Zinc Chloride (230 μM) into Cartridges

A 10 ml vial commercial solution of Humulin is collected and introduced into 3 mL pen injector glass cartridges.

### EXAMPLE CA3

### Preparation of a Solution of Pramlintide at 0.6 mg/mL and Human Insulin at 100 IU/mL Containing Co-Polyamino Acid BB15 (3.1 mg/mL), m-cresol (29 mM), Glycerol (35 mM), Mannitol (2.6% w/v), Tris (18.75 mM), Sodium Acetate (18 mM), and Zinc Chloride (260 μM) at pH 6.2

3 mL glass vials are filled with 0.5 mL of a solution containing 10 mg/mL of co-polyamino acid BB 15 and 60 mM of tris at pH 8.3. The solution is freeze-dried to obtain 3 mL vials containing 5 mg of co-polyamino acid BB15 and 30 μmol tris.

A solution of human insulin at 500 IU/mL containing 23 mM m-cresol, 174 mM glycerol and 260 μM zinc chloride at pH 7.4 is prepared by adding concentrated solutions of excipients (m-cresol, glycerol, zinc chloride) to a concentrated solution of human insulin concentrated to 760 IU/mL.

The following are introduced successively into the vial containing 5 mg of co-polyamino acid BB15 and 30 μmol tris:
0.96 mL of pramlintide solution at 1 mg/mL at pH 4 described in EXAMPLE CA1;
0.32 mL sterile water for injection;
0.32 mL of human insulin concentrated at 500 IU/mL containing 23 mM m-cresol, 174 mM glycerol and 260 μM zinc chloride at pH 7.4.

The clear solution is filtered (0.22 μM) and introduced into 3 mL injector pen glass cartridges.

### EXAMPLE CA4

### Putting a Solution of Pramlintide 0.6 mg/mL and Human Insulin 100 IU/mL in the Presence of Co-Polyamino Acid AB24 at 2.4 mg/mL, m-cresol (29 mM), Glycerol (174 mM) and Zinc Chloride (229 μM) at pH 6.6 into Cartridges

The solution of pramlintide at 0.6 mg/mL and human insulin 100 IU/mL in the presence of co-polyamino acid AB24 at 2.4 mg/mL, m-cresol (29 mM), glycerol (174 mM) and zinc chloride (229 μM) at pH 6.6 described in example CM11 is filtered (0.22 μm) and introduced into 3 mL glass cartridges for pen injection.

### EXAMPLE CA5

### (Pump Stability): Preparation of a Solution of Pramlintide at 0.6 mg/mL and Insulin Lispro 100 IU/mL pH 6.6 Containing Co-polyamino Acid AB24, m-cresol (29 mM), Glycerine (174 mM) and Zinc Chloride (300 μM)

A concentrated solution of co-polyamino acid AB24 and excipients is prepared by adding concentrated solutions of excipients (m-cresol, glycerol, zinc chloride) to a concentrated solution of co-polyamino acid AB24.

A concentrated solution of human amylin at 10 mg/mL then a solution of insulin lispro at 200 IU/mL are added to this concentrated co-polyamino acid AB24 solution and excipients in order to obtain the intended final composition. The final pH is adjusted to 6.6 by addition of NaOH/HCl.

### EXAMPLE CA6

### Preparation of a Solution of Human Insulin at 100 IU/mL Containing Co-Polyamino Acid BB15 (10 mg/mL), m-cresol (29 mM), Glycerol (174 mM) and Zinc Chloride (229 μM) at pH 6.6

A concentrated solution of BB15 co-polyamino acid and excipients is prepared by adding concentrated solutions of excipients (m-cresol, glycerol, zinc chloride) to a concentrated solution of co-polyamino acid BB15.

A solution of human insulin at 800 IU/mL is added to this concentrated co-polyamino acid solution BB15 and excipients in order to obtain the intended final composition. The final pH is adjusted to 6.6 by addition of NaOH/HCl. The clear solution is filtered (0.22 μM) and introduced into 3 mL injector pen glass cartridges.

### EXAMPLE CA7

### Preparation of a Solution of Pramlintide at 0.6 mg/mL and Human Insulin at 100 IU/mL Containing Co-Polyamino Acid BB15 (10 mg/mL), m-cresol (29 mM), Glycerol (174 mM) and Zinc Chloride (229 μM) at pH 6.6

A concentrated solution of co-polyamino acid BB15 and excipients is prepared by adding concentrated solutions of excipients (m-cresol, glycerol, zinc chloride) to a concentrated solution of co-polyamino acid BB15.

A concentrated solution of pramlintide at 10 mg/mL, then a concentrated solution of human insulin at 500 IU/mL are added to this concentrated co-polyamino acid solution BB15 and excipients in order to obtain the intended final composition. The final pH is adjusted to 6.6 by addition of NaOH/HCl. The clear solution is filtered (0.22 μM) and introduced into 3 mL glass cartridges

### D. Physical Chemistry

### DI: Results of Visual Observations on the Mixture and ThT Fibrillation Measurements

### Principle

The poor stability of a peptide can lead to the formation of amyloid fibrils, defined as ordered macromolecular structures. These may result in the formation of a gel within the sample.

The thioflavin fluorescence monitoring test T(ThT) is used to analyze the physical stability of the solutions. Thioflavin is a small probe molecule having a characteristic fluorescence signature when it binds to amyloid-type fibrils (Naiki et al. (1989) Anal. Bio Chem. 177, 244-249; LeVine (1999) Methods. Enzymol. 309, 274-284).

This method tracks fibril formation at low concentrations of ThT in undiluted solutions. This monitoring is performed under accelerated stability conditions: with stirring and at 37° C.

### Experimental Conditions

Samples were prepared just before the start of the measurement. The preparation of each composition is described in the corresponding example. Thioflavin T was added to the composition from a concentrated stock solution in order to induce a negligible dilution of the composition. The concentration of Thioflavin T in the composition is 1, 2 or 40 μM depending on the composition type: 40 μM in the case of human amylin compositions at 0.6 mg/mL, 2μM in the case of pramlintide compositions at 0.9 mg/mL and 0.6 mg/mL and 1 μM in pramlintide compositions at 0.4 mg/mL. This concentration is indicated in the legend relating to the latency time results table for each type of composition.

A 150 μL volume of the composition was introduced into a well of a 96-well plate. Each composition was analyzed in three tests (triplicate) within the same plate. The plate was sealed with a transparent film in order to prevent evaporation of the composition.

This plate was then placed in the enclosure of a plate reader (EnVision 2104 Multilabel, Perkin Elmer or Xenius XM, Safas). The temperature is adjusted to 37° C., and lateral agitation at 960 rpm with an amplitude of 1 mm is applied.

A reading of the fluorescence intensity in each well is made with an excitation wavelength of 442 nm, and an emission wavelength of 482 nm over time.

The fibrillation process is manifested by a strong increase in fluorescence after a delay called latency time.

For each well, this delay was determined graphically from the intersection between the baseline of the fluorescence signal and the slope of the fluorescence curve as a function of time determined during the strong initial increase in fluorescence. The reported latency time value corresponds to the average of latency time measurements made on three wells.

An example of a graphic determination is shown in FIG. 1.

### EXAMPLE D1

### Stability of Solutions of Human Amylin at 0.6 mg/mL at pH 7.4 in the Presence of Co-Polyamino Acid BB15 at Different Concentrations

TABLE 22
ThT (40 μM) latency time measurement
of solutions CV1 and CV5 to CV10.

| | | |
|---|---|---|
| BB15/human | Co-polyamino acid | |
| amylin ratio | BB15 concentration | Latency |

| Solution | mol/mol | mg/mL | mM | time (h) |
|---|---|---|---|---|
| CV1 | — | — | — | <0.02 |
| CV5 | 5 | 3 | 0.73 | >1 |
| CV6 | 6 | 3.6 | 0.88 | >5 |
| CV7 | 7 | 4.2 | 1.03 | >30 |
| CV8 | 8 | 4.8 | 1.17 | >54 |
| CV9 | 9 | 5.4 | 1.32 | >54 |
| CV10 | 10 | 6 | 1.47 | >72 |

The latency time of a solution of human amylin at pH 7.4 (CV1), without co-polyamino acid, is less than 0.02 hours; CV5 to CV10 solutions according to the invention, containing BB15/human amylin molar ratios greater than 5, make it possible to obtain latency times greater than one hour, a molar ratio of 10 making it possible to obtain latency times greater than 72 hours.

### EXAMPLE D2

### Stability of Solutions of Human Zmylin at 0.6 mg/mL and of Human Insulin at 100 IU at pH 7.4 in the Presence of Co-Polyamino Acid BB15 at Different Concentrations

TABLE 23
ThT (40 uM) latency time measurement
of solutions CX6, CX10 and CX11.

| | | |
|---|---|---|
| BB15/human | Co-polyamino acid | |
| amylin ratio | BB15 concentration | Latency |

| Solution | mol/mol | mg/mL | mM | time (h) |
|---|---|---|---|---|
| CX1 | — | — | — | * |
| CX6 | 6 | 3.6 | 0.88 | >0.1 |
| CX10 | 10 | 6 | 1.47 | >0.5 |
| CX11 | 17.5 | 10.5 | 2.57 | >5 |

* Latency time not measured due to turbid solution

The solution of human amylin and of human insulin at pH 7.4 (CX1) is turbid. Co-polyamino acid BB15 makes it possible to obtain a clear solution containing human amylin in the presence of human insulin at pH 7.4 with latency times greater than 0.1 hour starting from a molar ratio BB15/amylin 6, and greater than 5 hours for a BB15/human amylin molar ratio of 17.5.

### EXAMPLE D3

### Stability of Pramlintide Solutions at 0.4 mg/mL at pH 7.4 in the Presence of Co-Polyamino Acid BB15 at Different Concentrations

TABLE 24
ThT (1 μM) latency time measurement of solutions CW1 to CW3.

| | | |
|---|---|---|
| Co-polyamino acid | BB15/pramlintide | |
| BB15 concentration | ratio | Latency |

| Solution | mg/mL | mM | mol/mol | time (h) |
|---|---|---|---|---|
| CW1 | — | | — | 0.7 |
| CW2 | 2.4 | 0.59 | 6 | >40 |
| CW3 | 4 | 0.98 | 10 | >63 |

Pramlintide solution at pH 7.4 (CW1) without co-polyamino acid has a short latency time. Co-polyamino acid BB15 makes it possible to obtain a solution containing pramlintide at pH 7.4 with latency times greater than 40 h from a BB15/pramlintide molar ratio of 6.

### EXAMPLE D4

### Stability of Pramlintide Solutions at 0.9 mg/mL at pH 7.4 in the Presence of Co-Polyamino Acid BB 15 at Different Concentrations

TABLE 25
ThT (2 μM) latency measurement of solutions CY1 to CY7

| | BB15/pramlintide | | | |
|---|---|---|---|---|
| | ratio | Concentration in BB15 | | Latency |
| Solution | mol/mol | mg/mL | mM | time (h) |
| CY1 | — | — | — | 0.7 |
| CY2 | 2 | 1.8 | 0.44 | >0.8 |
| CY3 | 3 | 2.7 | 0.66 | >4 |
| CY4 | 4 | 3.6 | 0.88 | >30 |
| CY5 | 5 | 4.5 | 1.10 | >63 |
| CY6 | 6 | 5.4 | 1.22 | >63 |
| CY7 | 10 | 9 | 1.32 | >63 |

Pramlintide solution at pH 7.4 (CY1) without a co-polyamino acid has a short latency time; the latency times of co-polyamino acid-containing solutions are greater than or equal to the latency times of the co-polyamino acid-free composition at a BB 15 co-polyamino acid/pramlintide molar ratio of 2:1.

### EXAMPLE D5

### Stability of Pramlintide Solutions at 0.9 mg/mL at pH 7.4 in the Presence of Different Co-Polyamino Acids

TABLE 26
ThT (2 μM) latency time measurement
of compositions CY1, CP2 to CP12.

| | | | | Ratio co- | |
|---|---|---|---|---|---|
| | Co- | Co-polyamino acid | | polyamino acid/ | |
| | polyamino | concentration | | pramlintide | Latency |
| Solution | acid | mg/mL | mM | mol/mol | time (h) |
| CY1 | — | — | | — | 0.7 |
| CP2 | BB15 | 5 | 1.22 | 5.4 | >63 |
| | | 10 | 2.45 | 10.8 | >63 |
| CP3 | BB14 | 10 | 1.96 | 8.6 | >15 |
| CP4 | AB17 | 10 | 1.22 | 9.8 | >63 |
| CP10 | BB18 | 5 | 0.72 | 3.2 | >63 |
| | | 10 | 1.44 | 6.3 | >63 |
| CP11 | BB9 | 5 | 1 | 4.4 | >50 |
| | | 10 | 2.01 | 8.8 | >63 |
| CP12 | BB2 | 5 | 1.27 | 5.6 | >10 |
| | | 10 | 2.54 | 11.2 | >63 |

Pramlintide solution at pH 7.4 (CY1) without co-polyamino acid has a short latency time. The co-polyamino acids of the invention make it possible to obtain latency times of more than 10 hours in the tested conditions.

### EXAMPLE D6

### Stability of Pramlintide Solutions at 0.6 mg/mL at pH 6.6 in the Presence of Co-Polyamino Acid BB 15 at Different Concentrations

TABLE 27
ThT (2 μM) latency time measurement
of solutions CH1 and CH2 to CH8.

| | | |
|---|---|---|
| BB15/pramlintide | Co-polyamino acid | |
| ratio | BB15 concentration | Latency |

| Solution | mol/mol | mg/mL | mM | time (h) |
|---|---|---|---|---|
| CH1 | — | — | — | 1 |
| CH2 | 2 | 1.3 | 0.29 | >4 |
| CH3 | 3 | 2 | 0.45 | >10 |
| CH4 | 4 | 2.7 | 0.61 | >50 |
| CH5 | 6 | 4 | 0.90 | >50 |
| CH6 | 8 | 5.3 | 1.19 | >50 |
| CH7 | 10 | 6.7 | 1.50 | >50 |
| CH8 | 15 | 10 | 2.24 | >50 |

Pramlintide solution at pH 6.6 (CH1) without co-polyamino acid has a short latency time; the latency times of the solutions containing a co-polyamino acid are greater than the latency times of the composition without co-polyamino acid.

### EXAMPLE D7

### Stability of Pramlintide Solutions at 0.6 mg/mL at pH 6.6 in the Presence of Different Co-Polyamino Acids

TABLE 28
ThT (2 μM) latency time measurement
of compositions CI1 to CI14.

| | | | | Ratio co- | |
|---|---|---|---|---|---|
| | Co- | Co-polyamino acid | | polyamino acid/ | |
| | polyamino | concentration | | pramlintide | Latency |
| Solution | acid | mg/mL | mM | mol/mol | time (h) |
| CI1 | BB20 | 1.3 | 0.3 | 2 | >10 |
| | | 2.6 | 0.6 | 4 | >20 |
| CI2 | BB21 | 1.3 | 0.6 | 4 | >10 |
| CI3 | AB22 | 2.4 | 0.3 | 2 | >10 |
| CI4 | BB24 | 2.9 | 0.6 | 4 | >10 |
| CI5 | BB25 | 1.5 | 0.3 | 2 | >50 |
| | | 3 | 0.6 | 4 | >50 |
| CI6 | AB23 | 3.4 | 0.23 | 2 | >10 |
| CI7 | AB28 | 2.3 | 0.3 | 2 | >10 |
| | | 4.7 | 0.6 | 4 | >10 |
| CI8 | AB24 | 1.2 | 0.15 | 1 | >10 |
| | | 2.4 | 0.3 | 2 | >50 |
| CI9 | AB25 | 1.3 | 0.15 | 1 | >10 |
| | | 2.6 | 0.3 | 2 | >10 |
| CI10 | AB26 | 1.5 | 0.3 | 2 | >10 |
| CI11 | AB27 | 1.3 | 0.15 | 1 | >5 |
| | | 2.7 | 0.3 | 2 | >10 |
| CI12 | AB31 | 1.3 | 0.15 | 1 | >10 |
| | | 2.5 | 0.3 | 2 | >10 |
| CI13 | AB29 | 8.9 | 1.15 | 7.6 | >5 |
| CI14 | AB32 | 1.3 | 0.15 | 1 | >10 |
| | | 2.5 | 0.3 | 2 | >10 |

Pramlintide solution at pH 6.6 (CH1) without co-polyamino acid has a short latency time. The co-polyamino acids of the invention make it possible to obtain latency times of more than 5 hours in the tested conditions.

### EXAMPLE D7A

### Stability of Pramlintide Solutions at 0.6 mg/mL at pH 6.6 in the Presence of Co-Polyamino Acid AB14 and Different Contents of Sodium Chloride and Zinc Chloride

TABLE 29
ThT (2 μM) latency time measurement of compositions CT1 to CT5

| | | Co-polyamino acid | | | | |
|---|---|---|---|---|---|---|
| | Co-polyamino | concentration | | [NaCl] | [ZnCl₂] | Latency time |
| Solution | acid | mg/mL | mM | (mM) | (mM) | (h) |
| CP6 | AB14 | 10 | 2.96 | — | — | <2 |
| CT1 | AB14 | 6.3 | 1.87 | — | 0.75 | 0.6 |
| CT2 | AB14 | 6.3 | 1.87 | 50 | — | >2 |
| CT3 | AB14 | 6.3 | 1.87 | 100 | — | >5 |
| CT4 | AB14 | 6.3 | 1.87 | 50 | 0.75 | >5 |
| CT5 | AB14 | 6.3 | 1.87 | 100 | 0.75 | >20 |

The solution of pramlintide at pH 6.6 and of co-polyamino acid AB14 has a longer latency time in the presence of sodium chloride or sodium and zinc chloride.

### EXAMPLE D7B

### Stability of Pramlintide Solutions at 0.6 mg/mL at pH 6.6 in the Presence of Different Co-Polyamino Acids and Different Contents of Sodium Chloride and Zinc Chloride

TABLE 30
ThT (2 μM) latency time measurement of compositions CS1 to CS11

| | | Co-polyamino acid | | | | |
|---|---|---|---|---|---|---|
| | Co-polyamino | concentration | | [NaCl] | [ZnCl₂] | Latency time |
| Solution | acid | mg/mL | mM | (mM) | (mM) | (h) |
| CS1 | AB15 | 7.8 | 1.6 | — | — | 3.5 |
| CS2 | AB15 | 11.7 | 2.3 | — | — | >30 |
| CS3 | AB15 | 3.9 | 0.8 | 50 | — | >10 |
| CS4 | AB15 | 6.3 | 1.3 | 50 | — | >50 |
| CS5 | AB15 | 7.8 | 1.6 | 50 | — | >30 |
| CS6 | AB15 | 3.9 | 0.8 | 100 | — | >50 |
| CS7 | AB16 | 12.4 | 1.5 | — | — | 8 |
| CS8 | AB16 | 16.7 | 2.1 | — | — | >50 |
| CS9 | AB16 | 7.4 | 0.9 | 50 | | >20 |
| CS10 | AB16 | 12.4 | 1.5 | 50 | | >50 |
| CS11 | AB16 | 7.4 | 0.9 | 50 | 1 | >30 |

Solutions of pramlintide at pH 6.6 and co-polyamino acid AB15 and AB16 have a longer latency time in the presence of sodium chloride or sodium chloride and zinc.

TABLE 30a
ThT (2 μM) latency time measurement of compositions
CS7 and CS12, CS14 to CS17 and CS19 to CS30.

| | | Co-polyamino acid | | Ratio co-polyamino | | | |
|---|---|---|---|---|---|---|---|
| | Co-polyamino | concentration | | acid/pramlintide | [NaCl] | [ZnCl₂] | Latency time |
| Solution | acid | mg/mL | mM | mol/mol | (mM) | (mM) | (h) |
| CS7 | AB16 | 12.4 | 1.5 | 10.5 | — | — | <10 |
| CS12 | AB16 | 12.4 | 1.6 | 10.5 | 25 | — | >30 |
| CS14 | AB16 | 10 | 1.3 | 8.4 | 50 | — | >30 |
| CS15 | AB16 | 10 | 1.3 | 8.4 | 100 | — | >60 |
| CS16 | AB34 | 6.1 | 1.5 | 10 | — | — | <5 |
| CS17 | AB34 | 6.1 | 1.5 | 10 | 100 | — | >40 |
| CS19 | AB33 | 5.7 | 1.5 | 10 | — | — | <5 |
| CS20 | AB33 | 5.7 | 1.5 | 10 | 50 | — | >10 |
| CS21 | AB33 | 5.7 | 1.5 | 10 | 50 | 1 | >35 |
| CS22 | AB33 | 5.7 | 1.5 | 10 | 100 | — | >35 |
| CS23 | AB33 | 5.7 | 1.5 | 10 | 100 | 1 | >40 |
| CS24 | AB36 | 6.3 | 1.6 | 10.4 | — | — | <2 |
| CS25 | AB36 | 5.1 | 1.3 | 8 | 50 | — | >60 |
| CS26 | AB35 | 5.9 | 1.5 | 10 | — | — | <10 |
| CS27 | AB35 | 3.6 | 0.91 | 6 | 25 | — | >43 |
| CS28 | AB34 | 6.1 | 1.5 | 10 | — | — | <1 |
| CS29 | AB34 | 6.1 | 1.5 | 10 | 50 | — | >10 |
| CS30 | AB34 | 6.1 | 1.5 | 10 | 100 | — | >40 |

The addition of salt, alone or in the presence of zinc, makes it possible to obtain both very good latency times and a significant reduction in co-polyamino acid concentrations in the compositions.

### EXAMPLE D8

### Stability of Pramlintide Solutions at 0.4 mg/mL and Human Insulin 100 IU/ml at pH 7.4 Containing Co-Polyamino Acid BB15

TABLE 31
ThT (1 μM) latency time measurement
of compositions CN1 to CN3.

| | | |
|---|---|---|
| Co-polyamino acid | Ratio | |
| BB15 concentration | BB15/pramlintide | Latency |

| Solution | mg/mL | mM | mol/mol | time (h) |
|---|---|---|---|---|
| CN1 | — | | — | * |
| CN2 | 2.4 | 0.59 | 6 | >19 |
| CN3 | 4 | 0.98 | 10 | >19 |

* Latency time not measured due to turbid solution.

Pramlintide and human insulin solution at pH 7.4 (CN1) without copolyamino acid is turbid.

Co-polyamino acid BB15 makes it possible to obtain a clear solution of pramlintide at 0.4 mg/mL and human insulin 100 IU/mL at pH 7.4 with latency times greater than 19 hours for molar ratios BB15/pramlintide greater than or equal to 6.

### EXAMPLE D9

### Stability of Pramlintide Solutions at 0.9 mg/mL and of Human Insulin 100 IU/mL at pH 7.4 in the Presence of Co-Polyamino Acid BB 15 at Different Concentrations

TABLE 32
ThT (2 μM) latency time measurement of
compositions CR1 to CR4 and CU3, CU7 and CU8.

| | | |
|---|---|---|
| BB15/pramlintide | Co-polyamino acid | |
| ratio | BB15 concentration | Latency |

| Solution | mol/mol | mg/mL | mM | time (h) |
|---|---|---|---|---|
| CR1 | — | — | — | * |
| CU3 | 2 | 1.8 | 0.44 | >0.5 |
| CR2 | 3 | 2.7 | 0.66 | >2 |
| CR3 | 4 | 3.6 | 0.88 | >6 |
| CR4 | 5 | 4.5 | 1.10 | >9 |
| CU7 | 6 | 5.4 | 1.32 | >9 |
| CU8 | 10 | 9 | 2.20 | >9 |

* Latency time not measured due to turbid solution.

A solution of pramlintide at 0.9 mg/mL and human insulin 100 IU/mL at pH 7.4 (CR1) without co-polyamino acid is turbid. The clear solutions of pramlintide at 0.9 mg/mL and insulin human 100 IU/mL at pH 7.4 in the presence of co-polyamino acid BB15 have latency times greater than 0.5 hour at molar ratio BB15/pramlintide of 2, which may be greater than 9 h for BB15/pramlintide molar ratios greater than 5.

### EXAMPLE D10

### Stability of Pramlintide Solutions at 0.9 mg/mL and Human Insulin 100 IU/mL at pH 7.4 in the Presence of Various Co-Polyamino Acids

TABLE 33
ThT (2 μM) latency time measurement of solutions CG2 to CG12.

| | | | | Ratio co- | |
|---|---|---|---|---|---|
| | Co- | Co-polyamino acid | | polyamino acid/ | |
| | polyamino | concentration | | pramlintide | Latency |
| Solution | acid | mg/mL | mM | mol/mol | time (h) |
| CR1 | | — | | — | * |
| CG2 | BB15 | 5 | 1.22 | 5.4 | >9 |
| | | 10 | 2.45 | 10.8 | >7 |
| CG3 | BB14 | 10 | 1.96 | 8.6 | >9 |
| CG4 | AB17 | 5 | 1.11 | 4.9 | >2 |
| | | 10 | 1.22 | 9.8 | >5 |
| CG5 | AB15 | 10 | 1.99 | 8.8 | >2 |
| CG10 | BB18 | 5 | 0.72 | 3.2 | >1 |
| | | 10 | 1.44 | 6.3 | >4 |
| CG11 | BB9 | 5 | 1 | 4.4 | >4 |
| | | 10 | 2.01 | 8.8 | >3 |
| CG12 | BB2 | 5 | 1.27 | 5.6 | >5 |
| | | 10 | 2.54 | 11.2 | >6 |

* Latency time not measured due to turbid solution.

Pramlintide and human insulin solution at pH 7.4 (CR1) is turbid. Co-polyamino acids make it possible to obtain latency times of more than 1 hour in the tested conditions.

### EXAMPLE D11

### Stability of Pramlintide Solutions at 0.9 mg/mL and of Insulin Lispro 100 IU/mL at pH 7.4 in the Presence of Co-Polyamino Acid BB15 at Different Concentrations

TABLE 34
ThT (2 μM) latency time measurement of solutions CD1 and CD3 to CD8.

| | BB15/pramlintide | Co-polyamino acid | | |
|---|---|---|---|---|
| | ratio | BB15 concentration | | |
| Solution | mol/mol | mg/mL | mM | Latency time (h) |
| CD1 | | — | — | * |
| CD3 | 2 | 1.8 | 0.44 | 0.8 |
| CD4 | 3 | 2.7 | 0.66 | >2 |
| CD5 | 4 | 3.6 | 0.88 | >7 |
| CD6 | 5 | 4.5 | 1.10 | >9 |
| CD7 | 6 | 5.4 | 1.22 | >9 |
| CD8 | 10 | 9 | 1.32 | >9 |

* Latency time not measured due to turbid solution.

The solution of pramlintide and insulin lispro at pH 7.4 (CD1) is turbid. Co-polyamino acids make it possible to obtain latency times of more than 0.8 hour in the tested conditions.

### EXAMPLE D12

### Stability of Pramlintide Solutions at 0.6 mg/mL and of Human Insulin 100 IU/mL at pH 6.6 in the Presence of Co-Polyamino Acid BB15 at Different Concentrations

TABLE 35
ThT (2 μM) latency time measurement of solutions CK1 and CK3 to CK8.

| | BB15/pramlintide | Co-polyamino acid | | |
|---|---|---|---|---|
| | ratio | BB15 concentration | | |
| Solution | mol/mol | mg/mL | mM | Latency time (h) |
| CK1 | — | — | — | * |
| CK3 | 3 | 2 | 0.45 | >0.5 |
| CK4 | 4 | 2.7 | 0.61 | >5 |
| CK5 | 6 | 4 | 0.90 | >5 |
| CK6 | 8 | 5.3 | 1.19 | >5 |
| CK7 | 10 | 6.7 | 1.50 | >5 |
| CK8 | 15 | 10 | 2.24 | >5 |

* Latency time not measured due to turbid solution.

Pramlintide and human insulin solution at pH 6.6 (CK1) is turbid. The clear solutions of pramlintide at 0.6 mg/mL and human insulin 100 IU/mL at pH 6.6 in the presence of co-polyamino acid BB15 have latency times greater than 0.5 hours at a BB15/pramlintide molar ratio of 3, which may be greater than 5 hours from a BB15/pramlintide ratio of 4.

### EXAMPLE D13

### Stability of Pramlintide Solutions at 0.6 mg/mL and Human Insulin 100 IU/mL at pH 6.6 in the Presence of Various Co-Polyamino Acids

TABLE 36
ThT (2 μM) latency time measurement of solutions CM1 to CM18.

| | | Co-polyamino | | Ratio | |
|---|---|---|---|---|---|
| | | acid | | co-polyamino | Latency |
| | Co-polyamino | concentration | | acid/pramlintide | time |
| Solution | acid | mg/mL | mM | mol/mol | (h) |
| CM1 | BB20 | 2.6 | 0.61 | 4 | >1 |
| | | 5.3 | 1.22 | 8 | >1 |
| CM2 | BB21 | 1.3 | 0.6 | 4 | >10 |
| CM3 | AB22 | 2.4 | 0.3 | 2 | >5 |
| CM4 | BB24 | 2.9 | 0.6 | 4 | >10 |
| CM5 | BB23 | 3 | 0.76 | 5 | >10 |
| CM6 | BB25 | 1.5 | 0.3 | 2 | >10 |
| CM7 | BB22 | 2.7 | 0.6 | 4 | >5 |
| CM8 | AB23 | 7.7 | 0.69 | 4.6 | >15 |
| CM9 | BB19 | 4.7 | 0.4 | 2.8 | >1 |
| CM10 | AB28 | 2.3 | 0.3 | 2 | >10 |
| CM11 | AB24 | 2.4 | 0.3 | 2 | >5 |
| CM12 | AB25 | 2.6 | 0.3 | 2 | >5 |
| CM13 | AB26 | 1.5 | 0.3 | 2 | >1 |
| | | 2.3 | 0.5 | 3 | >10 |
| CM14 | AB27 | 1.3 | 0.15 | 1 | >1 |
| | | 2.7 | 0.3 | 2 | >5 |
| CM15 | AB30 | 1.2 | 0.15 | 1 | >1 |
| | | 2.3 | 0.3 | 2 | >10 |
| CM16 | AB31 | 1.3 | 0.15 | 1 | >1 |
| | | 2.5 | 0.3 | 2 | >10 |
| CM17 | AB29 | 5.9 | 0.8 | 5 | >1 |
| | | 8.9 | 1.15 | 7.6 | >5 |
| CM18 | AB32 | 2.5 | 0.3 | 2 | >1 |

Pramlintide and human insulin solution at pH 6.6 (CK1) is turbid. Co-polyamino acids produce latency times of more than 1 hour in the tested conditions.

### EXAMPLE D13A

### Stability of Pramlintide Solutions at 0.6 mg/mL and Human Insulin 100 IU/mL at pH 6.6 in the Presence of Different Co-Polyamino Acids and Different Contents of Sodium Chloride and Zinc Chloride

TABLE 37
ThT (2 μM) latency time measurement of solutions CQ1 to CQ12

| | | Co-polyamino acid | | | | |
|---|---|---|---|---|---|---|
| | Co-polyamino | concentration | | [NaCl] | [ZnCl₂] | Latency time |
| Solution | acid | mg/mL | mM | (MM) | (MM) | (h) |
| CQ1 | AB14 | 6.3 | 1.87 | 100 | 1 | >5 |
| CQ2 | AB15 | 7.8 | 1.6 | — | 0.23 | >2 |
| CQ3 | AB15 | 11.7 | 2.3 | — | 0.23 | >2 |
| CQ6 | AB15 | 7.8 | 1.6 | 50 | 0.23 | >5 |
| CQ7 | AB15 | 3.9 | 0.8 | 100 | 0.23 | >2 |
| CQ9 | AB15 | 7.8 | 1.6 | 100 | 0.23 | >5 |
| CQ10 | AB16 | 7.4 | 0.9 | 50 | 0.23 | >1 |
| CQ12 | AB16 | 7.4 | 0.9 | 50 | 1 | >2 |

Solutions of pramlintide and human insulin at pH 6.6 in the presence of co-polyamino acids of AB14, AB15 and AB16, sodium chloride and zinc have latency times longer than 1 hour in the tested conditions. The addition of sodium chloride or sodium and zinc chloride makes it possible to increase latency times.

### EXAMPLE D14

### Stability of Compositions with Variable Pramlintide Concentrations and Human Insulin at 100 IU/mL in the Presence of Co-Polyamino Acid AB24, m-cresol (29 mM), Glycerol (174 mM) and Zinc Chloride (229 μM) at pH 6.6

TABLE 38
ThT (2 μM) latency time measurement of solutions CF2 to CF6.

| | | Co-polyamino | | Ratio | |
|---|---|---|---|---|---|
| | Concentration | acid concentration | | co-polyamino | Latency |
| | of pramlintide | AB24 | | acid/pramlintide | time |
| Solution | (mg/mL) | mg/mL | mM | mol/mol | (h) |
| CF2 | 0.9 | 5.4 | 0.67 | 3 | >10 |
| CF3 | 0.8 | 4.8 | 0.6 | 3 | >10 |
| CF4 | 0.6 | 3.6 | 0.45 | 3 | >5 |
| CF5 | 0.3 | 1.8 | 0.22 | 3 | >5 |
| CF6 | 0.2 | 1 | 0.125 | 2.5 | >5 |

Pramlintide solutions of varying concentration levels and human insulin at 100 IU/mL at pH 6.6 are turbid (EXAMPLEs CF1A-E). Pramlintide solutions of varying concentration levels and human insulin at 100 IU/mL at pH 6.6 in the presence of co-polyamino acid AB24 have latency times greater than 5 hours in the tested conditions.

### D II: Study of the Stability of the Compositions According to the Invention

### D II A: Preparation of Compositions

Composition D1: Preparation of Pramlintide Solution at 0.9 mg/mL Containing m-cresol (29 mM) and Glycerol (174 mM) at pH 6.6

By a process similar to that used in example CH1, a pramlintide solution at 0.9 mg/mL containing m-cresol (29 mM) and glycerol (174 mM) at pH 6.6 is obtained. The solution is clear.

Composition D2: Preparation of a Pramlintide Solution at 0.9 mg/mL Containing Co-Polyamino Acid BB15, m-cresol (29 mM) and Glycerol (174 mM) at pH 6.6

A 0.9 mg/mL pramlintide solution and a 10 mg/mL co-polyamino acid BB15 solution containing m-cresol (29 mM) and glycerol (174 mM) at pH 6.6 is obtained by a process similar to that used in example CHO. The solution is clear.

### D II B: Visual Inspection Procedure:

Vials or 3 mL cartridges filled with 1 mL of formulation are visually inspected for the appearance of visible particles or turbidity. This inspection is carried out according to the recommendations of the European pharmacopoeia (EP 2.9.20): the vials are subjected to a lighting of at least 2000 lux and are observed on a white background and a black background. The number of weeks or months of stability corresponds to the time from which the solutions contain visible particles or are turbid.

These results are in agreement with the US pharmacopoeia (USP <790>).

### D II C: Dosage Procedure for the Formulations:

Quantification of the purity of pramlintide and insulin and the recovery of native peptide is carried out by reverse phase HPLC equipped with a 4.6×150 mm CA18 column with a particle size of 3.5 μm. Pramlintide is detected at a wavelength of 214 nm and insulin is detected at a wavelength of 276 nm. The elution is carried out in an aqueous mobile phase with a linear gradient of acetonitrile.

Pramlintide or insulin (%) recovery at time t represents the ratio between pramlintide peak area or insulin peak area at time t and the initial pramlintide peak area.

The purity of pramlintide and insulin (%) represents the ratio of peak absorbance range of pramlintide or insulin to the total area of the set of peaks including pramlintide and its impurities.

D II D: Physical Stability in Cartridges at 37° C. of Pramlintide Solutions at 0.9 mg/mL in the Presence of Co-Polyamino Acid BB15, m-cresol (29 mM) and Glycerol (174 mM) at pH 6.6 or pH 7.4

D1, CY1, D2 and CY7 solutions are filtered (0.22 μm). 1 mL of solution is introduced into a 3 mL glass cartridge for auto-injector pen. The cartridges are placed in a static oven at 37° C. The cartridges are observed weekly.

TABLE 39
Physical stability results of 0.9 mg/mL pramlinitide compositions in
the presence of co-polyamino acid BB15 at 37° C. in cartridges.

| | Co-polyamino acid BB15 | | Physical stability |
|---|---|---|---|
| | concentration | | 37° C. in cartridges |
| Solution | mg/mL | pH | (weeks) |
| D1 | — | 6.6 | <1 |
| CY1 | — | 7.4 | <1 |
| D2 | 10 | 6.6 | >4 |
| CY7 | 10 | 7.4 | >4 |

Pramlintide solutions at 0.9 mg/mL at pH 6.6 and pH 7.4 have a physical stability at 37° C. in cartridge less than one week.

Pramlintide solutions at 0.9 mg/mL at pH 6.6 and pH 7.4 in the presence of BB15 co-polyamino acid have a physical stability at 37° C. in cartridges of at least 4 weeks.

### EXAMPLE D II C

### Chemical Stability in Cartridges at 37° C. of Pramlintide Solutions at 0.9 mg/mL in the Presence of Co-Polyamino Acid BB15, m-cresol (29 mM) and Glycerol (174 mM) at pH 6.6 and 7.4

The solutions described in example D II D are analyzed by RP-HPLC chromatography.

TABLE 40
Results of the chemical stabilities of pramlintide compositions
at 0.9 mg/mL in the presence of co-polyamino acid BB15.

| | Co-polyamino | | | | |
|---|---|---|---|---|---|
| | acid BB15 | | Recovery | Purity | |
| | concentration | | pramlintide (%) | pramlintide (%) | |
| Solution | mg/mL | pH | 32 days-37° C. | T0 | 32 days 37° C. |
| D1 | — | 6.6 | <60 | 97.2 | <50 |
| CY1 | — | 7.4 | <20 | 94.7 | <50 |
| D2 | 10 | 6.6 | >90 | 97.8 | >85 |
| CY7 | 10 | 7.4 | >60 | 98.6 | >60 |

Pramlintide solutions at 0.9 mg/mL at pH 6.6 and pH 7.4 have a pramlintide recovery of less than 60% and pramlintide purity is less than 50%% after 32 days at 37° C. in cartridge.

Pramlintide solutions at 0.9 mg/mL at pH 6.6 and pH 7.4 in the presence of co-polyamino acid BB15 have a recovery greater than 65% at pH 7.4 and can be greater than 90% at pH 6.6 after 32 days at 37° C. in cartridges. In the presence of co-polyamino acid BB15, pramlintide purity is greater than 65% at pH 7.4 and can be greater than 85% at pH 6.6.

### EXAMPLE D II E

### Physical Stability in Vial and Cartridge at 30° C. of Pramlintide Solutions at 0.9 mg/mL and at 0.6 mg/mL in the Presence of Co-Polyamino Acid BB15, m-cresol (29 mM) and Glycerol (174 mM) at pH 6.6

D1, CH1, D2 and CH8 solutions are filtered (0.22 μm). 1 mL of solution is introduced into 3 mL glass cartridges for auto-injector pen and in 3 mL glass vials. Cartridges and vials are placed in an oven at 30° C. static and are observed every 2 weeks.

TABLE 41
Results of the physical stabilities in vial and in cartridge at 30° C.
of pramlintide compositions at 0.9 and 0.6 mg/mL in the presence
of co-polyamino acid BB15.

| | | | | Physical | Physical |
|---|---|---|---|---|---|
| | | | | stability | stability |
| | Co-polyamino acid | Concentration | | 30° C. | 30° C. in |
| | concentration BB15 | pramlintide | | in vial | cartridge |
| Solution | mg/mL | (mg/mL) | pH | (week) | (week) |
| D1 | — | 0.9 | 6.6 | <7 | <2 |
| CH1 | — | 0.6 | 6.6 | <7 | — |
| D2 | 10 | 0.9 | 6.6 | >12 | >12 |
| CH8 | 10 | 0.6 | 6.6 | >12 | >12 |

Pramlintide solutions at 0.9 mg/mL and 0.6 mg/mL at pH 6.6 have a physical stability in a vial of less than 7 weeks at 30° C. The physical stability in a cartridge of pramlintide solution at 0.9 mg/mL pH 6.6 is less than 2 weeks.

Pramlintide solutions at 0.9 mg/mL and 0.6 mg/mL at pH 6.6 in the presence of co-polyamino acid BB15 exhibit a physical stability at 30° C. of above 12 weeks in vial and in cartridge.

### EXAMPLE D II F

### Chemical Stability in Vial at 30° C. of Pramlintide Solutions at 0.9 mg/mL and at 0.6 mg/ml in the Presence of Co-Polyamino Acid BB15, m-cresol (29 mM) and Glycerol (174 mM) at pH 6.6

The solutions described in example D II E are analyzed by RP-HPLC chromatography.

TABLE 42
Results of the chemical stabilities in vial at 30° C. of pramlintide
compositions at 0.9 and 0.6 mg/mL in the presence of co-polyamino
acid BB15 at pH 6.6.

| | Co-polyamino | | Recovery | | |
|---|---|---|---|---|---|
| | acid | | pramlintide | Purity | |
| | concentration | Concentration | (%) | pramlintide (%) | |
| | BB15 | pramlintide | 5 weeks | | 5 weeks |
| Solution | mg/mL | (mg/mL) | 30° C. | T0 | 30° C. |
| D1 | — | 0.9 | <70 | 97.2 | <60 |
| D2 | 10 | 0.9 | >95 | 97.8 | >90 |
| CH8 | 10 | 0.6 | >95 | 94.6 | >90 |

Pramlintide solution at 0.9 mg/mL at pH 6.6 has a pramlintide recovery of less than 70% and pramlintide purity of less than 60% after 5 weeks at 30° C. in vial.

Pramlintide solutions at 0.9 mg/mL and 0.6 mg/mL at pH 6.6 in the presence of co-polyamino acid BB 15 have a pramlintide coverage of greater than 95% and pramlintide purity is greater than 90% after 5 weeks at 30° C.

### EXAMPLE D II F

### Chemical Stability in Vial at 30° C. and Cartridge at 30° C. of Pramlintide Solutions at 0.6 mg/mL and Insulin 100 IU/mL at pH 6.6 in the Presence of Co-Polyamino Acid BB15, m-cresol (29 mM), Glycerol (174 mM) and Zinc at pH 6.6

The CK8 solution is filtered (0.22 μm). 1 mL of solution is introduced into 3 mL glass cartridges for auto-injector pen and in 3 mL glass vials. Cartridges and vials are placed in an oven at 30° C. static and are observed every 2 weeks.

TABLE 43
Results of the chemical stabilities in vial and in cartridge at 30° C.
of pramlintide compositions at 0.6 mg/mL, insulin 100 IU/mL in the
presence of co-polyamino acid BB15 at pH 6.6.

| | Co-polyamino | | | Physical | Physical |
|---|---|---|---|---|---|
| | acid | | Concen- | stability | stability |
| | concentration | Concentration | tration | 30° C. | 30° C. in |
| | BB15 | pramlintide | Insulin | in vial | cartridge |
| Solution | mg/mL | (mg/mL) | (IU/mL) | (week) | (week) |
| CK1 | — | 0.6 | 100 | * | * |
| CK8 | 10 | 0.6 | 100 | >3 | >12 |

* turbid solution as soon as it is prepared.

The solution of pramlintide at 0.6 mg/mL and insulin at 100 IU/mL at pH 6.6 is turbid.

The solution of pramlintide at 0.6 mg/mL and human insulin at 100 IU/mL at pH 6.6 in the presence of co-polyamino acid BB15 has physical stability at 30° C. of 3 weeks in vial and greater than 12 weeks in cartridge.

### EXAMPLE D II G

### Chemical Stability in Vial at 30° C. of a Pramlintide Solution at 0.6 mg/mL and Insulin 100 IU/mL at pH 6.6 in the Presence of Co-Polyamino Acid BB15, m-cresol (29 mM), Glycerol (174 mM) and Zinc

The solution described in example D II F is analyzed by RP-HPLC chromatography.

TABLE 44
Results of chemical stability in vial at 30° C. of
a composition of pramlintide at 0.6 mg/mL, of insulin 100
IU in the presence of co-polyamino acid BB15 at pH 6.6.

| | Recovery | Purity | | Recovery | Purity | |
|---|---|---|---|---|---|---|
| | pramlintide | pramlintide | | Insulin | Insulin | |
| | (%) | (%) | | (%) | (%) | |
| | 5 weeks | | 5 weeks | 5 weeks | | 5 weeks |
| Solution | 30° C. | T0 | 30° C. | 30° C. | T0 | 30° C. |
| CK8 | >90 | 96.8 | >90 | >90 | 97.9 | >90 |

The solution of pramlintide at 0.6 mg/mL and insulin at 100 IU/mL at pH 6.6 is turbid.

The solution of pramlintide at 0.6 mg/mL and of insulin at 100 IU/mL at pH 6.6 in the presence of co-polyamino acid BB15 has a pramlintide recovery greater than 90% and pramlintide purity is greater than 90% after 5 weeks at 30° C. in a vial. Insulin recovery is greater than 90% and insulin purity is greater than 90% after 5 weeks at 30° C. in vial.

### EXAMPLE D II H

### Physical Stability in Vial at 30° C. and Cartridge at 30° C./37° C. of Pramlintide Solutions at 0.6 mg/mL and Insulin 100 IU/mL at pH 6.6 in the Presence of Co-Polyamino Acid AB24 at 2.4 mg/mL, m-cresol (29 mM), Glycerol (174 mM) and Zinc at pH 6.6

CM11 solution is filtered (0.22 μm). 1 mL of solution is introduced into 3 mL glass cartridges for auto-injector pen and in 3 mL glass vials. Cartridges and vials are placed in an oven at 30° C. static and are observed every 2 weeks. Cartridges are also placed in an oven at 37° C. under static conditions, then observed every week.

TABLE 45
Results of physical stabilities in vial at 30° C. and in cartridge
at 30 and 37° C. of pramlintide compositions at 0.6 mg/mL, insulin
100 IU/mL and in the presence of AB24 co-polyamino acid at pH 6.6.

| | | | | Physical | Physical | Physical |
|---|---|---|---|---|---|---|
| | | Concentration | Concentration | stability | stability | stability |
| | Co-polyamino | pramlintide | Insulin | 30° C. in | 30° C. in | 37° C. in |
| Solution | mg/mL | (mg/mL) | (IU/mL) | vial | cartridge | cartridge |
| CK1 | — | 0.6 | 100 | * | * | * |
| CM11 | 2.4 | 0.6 | 100 | >9 | >12 | >9 |

* turbid solution as soon as it is prepared.

The solution of pramlintide at 0.6 mg/mL and insulin at 100 IU/mL at pH 6.6 is turbid.

The solution of pramlintide at 0.6 mg/mL and human insulin at 100 IU/mL at pH 6.6 in the presence of co-polyamino acid AB24 has physical stability at 30° C. of above 9 weeks in vial and greater than 12 weeks in cartridge. Physical stability at 37° C. cartridge is greater than 9 weeks.

### EXAMPLE D II I

### Chemical Stability in Vial and Cartridge at 30° C. of Pramlintide Solutions at 0.6 mg/mL and Insulin 100 IU/mL at pH 6.6 in the Presence of Co-Polyamino Acid AB24 at 2.4 mg/mL, m-cresol (29 mM), Glycerol (174 mM) and Zinc at pH 6.6

The solution described in example D II H is analyzed by RP-HPLC chromatography.

TABLE 46
Results of the chemical stabilities in vial and cartridge at
30° C. of pramlintide compositions at 0.6 mg/mL, insulin
100 IU in the presence of AB24 co-polyamino acid at pH 6.6.

| | Solution | | | | | |
|---|---|---|---|---|---|---|
| | pramlintide | pramlintide | | Insulin | Insulin | |
| | Recovery | Purity | | Recovery | Purity | |
| | (%) | (%) | | (%) | (%) | |
| | 9 weeks | | 9 weeks | 9 weeks | | 9 weeks |
| CM11 | 30° C. | T0 | 30° C. | 30° C. | T0 | 30° C. |
| Vial | >88 | 96.7 | >90 | >90 | 97.4 | >90 |
| Cartridge | >88 | 96.9 | >85 | >90 | 97.7 | >90 |

The solution of pramlintide at 0.6 mg/mL and of human insulin at 100 IU/mL at pH 6.6 in the presence of co-polyamino acid AB24 has a pramlintide recovery greater than 88% and a purity greater than 90 and 85% respectively, after 9 weeks of storage at 30° C. in vial and in cartridge. Under these conditions insulin recovery is greater than 90% and insulin purity greater than 90% in vial and in cartridge.

### EXAMPLE D II J

### Physical Stability in Cartridge at 4° C. of Solutions of Pramlintide at 0.6 mg/mL at pH 6.6 in the Presence of Co-Polyamino Acid BB15, m-cresol (29 mM), Glycerol (174 mM) at pH 6.6

The CH8 solution is filtered (0.22 μm). 1 mL of solution is introduced into 3 mL glass cartridges for auto-injector pen. The cartridges are placed in a refrigerator at 4° C.

TABLE 47
Results of the cartridge physical stability at 4° C. of a pramlintide
composition at 0.6 mg/mL and in the presence of BB15 co-polyamino
acid at pH 6.6.

| | | | | Physical |
|---|---|---|---|---|
| | Co-polyamino acid | Concentration | | stability |
| | concentration BB15 | pramlintide | | 4° C. in cartridge |
| Solution | mg/mL | (mg/mL) | pH | (month) |
| CH8 | 10 | 0.6 | 6.6 | >6 |

The solution of pramlintide at 0.6 mg/mL and of human insulin at 100 IU/mL at pH 6.6 in the presence of co-polyamino acid BB 15 has a physical stability in cartridge of over 6 months.

### EXAMPLE D II K

### Physical Stability in Cartridge at 4° C. of Pramlintide Solutions at 0.6 mg/mL at pH 6.6 and pH 7.4 in the Presence of DMPG at 4.5 mM

CZ0 and CZ1 solutions are filtered (0.22 μM). 1 ml of solution is introduced into 3 mL glass cartridges for auto-injector pen. The cartridges are placed in a refrigerator at 4° C.

TABLE 48
Results of the physical stabilities in cartridge at 4° C. of pramlintide
compositions at 0.6 mg/mL and in the presence of DMPG at
pH 6.6 and 7.4.

| | | | | | Physical |
|---|---|---|---|---|---|
| | | | | | stability |
| | | Concentration | | | at 4° C. in |
| | DMPG | pramlintide | | | cartridge |
| Solution | (MM) | (mg/mL) | pH | Excipients | (month) |
| CZ0 | 4.5 | 0.6 | 6.6 | m-cresol 29 mM | <1.5 |
| | | | | glycerol 174 mM | |
| CZ1 | 4.5 | 0.6 | 7.4 | 30 mM phenol | <1.5 |
| | | | | Glycylglycine | |
| | | | | 8 mM pH 7.4 | |
| | | | | Glycerol 174 mM | |

Pramlintide solutions at 0.6 mg/mL at pH 6.6 and pH 7.4 have a physical stability at 4° C. in cartridge of less than 1.5 months (turbid solutions).

### EXAMPLE D II L

### Pump Stability of Solutions of Pramlintide at 0.6 mg/mL and of Human Insulin at 100 IU/mL at pH 6.6 in the Presence of Co-Polyamino Acid AB24 at 3.6 mg/mL

The CF4 solution consisting of 0.6 mg/mL of pramlintide and 100 IU/mL of human insulin at pH 6.6 in the presence of co-polyamino acid AB24 at 3.6 mg/mL is filtered (0.22 μm) and introduced into a 3 mL insulin pump reservoir (Minimed 530G system manufactured by Medtronic). The pump is equipped with an infusion set (Quick set Paradigm 9/100 manufactured by Medtronic).

The insulin pump is placed in an oven at 37° C. on an orbital stirrer set to a speed of 100 rpm. The pump is set at a basal flow rate of 0.8 IU/h. 6 IU bolus injections are performed 3 times per day for a total duration of 8 days.

Table 49 provides the results of MFI (Micro-Flow Imaging) measurements and dosages carried out by RP-HPLC on the fractions collected between the 7^{th} day and the 8^{th} day of stability testing.

TABLE 49
Results of the chemical stability in pump at 37° C. of pramlintide compositions
at 0.6 mg/mL, insulin 100 IU/mL and in the presence of co-polyamino acid AB24 at pH 6.6.

| | Solution | | | | | | |
|---|---|---|---|---|---|---|---|
| | | pramlintide | pramlintide | | Human insulin | | |
| | Particles subvisible | Recovery | Purity | | Recovery | Insulin Purity | |
| | (Micro Flow Imaging, MFI) | (%) | (%) | | (%) | (%) | |
| | 1 week | 1 week | | 1 week | 1 week | | 1 week |
| | 37° C. | 37° C. | T0 | 37° C. | 37° C. | T0 | 37° C. |
| CM11 | particles > 10 μM < 6000 | >95 | 97.3 | >95 | >95 | 97.4 | >95 |
| | particles per container * | | | | | | |
| | particles > 25 μM < 600 | | | | | | |
| | particles per container * | | | | | | |

* Standard USP <788> on the number of subvisible particles in the products for parenteral injections.

After one week of pump stability at 37° C., the solution of pramlintide at 0.6 mg/mL and of human insulin at 100 IU/mL at pH 6.6 in the presence of co-polyamino acid AB24 is clear and has a number of subvisible particles in accordance with the USP standard <788>. Under these conditions, pramlintide and insulin recovery and purity are greater than 95%.

### EXAMPLE D II M

### Pump Stability of Solutions of Pramlintide at 0.6 mg/mL and of Insulin Lispro at 100 IU/mL at pH 6.6 in the Presence of Co-Polyamino Acid AB24 at 3.6 mg/mL

The CA5 solution composed of 0.6 mg/mL of pramlintide and 100 IU/ml of insulin lispro at pH 6.6 in the presence of co-polyamino acid AB24 at 3.6 mg/mL is filtered (0.22 μm) and subjected to a pump stability test by a protocol identical to that described in EXAMPLE D II L.

TABLE 50
Results of the chemical stability in pump at 37° C. of pramlintide compositions
at 0.6 mg/mL, insulin 100 IU/mL and in the presence of co-polyamino acid AB24 at pH 6.6.

| | | pramlintide | pramlintide | | lispro | Insulin | |
|---|---|---|---|---|---|---|---|
| | Particles subvisible | Recovery | Purity | | Recovery | Purity | |
| | (Micro Flow Imaging, MFI) | (%) | (%) | | (%) | (%) | |
| | 1 week | 1 week | | 1 week | 1 week | | 1 week |
| Solution | 37° C. | 37° C. | T0 | 37° C. | 37° C. | T0 | 37° C. |
| CA5 | particles > 10 μM <6000 | >95 | 97.2 | >95 | >95 | 99.3 | >95 |
| | particles per container * | | | | | | |
| | particles > 25 μM < 600 | | | | | | |
| | particles per container * | | | | | | |

* USP <788> criterion concerning the number of subvisible particles in the products for parenteral injections.

After one week of pump stability at 37° C., the solution of pramlintide at 0.6 mg/mL and of insulin lispro at 100 IU/mL at pH 6.6 in the presence of co-polyamino acid AB24 is clear and has a number of subvisible particles in accordance with the USP standard <788>. Under these conditions, pramlintide and insulin lispro recovery and purity are greater than 95%.

### E. Pharmacodynamy and Pharmacokinetics

### E1: Measurement Protocol of the Pharmacokinetics of Pramlintide and Insulin Formulations.

Domestic pigs weighing approximately 50 kg, previously catheritized at the jugular, are fasted 2.5 hours before the start of the experiment. In the hour preceding the injection of pramlintide and insulin formulations, 3 blood samples are collected in order to determine the basal level of pramlintide.

The injection of the formulations at the dose of 1.125 μg/kg for pramlintide and 0.1875 IU/kg for insulin is carried out subcutaneously in the flank of the animal using an insulin pen (Novo, Sanofi or Lilly) equipped with a 31 G needle.

Blood samples are then collected every 4 minutes for 20 minutes then every 10 to 60 minutes for up to 3 hours. After each sample collection, the catheter is rinsed with a diluted heparin solution.

The blood thus drawn is collected in a K2EDTA tube and centrifuged to isolate plasma. Pramlintide levels in the plasma samples are measured by sandwich ELISA enzyme immunoassay for each animal.

The pharmacokinetic curves expressed in basal level delta are then plotted.

The following pharmacokinetic parameters were then determined by non-compartmental analysis with Phoenix WinNonlin software:
tₘₐₓ pramlintide corresponding to the time required to reach the maximum concentration of pramlintide in plasma;
AUC_{Pram 0-30 min} corresponding to the area under the pramlintide concentration curve based on time between 0 and 30 minutes post-administration;
AUC_{Pram 60-180 min} corresponding to the area under the pramlintide concentration curve based on time between 60 and 180 minutes post-administration;
Cₗₐₛₜ corresponding to the last concentration of quantifiable pramlintide in plasma;
tₗₐₛₜ corresponding to the time Cₗₐₛₜ is observed.

Tₘₐₓ is currently used to evaluate the start of absorption. AUC_{Pram 0-30 min} is currently used to evaluate early exposure to pramlintide in plasma. AUC_{Pram 60-180 min} makes it possible to evaluate the late exposure to pramlintide in plasma. Cₗₐₛₜ and tₗₐₛₜ making it possible to study late concentration levels.

### E2: Pharmacokinetic Results of Pramlintide of Pramlintide and Insulin Formulations Examples CA2 and CA3

| | Rh- | co-polyamino | Pramlintide | Number of |
|---|---|---|---|---|
| Example | Insulin | acid | (mg/mL) | pigs |
| CA1/CA2 | 100 | — | 1 | 8 |
| (double | | | | |
| CA3 | 100 | BB15 | 0.6 | 10 |

Pramlintide pharmacokinetic results obtained with the compositions described in examples CA1/CA2 and CA3 are shown in FIG. 2. The analysis of these profiles indicates that the composition of example CA3 comprising co-polyamino acid BB15, 100 IU/mL insulin and 0.6 mg/mL pramlintide (curve plotted with the squares corresponding to example CA3), makes it possible to obtain a pramlintide absorption which is slower than that of the composition of the double-injection example comprising only pramlintide and insulin (curve drawn with the triangles corresponding to the CA1/CA2) double injection example). The pharmacokinetic parameters of pramlintide are reported in the following table:

| | tₘₐₓ | | | Cₗₐₛₜ | tₗₐₛₜ |
|---|---|---|---|---|---|
| | pramlintide | AUC_{Pram 0-30 min} | AUC_{Pram 60-180 min} | pramlintide | pramlintide |
| Example | (min) | (min * pmol/L) | (min * pmol/L) | (min) | (min) |
| CA1/CA2 | 20 ± 10 | 2076 ± 1596 | 3286 ± 1951 | 12 ± 5 | 153 ± 40 |
| CA3 | 77 ± 44 | 1006 ± 885 | 5989 ± 3146 | 28 ± 24 | 168 ± 25 |

### E3: Pharmacokinetic Results of Pramlintide of Pramlinitide and Insulin Formulations of Examples CA1/CA2 and CA4.

| | Rh- | co-polyamino | Pramlintide | Number of |
|---|---|---|---|---|
| Example | Insulin | acid | (μg/mL) | pigs |
| CA1/CA2 | 100 | — | — | 8 |
| CA4 | 100 | AB24 | 0.6 | 12 |

The pharmacokinetic results of pramlintide obtained with the compositions described in examples CA1/CA2 and CA4 are shown in FIG. 3. The analysis of these profiles indicates that the composition of example CA4 comprising co-polyamino acid AB24, 100 IU/mL of insulin and 0.6 μg/mL of pramlintide (curve plotted with the squares corresponding to example CA4) makes it possible to obtain a pramlintide absorption which is slower than that of the composition of the double-injection example comprising only pramlintide and insulin (curve drawn with the triangles corresponding to the double injection example CA1/CA2). The pharmacokinetic parameters of pramlintide are reported in the following table:

| | tₘₐₓ | | | Cₗₐₛₜ | tₗₐₛₜ |
|---|---|---|---|---|---|
| | pramlintide | AUC_{Pram 0-30 min} | AUC_{Pram 60-180 min} | pramlintide | pramlintide |
| Example | (min) | (min * pmol/L) | (min * pmol/L) | (min) | (min) |
| CA1/CA2 | 20 ± 10 | 2076 ± 1596 | 3286 ± 1951 | 12 ± 5 | 153 ± 40 |
| CA4 | 68 ± 23 | 749 ± 316 | 8064 ± 2963 | 34 ± 22 | 175 ± 17 |

1. A composition in the form of an injectable aqueous solution, the pH of which is comprised from 6.0 to 8.0, comprising at least:
a) amylin, an amylin receptor agonist or an amylin analogue;
b) a co-polyamino acid bearing carboxylate charges and hydrophobic radicals Hy, said co-polyamino acid being constituted of glutamic or aspartic units and said hydrophobic radicals Hy being according to formula I below:
*GpRᵣGpAₐGpC)ₚ Formula I
wherein
GpR is a radical according to formulas II, II′ or II″: 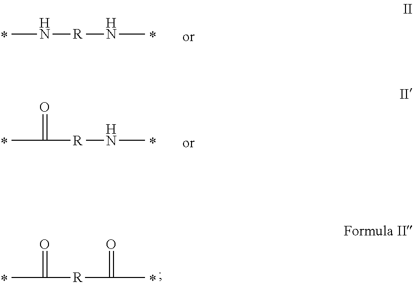
GpA is a radical according to formulas III or III′: 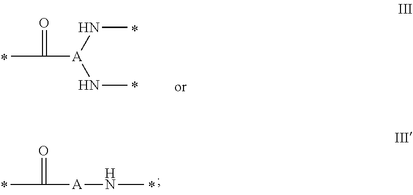
GpC is a radical according to formula IV: 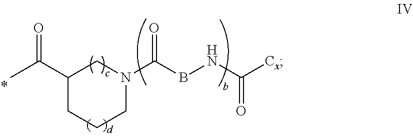
* indicate the attachment sites of the various groups;
a is an integer equal to 0 or 1;
b is an integer equal to 0 or 1;
p is an integer equal to 1 or 2 and
if p is equal to 1 then a is equal to 0 or 1 and GpA is a radical according to formula III′ and,
if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
if r is equal to 0, then the hydrophobic radical according to formula I is bound to the co-polyamino acid via a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
if r is equal to 1 or 2, then the hydrophobic radical according to formula I is bound to the co-polyamino acid:
via a covalent bond between a nitrogen atom of the hydrophobic radial and a carbonyl of the co-polyamino acid, thus forming an amide function from the reaction of an amine function of the precursor of the hydrophobic radical and an acid function borne by the precursor of the co-polyamino acid or
via a covalent bond between a carbonyl of the hydrophobic radical and a nitrogen atom in N-terminal position of the co-polyamino acid, thus forming an amide function from the reaction of an acid function of the precursor of the hydrophobic radical and an amine function in N-terminal position borne by the precursor of the co-polyamino acid;
R is a radical chosen among the group consisting of a linear or branched divalent alkyl radical comprising from 1 to 12 carbon atoms, a divalent linear or branched alkyl radical comprising from 1 to 12 carbon atoms bearing one or more —CONH2 functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms;
more precisely, R is a radical chosen among the group consisting of:
a divalent alkyl radical, linear or branched, comprising from 2 to 12 carbon atoms if GpR is a radical according to formula II, from 1 to 11 carbon atoms if GpR is a radical according to formula II' or from 0 to 10 carbon atoms if GpR is a radical according to formula II″;
a divalent, linear or branched alkyl radical, comprising from 2 to 11 carbon atoms if GpR is a radical according to formula II, from 1 to 11 carbon atoms if GpR is a radical according to formula II' or from 0 to 10 carbon atoms if GpR is a radical according to formula II″, said alkyl radical bearing one or more functions —CONH₂, and
an unsubstituted ether or polyether radical comprising from 4 to 14 carbon atoms and from 1 to 5 oxygen atoms;
A is a radical chosen among the group consisting of an unsubstituted ether or polyether radical comprising from 4 to 14 carbon atoms and from 1 to 5 oxygen atoms or a linear or branched alkyl radical comprising from 1 to 8 carbon atoms and optionally substituted with a radical derived from a saturated, unsaturated or aromatic ring;
B is a linear or branched alkyl radical, optionally comprising an aromatic ring, comprising from 1 to 9 carbon atoms;
C, is a linear or branched monovalent alkyl radical, optionally comprising a cyclic part, wherein x indicates the number of carbon atoms and:
if p is equal to 1, x is comprised from 9 to 25 (9≤x≤25):
if p is equal to 2, x is comprised from 9 to 15 (9≤x≤15),
the ratio i between the number of hydrophobic radicals and the number of glutamic or aspartic units being comprised between 0<i≤0.5;
when several hydrophobic radicals are borne by a co-polyamino acid they are thereforeidentical or different,
the degree of polymerization DP of glutamic or aspartic units is comprised from 5 to 250;
the free acid functions being in the form of an alkaline cation salt chosen among the group consisting of Na+ and K+;
characterized in that the composition does not comprise a basal insulin the isoelectric point pI of which is comprised from 5.8 to 8.5.

2. The composition according to claim 1, wherein said hydrophobic radicals are chosen among the hydrophobic radicals according to formula I wherein p=1, represented by formula V below:
*GpRᵣGpAₐGpC formula V
GpR, GpA, GpC, r and a as defined in claim 1.

3. The composition according to claim 1, wherein the said hydrophobic radicals are chosen among the hydrophobic radicals according to formula I wherein a=1 and p=2, represented by formula VI below:
*GpRᵣGpAGpC)₂ Formula VI
wherein GpR, GpA, GpC, r and a as defined in claim 1.

4. The composition according to claim 1, wherein the co-polyamino acid bearing carboxylate charges and hydrophobic radicals is chosen among the co-polyamino acids according to formula VII below: 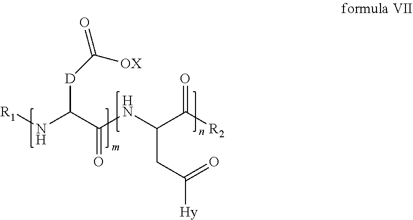
wherein,
D represents, independently, either a —CH₂— group (aspartic unit) or a —CH₂—CH₂-group (glutamic unit),
Hy is a hydrophobic radical chosen among hydrophobic radicals according to formula I, V or VI,
R₁ is a hydrophobic radical chosen among hydrophobic radicals according to formulas I, V or VI, or a radical chosen among the group consisting of a H, a C2 to C10 linear acyl group, a C3 to C10 branched acyl group, benzyl, a terminal “amino acid” unit and a pyroglutamate,
R2 is a hydrophobic radical chosen among hydrophobic radicals according to formula I, V or VI, or a radical —NR′R″, R′ and R″ identical or different chosen among the group consisting of H, linear or branched alkyls or cyclical in C2 to C10, benzyl and said R′ and R″ alkyls may form together one or more saturated carbon rings, unsaturated and/or aromatic and/or may contain heteroatoms, chosen among the group consisting of O, N and S,
X represents a H or a cationic entity chosen among the group consisting of metal cations;
n+m represents the degree of polymerization DP of the co-polyamino acid, namely the average number of monomeric units per co-polyamino acid chain and 5≤n+m≤250.

5. The composition according to claim 4, wherein the co-polyamino acid bearing carboxylate charges and hydrophobic radicals is chosen among the co-polyamino acids according to formulas VII, wherein R₁═R′₁ and R₂═R′₂, according to formula VIIa below: 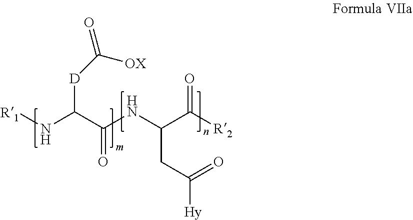
wherein,
m, n, X, D and Hy as defined in claim 4,
R′1 is a radical chosen among the group consisting of H, linear C2-C10 acyl group, branched C3-C10 acyl group, benzyl, terminal “amino acid” unit and pyroglutamate,
R′2 is an identical or different radical —NR′R″, R′ and R″ chosen among the group consisting of H, linear or branched or cyclic C2 to C10 alkyls, benzyl and said R′ and R″ alkyls which can jointly form one or more saturated, unsaturated and/or aromatic carbon rings and/or which may contain heteroatoms chosen among the group consisting of O, N and S.

6. The composition according to claim 4, wherein the co-polyamino acid bearing carboxylate charges and hydrophobic radicals is chosen among the co-polyamino acids according to formula VII wherein n=0 according to formula VIIb below: 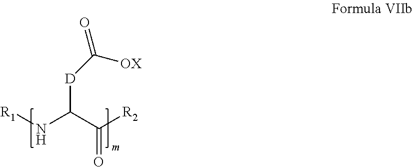
wherein m, X, D, R₁ and R₂ as defined in claim 4 and at least R₁ or R₂ is a hydrophobic radical according to formula I, V or VI.

7. The composition according to claim 1, wherein the molar ratio of co-polyamino acid/amylin, amylin receptor agonist or amylin analogue is greater than or equal to 1.

8. The composition according to claim 1, wherein the amylin, amylin receptor agonist or amylin analogue is amylin.

9. The composition according to claim 1, wherein the amylin, amylin receptor agonist or amylin analogue is pramlintide.

10. The composition according to claim 1, wherein it further comprises a prandial insulin.

11. The composition according to claim 1, wherein the co-polyaminoamide/insulin molar ratio is greater than or equal to 1.

12. The composition according to claim 1, wherein the said composition has a stability measured by ThT greater than that of a reference composition comprising amylin, an amylin receptor agonist or amylin analogue but not comprising a co-polyamino acid bearing carboxylate charges and hydrophobic radicals Hy.
